(19) United States
(12) Patent Application Publication
(10) Pub. No.: US 2009/0017052 A1
(43) Pub. Date: Jan. 15, 2009

(54) Methods of determining lethality of pathogens and malignancies involving replikin peak genes

(76) Inventors: Samuel Bogoch, New York, NY (US); Elenore S. Bogoch, New York, NY (US); Samuel Winston Bogoch, Oakland, CA (US); Anne Elenore Borsanyi, Brookline, MA (US)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(21) Appl. No.: 12/010,027

(22) Filed: Jan. 18, 2008

Related U.S. Application Data
(60) Provisional application No. 60/991,676, filed on Nov. 30, 2007.
(60) Provisional application No. 60/982,336, filed on Oct. 24, 2007.
(60) Provisional application No. 60/982,333, filed on Oct. 24, 2007.
(60) Provisional application No. 60/982,338, filed on Oct. 24, 2007.
(60) Provisional application No. 60/935,816, filed on Aug. 31, 2007.
(60) Provisional application No. 60/935,499, filed on Aug. 16, 2007.
(60) Provisional application No. 60/954,743, filed on Aug. 8, 2007.
(60) Provisional application No. 60/898,097, filed on Jan. 30, 2007.
(60) Provisional application No. 60/880,966, filed on Jan. 18, 2007.

(51) Int. Cl. A61K 39/00; C12Q 1/70; C12Q 1/68; C07H 21/04; G06G 7/50; A61P 31/00; C07K 7/00; C12Q 1/02; C07H 1/00; A61K 31/7088

(52) U.S. Cl. 424/185.1; 435/5; 435/6; 536/23.5; 536/23.7; 536/23.72; 703/11; 435/29; 536/25.4; 514/44; 424/204.1; 424/206.1; 424/234.1; 424/277.1; 424/221.1; 424/248.1; 424/243.1; 424/272.1; 530/327

(57) ABSTRACT

The present invention provides methods of identifying lethal, virulent and rapidly replicating viruses, organisms, and malignancies comprising comparing Replikin concentrations among different viruses, organisms, or malignancies. The present invention further provides isolated Replikin Peak Genes associated with increased lethality, virulence and rapid replication, for diagnostic, therapeutic and predictive purposes.

The instant application contains a “lengthy” Sequence Listing which has been submitted via CD-R in lieu of a printed paper copy, and is hereby incorporated by reference in its entirety. Said CD-R, recorded on Jan. 17, 2008, are labeled CRF, “Copy 1” and “Copy 2”, respectively, and each contains only one identical 1.36 Mb file (27129301.txt).

This application claims priority to U.S. Provisional Appln. Ser. No. 60/991,676, filed Nov. 30, 2007, U.S. application Ser. No. 11/923,559, filed Oct. 24, 2007, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, and U.S. Provisional Appln. Ser. No. 60/880,966, filed Jan. 18, 2007, each of which is incorporated herein by reference in its entirety. This application additionally incorporates herein by reference: U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, U.S. application Ser. No. 10/189,437, filed Jul. 8, 2002, U.S. application Ser. No. 10/105,232, filed Mar. 26, 2002, now U.S. Pat. No. 7,189,800, U.S. application Ser. No. 09/984,057, filed Oct. 26, 2001, and U.S. application Ser. No. 09/984,056, filed Oct. 26, 2001, now U.S. Pat. No. 7,176,275, each in its entirety.

### TECHNICAL FIELD OF THE INVENTION

This invention relates generally to identifying virulent and lethal strains of pathogenic viruses, pathogenic organisms and malignancies through identifying concentrations of the class of small peptides known as Replikins, and to diagnosis, prevention and treatment of disease from such virulent and lethal pathogens and malignancies.

### BACKGROUND OF THE INVENTION

Rapid replication is characteristic of virulence in, among other things, certain bacteria, viruses and malignancies. The inventors have described a quantitative chemistry common to rapid replication in different organisms, viruses and malignancies. The chemistry of rapid replication described by the inventors is present in a family of conserved small protein sequences related to rapid replication called Replikins. A correlation between increased concentrations of Replikin sequences and increased replication and virulence has been observed in a range of viruses and organisms. Replikin sequences, therefore, offer new targets for developing effective methods of predicting and treating viral outbreaks.

### Replikin Sequences in Malignancies and Viral and Bacterial Pathogens

A Replikin sequence is an amino acid sequence of 7 to about 50 amino acids comprising a Replikin motif. A Replikin motif comprises (1) at least one lysine residue located at a first terminus of the motif and at least one lysine residue or at least one histidine residue located at a second terminus of the motif; (2) a first lysine residue located six to ten residues from a second lysine residue; (3) at least one histidine residue; and (4) at least 6% lysine residues. A Replikin sequence may comprise a terminal lysine and may further comprise a terminal lysine or a terminal histidine. A Replikin peptide or Replikin protein is a peptide or protein consisting of a Replikin sequence.

The inventors have identified Replikin sequences in oncogenic cells and in viral and organismal proteins associated with rapid replication and virulence. Additionally, higher concentrations of Replikin sequences in the genomic code have now been associated with a variety of infectious and pathogenic agents including human cancer, HIV, plant viruses, and a range of pathogenic animal and human viruses. Further, the correlation between the concentration of Replikin sequences in viral or organismal proteins and major outbreaks of disease and the correlation between the concentration of Replikin sequences in malignancies and poor prognoses are both significant.

Replikin sequences have been observed to be conserved in human cancers generally and in many pathogenic organisms and viruses, including conservation in both intrastrain and interstrain influenza viruses, for as long as 90 years based on data going back to the 1917-18 flu pandemic. Concentration of Replikin sequences in viral genomes has been shown to increase prior to strain-specific outbreaks and increased mortality in SARS, in influenza, in H5N1 bird flu and now in many other viral and non-viral pathogens. An increase in concentration of production of proteins containing Replikin sequences also has been shown in cancer as replication increases.

Within the last century there have been three influenza pandemics, each strain specific: H1N1 in 1918; H2N2 in 1957; and H3N2 in 1968. The inventors have established that prior to each pandemic there was a strain-specific increase in the concentration of Replikin sequences within the strain. The strain-specific increase in Replikin concentration was followed by a decrease in Replikin concentration and several years later a rebound increase in Replikin concentration associated with a strain-specific rebound epidemic. The Replikin algorithm provided the first chemistry that correlated with influenza epidemics and pandemics.

A similar correlation between the outbreaks of H5NI (Bird Flu) between 1997 and 2007 and the concentration of Replikin sequences in the viral proteins has been demonstrated. Likewise, a correlation has been established between the global outbreak of SARS coronavirus in 2003 and an increase in the concentration of Replikin sequences in the proteins of coronavirus. In another study, Replikins in two strains of human HIV-1 virus demonstrated that the Replikin concentration in the rapidly replicating strain was six fold greater than that of the slowly replicating strain. No instances of rapid replication have been observed in all the viruses and organisms examined wherein the Replikin concentration did not significantly increase as compared to the Replikin concentration in the dormant state.

The Replikin algorithm was initially discovered in Glycoprotein 10B, a membrane glycoprotein isolated from brain glioblastoma multiforme, lymphoma and breast cancer cells (U.S. Pat. No. 6,242,578 B1). A constituent peptide of Aglyco 10B, malignin, was observed to be enriched in cell membranes tenfold during anaerobic replication while cell number was observed to increase only five-fold. This increase in membrane concentration of the malignin protein in rapid replication of glioma cells suggested an integral relationship of the Replikins in malignin to replication of the glioblastoma multiforme.

Hydrolysis and mass spectrometry of malignin yielded a 16-mer peptide that included the Replikin sequence kagvaflhkk (SEQ ID NO: 3658). This peptide, which is absent from the normal human genome, was assumed to be acquired. Homologues of the Replikin sequence were found in all tumor viruses (that is viruses that cause cancer), and in replicating proteins of algae, plants, fungi, viruses and bacteria.

When the glioma Replikin was synthesized in vitro and administered as a synthetic vaccine to rabbits, abundant antimalignin antibody was produced. This production of abundant antimalignin antibody established that the peptide alone is an epitope, that is, it is a sufficient basis for an immune response observed in cancer patients wherein antimalignin antibodies are naturally produced. A 16-mer peptide containing the glioma Replikin produced both IgM and IgG forms of the antibody.

A study of 8,090 serum specimens from cancer patients and controls demonstrated that the concentration of antimalignin antibody increases with age in healthy individuals, as the incidence of cancer in the population increases, and increases further two to three-fold in early malignancy, regardless of cell type. In vitro this antibody was observed to be cytotoxic to cancer cells at picograms (femtomoles) per cancer cell, and in vivo the concentration of antimalignin antibody related quantitatively to the survival of cancer patients. As shown in glioma cells, the stage in cancer at which cells have only been transformed to the immortal malignant state but remain quiescent or dormant, now can be distinguished from the more active life-threatening replicating state which is characterized by the increased concentration of Replikins.

Using the sequence of the glioma Replikin peptide (kagvaflhkk) (SEQ ID NO: 3658) as a template, and constructing a “3-point-recognition” method to visually scan protein sequences of several different organisms, a new class of peptides, the Replikins, was revealed in organisms as diverse as algae, yeast and viruses. Surprisingly, these peptides were found to be concentrated in larger “replicating” and “transforming” proteins.

An infrequent occurrence of homologues was observed in “virus peptides” as a whole (1.5%), and in other peptides not designated as associated with malignant transformation or replication such as “brain peptides” and “neuropeptides” (together 8.5%). A surprisingly high frequency of occurrence of homologues was identified in tumor viruses, transforming proteins and cancer cell proteins. For example, 100% of identified tumor viruses contain Replikin sequences. 85% of transforming proteins contained Replikin sequences and 97% of cancer proteins contained Replikin sequences.

Further, Replikins were identified in such proteins as Saccharomyces cerevisiae replication binding protein; the replication associated protein A of maize streak virus; the replication-associated protein of Staphylococcus aureus; the DNA replication protein of bovine herpes virus 4; and the mealigrid herpes virus 1 replication binding protein. Replikin-containing proteins also are associated frequently with redox functions, and protein synthesis or elongation, as well as with cell replication.

The highest concentration of Replikin sequences in an organism or virus that had been analyzed and reported was 111 Replikin sequences per 100 amino acids in the extraordinarily-rapidly-replicating parasitic protozoa Plasmodium falciparum (reportedly responsible for 90% of malarial deaths in humans) (herein sometimes referred to as malaria). P. falciparum has been observed to replicate 11,000 times in 48 hours during passage of the parasite from liver to blood in the host.

A significant feature of Replikin sequences observed in P. falciparum was a marked overlapping of Replikin structures throughout malarial proteins. For example, there are nine overlapping Replikins in the 39 amino acid sequence of SEQ ID NO. 3667 (Replikin concentration=23.1/100 amino acids); and 15 overlapping Replikins in the 41 amino acids of SEQ ID NO. 3668 (Replikin concentration=36.6/100 amino acids). Both of these overlapping Replikin structures occur in blood stage trophozoites and schizonts. This mechanism of lysine multiples was also seen in the Replikins of cancer proteins such as in gastric cancer transforming protein, ktkkgnrvsptmkvth (SEQ ID NO: 3669), and in transforming protein P21B (K-RAS 2B) of lung, khkekmskdgkkkkkks (SEQ ID NO: 3670).

### Replikin Scaffolds

In monitoring Replikin sequences in influenza virus, the inventors have additionally identified a sub-family of conserved Replikin sequences known as Replikin Scaffolds or Replikin Scaffold sequences. Replikin Scaffolds were initially identified in conserved structures in particularly virulent influenza viruses. Included among these strains were the viruses causing the pandemics of 1918, 1957, 1968 and virulent strains of the H5N1 “bird flu” strain of influenza virus. Analogues of Replikin Scaffold sequences have since been identified in the virulent and rapidly replicating SARS coronavirus. See U.S. Published Application No. 2007/0026009.

Scaffolding of Replikin sequences homologous but not identical to the algorithm of the identified Replikin Scaffold has also been identified in P. falciparum. Replikin scaffolding in general has been related to an increase in Replikin concentrations in pathogenic genomes where it has been identified. In P. falciparum, scaffolding contributes significantly to the very high Replikin concentration noted in the proteins of the protozoa.

### Influenza

Virulent and lethal outbreaks of influenza are a continuing challenge to world health and the medical practitioner is increasingly aware of the continued threat of virulent and lethal influenza pandemics that require new methods of predicting virulence and lethality and will require new methods and compounds for treatment. Influenza is an acute respiratory illness of global importance. Despite international attempts to control influenza virus outbreaks through vaccination, influenza infections remain an important cause of morbidity and mortality. Worldwide influenza pandemics have occurred at irregular and previously unpredictable intervals throughout history and it is expected that influenza pandemics will continue to occur in the future. The impact of pandemic influenza is substantial in terms of morbidity, mortality and economic cost.

Influenza vaccines remain the most effective defense against influenza virus, but because of the ability of the virus to mutate, and the availability of non-human host reservoirs, it is expected that influenza will remain an emergent or re-emergent infection. Global influenza surveillance indicates that influenza viruses may vary within a country and between countries and continents during an influenza season. Virologic surveillance is of importance in monitoring antigenic shift and drift. Disease surveillance is also important in assessing the impact of epidemics. Both types of information have provided the basis of vaccine composition and use of antivirals. However, traditionally there has been only annual post hoc hematological classification of the increasing number of emerging influenza virus strains, and no specific chemical structure of the viruses was identified as an indicator of approaching influenza epidemic or pandemic. Until recently, the only basis for annual classification of influenza virus as active, inactive or prevalent in a given year was the activities of the virus hemagglutinin and neuraminidase proteins.

There is a need in the art for methods of predicting increases in virulence and lethality of influenza prior to outbreaks. There is likewise a need in the art for methods of preventing and treating outbreaks caused by virulent strains of influenza. Because of the annual administration of influenza vaccines and the short period of time when a vaccine can be administered, strategies directed at improving vaccine coverage are of critical importance.

### Equine Influenza Virus

Equine influenza is a common upper respiratory disease of the horse currently caused by the H3N8 strain of equine influenza virus (EIV). Typical symptoms of equine influenza include a dry hacking cough, nasal discharge, and fever. The viral disease is considered enzootic in Europe, the United States and parts of Asia. Significant outbreaks have also been observed in South America, China, and India.

The first outbreak of equine influenza in Japan since 1972 was recently reported and 2007 saw the first ever report of equine influenza in Australia. So far, no fatalities have been reported. Equine influenza is, however, sometimes fatal in young foals.

Quarantine has been thought to be the best prevention against the spread of equine influenza. South Africa, Australia and Japan have used quarantine of imported horses to stop the spread of, among other diseases, equine influenza. The quarantine practice has apparently not been fully successful suggesting possible incidental transfer of the disease through human handlers of the horses.

The influenza virus is highly mutable and, as a result, development of long-term therapies has been difficult. Vaccines generally have needed to be updated as virulent mutants of the virus have arisen. Annual review of worldwide outbreaks of the virus provides data for recommended production of vaccines against the most relevant strains of virus. Significant time elapses between identification of the most relevant strains and commercialization of vaccines.

There is a need in the art for methods of identifying emerging equine influenza viruses prior to outbreaks so that preventive measures may be taken against such emerging viruses. There is likewise a need in the art for methods of preventing and treating outbreaks caused by virulent strains of EIV including vaccines.

### Foot and Mouth Disease

Foot and Mouth Disease is a highly contagious and sometimes fatal viral disease of cattle, pigs and other animals including bovids with cloven hooves cause by foot and mouth disease virus (FMDV). FMDV is a single-stranded RNA aphthovirus of the Picornaviridae virus family. There are said to be seven different FMDV serotypes: 0, A, C, SAT-1, SAT-2, SAT-3, and Asia-1.

There is a need in the art for methods of predicting increases in virulence of FMDV prior to outbreaks of Foot and Mouth Disease. There is likewise a need in the art for methods of preventing and treating outbreaks of Food and Mouth Disease caused by virulent strains of FMDV.

### West Nile Virus

West nile virus (WNV), in a small percentage of infected humans, causes encephalitis and other serious neuroinvasive diseases. In about four percent of reported cases of WNV infection, the resulting neuroinvasive disease results in death. WNV is flaviviridae virus that was first observed in North America in 1999 and is now considered endemic in the United States. The virus is spread to humans through mosquito (and related insect) bites. Infection with WNV causes diseases such as encephalitis, meningitis and meningoencephalitis in less than about one percent of infected humans. In about 20 percent of infected humans, less severe illness, characterized by fever, headache, tiredness, aches and sometimes rashes, may occur. Of the total number of U.S. cases of WNV infection reported, about four percent have resulted in death.

WNV is a single-stranded sense RNA virus and is a member of the Japanese encephalitis virus antigenic complex, which includes several medically important viruses associated with human encephalitis: Japanese encephalitis, St. Louis encephalitis, Murray Valley encephalitis, and Kunjin encephalitis, an Australian subtype of WNV.

Since introduction of the disease to the United States in 1999, there have been more than 16,000 reported cases of WNV in humans and more than 650 reported deaths. In addition, more than 21,000 cases have been reported in horses. Currently, the only available approved strategies to combat WNV in humans are nationwide active surveillance in conjunction with mosquito control efforts and individual protection with insect repellents. There is a need in the art, therefore, for methods of predicting increases in virulence of WNV prior to epidemics. There is likewise a need in the art for methods of preventing and treating outbreaks caused by virulent strains of WNV.

### Viral Diseases in Pigs

Two severe viral diseases now endemic in swine in many countries and presently causing great economic losses worldwide are Porcine Reproductive and Respiratory Syndrome (PRRS) and Porcine Circovirus Associated Diseases (PCVAD), caused by porcine reproductive and respiratory syndrome virus (PRRSV) and porcine circovirus (PCV), respectively. Each disease has a significant impact on the hog industry and, in both diseases, current control measures are proving inadequate.

PRRS is a relatively recently recognized disease in pigs. The infectious virus is classified in the family Arteriviridae and order Nidovirales and did not have a standardized name in the past but is now known as porcine reproductive and respiratory syndrome virus (PRRSV). The disease is characterized by reproductive failure, death in young pigs and mild respiratory disease.

The pig is the only known host for PRRSV but evidence suggests that another host or hosts may have existed prior to identification of PRRS in the United States in 1987 and Europe in 1990. PRRS is now endemic in the United States and many European countries. Evidence of infection (whether serological or virological or both) has been found in Japan, Korea, the Philippines, Vietnam, South America and the Caribbean.

The disease has been associated with reproductive failure in sows and respiratory disease in all stages of pig development. Clinical signs of the disease include: fever, anorexia, depression, reduced conception rates, abortion, week piglets, respiratory distress and increased rates of other endemic diseases.

PRRSV is a positive-sense single-stranded small envelope RNA virus with at least nine open reading frames (ORFs) in its genome encoding about 20 putative proteins: ORF 1a and 1b encode replication proteins; ORF 2a and 2b encode unknown structure proteins; ORF 3, 4 and 5 encode envelope proteins; ORF 6 encodes membrane proteins and ORF 7 encodes nucleocapsid proteins.

Two types of PRRSV have been identified: European (Type I) and North American (Type II). The two types share about 60% sequence identity. PRRSV strains are known to differ markedly in pathogenicity. In 2006, highly pathogenic outbreaks of PRRSV occurred in China and Mexico. The cost of PRRSV infection to the U.S. pork industry has been estimated at between $560 million and $761 million annually. PRRSV infection has been associated with a reduction in the number of pigs weaned per litter, a reduction in birthing rate, increased mortality, reduced feed conversion and reduced average daily weight gain.

Porcine Circovirus Associated Diseases (PCVAD) have also only recently been recognized in pigs (1996). PCVAD is a term used to define the entire range of disease associated with porcine circovirus (PCV) infection. The range of disease in pigs includes: Postweaning Multisystemic Wasting Syndrome (PMWS); respiratory illness; pneumonia; diarrhea; reproductive disorders and high mortality. PCVAD symptoms may include detection of PCV within lesions that form on growing pigs, inflammation in, for example, the spleen, thymus, intestines, lymph nodes, lung, kidney, liver, and tonsils, and depletion of lymphoid cells. PCV infection is thought to pose no apparent risk to human health. PCVAD is presently severely affecting the Canadian swine industry.

Two antigenically distinct types of PCV have been identified. Porcine Circovirus 1 (PCV1), which may be non-pathogenic, and Porcine Circovirus 2 (PCV2), which appears to be the strain that causes PCVAD. PCV1 and PCV2 share about 65% amino acid identity in open reading frame 2 of the virus genome.

The incidence of PCV infection associated disease has increased by 4% between 2000 and 2006 in Canada and new outbreaks have been observed in Western Canada. In some studies, more than 80% of Canadian pigs have been found to be infected with PCV2 at slaughter. In infected herds, an increase in mortality rates has also been observed. As incidence of PCV infection has increased, pork production has decreased due to pig death and decreased productivity. Production in Canada in 2006 is expected to decrease 1.5 percent below 2005 production due to PCV-influenced disease.

There is a need in the art for methods of predicting increases in virulence of PRRSV and PCV prior to outbreaks. There is likewise a need in the art for methods of preventing and treating outbreaks caused by virulent strains of PRRSV and PCV.

### WSSV and TSV Shrimp Pathogens

White spot syndrome virus (WSSV) (also known as white spot baculoform virus) and taura syndrome virus (TSV) are global lethal pathogens in shrimp.

Taura syndrome is a viral disease in shrimp that significantly impacts the shrimp farming industry worldwide. Taura Syndrome is caused by the taura syndrome virus (TSV), which is a member of the Discistroviridae family in the genus Cripavirus that has a single positive stranded genome of about 10,000 nucleotides. The genome contains two open reading frames (ORF). ORF 1 reportedly contains coding for a helicase, a protease and an RNA-dependent RNA polymerase. ORF2 reportedly contains coding for three capsid proteins.

Taura syndrome is now considered endemic in the Americas and outbreaks have been observed in Asia. Infected shrimp generally have a red tail, are anorexic and erratic in their behavior, tail muscles may become opaque and the cuticle may become soft. Mortality rates between 5% and 95% have been observed during the acute phase of the disease. Shrimp that survive outbreaks of TSV seem to be refractory to reinfection while remaining infectious.

White spot syndrome (WSS) is a highly contagious and lethal viral infection of shrimp often destroying entire farm populations within several days of observation of the first symptoms. The first reported epidemic of the disease was in Taiwan in 1992 and the disease is now known to be present in all shrimp growing regions globally except Australia. The virus has a wide host range including most cultured penaeid shrimp including Fenneropenaeus indicus, Penaeus monodon, Litopenaeus vannamei, and Marsupenaeus japonicas, other non-penaeid shrimp, crabs, spiny lobsters and others.

WSSV is a rod-shaped double-stranded DNA virus. The complete DNA sequence of WSSV genome has reportedly been assembled into a circular sequence of 292,967 base pairs. Clinical signs of WSSV infection include white spots on the carapace, often reddish discoloration, and reduction in food consumption and loss of energy. There is a need in the art for methods of preventing and treating viral infections of shrimp such as TSV and WSSV by manipulating the replicating function of Replikin sequences and for identifying molecular targets related to the replicating function of Replikin sequences for treatment of virulent viral.

### SUMMARY OF THE INVENTION

The present invention provides a method of identifying a first virus, first organism or first malignancy with a higher lethality than at least one second virus of the same species as the first virus, second organism of the same species as the first organism or second malignancy of the same species as the first malignancy which comprises comparing the Replikin Count of the Replikin Peak Gene of the first virus, first organism or first malignancy to the Replikin Count of the Replikin Peak Gene of at least one second virus, second organism, or second malignancy to determine that the virus, organism or malignancy with the higher Replikin Count is the more lethal.

In one embodiment, the first malignancy is a lung malignancy, a brain malignancy, a breast malignancy, an ovarian malignancy or a lymph malignancy. In a specific embodiment, the first malignancy is a non-small cell lung carcinoma.

In another embodiment, the first organism is a Mycobacterium tuberculosis, Mycobaterium mucogenicum, Staphylococcus aureus, or Plasmodium falciparum.

In a further embodiment, the virus is influenza virus, foot and mouth disease virus, west nile virus, porcine respiratory and reproductive syndrome virus, porcine circovirus, white spot syndrome virus, taura syndrome virus, coronavirus, ebola virus, gemini leaf curl virus, hemorrhagic septicemia virus, or tobacco mosaic virus.

In a specific embodiment, the first virus is a strain of Influenza A virus of H1N1, H2N2, H3N2, H5N1, or H3N8.

In a further embodiment, said at least one Replikin sequence within the protein or protein fragment of the identified Replikin Peak Gene is isolated from influenza A strain H5N1 and is selected from the group consisting of SEQ ID NOS: 1685-1691, SEQ ID NOS: 1702-1717. In a further embodiment, said at least one Replikin sequence within the protein or protein fragment of the identified Replikin Peak Gene is isolated from equine influenza virus (H3N8) and is selected from the group consisting of SEQ ID NOS: 547-562.

The present invention further provides a isolated or synthesized Replikin Peak Gene of a virus, organism or malignancy wherein said Replikin Peak Gene is identified as the portion of the genome, protein or protein fragment of a virion of the virus, a cell of the organism or a malignant cell of the malignancy consisting of the highest number of continuous Replikin sequences per 100 amino acids as compared to other portions of the genome, protein or protein fragment of the virion of the virus, the cell of the organism or the malignant cell of the malignancy.

In one embodiment, the isolated or synthesized Replikin Peak Gene is the portion of a protein or protein fragment consisting of the highest number of continuous Replikin sequences per 100 amino acids as compared to all other proteins or protein fragments in the virion of the virus, in the cell of the organism or in the malignant cell of the malignancy.

In a specific embodiment, the isolated or synthesized Replikin Peak Gene is isolated from a lung malignancy, a brain malignancy, a breast malignancy, an ovarian malignancy, or a lymph malignancy. In another specific embodiment, the isolated or synthesized Replikin Peak Gene is isolated from a non-small cell lung carcinoma or glioblastoma multiforme.

In yet another embodiment, the isolated or synthesized Replikin Peak Gene of is isolated from Mycobacterium tuberculosis, Mycobacterium mucogenicum, Staphylococcus aureus, or Plasmodium falciparum.

According to a further embodiment, the isolated or synthesized Replikin Peak Gene is isolated from influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

In one embodiment, the isolated or synthesized Replikin Peak Gene is from influenza virus, particularly an Influenza A virus. In a specific embodiment, the Influenza A virus is a strain H1N1, H2N2, H3N2, H5N1 or H3N8. In another specific embodiment, the Replikin Peak Gene is isolated from the pB1 gene area of an influenza virus.

According to another embodiment, the isolated or synthesized Replikin Peak Gene is from foot and mouth disease virus. In a specific embodiment, the isolated or synthesized Replikin Peak Gene is identified within the VP1 gene of a foot and mouth disease virus.

In yet another embodiment, the isolated or synthesized Replikin Peak Gene is from a west nile virus. In a specific embodiment, the isolated or synthesized Replikin Peak Gene is isolated from the envelope protein of west nile virus.

In a further embodiment, the isolated or synthesized Replikin Peak Gene is from a porcine respiratory and reproductive syndrome virus. In a specific embodiment, the isolated or synthesized Replikin Peak Gene is isolated from a nucleocapsid protein of a porcine respiratory and reproductive syndrome virus.

In yet another embodiment, the isolated or synthesized Replikin Peak Gene is from a porcine circovirus. In a specific embodiment, the isolated or synthesized Replikin Peak Gene is isolated from a replicase protein of a porcine circovirus.

In still a further embodiment, the isolated or synthesized Replikin Peak Gene is from a white spot syndrome virus. In a specific embodiment, the isolated or synthesized Replikin Peak Gene is isolated from a ribonucleotide reductase protein of a white spot syndrome virus.

In yet another embodiment, the isolated or synthesized Replikin Peak Gene is from a tobacco mosaic virus.

In a further embodiment, the isolated or synthesized Replikin Peak Gene is from a hemorrhagic septicemia virus in fish. In a specific embodiment, the isolated or synthesized Replikin Peak Gene is isolated from a glycoprotein in a hemorrhagic septicemia virus.

In another specific embodiment, the isolated or synthesized Replikin Peak Gene comprises a sequence of SEQ ID NO: 1741, SEQ ID NO: 3664, SEQ ID NO: 3660, SEQ ID NO: 3665, SEQ ID NO: 1996, SEQ ID NO: 1665, SEQ ID NO: 1684, SEQ ID NO: 1701, SEQ ID NO: 546, SEQ ID NO: 124, SEQ ID NO: 130, SEQ ID NO: 311, SEQ ID NOS: 341-344, SEQ ID NO: 286, SEQ ID NO: 287, SEQ ID NO: 288, SEQ ID NO: 289, SEQ ID NO: 290, SEQ ID NOS: 233-238, SEQ ID NO: 415, SEQ ID NO: 421, SEQ ID NO: 438, SEQ ID NO: 451, SEQ ID NO: 462, SEQ ID NO: 498, SEQ ID NO: 669, SEQ ID NO: 1168, SEQ ID NO: 1531, SEQ ID NO: 1548, or SEQ ID NO: 1939.

The present invention further provides an immunogenic composition comprising the isolated or synthesized Replikin Peak Gene. In a specific embodiment, the immunogenic composition comprises a Replikin sequence of SEQ ID NOS: 2902-2925, SEQ ID NOS: 2312-2544, SEQ ID NOS: 2701-2711, SEQ ID NOS: 2713-2718, SEQ ID NOS: 3282-3285, 3287-3291, 3293, 3295, 3297, 3299, 3300, 3302, 3304, 3306, and 3308, SEQ ID NOS: 1685-1691, SEQ ID NOS: 1702-1717, SEQ ID NO: 106, SEQ ID NO: 112, SEQ ID NO: 113, SEQ ID NOS: 125-129, SEQ ID NOS: 131-156, SEQ ID NOS: 233-244, SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-414, SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484 and SEQ ID NOS: 487-492, SEQ ID NOS: 547-562. SEQ ID NOS: 663-667, SEQ ID NOS: 670-1166, SEQ ID NOS: 1169-1529, SEQ ID NOS: 1532-1542, SEQ ID NO: 1548, SEQ ID NOS: 3788-3823), or SEQ ID NOS 1637-1663.

A non-limiting embodiment of the present invention provides computer readable medium having stored thereon instructions which, when executed, cause the processor to perform a method for identifying a Replikin Peak Gene of a virus, organism or malignancy comprising identifying, within amino acid sequences or nucleic acid sequences that encode amino acid sequences of said virus, organism or malignancy, the portion of the genome, or protein or protein fragment of said virus, said organism or said malignancy consisting of the highest number of continuous Replikin sequences per 100 amino acids as compared to other portions of the genome, or protein or protein fragment of the malignancy, organism or virus.

In one embodiment, the computer readable medium comprises instructions which, when executed, cause the processor to perform a method for predicting an increase in lethality or virulence of said virus, organism or malignancy that comprises said identified Replikin Peak Gene or an outbreak of said virus or organism that comprises said identified Replikin Peak Gene by: (1) determining that the Replikin Count of said Replikin Peak Gene or that the Replikin Count of a protein or gene area comprising said Replikin Peak Gene is higher than another Replikin Peak Gene or a protein or gene area comprising said other Replikin Peak Gene identified within the genome or within a protein or protein fragment of at least one other virus of the same species as said virus, at least one other organism of the same species as said organism or at least one other malignancy of the same type as said malignancy wherein said other virus, said other organism or said other malignancy is isolated at an earlier time point than said virus, said organism or said malignancy, and (2) predicting an increase in lethality or virulence of said virus, organism or malignancy or predicting an outbreak of said virus or organsism.

The invention also provides a method of predicting the strain, the host or the geographic region of an outbreak or increase in lethality or virulence of a virus or organism by (1) identifying a Replikin Peak Gene or a protein or gene area comprising a Replikin Peak Gene within the genome of a first virus or organism of a first strain, from a first host, or isolated from a first geographic region or within a protein or protein fragment of the first virus or organism that has a higher Replikin Count than a Replikin Peak Gene or protein or gene area comprising a Replikin Peak Gene identified within the genome or within a protein or protein fragment of at least one second virus of the same species as the first virus or at least one second organism of the same species as the first organism wherein said first virus or said first organism is isolated at a later time point than said first virus or said first organism and is the same strain, from the same or another host or isolated from the same or another geographic region as the first virus or first organism, and (2) predicting an outbreak or an increase in lethality or virulence of said first strain, in said first host or within said first geographic region of said first virus or organism.

In one embodiment, the protein or gene area comprising said Replikin Peak Gene within the genome of a first virus or organism is identified as having a higher Replikin Count than said protein or gene area comprising a Replikin Peak Gene identified within the genome or within a protein or protein fragment of said at least one second virus or organism.

In another embodiment, the first virus or first organism is isolated at least six months to three years later than the second organism or said second virus. In a specific embodiment, the first organism or first virus is Mycobacterium tuberculosis, Mycobaterium mucogenicum, Staphylococcus aureus, and Plasmodium falciparum, influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus in tomato plants, hemorrhagic septicemia virus, or tobacco mosaic virus. In another embodiment, the Staphylococcus aureus is methicillin-resistant.

In a further embodiment, the influenza virus a strain of Influenza A virus. In a specific embodiment, the first virus is an influenza virus of the strain H1N1, H2N2, H3N2, H5N1 or H3N8.

In a further embodiment of the invention, the protein or gene area comprising the Replikin Peak Gene is the pB1 gene area of the influenza virus.

In yet another embodiment, the protein or gene area is a nucleocapsid protein of porcine respiratory and reproductive syndrome virus.

In a further embodiment, the protein or gene area is an envelope protein of west nile virus.

In a further embodiment, the protein or gene area is a VP1 protein of foot and mouth disease virus.

In still another embodiment, the protein or gene area is an ATP-ase of Plasmodium falciparum.

In yet a further embodiment, the protein or gene area is a replicase protein of porcine circovirus.

In another embodiment, the protein or gene area is a ribonucleotidease of said white spot syndrome virus.

The present invention further provides a method of identifying a first virus, organism or malignancy associated with higher lethality, higher virulence or more rapid replication than a second virus of the same species as the first virus, a second organism of the same species as the first organism or a second malignancy of the same type as the first malignancy comprising identifying a Replikin Peak Gene encoded within the genome of at least one virion of the first virus, or at least one cell of the first organism, or at least one malignant cell of the first malignancy, or within a protein or protein fragment of at least one virion of the first virus, or at least one cell of the first organism, or at least one malignant cell of the first malignancy that has a higher Replikin Count than a Replikin Peak Gene identified encoded within the genome of at least one virion of the second virus, or at least one cell of the second organism, or at least one malignant cell of the second malignancy or within a protein or protein fragment of at least one virion of the second virus, or at least one cell of the second organism, or at least one malignant cell of the second malignancy wherein said first virus, first organism or first malignancy has higher lethality, higher virulence or more rapid replication than said second virus, second organism or second malignancy, and wherein the Replikin Peak Gene is defined as a protein or protein fragment having the highest concentration of continuous Replikin sequences per 100 amino acids as compared to the remaining proteins or protein fragments in the same virion of the virus, the same cell of the organism, or the same malignant cell, or the portion of the genome encoding the protein or protein fragment.

Further provided is a method of identifying a first virus, first organism or first malignancy with a higher lethality than at least one second virus of the same species as the first virus, second organism of the same species as the first organism or second malignancy of the same species as the first malignancy comprising comparing the Replikin Count of the whole genome of a virus, organism or malignancy to the Replikin Count of the whole genome of at least one second virus, second organism, or second malignancy to determine that the virus, organism or malignancy with the higher Replikin Count is the more lethal.

According to a specific embodiment, the first virus is a coronavirus, a foot and mouth disease virus, a white spot syndrome virus, a taura syndrome virus, a porcine circovirus, or an influenza virus.

In one specific embodiment, the first virus is an H5N1 strain of influenza virus.

In another specific embodiment, the influenza virus is an Influenza A virus. In a further specific embodiment, the Influenza A virus is H1N1, H2N2, H3N2, H5N1 or H3N8.

According to another embodiment, the Replikin Peak Gene is isolated from the pB1 gene area of an influenza virus.

The present invention also provides method for obtaining an isolated or synthesized Replikin Peak Gene of a virus, organism or malignancy for diagnosis, prevention or treatment of an infection of said virus or said organism or for diagnosis, prevention or treatment of said malignancy comprising: (1) obtaining a plurality of isolates of virus of the same species, a plurality of organisms of the same species, or a plurality of malignancies of the same type; (2) analyzing the protein sequences or protein sequence fragments of each individual isolate of the plurality of isolates of virus, a cell of each individual organism of the plurality of organisms, or a malignant cell of each individual malignancy of the plurality of malignancies for the presence and concentration of Replikin sequences; (3) identifying the protein sequence or the protein sequence fragment having the highest concentration of continuous Replikin sequences in the malignant cell of each individual malignancy, the cell of each individual organism or each individual virus isolate; (4) selecting the protein sequence or protein sequence fragment having the highest concentration of continuous Replikin sequences among the plurality of isolates of virus, the plurality of organisms, or the plurality of malignancies; (5) identifying the amino acid sequence of the selected protein sequence or protein sequence fragment as the Replikin Peak Gene of the plurality of virus isolates, organisms or malignancies; and (6) isolating or synthesizing the identified Replikin Peak Gene of at least one of the plurality of virus isolates, organisms or malignancies wherein the isolated or synthesized identified Replikin Peak Gene is useful for diagnosis, prevention or treatment of said infection of said virus or said organism or said malignancy.

Further provided is an immunogenic composition comprising at least one isolated or synthesized Replikin Peak Gene isolated according to the above method. In a specific embodiment, the immunogenic composition is isolated from an emerging strain of a virus or organism, and optionally further comprises a pharmaceutically acceptable carrier.

The present invention also provides a vaccine comprising at least one isolated or synthesized Replikin Peak Gene. In a specific embodiment, the vaccine comprises a Replikin Peak Gene isolated from an emerging strain of virus or organism. In another specific embodiment, the vaccine comprises SEQ ID NO: 1741, SEQ ID NO: 3664, SEQ ID NO: 3660, SEQ ID NO: 3665, SEQ ID NO: 1996, SEQ ID NO: 1665, SEQ ID NO: 1684, SEQ ID NO: 1701, SEQ ID NO: 546, SEQ ID NO: 124, SEQ ID NO: 130, SEQ ID NO: 311, SEQ ID NOS: 341-344, SEQ ID NO: 286, SEQ ID NO: 287, SEQ ID NO: 288, SEQ ID NO: 289, SEQ ID NO: 290, SEQ ID NOS: 233-238, SEQ ID NO: 415, SEQ ID NO: 421, SEQ ID NO: 438, SEQ ID NO: 451, SEQ ID NO: 462, SEQ ID NO: 498, SEQ ID NO: 669, SEQ ID NO: 1168, SEQ ID NO: 1531, SEQ ID NO: 1548, positions 81-204 of SEQ ID NO: 3787, or SEQ ID NO: 1939.

In yet a further embodiment, the vaccine comprises a Replikin Peak Gene isolated from a virus.

In a specific embodiment, the virus is influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

In one embodiment, the Replikin Peak Gene in the vaccine is isolated from Influenza A, or specifically strains H1N1, H2N2, H3N2, H5N1 or H3N8.

In another embodiment, the vaccine comprises a Replikin Peak Gene isolated from an organism.

In a further embodiment, Replikin Peak Gene is isolated from Mycobacterium tuberculosis, Mycobaterium mucogenicum, Staphylococcus aureus, or Plasmodium falciparum. In a specific embodiment, the Staphylococcus aureus is methicillin-resistant.

In still another embodiment, the Replikin Peak Gene is isolated from a malignancy.

In a specific embodiment, the Replikin Peak Gene is isolated from a lung malignancy, a brain malignancy, a breast malignancy or a lymph malignancy. In another embodiment, the Replikin Peak Gene is isolated from a non-small cell lung carcinoma. In a further embodiment, the Replikin Peak Gene is isolated from glioblastoma multiforme.

The present invention further provides an immunogenic composition comprising a Replikin Peak Gene, optionally in combination with a pharmaceutically acceptable carrier. In one embodiment, the immunogenic composition comprises SEQ ID NO: 1741, SEQ ID NO: 3664, SEQ ID NO: 3660, SEQ ID NO: 3665, SEQ ID NO: 1996, SEQ ID NO: 1665, SEQ ID NO: 1684, SEQ ID NO: 1701, SEQ ID NO: 546, SEQ ID NO: 124, SEQ ID NO: 130, SEQ ID NO: 311, SEQ ID NOS: 341-344, SEQ ID NO: 286, SEQ ID NO: 287, SEQ ID NO: 288, SEQ ID NO: 289, SEQ ID NO: 290, SEQ ID NOS: 233-238, SEQ ID NO: 415, SEQ ID NO: 421, SEQ ID NO: 438, SEQ ID NO: 451, SEQ ID NO: 462, SEQ ID NO: 498, SEQ ID NO: 669, SEQ ID NO: 1168, SEQ ID NO: 1531, SEQ ID NO: 1548, or SEQ ID NO: 1939.

The present invention further provides an isolated or synthesized Replikin sequence isolated from a protein or protein fragment a Replikin Peak Gene or isolated from a protein comprising a Replikin Peak Gene.

In one embodiment, the Replikin sequence is from a Replikin Peak Gene isolated from Mycobacterium tuberculosis, Mycobacterium mucogenicum, Staphylococcus aureus, or a Plasmodium falciparum. In a specific embodiment, the Replikin sequence is from a Replikin Peak Gene isolated from Mycobacterium mucogenicum. In a further embodiment, the Replikin Peak Gene is SEQ ID NOS: 2902-2925. In another specific embodiment, the Replikin sequence is from a Replikin Peak Gene isolated from Plasmodium falciparum. In a further embodiment, the Replikin Peak Gene is one of SEQ ID NOS: 2312-2544, SEQ ID NOS: 2701-2711, SEQ ID NOS: 2713-2718, SEQ ID NOS: 3282-3285, 3287-3291, 3293, 3295, 3297, 3299, 3300, 3302, 3304, 3306, or SEQ ID NO: 3308.

In another embodiment, the Replikin sequence is from a Replikin Peak Gene isolated from influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

In a specific embodiment, the influenza virus is Influenza A virus. In another specific embodiment, the Influenza A virus is H1N1, H2N2, H3N2, H5N1 or H3N8. In a further specific embodiment, the Influenza A virus is H5N1 and the Replikin sequence is one of SEQ ID NOS: 1685-1691, SEQ ID NOS: 1702-1716 or SEQ ID NO: 1717. In a further specific embodiment, the Influenza A virus is H3N8 and the Replikin sequence is one of SEQ ID NOS: 547-561 or SEQ ID NO: 562.

In another embodiment, the Replikin sequence is from a Replikin Peak Gene isolated from foot and mouth disease virus. In a specific embodiment, the Replikin sequence from the foot and mouth disease virus is one of SEQ ID NO: 106, SEQ ID NO: 112, SEQ ID NO: 113, SEQ ID NOS: 125-129, SEQ ID NOS: 131-155 or SEQ ID NO: 156.

In still another embodiment, the Replikin sequence is from a Replikin Peak Gene isolated from west nile virus. In a specific embodiment, the Replikin sequence from the west nile virus is one of SEQ ID NOS: 233-243 or SEQ ID NO: 244.

In a further embodiment, the Replikin sequence is from a Replikin Peak Gene isolated from porcine reproductive and respiratory virus. In a specific embodiment, the Replikin sequence from porcine reproductive and respiratory virus is one of SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-413 or SEQ ID NO: 414.

In another embodiment, the Replikin sequence is from a Replikin Peak Gene isolated from porcine circovirus. In a specific embodiment, the Replikin sequence from porcine circovirus is one of SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484, SEQ ID NOS: 487-491 or SEQ ID NO: 492.

In still a further embodiment, the Replikin sequence is from a Replikin Peak Gene isolated from white spot syndrome virus. In a specific embodiment, the Replikin sequence from white spot syndrome virus is one of SEQ ID NOS: 663-667, SEQ ID NOS: 670-1166, SEQ ID NOS: 1169-1529, SEQ ID NOS: 1532-1542 and SEQ ID NO: 1548.

According to the present invention provided is a vaccine for prevention and/or treatment of an viral or organismal infection or a malignancy wherein the vaccine comprises at least one isolated or synthesized Replikin sequence within a protein or protein fragment of a Replikin Peak Gene or a protein comprising a Replikin Peak Gene identified in said virus, organism, or malignancy.

In a further embodiment, the at least one isolate or synthesized Replikin sequence in the vaccine is one of SEQ ID NOS: 2902-2925, SEQ ID NOS: 2312-2544, SEQ ID NOS: 2701-2711, 2713-2718, SEQ ID NOS: 3282-3285, 3287-3291, 3293, 3295, 3297, 3299, 3300, 3302, 3304, 3306, 3308, SEQ ID NOS: 1685-1691, SEQ ID NOS: 1702-1717, SEQ ID NOS: 547-562, SEQ ID NO: 106, SEQ ID NO: 112, SEQ ID NO: 113, SEQ ID NOS: 125-129, and SEQ ID NOS: 131-156, SEQ ID NOS: 233-244, SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-414, SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484 SEQ ID NOS: 487-492, SEQ ID NOS: 663-667, SEQ ID NOS: 670-1166, SEQ ID NOS: 1169-1529, SEQ ID NOS: 1532-1542, SEQ ID NO: 1548, SEQ ID NOS: 1637-1662, or SEQ ID NO: 1663.

In one embodiment, the vaccine is for prevention and/or treatment of a viral infection. In a specific embodiment, the vaccine is for a viral infection is caused by influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

In another specific embodiment, the influenza virus is Influenza A virus. In a further specific embodiment, the Influenza A virus is H1N1, H2N2, H3N2, H5N1 or H3N8 Influenza A virus.

In another specific embodiment, the virus is hemorrhagic septicemia virus.

In another embodiment, the vaccine is for prevention and/or treatment of an organismal infection.

In one specific embodiment, the organismal infection is caused by Mycobaterium mucogenicum, Mycobacterium tuberculosis, Staphylococcus aureus, or Plasmodium falciparum. In a further specific embodiment, the Staphylococcus aureus is methicillin-resistant.

In another embodiment, the vaccine is for prevention of a malignancy.

In one specific embodiment, the malignancy is a lung malignancy, a brain malignancy, a breast malignancy, an ovarian malignancy, or a lymph malignancy. In a further specific embodiment, the malignancy is non-small cell lung carcinoma or glioblastoma multiforme.

The invention also provides an immunogenic compound comprising at least one isolated or synthesized Replikin sequence within the protein or protein fragment of a Replikin Peak Gene or within a protein comprising a Replikin Peak Gene wherein said Replikin Peak Gene is identified in a virus, an organism or a malignancy, optionally further comprising a pharmaceutically acceptable carrier.

In another aspect, the present invention provides a method of stimulating the immune system, comprising administering in an animal at least one isolated or synthesized Replikin sequence identified within a protein or protein fragment of a Replikin Peak Gene or within a protein or gene area comprising a Replikin Peak Gene identified in a virus, organism, or malignancy. In a specific embodiment, the animal is a human.

The invention further provides an antibody to at least one isolated or synthesized Replikin sequence within a protein or protein fragment of Replikin Peak Gene or within protein or gene area comprising a Replikin Peak Gene.

Also provided by the present invention is a method of identifying a lethal strain of malignancy, organism or virus comprising: (1) obtaining a plurality of isolates of said malignancy, organism or virus; (2) identifying the Replikin Peak Gene in each isolate of the plurality of isolates of said malignancy, organism or virus; (3) analyzing the amino acid sequence of a protein or protein fragment of the Replikin Peak Gene of each isolate of the plurality of isolates for the presence and concentration of Replikin sequences; (4) comparing the concentrations of Replikin sequences in each of the proteins or protein fragments of the Replikin Peak Gene of each isolate of the plurality of isolates to the concentration of Replikin sequences in each of the proteins or protein fragments of the Replikin Peak Gene of each of the other isolates of the plurality of isolates; and (5) identifying the isolate having the highest concentration of continuous Replikin sequences in the protein or protein fragment of the Replikin Peak Gene as a virulent or lethal strain of said malignancy, organism or virus.

Further provided is a method of selecting a peptide from a malignancy, organism or virus for inclusion in a preventive or therapeutic vaccine or immunogenic compound for a malignancy, organism or virus comprising identifying at least one difference in the amino acid sequence of an otherwise conserved Replikin sequence or Replikin Peak Gene between at least two isolates of said malignancy, organism or virus and correlating the identified at least one difference in the amino acid sequence with the highest virulence, morbidity or host mortality among the at least two isolates and selecting an otherwise conserved Replikin sequence, Replikin Peak Gene or Replikin sequence within a Replikin Peak Gene having the identified at least one amino acid sequence difference as the peptide for inclusion in a preventive or therapeutic vaccine or immunogenic compound.

In one embodiment, the method further comprises predicting the isolate comprising the selected conserved Replikin sequence or Replikin Peak Gene having the at least one difference in the amino acid sequence to be lethal isolate of said malignancy, organism or virus.

In a specific embodiment, the malignancy, organism or virus is a malignancy.

In another specific embodiment, the malignancy is a lung malignancy, a brain malignancy, a breast malignancy or a lymph malignancy. In a further specific embodiment, the malignancy is a non-small cell lung carcinoma or a glioblastoma multiforme.

In another aspect, the malignancy, organism or virus is an organism.

In a first specific embodiment, the organism is Mycobacterium tuberculosis, Mycobaterium mucogenicum, Staphylococcus aureus, or Plasmodium falciparum. In another specific embodiment, the Staphylococcus aureus is methicillin-resistant.

In another aspect, the malignancy, organism or virus is a virus.

In one specific embodiment, the virus is influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus or tobacco mosaic virus.

The invention further provides a method of determining a source of a case of lung malignancy comprising identifying at least one peptide in a Replikin Peak Gene of a lung cancer cell that is also present in a Replikin Peak Gene of an isolate of tobacco mosaic virus, wherein the peptide is involved with the source of the lung malignancy.

In one embodiment, a plurality of peptides is identified in the Replikin Peak Gene of the lung cancer cell wherein each one of the plurality of peptides is also identified in the Replikin Peak Gene of an isolate of tobacco mosaic virus.

In another embodiment, the at least one peptide in the Replikin Peak Gene of the lung cancer cell and the at least one peptide in Replikin Peak Gene of the isolate of tobacco mosaic virus is a peptide of about 10 amino acids or less comprising at least two lysines and at least one histidine.

In a further embodiment, the at least one peptide in the Replikin Peak Gene of the lung cancer cell and the at least one peptide in Replikin Peak Gene of the isolate of tobacco mosaic virus is a peptide of about 10 amino acids or less comprising at least three lysines and at least one histidine.

In yet another embodiment, the at least one peptide in the Replikin Peak Gene of the lung cancer cell and the at least one peptide in the Replikin Peak Gene of the isolate of tobacco mosaic virus is about 7 amino acids or less comprising at least three lysines and at least one histidine.

In a further embodiment, the at least one peptide in the Replikin Peak Gene of the lung cancer cell and the at least one peptide in the Replikin Peak Gene of the isolate of tobacco mosaic virus is about 4 amino acids comprising three lysines and one histidine.

In a specific embodiment, the at least one peptide in the Replikin Peak Gene of the lung cancer cell and in the Replikin Peak Gene of the isolate of tobacco mosaic virus is KHKK (SEQ ID NO: 1584).

In another embodiment, the more than one KRKK (SEQ ID NO: 1584) peptide is identified in the Replikin Peak Gene of the lung cancer cell and in the Replikin Peak Gene of the isolate of tobacco mosaic virus.

In one specific embodiment, at least 10 KHKK (SEQ ID NO: 1584) peptides are identified in the Replikin Peak Gene of the lung cancer cell and at least 10 KHKK (SEQ ID NO: 1584) peptides are identified in the Replikin Peak Gene of the isolate of tobacco mosaic virus.

In another specific embodiment, at least 20 KHKK (SEQ ID NO: 1584) peptides are identified in the Replikin Peak Gene of the lung cancer cell and at least 20 KHKK (SEQ ID NO: 1584) peptides are identified in the Replikin Peak Gene of the isolate of tobacco mosaic virus.

In a third specific embodiment, at least 30 KHKK (SEQ ID NO: 1584) peptides are identified in the Replikin Peak Gene of the lung cancer cell and at least 30 KHKK (SEQ ID NO: 1584) peptides are identified in the Replikin Peak Gene of the isolate of tobacco mosaic virus.

In a fourth specific embodiment, at least 50 KHKK (SEQ ID NO: 1584) peptides are identified in the Replikin Peak Gene of the lung cancer cell and at least 50 KHKK (SEQ ID NO: 1584) peptides are identified in the Replikin Peak Gene of the isolate of tobacco mosaic virus.

The present invention further provides a method of identifying a first case of malignancy of the lung having a higher rate of replication, aggressive growth pattern or lethality as compared with a second case of malignancy of the lung comprising identifying a Replikin Peak Gene in a malignant cell from a first case of malignancy of the lung that has a higher Replikin Count in the Replikin Peak Gene than a Replikin Peak Gene identified in a malignant cell from a second case of malignancy of the lung.

In one embodiment, first and second cases of malignancy of the lung are non-small cell lung malignancies.

Further provided is an isolated or synthesized Replikin Peak Gene in a lung malignancy for diagnosis, prevention or treatment of lung cancer by the method comprising: (1) obtaining at least one malignant cell from a lung malignancy; (2) analyzing the protein sequences or protein sequence fragments of the at least one malignant cell for the presence and concentration of Replikin sequences; (3) identifying the protein sequence or the protein sequence fragment having the highest concentration of continuous Replikin sequences in the at least one malignant cell; (4) selecting the protein sequence or protein sequence fragment having the highest concentration of continuous Replikin sequences; (5) identifying the amino acid sequence of the selected protein sequence or protein sequence fragment as the Replikin Peak Gene; and (6) isolating or synthesizing the identified Replikin Peak Gene of the at least one malignant cell, wherein the isolated or synthesized identified Replikin Peak Gene is useful for diagnosis, prevention or treatment of lung cancer.

In one aspect, the lung malignancy is a non-small cell lung malignancy.

In another aspect, at least one isolated or synthesized Replikin sequence within the protein or protein fragment of the identified Replikin Peak Gene for diagnosis, prevention or treatment of lung cancer.

In a specific embodiment, the at least one isolated or synthesized Replikin sequence within the protein or protein fragment of the identified Replikin Peak Gene is one of SEQ ID NOS: 1585-1635 of SEQ ID NO: 1636.

The invention also provides an immunogenic composition for prevention and treatment of lung cancer, wherein the immunogenic composition comprises at least one isolated or synthesized Replikin sequence within the protein or protein fragment of the identified Replikin Peak Gene.

Also provided is method of stimulating the immune system, comprising administering in an animal the at least one isolated or synthesized Replikin sequence identified within the Replikin Peak Gene of the lung malignancy for prevention, treatment or diagnosis of lung cancer in an animal. In a specific embodiment, the animal is a human.

In another embodiment, the present invention provides a method of identification of a lethal form of lung cancer comprising: (1) obtaining at least one malignant cell from a plurality of lung tumors; (2) identifying the Replikin Peak Gene in the at least one malignant cell of each of the plurality of lung tumors; (3) analyzing the amino acid sequence of a protein or protein fragment of the Replikin Peak Gene in the at least one malignant cell of each of the plurality of lung tumors for the presence and concentration of Replikin sequences; (4) comparing the concentrations of Replikin sequences in each of the proteins or protein fragments of the Replikin Peak Gene in the at least one malignant cell of each of the plurality of lung tumors; and (5) identifying the lung tumor having the highest concentration of continuous Replikin sequences in the protein or protein fragment of the Replikin Peak Gene as a lethal form of lung cancer.

In a further embodiment, the present invention provides a method of identification of a more lethal form of lung cancer among at least two lung cancers, comprising: (1) obtaining at least one malignant cell from each of at least two lung cancers; (2) identifying the Replikin Peak Gene in the at least one malignant cell of each of the at least two lung cancers; (3) analyzing the amino acid sequence of a protein or protein fragment of the Replikin Peak Gene in the at least one malignant cell of each of the at least two lung cancers for the presence and concentration of Replikin sequences; (4) comparing the concentrations of Replikin sequences in each of the proteins or protein fragments of the Replikin Peak Gene in the at least one malignant cell of each of the at least two lung cancers; and (5) identifying the lung cancer having the highest concentration of continuous Replikin sequences in the protein or protein fragment of the Replikin Peak Gene as the more lethal form of lung cancer.

The invention further provides a method of determining an expected increase in lethality or virulence of a virus or organism which method comprises: (1) obtaining a plurality of isolates of said virus or organism wherein each isolate is isolated within a known time period and wherein at least two of said isolates is isolated about six months to about 5 years later than at least two other of said isolates; (2) identifying a Replikin Peak Gene in each isolate of said plurality of isolates; (3) analyzing the identified Replikin Peak Gene of each isolate of the plurality of isolates to determine the Replikin Count of each Replikin Peak Gene of each isolate of the plurality of isolates, or analyzing a protein, protein fragment, or gene area comprising the identified Replikin Peak Gene of each isolate of the plurality of isolates to determine the Replikin Count of the protein, protein fragment, or gene area of the plurality of isolates; (4) determining a mean Replikin Count within the Replikin Peak Gene or within the protein, protein fragment, or gene area comprising said identified Replikin Peak Gene for each known time period; (5) comparing the mean Replikin Count within the Replikin Peak Gene or within the protein, protein fragment, or gene area for each known time period one to another; (6) identifying an increase in the mean Replikin Count between at least two known time periods; and (7) identifying an expected increase in lethality or virulence of said virus, or organism within about six months to about three years following said identified increase in the mean Replikin Count.

In one specific embodiment, the known time period is about 1 year. In another specific embodiment, the increase in mean Replikin Count occurs over one year. In a further specific embodiment, the increase in mean Replikin Count occurs over three years. In another embodiment, the increase in mean Replikin Count is significant between at least two known time periods. In a further embodiment, the increase in mean Replikin Count has a significance of p=<0.001.

### BRIEF DESCRIPTION OF THE DRAWINGS

FIG. 1 illustrates the localization of the pB1 gene area as the Replikin Peak Gene in the genome of the H5N1 strain of influenza virus. Replikin Peak Genes are the places in the genome where Replikin sequences are continuous and most concentrated. The pB1 gene area comprises a Replikin Peak Gene in the H5N1 genome and the Replikin Count of the pB1 gene area correlates increases in virulence and mortality. Dark gray columns represent mean Replikin Counts for designated gene areas in isolates of H5N1 virus isolated during the given year. Light gray columns represent standard deviation from the mean in the population of isolates in a given year. Standard deviation of the means is shown in light gray columns on top of the means, rather than in the usual ‘T’ symbols. This style is used to emphasize the diverse expanding virus population with regard to the Replikin Count. Replikin Counts for isolates of H5N1 virus isolated in years 2003 through 2006 with genetic information publicly available at pubmed.com were determined separately by analysis of the number of Replikin sequences observed in each of the eight genome areas of human H5N1 influenza virus for isolates in a given year. The eight genome areas that have been identified are nucleocapsid, matrix, pB2, neuraminidase, pA, NS, hemagglutinin, and pB1 gene areas.

FIG. 2 illustrates an increase in Replikin Count before and accompanying each influenza A pandemic and outbreak since 1918 and low Replikin Counts during quiescent periods of influenza A infection and continually in non-lethal Influenza B. The graph provides annual Replikin Counts from 1917-2007 for all Replikin Peak Genes isolated in silico in the pB1 gene area of influenza strains having amino acid or nucleic acid sequences publicly available at PubMed. Data is provided (1) for non-lethal human Influenza B between 1940 and 2007 (thick dashed medium gray line) and (2) for both the lethal and non-lethal periods of human Influenza A viruses between 1917 and 2007. Human Influenza A strains are (1) H1N1 (thick medium gray line), (2) H2N2 (thin light gray line), (3) H3N2 (thin medium gray line), and (4) H5N1 (thin black line grey). H5N1 strains isolated from chicken are illustrated by a thick medium gray line. The total number of sequences analyzed for the data (N) is 14,227. Listed pandemics, epidemics and outbreaks are the 1918 H1N1 pandemic, the 1930's H1N1 epidemic, the 1957H2N2 pandemic, the 1968H3N1 pandemic, the 1977-78H3N2 outbreaks and the H5N1 outbreaks of 1997, 2001-2004 and 2007. Over a ninety year period, pandemics, epidemics and outbreaks are associated with Replikin Counts of four or above in the RPG of influenza strains. Over the same period constant low Replikin Counts of less than four may be observed during quiescent non-lethal periods of influenza A infections and low Replikin Counts of less than four may be observed in non-lethal Influenza B.

FIG. 3 illustrates successive “emerging” strains of influenza virus between 1930 and 2007. Mean Replikin Counts per year of isolation of various strains of influenza are provided for the polymerase area (marked with circles), the pB1 area (marked with triangles), and the pB1-F2 area (marked with squares). Data for H1N1 and H3N2 continue through 2007. Gaps represent years where no data was available for these genomic areas on PubMed. Dramatic increases in Replikin Count may be observed just before outbreak in the rebound epidemic of H1N1 beginning in the 1930's, in the pandemics of H2N2 and H2N3, which occurred in 1957 and 1968, respectively, and the outbreaks of H5N1 between 1997 and 2007. The largest increase in Replikin Count may be observed in the pB1-F2 area of the genome, which is contained within the pB1 area of the genome. The next largest increase in Replikin Count may be observed in the pB1 area of the genome, which is contained in the polymerase area of the genome. The smallest increase in Replikin Count may be observed in the polymerase area of the genome. It may be observed, therefore, that the Replikin Count becomes magnified as measured within the pB1 area as compared to the polymerase area and within the pB1-F2 area as compared to the pB1 area.

FIG. 4 illustrates the relationship of Replikin Count of the Replikin Peak Gene pB1 gene area in human H5N1 to percent human mortality between 2003 and 2007 in human cases of H5N1 infection. An increase in Replikin Count in the pB1 gene area of H5N1 is observed to be quantitatively related to higher mortality in the host. In the graph, (1) light gray represents the mean Replikin Count of whole virus isolates at a given year, (2) medium gray represents the mean Replikin Count in the pB1 area of publicly available sequences of isolates of human H5N1 at a given year, (3) the colorless bars represent the standard deviation from the mean of Replikin Count in a given year, and (4) black represents ten times the percent mortality of identified human cases of H5N1 infection in the given year.

FIG. 5 illustrates a 2005 through 2007 upregulation of human H5N1 in humans as compared to H5N1 in goose, duck and chicken. Dark grey represents mean Replikin Count in the Replikin Peak Gene pB1 gene area of H5N1 isolates from goose, duck, chicken and human in isolates from 2001 through 2006 where data was publicly available at www.pubmed.com. Light grey represents standard deviation from the mean.

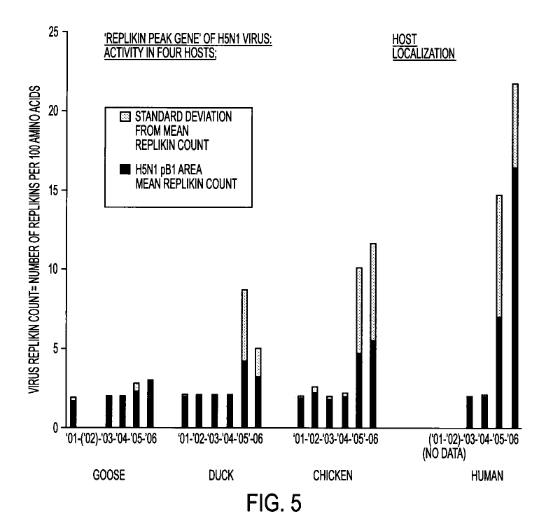

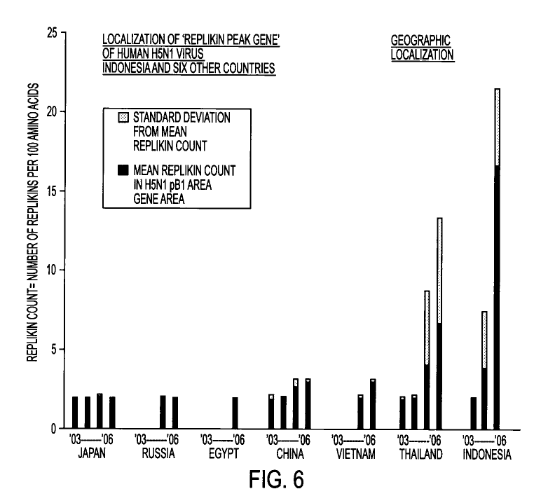

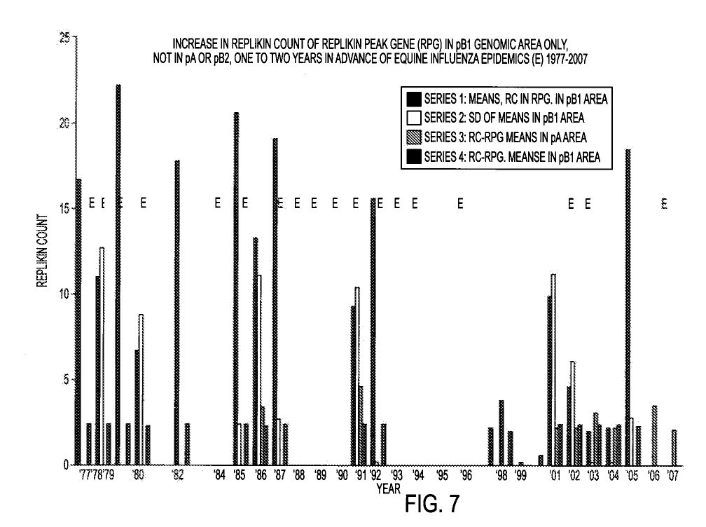

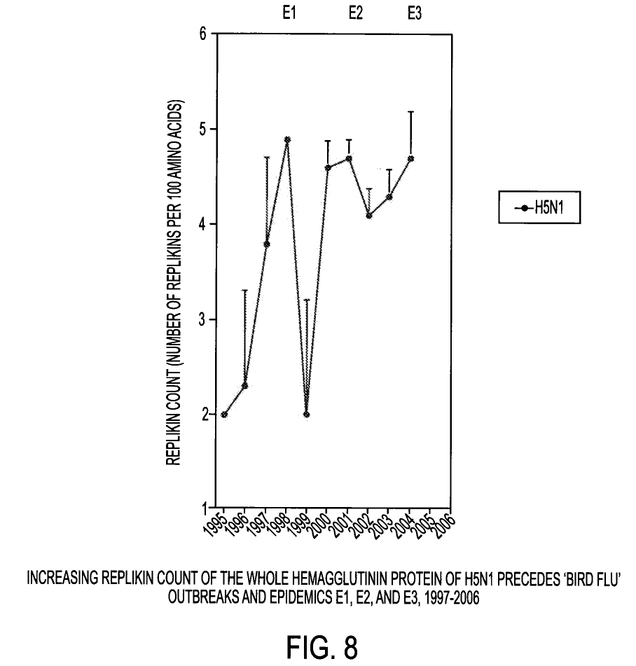

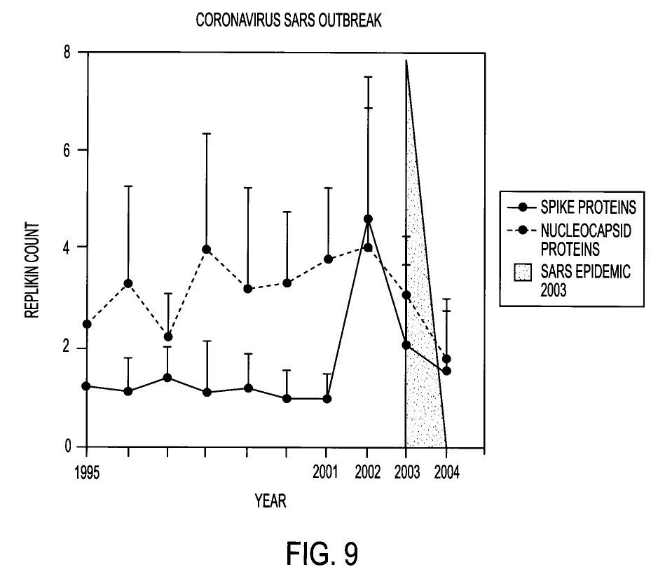

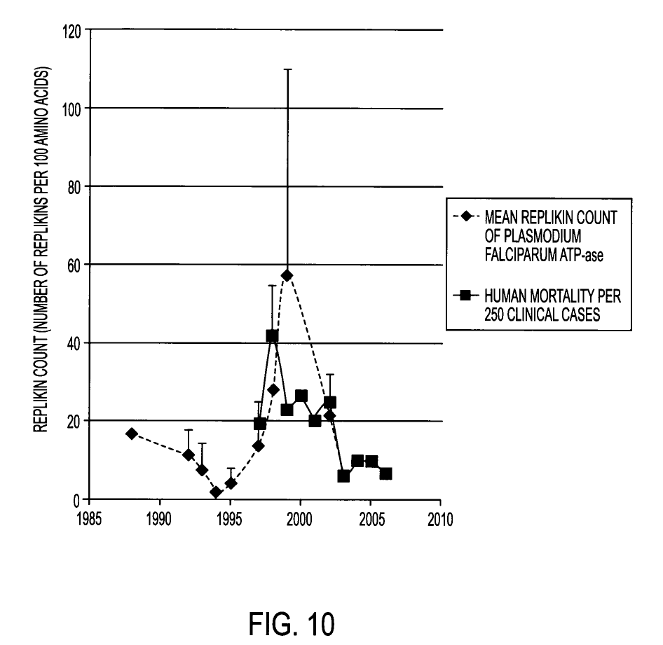

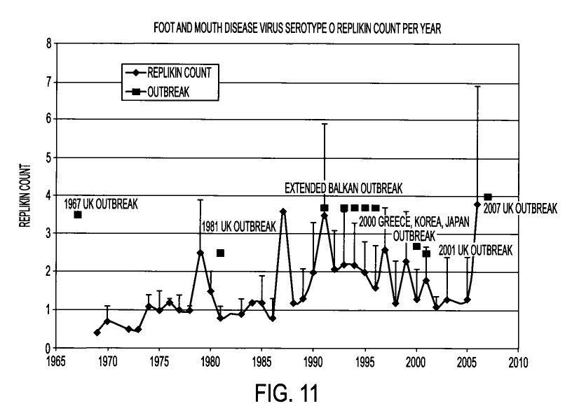

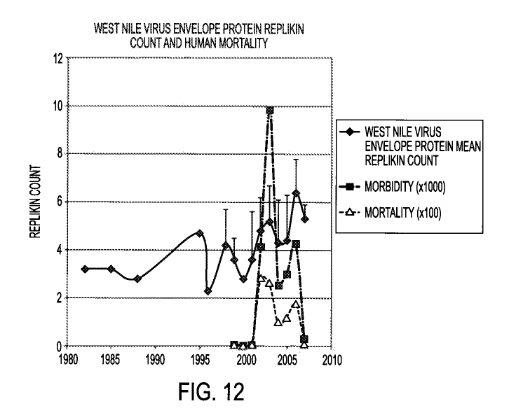

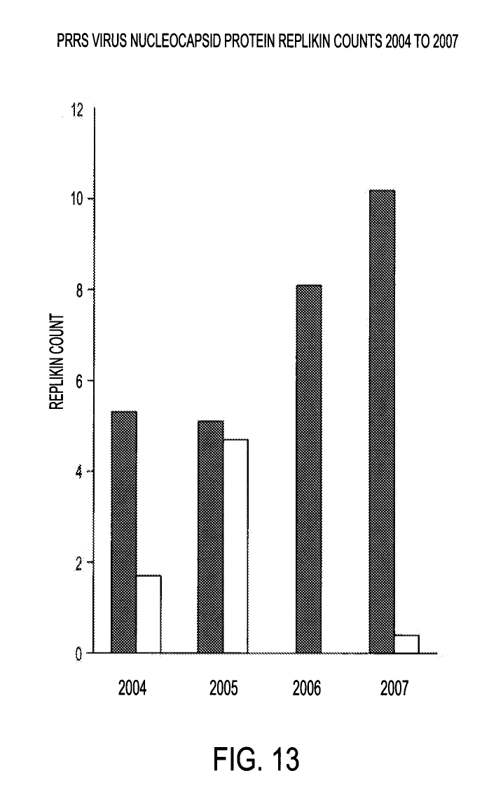

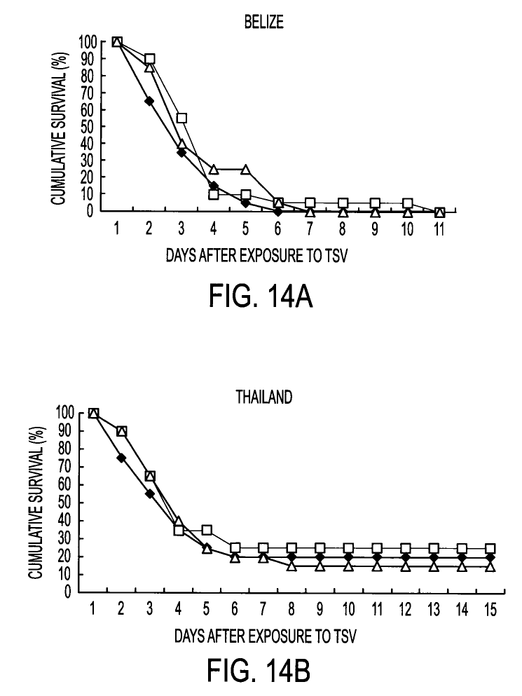

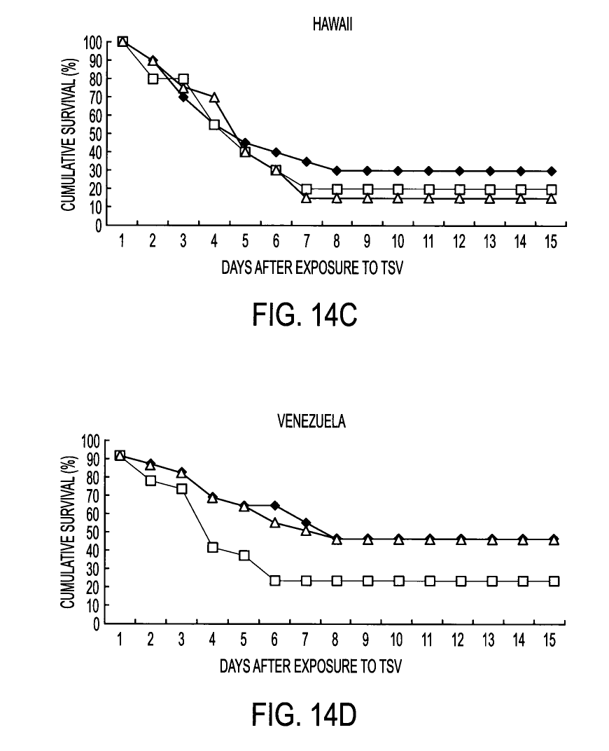

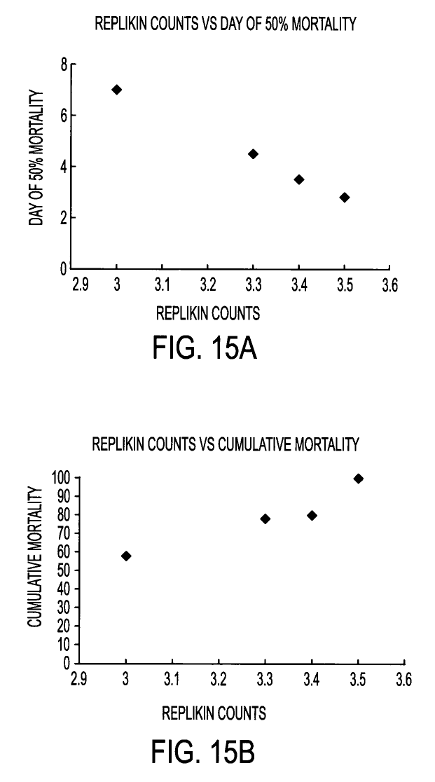

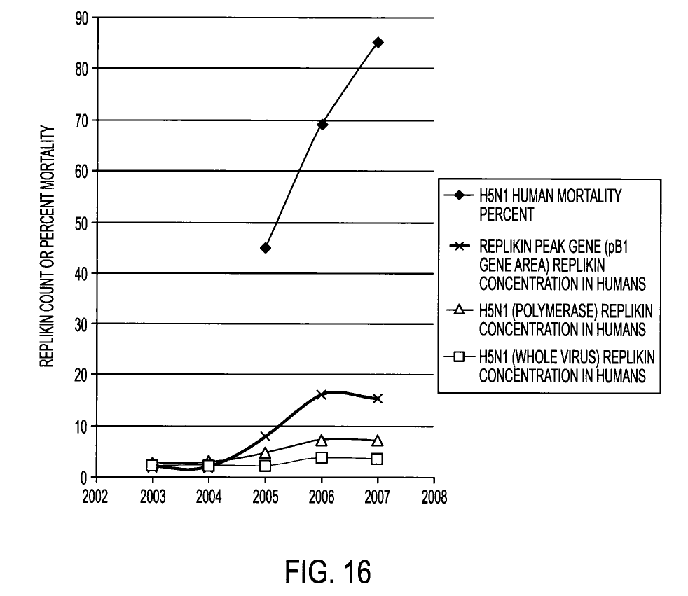

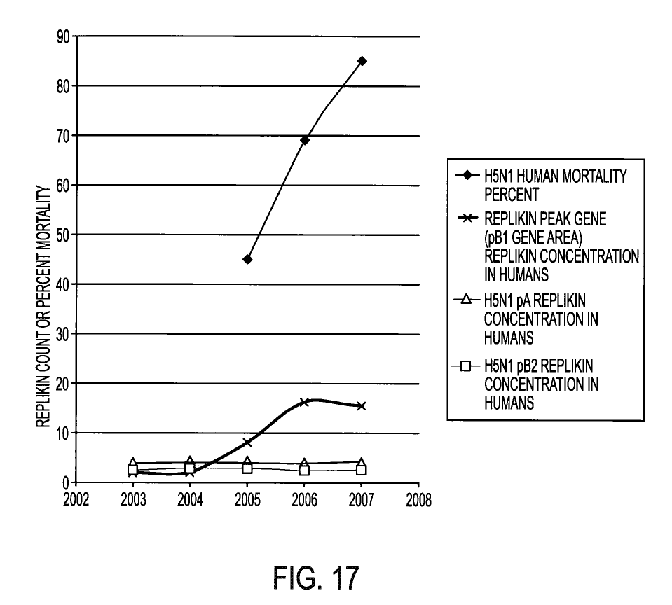

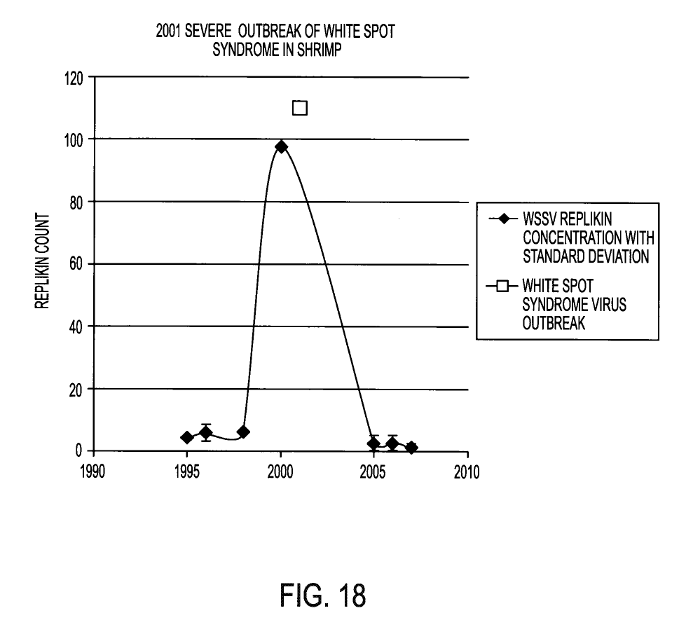

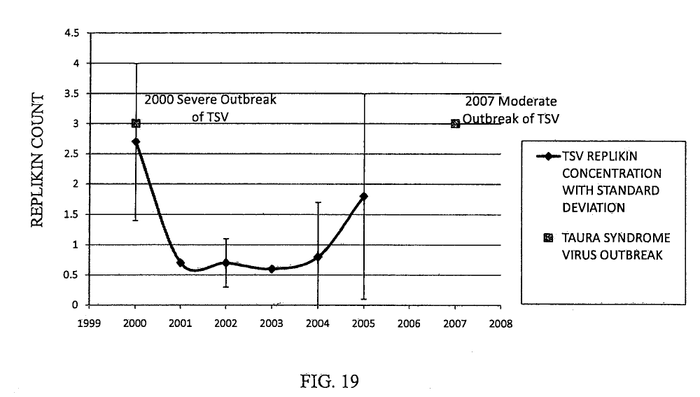

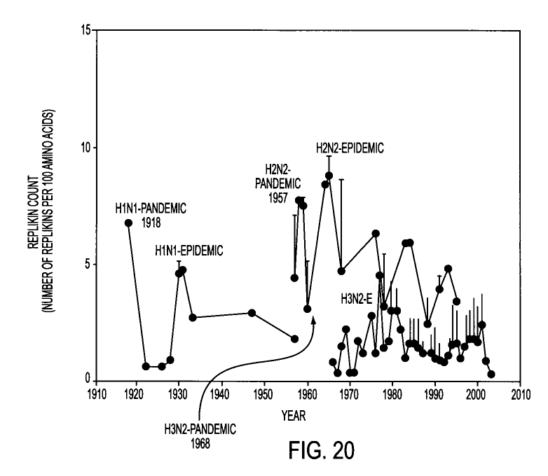

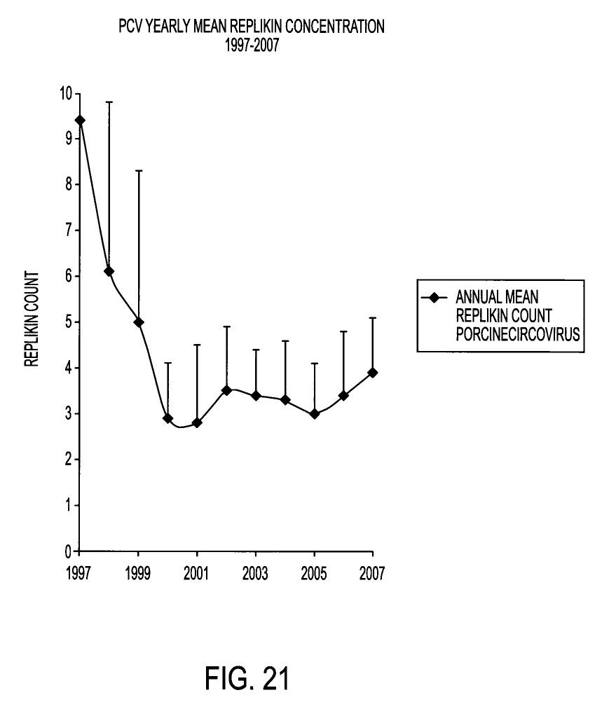

Replikin analysis was performed separately for H5N1 Replikin Peak Genes of each host group, namely, goose, duck, chicken and human. Low levels of Replikin count, below 4, were observed in each host group until 2005-2006. In 2005-2006 epidemics began to increase in Asian countries. While duck H5N1 counts decreased in 2006, they continued to increase in chicken H5N1 in 2006. Human RPG activity was upregulated in 2005-2006 and overtook RPG activity in chickens. This transition of Replikin Count increase from duck to chicken to human is in agreement with epidemiological evidence of the order of transfer of the virus between hosts. Changes in Replikin Count in the Replikin Peak Gene of the H5N1 isolates as in FIG. 5 allow for identification of those hosts in which the influenza virus strain is more virulent than other hosts.

FIG. 6 illustrates localization of human H5N1 isolates having the highest lethality by measuring mean Replikin Counts in isolates of human H5N1 from different geographic areas isolated in a given year. FIG. 6 is a bar graph depicting the number (with standard deviation) of Replikins per 100 amino acids in the pB1 gene area (Replikin Peak Gene) of H5N1 influenza virus strains identified annually in humans in Japan, Russia, Egypt, China, Vietnam, Thailand and Indonesia between 2003 and 2006.

Replikin analysis was performed separately for human H5N1 RPGs of each country. The results are shown for the Replikin Count for all data available on PubMed each year from 2003-2006. Low levels of Replikin count, below 4, were observed in each host group until 2005-2006, when human H5N1 increased in Asian countries. Human RPG activity was upregulated in 2005-2006 most prominently in Indonesia. The country most likely to first experience the increased human mortality was predicted in 2006 to be Indonesia. This prediction was proven correct in 2007 where incidence of human morbidity and mortality in the Indonesian outbreak were exceptionally high and evidence of possible human to human transmission was observed. Changes in Replikin Count in the Replikin Peak Gene of the H5N1 isolates such as in FIG. 6 allow for identification of those geographic areas in which the influenza virus strain is more virulent than other geographic areas.

FIG. 7 illustrates a relationship between Replikin Counts of Replikin Peak Genes identified within the pB1, pB2, and pA genomic areas of equine influenza 1977-2007 and epidemics of equine encephalitis caused by H3N8 equine influenza. Series 1 reflects the mean Replikin Count identified in the Replikin Peak Gene in the pB1 area of the genome. Series 2 reflects the standard deviation from mean Replikin Count in the pB1 gene area. Series 3 reflects the Replikin Count identified in the Replikin Peak Gene in the pA gene area of the genome, which neighbors the pB1 gene area. Series 4 reflects the Replikin Count identified in the Replikin Peak Gene in the pB2 gene area of the genome, which also neighbors the pB1 gene area. Replikin Count increases in the pB1 gene area are observed to occur one to three years before epidemic outbreaks while no increase in Replikin Count is observed in the pB2 and pA gene areas.

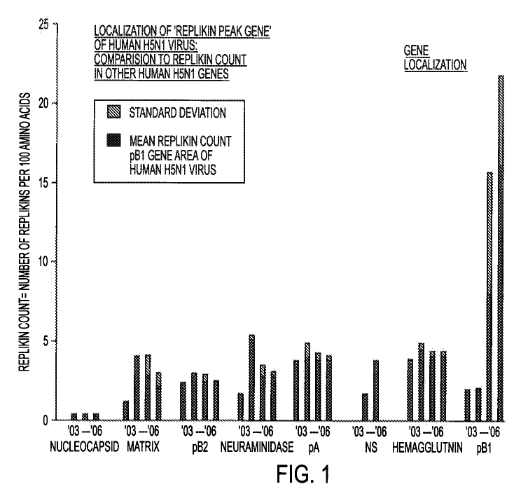

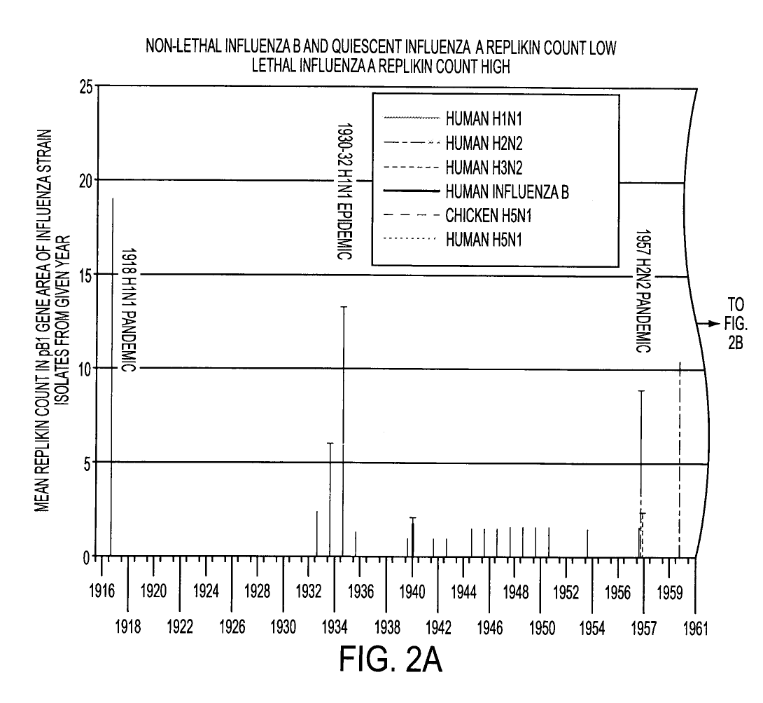

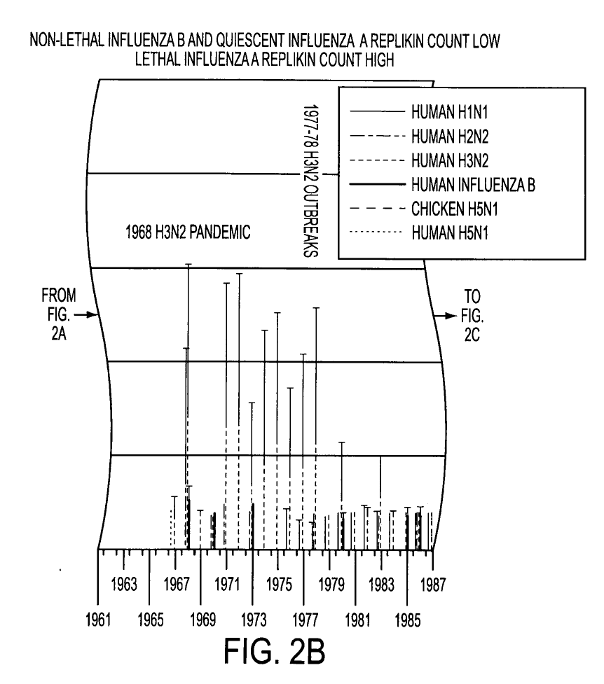

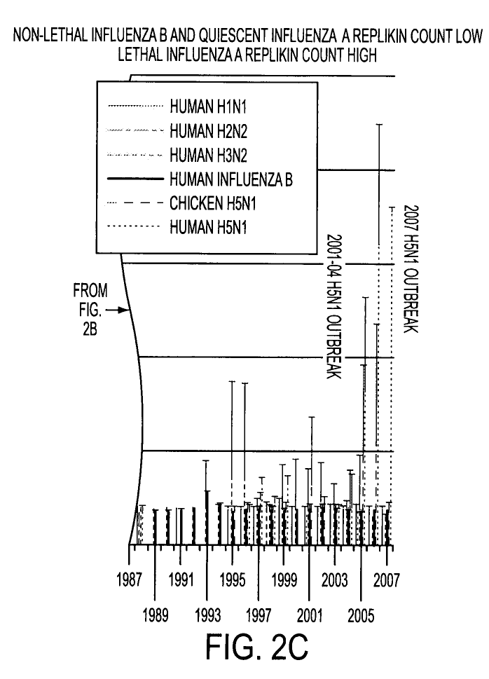

FIG. 8 illustrates an increasing Replikin concentration of the whole hemagglutinin protein in the H5N1 strain of influenza virus that preceded three “Bird Flu” Epidemics between 1997 and 2004. In H5N1 influenza, the increasing strain-specific Replikin concentration (Replikin Count, Means+/−SD) 1995 to 1997 preceded the Hong Kong H5N1 epidemic of 1997 (E1); the increase from 1999 to 2001 preceded the epidemic of 2001 (E2); and the increase from 2002 to 2004 preceded the epidemic in 2004 (E3). The decline in 1999 occurred with the massive culling of poultry in response to the E1 epidemic in Hong Kong. FIG. 8 demonstrates that although Replikin Count increases in RPGs occur in ranges four to eight fold greater than the increases which can be observed in whole proteins or genomes (see, e.g., FIGS. 1 and 2), changes in the Replikin Counts of whole proteins or genomes have the advantage of completeness and may be large enough to be detected and statistically significant.

FIG. 9 illustrates an increase in Replikin Count in spike and nucleocapsid coronavirus proteins preceding the SARS coronavirus epidemic of 2003. The x-axis indicates the year and the y-axis indicates the Replikin Count. The appearance of the SARS outbreak and the eight countries involved in the outbreak is shown by the conical shaded area. The solid black symbols represent the mean Replikin concentration for spike coronavirus proteins and the vertical black bars represent the standard deviation of the mean.

Although SARS was first identified in 2003, Applicants wondered whether the emergence of the SARS strain of coronavirus might have been presaged in the activity of the whole group of coronaviruses. The pre-pandemic increase in both nucleocapsid and spike coronavirus proteins is in accord with, and might have served as a warning of, the finding that a coronavirus would be responsible for the 2003 first SARS emergence. It may be seen that the Replikin Count rose between 1995 and 2002, consistent with the SARS coronavirus outbreak, which emerged at the end of 2002 and persisted into 2003. The decline in Replikin Count correctly signaled the end of the SARS outbreak and had already begun its return to pre-outbreak levels when the outbreak emerged. A similar decline occurred on termination of Influenza A epidemics and pandemics (FIG. 2). As also seen in FIG. 2, however, this decline has not occurred in the case of H5N1 in 2006 and 2007, so that the ongoing H5N1 outbreak may be assumed not to be over.

FIG. 10 illustrates that mortality rates in humans from Plasmodium falciparum correlate with Replikin Count in the P. falciparum ATP-ase enzyme. High malaria morbidity and mortality rates occurred in the late 1990s and was thought to be due to adaptation of the microorganism and decreased effectiveness of anti-materials. ATP-ase is a primary target of arteminisin treatment of malaria. With increased use of arteminisin, and improved public health measures, morbidity and mortality rates declined from 1998 to 2006. The Replikin Count of P. falciparum ATP-ase increased from 1997 to 1998 along with an increase in mortality per 250 malaria cases. The Replikin Count of P. falciparum ATP-ase decreased along with mortality rates from 1998 to 2006. Mortality rates per 250 cases for 1997 to 2006 were as follows: 1997 mortality rates was 7.7; 1998 mortality rate was 6.6; 1999 mortality rate was 9.1; 2000 mortality rate was 10.5; 2001 mortality rate was 8.1; 2002 mortality rate was 9.9; 2003 mortality 2.5; 2004 mortality rate was 4; 2005 mortality rate was 3.9; 2006 mortality rate was 2.6. Mortality rates declared by the World Health Organization, see www.who.int.

FIG. 11 illustrates a relationship between Replikin Counts observed in the VP1 protein (Replikin Peak Gene) of isolates of publicly-available foot and mouth disease virus serotype-O between 1969 and 2006 and certain observed outbreaks of Foot and Mouth Disease. Standard deviations are represented by vertical light grey capped lines above mean Replikin Counts. Observed European and UK outbreaks of Foot and Mouth Disease are noted including outbreaks in the UK in 1967, 1981, 2001 and 2007, in Baltic states in 1991 and 1993 through 1996, and Japan, Korea and Greece in 2000. Increases in Replikin Counts from baseline values between 1969 and 1978 preceded repeated increased Replikin counts 1979 forward, which in turn preceded outbreaks of foot and mouth disease 1981 to 2007.

FIG. 12 illustrates a relationship between Replikin Counts observed in the envelope protein of isolates of west nile virus and total human morbidity and mortality. The data for FIG. 12 is contained in Table 10. A correlation between Replikin Count in the envelope protein (the protein containing the RPG of the virus), and Morbidity and Mortality is demonstrated. FIG. 12 is a graph comparing (1) the concentration of Replikin (Replikin Count) of publicly available sequences of the envelope protein of isolates of west nile virus between 1982 and 2007 (with standard deviation bars for each data point), (2) total morbidity reported in the United States on a year by year basis by the Center for Disease Control (total U.S. morbidity is the value denoted on the y-axis times 100) between 1999 and 2007, and (3) total mortality resulting from WNV infection reported in the United States on a year by year basis by the Center for Disease Control between 1999 and 2007.

FIG. 13 illustrates Replikin Counts in the nucleocapsid protein of the porcine respiratory and reproductive syndrome virus (PRRSV) in isolates from 2004 through 2007. Mean Replikin Count is shown in grey columns. Standard deviation from the mean is shown in colorless columns. The Replikin Count of PRRSV nucleocapsid protein is seen to increase between 2004 and 2007. This increase correlates with a major outbreak of PRRSV in China. Standard deviation from the mean in 2005 is considerably larger than other years demonstrating a marked increase in Replikin Count was occurring in 2005 and measured as an increase in mean Replikin Count in 2006. The large standard deviation observed in 2005 indicates that more members of the class had increasing Replikin Counts. Standard deviation in 2005 was an early warning prior to the increase in the mean in 2006 and 2007. A similar phenomenon is observable in FIG. 7.

FIG. 14 illustrates a correlation between cumulative survival of Litopenaeus vannamei shrimp challenged with four different taura syndrome virusisolates over 15 days (unless 100% mortality occurred prior to 15 days) and the Replikin concentration of Open Reading Frame 1 (ORF1) of each isolate. Translated amino acid sequences of ORF 1 of the genome of individual isolates of TSV from Belize, Thailand, Hawaii and Venezuela were analyzed for Replikin Count. Replikin Count was determined to be 3.5 for the Belize isolate, 3.4 for the Thailand isolate, 3.3 for the Hawaii isolate and 3.0 for the Venezuela isolate. Graph A illustrates observed percent survival in three trials of shrimp challenged with the Belize isolate of TSV. In one trial, total mortality was observed on day 6. In the other trials, total mortality was observed on day 11. Graphs B, C and D illustrate observed percent survival of shrimp challenged with the Thailand isolate, the Hawaii isolate and the Venezuela isolate, respectively, each in three trials over 15 days. In the Thailand isolate, a mean of 80% percent mortality was observed on day 15. In the Hawaii isolate, a mean of 78.3% mortality was observed on day 15. In the Venezuela isolate, a mean of 58.3% mortality was observed on day 15.

FIG. 15A illustrates a direct sequential correlation between Replikin Count in isolates of taura syndrome virus (TSV) collected from Belize, Thailand, Hawaii and Venezuela, respectively, and mean number of days to 50% mortality in Litopenaeus vannamei shrimp challenged with the respective TSV isolates beginning on day one through day three. Statistical differences between the Replikin concentration for each isolate are significant at a level of p<0.001.

FIG. 15B illustrates a direct correlation between Replikin Count in isolates of taura syndrome virus (TSV) collected from Belize, Thailand, Hawaii and Venezuela, respectively, and mean cumulative survival of Litopenaeus vannamei shrimp at 15 days after challenge with the respective TSV isolate. Statistical differences between the Replikin concentrations for each isolate are significant at a level of p<0.001.

FIG. 16 illustrates a magnification of the effect of increases in Replikin Count on human mortality from H5N1 infections when Replikin concentration is observed in the pB1 gene area (containing a RPG) as compared to the polymerase gene or as compared to the entire genome of the H5N1 virus. In FIG. 16, a correlation is established between human mortality and (1) mean concentration of Replikin sequences in the whole genome, (2) mean concentration of Replikin sequences in the polymerase gene, and (3) mean concentration of Replikin sequences in the Replikin Peak Gene (pB1 gene area) of H5N1 influenza strains. Replikin concentration in the Replikin Peak Gene (pB1 gene area) of the H5N1 genome is seen to correlate most significantly with human mortality as compared to Replikin Counts in the whole genome and the polymerase gene.

FIG. 17 illustrates a significant eight-fold increase in Replikin concentration in the pB1 gene area (Replikin Peak Gene) of isolates of H5N1 from 2003 through the first quarter of 2007 (that correlates with an increase in host mortality in humans), while no significant increase is observed in neighboring gene areas of the pB1 gene area, namely, the pA gene area and the pB2 gene area. FIG. 17 graphically compares percent human mortality from H5N1 infections in years 2005 through the first quarter of 2007 to mean concentration of Replikin sequences in (1) the pB1 gene area, (2) the pB2 gene area, and (3) the pA gene area, respectively, of H5N1 influenza strains isolated in 2003 through the first quarter of 2007.

FIG. 18 illustrates a correlation between the mean Replikin Count and standard deviation of Replikin sequences observed in publicly available amino acid sequences of white spot syndrome virus (WSSV) isolated between 1995 and 2007 and a significant outbreak of WSSV in 2001. The remarkably high Replikin concentration in 2000 of 97.6 predicts the 2001 outbreak. Furthermore, an even more remarkable Replikin concentration of 103.8 was observed in a ribonucleotide reductase protein sequence from a 2000 isolate of WSSV wherein a Replikin Peak Gene was identified with an even higher Replikin concentration of 110.7.

FIG. 19 illustrates a correlation between increased Replikin Count in the genome of taura syndrome virus and outbreaks of the virus in 2000 and 2007 in shrimp. taura syndrome virus peptide sequences available at www.pubmed.com were analyzed by the inventors for mean Replikin concentration in the publicly available sequences. FIG. 19 is a graph comparing mean Replikin concentration for each year in which peptide sequences were publicly available between 2000 and 2005 (with standard deviation) and dates of significant outbreaks of taura syndrome virus. Significant outbreaks of the disease are noted at years 2000 and 2007. It may be observed from the graph that outbreaks of the virus occur following an increase in Replikin concentration. In year 2000, TSV had a Replikin Count of 2.7. Between 2001 and 2004, TSV had a lower mean Replikin Count, as low as 0.7, and an identified Replikin Scaffold disappeared. In 2005 the Replikin Scaffold reappeared, with an increase in lysines and histidines, and a commensurate increase in Replikin concentration to 1.8, followed by an increase in TSV outbreaks in 2006-2007.

FIG. 20 illustrates the total hemagglutinin Replikin Counts in the three influenza pandemics of the last century. Strain-specific high Replikin Counts accompany each of the three pandemics of the last century: 1918, 1957, and 1968. In each case this peak is followed by a decline (likely due to immunity in the hosts), then by a recovery and a “rebound” epidemic. The probability is very low that these correlations are due to chance, since they are specific for each strain, specific for each of the three pandemic years out of the century, specific for each post-pandemic decline, and specific for each rebound epidemic. Example 13 provides an example of analysis of hemagglutinin Replikin Counts in publicly available sequences between 1918 and 2007.

FIG. 21 illustrates an annual mean Replikin Count observed in isolates of porcine circovirus (PCV) having publicly available accession numbers on a year by year basis between 1997 and 2007 (with standard deviation bars for each Replikin Count data point) and demonstrates a correlation between increases in Replikin Count from 2000 through 2007 and reported increased in morbidity and mortality in Canada between 2000 and 2006 and an outbreak in China in 2007.

### DETAILED DESCRIPTION OF THE INVENTION

### Definitions

As used herein, a Replikin Peak Gene (RPG) (or sometimes a Replikin Peak Gene Area-RPGA) is to mean a segment of a genome, protein, segment of protein, or protein fragment in which an expressed gene or gene segment has a highest concentration of continuous, non-interrupted and overlapping Replikin sequences (number of Replikin sequences per 100 amino acids) when compared to other segments or named genes of the genome. Generally, a whole protein or gene or gene segment that contains the amino acid portion having the highest concentration of continuous Replikin sequences is also referred to as the Replikin Peak Gene. More than one RPG may be identified within a gene, gene segment, protein, or protein fragment. An RPG may have a terminal lysine or a terminal histidine, two terminal lysines, or a terminal lysine and a terminal histidine. For diagnostic, therapeutic and preventive purposes, an RPG may have a terminal lysine or a terminal histidine, two terminal lysines, or a terminal lysine and a terminal histidine or may likewise have neither a terminal lysine nor a terminal histidine so long as the terminal portion of the RPG contains a Replikin sequence or Replikin sequences defined by the definition of a Replikin sequence, namely, an amino acid sequence having about 7 to about 50 amino acids comprising:
(1) at least one lysine residue located six to ten amino acid residues from a second lysine residue;
(2) at least one histidine residue; and
(3) at least 6% lysine residues.
   Further, for diagnostic, therapeutic, preventive and predictive purposes, an RPG may include the protein or protein fragment that contains an identified RPG. For example, an RPG is herein identified in the pB1 gene area of H5N1. For predictive purposes, a Replikin Count in the RPG may be used to track changes in virulence and lethality. Likewise the RPG may be used as an immunogenic compound or as a vaccine. Additionally, however, as described herein, a Replikin Count in the pB1 gene area of influenza strains (like, for example, H5N1, H1N1 and H3N8), which contains but is not limited to an identified RPG having highest concentration of continuous, non-interrupted and overlapping Replikin sequences, is particularly useful for predicting changes in lethality and virulence. Other examples of predictive use of Replikin Counts in proteins in which RPGs have been identified are the VP1 protein of foot and mouth disease virus, the envelope protein in the west nile virus, and the nucleocapsid protein in porcine respiratory and reproductive syndrome virus, among many other viruses and organisms. Whole proteins or protein fragments containing RPGs are likewise useful for diagnostic, therapeutic and preventive purposes, such as, for example, to be included in immunogenic compounds, vaccines and for production of therapeutic or diagnostic antibodies.

As used herein, a Replikin sequence is an amino acid sequence having about 7 to about 50 amino acids comprising:
(1) at least one lysine residue located six to ten amino acid residues from a second lysine residue;
(2) at least one histidine residue; and
(3) at least 6% lysine residues.

A Replikin sequence may comprise a terminal lysine and may further comprise a terminal lysine or a terminal histidine. A Replikin peptide or Replikin protein is a peptide or protein consisting of a Replikin sequence. A Replikin sequence may also be described as a Replikin sequence of about 7 to about 50 amino acids comprising or consisting of a Replikin motif wherein the Replikin motif comprises:
(1) at least one lysine residue located at a first terminus of said isolated influenza virus peptide and at least one lysine residue or at least one histidine residue located at a second terminus of said isolated influenza virus peptide;
(2) a first lysine residue located six to ten residues from a second lysine residue;
(3) at least one histidine residue; and
(4) at least 6% lysine residues.
   For the purpose of determining Replikin concentration, a Replikin sequence must have a lysine residue at one terminus and a lysine or a histidine residue at the other terminus.

The term “Replikin sequence” can also refer to a nucleic acid sequence encoding an amino acid sequence having about 7 to about 50 amino acids comprising:
(1) at least one lysine residue located six to ten amino acid residues from a second lysine residue;
(2) at least one histidine residue; and
(3) at least 6% lysine residues,
   wherein the amino acid sequence may comprise a terminal lysine and may further comprise a terminal lysine or a terminal histidine.

As used herein, “animal” includes mammals, such as humans.

As used herein, the term “peptide” or “protein” refers to a compound of two or more amino acids in which the carboxyl group of one amino acid is attached to an amino group of another amino acid via a peptide bond. As used herein, “isolated” or “synthesized” peptide or biologically active portion thereof refers to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well-known in the art that, due to redundancy in the genetic code, individual nucleotides can be readily exchanged in a codon and still result in an identical amino acid sequence. As will be understood by one of skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

As used herein, “reservoir” is any source of Replikin sequences that may be shared with a virus, organism or malignancy including any host of a virus, organism or malignancy, any food source of a host of the virus, organism or malignancy, any vector of virus, organism or malignancy, or any substance wherein the genetic information of a virus, organism or malignancy that may be shared, mingled, mixed, exchanged, or come into the proximity of the Replikin sequences of the reservoir.

As used herein, “different time periods” or “different time points” is any two time periods or points that may be differentiated one from another. For example, an isolate of virus isolated during the year 2004 is isolated in a different time period than an isolate of the same virus isolated during the year 2005. Likewise, an isolate of virus isolated in May 2004 is isolated in a different time period than an isolate of the same virus isolated in June 2004. When comparing Replikin concentrations of different isolates, it is preferred to use comparable time periods for comparison. For example, an isolate from 2004 is preferably compared to at least one other isolate from some other year such as 2002 or 2005. Likewise, an isolate from May 2004 is preferably compared to at least one isolate from some other month of some year, for example, an isolate from December 2003 or from June 2004. An isolate is any virus isolated from a natural source wherein a natural source includes, but is not limited to, a reservoir of a virus, a vector of a virus or a host of a virus. “Obtaining” an isolate is any action by which an amino acid or nucleic acid sequence within an isolate is obtained including, but not limited to, isolating an isolate and sequencing any portion of the genome or protein sequences of the isolate, obtaining any nucleic acid sequence or amino acid sequence of an isolate from any medium, including from a database such as PubMed, wherein the nucleic acid sequence or amino acid sequence may be analyzed for Replikin concentration, or any other means of obtaining the Replikin concentration of a virus isolated from a natural source at a time point.

As used herein, an earlier-arising virus or organism or a virus or organism isolated at an earlier time period is a specimen of a virus or organism collected from a natural source of the virus or organism on a date prior to the date on which another specimen of the virus or organism was collected from a natural source. For viruses, a natural source includes, but is not limited to, a reservoir of a virus, a vector of a virus, or a host of the virus. A later-arising virus or organism or a virus or organism isolated at a later time period is a specimen of a virus or organism collected from a natural source of the virus (including, but not limited to, a reservoir, a vector, or a host) or a natural source of the organism on a date subsequent to the date on which another specimen of the virus or organism was collected from a natural source.

As used herein, “emerging strain” refers to a strain of a virus identified as having an increased or increasing concentration of Replikin sequences in one or more of its protein sequences relative to the concentration of Replikins in other strains of such organism. The increased or increasing concentration of Replikins occurs over a period of preferably at least about six months, at least about one year or at least about three years, but may be a much shorter period of time for highly mutable viruses. An emerging strain of virus indicates an increase in lethality, virulence or replication.

As used herein, “bird” is any avian species including migratory and domestic birds, wherein said migratory and domestic birds includes, for example, chickens, ducks of all kinds, geese, pigeons, gulls, seabirds etc.

As used herein, “outbreak” is an increase in virulence, morbidity or mortality in a viral disease as compared to a baseline of an earlier occurring epidemiological pattern of infection in the same viral disease. One of ordinary skill in the art will know how to determine an epidemiological baseline. As used herein, “morbidity,” is the number of cases of a disease caused by the virus, either in excess of zero cases in the past or in excess of a baseline of endemic cases in the past. Therefore the baseline of endemic cases, in epidemiological terms, may, for example, relate to whether no or some cases were present in a geographic region in the immediate past. The past, in epidemiological terms, may mean more than one year and can mean several years or more as understood by one of ordinary skill in the art. The past may also mean less than one year as determined by one of ordinary skill in the art. In the case of annually-recurrent common influenza, for example, the baseline reflects an annual recurrence of common influenza.

As used herein, “mutation” refers to a change in the structure and properties of a virus or organism caused by substitution of amino acids. In contrast, the term “conservation” as used herein, refers to conservation of particular amino acids due to lack of substitution. A “point mutation” may refer to a change in a single amino acid residue or may refer to a change in a small number of amino acid residues.

As used herein, “segment” or “portion” of a genome, protein or protein fragment refers to any nucleic acid sequence of any size within a genome or any amino acid sequence of any size within a protein or protein fragment wherein the termini of the nucleic acid sequence may be any two nucleic acid residues within the genome and the termini of the amino acid sequence may be any two amino acid residues within the protein or protein fragment.

As used herein, “Replikin Count” or “Replikin Concentration” refers to the number of Replikins per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration.

As used in this patent application, the term “continuous Replikin sequences” means a series of two or more Replikin sequences that are overlapped or are directly covalently linked.

As used herein a “Replikin Scaffold” refers to a series of conserved Replikin peptides wherein each of said Replikin peptide sequences comprises about 16 to about 34 amino acids, and preferably about 27 to about 33 amino acids and further comprises: (1) a terminal lysine and optionally a lysine immediately adjacent to the terminal lysine; (2) a terminal histidine and optionally a histidine immediately adjacent to the terminal histidine; (3) a lysine within 6 to 10 amino acid residues from another lysine; and (4) about 6% lysine. “Replikin Scaffold” also refers to an individual member or a plurality of members of a series of Replikin Scaffolds.

In an influenza virus, a Replikin Scaffold may refer to a Replikin peptide sequence comprising about 16 to about 34 amino acid residues, and in a preferred embodiment about 28 to about 30 amino acid residues. In a white spot syndrome virus, a Replikin Scaffold may refer to a Replikin peptide sequence comprising about 16 to about 34 amino acid residues, and in a more preferred embodiment about 29 to about 31 amino acid residues. In a taura syndrome virus, a Replikin Scaffold may refer to a Replikin peptide sequence comprising about 16 to about 34 amino acid residues, and in a more preferred embodiment about 29 to about 33 amino acid residues.

I. Replikin Count in Replikin Peak Gene is Predictive of and Related to Virulence and Lethality in Malignancies, Influenza and Other Pathogens and Replikin Peak Genes and Associated Replikin Sequences are Useful for Diagnostic, Therapeutic and Predictive Purposes

A virus Replikin gene related to lethality and virulence was first identified by Applicants in human H5N1 Influenza And was labeled a Replikin Peak Gene. Replikin Peak Genes were subsequently isolated in silico in numerous other viruses, bacteria, and protozoa. Replikin Peak Genes have now been associated with lethality in plant, fish, crustacea and vertebrate hosts. Because of their association with lethality, virulence and rapid replication, Replikin Peak Genes are now available as excellent targets for therapeutic and preventive treatments for a wide range of malignancies and pathogens.

Replikins, a class of peptides related to rapid replication, are 7 to 50 amino acids long, containing at least 2 lysine groups 6 to 10 amino acids apart, at least 1 histidine group, and at least 6% lysine. The phenomenon of the association of Replikins with rapid replication and virulence has been fully described in U.S. Pat. No. 7,189,800, U.S. Pat. No. 7,176,275, U.S. application Ser. No. 11/355,120, U.S. application Ser. No. 10/860,050 and U.S. application Ser. No. 10/105,232. Both Replikin concentration (number of Replikins per 100 amino acids) and Replikin composition have been correlated with the functional phenomenon of rapid replication.

Using an algorithm constructed to identify, count, and track Replikin sequences historically, Replikins were analyzed in 130,488 protein and genome sequences, representing all the accession numbers for common strains of influenza and some other lethal virus isolations published between 1917 and 2007 and reported on PubMed. Genomic areas with the highest concentration of continuous Replikins were isolated and named Replikin Peak Genes (RPGs).

Analysis of all publicly available protein and genome sequences for lethal Influenza A strains, including HSN 1, revealed 10,182 RPGs. RPGs were found to be present in isolates from all outbreaks of lethal influenza between 1917 and 2007 and the number of Replikin sequences per 100 amino acids (Replikin Count) in the identified RPGs was consistently observed to be above four and increased to as high as 29. In a significant control in Influenza B virus, which is non-lethal in humans, the Replikin Count in all 371 RPGs in Influenza B between 1940 and 2006 were found never to exceed four. Replikin Counts below four in the RPG of Influenza B virus contrasts with lethal Influenza A strains (with Replikin Counts as high as 29). RPG Replikin Counts during quiescent (or non-lethal) periods of Influenza A, were regularly observed to be four or below.

Replikin Counts below four for non-lethal isolates of influenza may be compared to highly lethal or virulent viruses such as ebola virus, which has been observed to have a Replikin Count of 32, Porcine Reproductive and Respiratory Virus (PRRSV) in pigs, which has been observed to have a Replikin Count of 43, gemini yellow leaf curl virus in tomato plants observed to have a Replikin Count of 56, hemorrhagic septicemia virus in fish observed to have a Replikin Count of 59, and white spot syndrome virus in shrimp, which has been observed to have a Replikin Count of 106. All of these viruses were observed to return to low counts during quiescent periods. Increased Replikin Counts in RPGs also were found in Mycobacterium tuberculosis (28), in methicillin-resistant Staphylococcus aureus (81), in Plasmodium falciparum (malaria) (153), and in lung cancer (261).

Analysis of Replikin Counts in genomic and proteonomic sequences alone prospectively correctly predicted: 1) the order of lethality in shrimp of four strains of taura syndrome virus (prediction was made blind in a laboratory study); 2) a 2007 increase in H5N1 percent mortality in humans; and 3) the country in which the increased percent mortality would occur most significantly, namely, Indonesia.

In addition to high Replikin Counts, analysis of rapidly replicating, virulent and lethal virus has revealed a series of conserved Replikin peptides associated with rapid replication, virulence and lethality known as Replikin Scaffolds. Replikin Scaffolds were observed in influenza virus strains where, for example, a 29-amino acid Replikin Scaffold has been conserved for 90 years in the genome of successive influenza virus strains. The scaffold has been present in each of the lethal influenza pandemics of 1918, 1957 and 1968 and in each lethal H5N1 outbreaks. Repeating signatures such as a KHKK (SEQ ID NO: 1584) signature has been observed in Replikin sequences within RPGs of lethal malignancies, viruses and organisms. The KHKK (SEQ ID NO: 1584) signature has been observed eleven times within the RPG of the protozoa that causes most malaria, P. falciparum. The KHKK (SEQ ID NO: 1584) signature has been observed 20 times within the RPG of a tobacco mocaic virus that induced exacerbated cell death in a pepper plant. The KHKK (SEQ ID NO: 1584) signature has been observed 57 times in non-small cell lung carcinoma within 52 Replikins observed within the 18 amino acid RPG identified in chromosome 9 of a non-small cell lung carcinoma. The presence of such a high number of KHKK (SEQ ID NO: 1584) signatures within the 18 amino acid RPG of the non-small cell lung carcinoma is explained by overlapping of the signatures. Overlapping of Replikin sequences and repeated signatures such as KHKK (SEQ ID NO: 1584) has now been associated with lethality, virulence and rapid replication. Together, these data indicate that a Replikin gene is quantitatively associated with lethal functions, and may be a mobile agent of lethality transferring between strains and species.

Whether Replikins can arise by synthesis de novo or are transferred from one organism or virus to another (or both) is yet to be determined. There is some beginning evidence for both. In one experiment, Replikin synthesis and/or transfer was facilitated in the laboratory in glioblastoma multiforme cells growing in tissue culture. The event, which facilitated the synthesis and/or transfer, was induced anoxia. Whether the anoxia stimulates increased rate of Replikin synthesis or membrane impairment facilitates Replikin transfer, or both, is yet to be determined.

Counting of Replikin sequences within a malignancy, a virus, a protozoon, a plant or an animal is aided by computer review of databases of gene and protein sequences. Bacteria were accepted as real when the light microscope permitted them to be seen as discrete entities, sufficiently discrete that they could be counted. Similarly, viruses were accepted as real when the electron microscope permitted them to be seen as discrete entities, sufficiently discrete that they could be counted. Likewise, Replikins can now be accepted as real since the “computer microscope” permits them to be seen as discrete entities, sufficiently discrete that they can be counted. Hence, the Replikin Count, or determination of number of Replikin Sequences in 100 amino acids in any given genomic or proteomic sequence, is facilitated on a large scale by computer analysis and comparison of Replikin Counts has provided the necessary evidence to associate increased Replikin Counts (in both whole genomes and Replikin Peak Genes) with lethality.

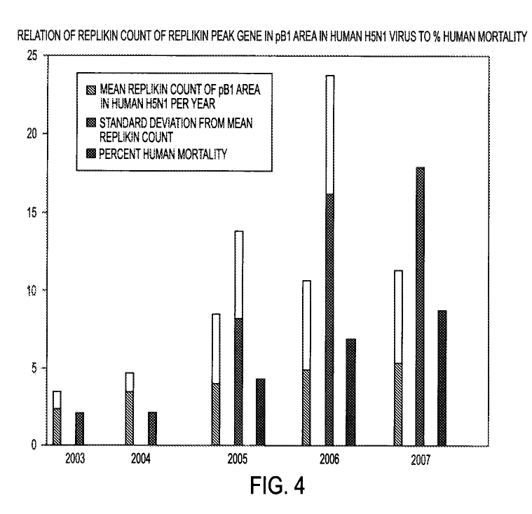

Visualization and counting of Replikin sequences in a wide range of genomes has now revealed that Replikin sequences are not scattered throughout the genome of lethal, virulent and rapidly replicating entities but, instead, are concentrated in particular areas of the genome. The concentration of Replikin sequences in a particular area of the genome has now been identified as a Replikin Peak Gene (RPG). Concentration of Replikin sequences in a RPG provides a magnification of the Replikin Count and a magnification of the developmental, growth and disease associations with the presence of Replikin Sequences. See, e.g., FIGS. 1, 3, 16 and 17. This magnification not only makes identification and counting easier, but facilitates the discovery of both the structural history and the functional associations of Replikins, as seen, for example, in the increase in Replikin Count of the RPG of human H5N1 with the increased percent human mortality between 2003 and 2007. FIGS. 4, 16 and 17.

The magnification effect of analyzing the Replikin Count of a Replikin Peak Gene as compared to Replikin Counts from other parts of a genome or the whole genome is demonstrated in FIGS. 16 and 17. There, mortality in humans from H5N1 infection correlates strongly with an increase in Replikin Count in the pB1 gene area (RPG) of the virus while correlating less strongly with an increase in Replikin Count in the polymerase gene or the whole genome of the virus.

By means of visual and software inspection, Applicants have analyzed 130,488 protein and genome sequences from common strains of influenza and other lethal viruses, isolated from 1917 to 2007 and accessible in PubMed. Replikin sequences in these 130, 488 sequences have been identified, counted and annually tracked. This extensive analysis revealed the Replikin Peak Gene that has not been found to be quantitatively related to lethality in several hosts, including plants, fish, crustacea and vertebrates, such as humans.

II. Prediction of Pathogenic Outbreaks and Lethal Malignancies

Prediction of epidemics and future outbreaks of viruses such as Influenza A (including H1N1, H2N2, H3N2, H3N8 and H5N1), foot and mouth disease virus, west nile virus, porcine reproductive and respiratory syndrome virus, porcine circovirus, white spot syndrome virus, taura syndrome virus, tobacco mosaic virus, coronavirus, and SARS virus, may be made, for example, by reviewing the Replikin concentration of isolates of a virus strain and comparing the Replikin concentration for a particular time period with Replikin concentrations from another time period. Prediction of outbreaks or increases in virulence or lethality of organism may also be made, for example, by reviewing the Replikin concentration of isolates of an organism and comparing the Replikin concentration for a particular time period with Replikin concentrations from another time period. Organisms for which outbreaks or increases in virulence and lethality may be predicted include, for example, P. falciparum, M mucogenicum and S. aureus.

The difference in time period may be, for example, one month, six months, one year, three years or more. Preferably, the difference in time period is six months to three years. Also preferably, the difference in time period is one year. A significant increase in Replikin concentration from one year to the next and preferably over one, two, three or five years provides predictive value of an emerging strain of virus or organism that may begin an outbreak. A viral or other pathogenic outbreak may be predicted within about six months to about one to about three-years from the observation of a significant increase in Replikin concentration. The outbreak is preferably predicted within about one to about two years. An outbreak of virus or other pathogen, therefore, may be predicted within 1 to about 2 years as demonstrated in FIGS. 2, 3, 7, 11 and 19 wherein an epidemic occurred at about 1 to about 2 years following each peak of the measured Replikin Count of the particular viruses and organisms.

Significant increases may be observed over a time period of more than one year, such as three, four, five or more years. An outbreak may likewise be predicted within about six months to about one year or more from the initial observation of an observable decrease in Replikin concentration following a significant increase.

The correlation between Replikin concentration and viral outbreaks noted throughout this application provide a method of predicting outbreaks of virus and other pathogens by monitoring increases or decreases in Replikin concentration in the RPG of isolates of the virus or other pathogen. Likewise, the lethality of an organism may be predicted by comparing the Replikin Count of the identified RPG of a malignancy with the Replikin Count of the identified RPG of another malignancy of the same type.

III. Replikin Peak Gene Sequences in Diagnostics and Therapies

High Replikin concentrations and RPGs have been shown to be related to rapid replication, viral outbreaks, epidemics, morbidity and host mortality in, for example, influenza virus strains, including H5N1, in SARS coronavirus, in shrimp in taura syndrome virus and in white spot syndrome virus, in foot and mouth disease virus, porcine reproductive and respiratory syndrome virus and in porcine circovirus, and in malignancies such as non-small cell lung carcinoma, among others. Because Replikin sequences in general (and particularly RPGs) are chemically defined, the sequences may be synthesized by organic chemistry rather than biological techniques, and thus are more specific, more reproducible and more reliable than other targets for diagnostics and therapeutics. The chemically defined Replikin sequences identified by the applicants are likewise potentially freer of adverse reactions that are characteristic of biologically derived vaccines and antibodies.

In addition to the diagnostic power of Replikin technology shown in these examples, it is clear that recognition for the first time of this class of virus peptides, and the discovery that they are related to rapid replication, virus outbreaks and high morbidity and mortality, makes the Replikins, and particularly the Replikin Peak Gene structures illustrated herein, new conserved prime targets for treatment and vaccines against these and other viruses.

Presence of the Replikin Peak Gene correlates with an increase in virulence in various species and an increase in mortality rate in humans in influenza virus, malaria and lung cancer and in pigs in PRRSV and porcine circovirus. Because an increase in virulence and mortality rate can be correlated with the Replikin Peak Gene (RPG), portions or fragments of the RPG are available as preferred targets for treatment with vaccines, antibodies or other blocking agents. Replikins in the gene are further preferred targets for identification of virulent strains of virus and other pathogens and for prediction of outbreaks of virus and other pathogens.

IV. Immunogenic Compounds, Vaccines, Antibodies and Blocking Agents

The observations of specific Replikins and their concentration in proteins of viral and organismal pathogens and malignancies provides the first specific quantitative early chemical correlates of outbreaks and provides for production and timely administration of vaccines tailored specifically to treat the prevalent emerging or re-emerging strain virus in a particular region of the world. By analyzing the protein sequences of isolates of a virus for the presence, concentration and/or conservation of Replikins, virus outbreaks and epidemics can be predicted and treatments developed. Furthermore, the severity of such outbreaks can be significantly lessened by administering a peptide immunogenic compound or vaccine based on the Replikin sequences identified herein or using the methods provided herein or Replikin sequences found to be most abundant or shown to be on the rise in virus isolates over a given time period, such as about one to about three years.

Vaccine products against SARS Replikin sequences and H5N1 influenza virus Replikin Scaffolds have been demonstrated by Applicants. See, e.g., U.S. application Ser. No. 11/355,120, filed Feb. 16, 2006 (Examples 6 and 7), incorporated herein by reference. Replikin sequences added to the feed source of shrimp have likewise imparted measurable resistance to challenges with taura syndrome virus. See Example 19. To date, all Replikin sequences tested in rabbit or chicken have induced an immune response and the glioma Replikin sequence (SEQ ID NO: 3658) has been identified and synthesized in peptides that induce and immune response and react with natural antibody responses in humans. See U.S. Pat. No. 6,638,505.

An immunogenic compound or peptide vaccine of the invention may include a single Replikin peptide sequence or may include a plurality of Replikin sequences observed in particular virus strains. Preferably, the peptide vaccine is a Replikin Peak Gene or a Replikin sequence isolated within a Replikin Peak Gene. Further, the peptide vaccine may be based on Replikin sequence(s) shown to be increasing in concentration over a given time period and conserved for at least that period of time. A vaccine may also include a conserved Replikin peptide(s) in combination with a new Replikin(s) peptide or may be based on new Replikin peptide sequences. The Replikin peptides can be synthesized by any method, including chemical synthesis or recombinant gene technology, and may include non-Replikin sequences, although vaccines based on peptides containing only Replikin sequences, Replikin Peak Genes or Replikin sequences identified within a Replikin Peak Gene are preferred. Preferably, vaccine compositions of the invention also contain a pharmaceutically acceptable carrier and/or adjuvant.

The immunogenic compounds and vaccines of the present invention can be administered alone or in combination with antiviral drugs, such as gancyclovir; interferon; interleukin; M2 inhibitors, such as, amantadine, rimantadine; neuraminidase inhibitors, such as zanamivir and oseltamivir; and the like, as well as with combinations of antiviral drugs.

The vaccine of the present invention may be administered to any animal capable of producing antibodies in an immune response. For example, the vaccine of the present invention may be administered to a rabbit, a chicken, a pig or a human. Because of the universal nature of Replikin sequences, a vaccine of the invention may be directed at a range of strains of virus or a particular strain of virus.

V. Increased Replikin Counts in Replikin Peak Gene of pB1 Area of Influenza A Strains Correlates with Pandemics and Lethal Outbreaks

Applicants have identified Replikin Peak Genes as a segment of a genome, protein, segment of protein, or protein fragment in which an expressed gene or gene segment has the highest concentration of continuous, non-interrupted and overlapping Replikin sequences (number of Replikin sequences per 100 amino acids) as compared to other segments or named genes of a genome. The inventors have likewise identified gene areas or proteins or protein fragments containing the highest concentration of continuous, non-interrupted and overlapping Replikin sequences (number of Replikin sequences per 100 amino acids) as Replikin Peak Genes.

Increased Replikin Counts in the Replikin Peak Gene identified in the pB1 gene area of influenza A strains has now been correlated by Applicants with pandemics and lethal outbreaks of influenza. These findings correspond to the Applicants' discovery that quantitative measurement of the concentration of Replikin peptides in proteins allows for correlation of Replikin peptide concentration per 100 amino acids with virulence, morbidity, mortality, epidemics and pandemics in malignancies, and organismal and viral infections. A correlation between increased Replikin Counts in the RPG of malignancies and pathogens has been established by Applicants in, for example, human pandemic influenza viruses, H5N1 (“Bird Flu”) influenza virus, white spot syndrome virus, foot and mouth disease virus, west nile virus, porcine reproductive and respiratory syndrome virus, porcine circovirus, equine influenza virus, tobacco mosaic virus, malaria and non-small cell lung malignancies, among others. An increase in Replikin Count in these pathogens and malignancies allows for prediction of increased lethality or virulence and prediction of forthcoming outbreaks of infections.

A. Replikin Peak Gene in H5N1 Associated with Lethal Outbreak

Applicants initially identified a Replikin Peak Gene in the pB1 gene area of the genome of the H5N1 strain of influenza virus (e.g., SEQ ID NO: 1684) and observed that outbreaks of the H5N1 virus and lethality in infections from the virus correlated with increases in Replikin Count in the identified Replikin Peak Gene. FIG. 1 illustrates the localization of the pB1 gene area as the Replikin Peak Gene in the genome of the H5N1 strain of influenza virus. The data for FIG. 1 is contained in Table 1. The eight genome areas identified in the H5N1 genome are the nucleocapsid, matrix, pB2, neuraminidase, pA, NS, hemagglutinin, and pB1 gene areas. The graph in FIG. 1 reveals that Replikin sequences were found to be most concentrated in the pB1 gene area of the H5N1 virus genome. When the inventors identified the Replikin Peak Gene (RPG) in the pB1 gene area, they discovered that an increase in Replikin Count in the RPG correlated with an increase in lethality in virus infectious. As such, “upregulation” of the RPG in H5N1 was observed in 2005 and 2006 as a significant increase in mean Replikin Count and standard deviation from the mean Replikin Count were observed and this upregulation correlated with increased lethality and virulence.

Table 1 provides mean Replikin Count and standard deviation from mean for publicly available sequences at PubMed for each of the eight gene areas in isolates of H5N1 between 2003 and 2006. Where no data is available for a given year, the year is not included in the table.

TABLE 1
H5N1 Influenza

| | | | | | |
|---|---|---|---|---|---|
| | | | Mean | | |
| | | No. of | Replikin | | |
| | | Isolates | Count | | |
| | | per | per | | |
| Year | PubMed Accession Number-Replikin Count | year | year | S.D. | Significance |

VI. Human H5N1 pB1 Area

| | | | | | |
|---|---|---|---|---|---|
| 2003 | BAE07199 15 | 1 | 2.0 | 0.0 | prev p < .30 |
| 2004 | ABE97546 15 ABE97545 15 ABE97544 15 ABE97543 15 | 91 | 2.0 | 0.1 | low p < .001, |
| | ABE97542 15 ABE97541 15 ABE97540 15 ABE97539 15 | | | | prev p < .30 |
| | ABE97538 15 ABE97537 15 ABE97536 15 AAV35116 15 | | | | |
| | AAV32652 15 AAV32644 15 ABE97535 15 ABE97534 15 | | | | |
| | ABE97533 15 ABE97532 15 ABE97531 15 ABE97530 15 | | | | |
| | ABE97529 15 ABE97528 15 ABE97527 15 ABE97526 15 | | | | |
| | ABE97525 15 ABE97524 15 ABE97523 15 ABE97522 15 | | | | |
| | ABE97521 15 ABE97520 15 ABE97519 15 ABE97518 15 | | | | |
| | ABE97517 15 ABE97516 15 ABE97515 15 ABE97514 15 | | | | |
| | ABE97513 15 ABE97512 15 ABE97511 15 ABE97509 15 | | | | |
| | ABE97508 15 ABE97507 15 ABE97506 15 ABE97505 15 | | | | |
| | ABE97504 15 ABE97503 15 ABE97502 15 ABE97501 15 | | | | |
| | ABE97500 15 ABE97499 15 ABE97498 15 ABE97497 15 | | | | |
| | ABE97496 15 ABE97495 15 ABE97494 15 ABE97493 15 | | | | |
| | ABE97492 15 ABE97491 15 ABE97490 15 ABE97489 15 | | | | |
| | ABE97488 15 ABE97487 15 ABE97486 15 ABE97485 15 | | | | |
| | ABE97484 15 ABE97483 15 ABE97482 15 ABE97481 15 | | | | |
| | ABE97480 15 ABE97479 15 ABE97478 15 ABE97477 15 | | | | |
| | ABE97476 15 ABE97475 15 ABE97474 15 ABE97473 15 | | | | |
| | ABE97472 15 ABE97471 15 ABE97470 15 ABE97469 15 | | | | |
| | ABE97468 15 ABE97467 15 ABE97466 15 ABE97465 15 | | | | |
| | ABE97464 15 ABE97463 15 ABE97462 15 ABE97461 15 | | | | |
| | ABE97460 15 ABE97510 15 AAV73985 3 | | | | |
| 2005 | ABI36230 16 ABI36225 16 ABI36220 16 ABI36216 16 | 13 | 8.0 | 7.7 | low p < .01, |
| | ABI36214 15 ABI36209 15 ABI36009 15 ABI36000 14 | | | | prev p < .005 |
| | ABG78564 15 ABF56656 27 ABG78565 14 ABD16290 15 | | | | |
| | ABC72648 15 | | | | |
| 2006 | ABK34974 14 ABL31777 16 ABL31774 21 ABL31763 21 | 48 | 16.1 | 5.7 | low p < .001, |
| | ABL31752 21 ABL31741 16 ABI49393 16 ABL07027 21 | | | | prev p < .001 |
| | ABL07016 16 ABL07005 16 ABI49404 16 ABI36472 16 | | | | |
| | ABI36461 16 ABI36452 16 ABI36441 16 ABI36430 16 | | | | |
| | ABI36420 16 ABI36408 16 ABI36397 16 ABI36386 16 | | | | |
| | ABI36375 16 ABI36364 16 ABI36353 15 ABI36342 15 | | | | |
| | ABI36331 15 ABI36320 15 ABI36309 16 ABI36303 16 | | | | |
| | ABI36292 16 ABI36283 16 ABI36271 16 ABI36268 16 | | | | |
| | ABI36265 16 ABI36261 16 ABI36257 16 ABI36252 16 | | | | |
| | ABI36249 16 ABI36244 14 ABI36241 14 ABI36236 14 | | | | |
| | ABI36232 14 ABI36195 16 ABI36184 16 ABI36174 16 | | | | |
| | ABI36163 16 ABI36152 16 ABI36141 16 ABI16502 14 | | | | |

Human H5n1 Hemagglutinin Area

| | | | | | |
|---|---|---|---|---|---|
| 2003 | BAE07201 22 | 1 | 3.9 | 0.0 | |
| 2004 | AAS65618 23 AAS65615 22 ABE97634 22 ABE97633 22 | 104 | 3.9 | 0.3 | low p < .001, |
| | ABE97632 22 ABE97631 26 ABE97630 22 ABE97629 22 | | | | prev p > .50 |
| | ABE97628 22 ABE97627 22 ABE97626 21 ABE97625 22 | | | | |
| | ABE97624 22 AAV34704 22 AAS89004 22 AAV32636 22 | | | | |
| | AAV65826 22 ABE97623 22 ABE97622 22 ABE97621 22 | | | | |
| | ABE97620 22 ABE97619 22 ABE97618 22 ABE97617 22 | | | | |
| | ABE97616 22 ABE97615 22 ABE97614 22 ABE97613 20 | | | | |
| | ABE97612 22 ABE97611 20 ABE97610 19 ABE97609 22 | | | | |
| | ABE97608 22 ABE97607 22 ABE97606 22 ABE97605 22 | | | | |
| | ABE97604 11 ABE97603 22 ABE97602 22 ABE97601 22 | | | | |
| | ABE97600 22 ABE97599 22 ABE97598 22 ABE97597 22 | | | | |
| | ABE97596 22 ABE97595 22 ABE97594 22 ABE97593 22 | | | | |
| | ABE97592 22 ABE97591 18 ABE97590 18 ABE97589 18 | | | | |
| | ABE97588 22 ABE97587 22 ABE97586 22 ABE97585 22 | | | | |
| | ABE97584 22 ABE97583 21 ABE97582 22 ABE97581 22 | | | | |
| | ABE97580 22 ABE97579 22 ABE97578 22 ABE97577 22 | | | | |
| | ABE97576 22 ABE97575 24 ABE97574 22 ABE97573 22 | | | | |
| | ABE97572 22 ABE97571 22 ABE97570 22 ABE97569 22 | | | | |
| | ABE97568 22 ABE97567 22 ABE97566 22 ABE97565 22 | | | | |
| | ABE97564 21 ABE97563 22 ABE97562 22 ABE97561 22 | | | | |
| | ABE97560 22 ABE97559 22 ABE97558 22 ABE97557 19 | | | | |
| | ABE97556 23 ABE97555 22 ABE97554 24 ABE97553 22 | | | | |
| | ABE97552 22 ABE97551 22 ABE97550 22 ABE97549 22 | | | | |
| | ABE97548 23 ABE97547 22 AAW59559 8 AAW59558 7 | | | | |
| | AAW59556 4 AAW59554 6 AAW59552 16 AAW59550 16 | | | | |
| | AAW59548 12 AAV73980 16 AAV73975 8 AAV73972 16 | | | | |
| 2005 | ABC59833 17 ABB00582 17 ABG78567 17 ABG78549 17 | 18 | 4.1 | 0.3 | low p < .001, |
| | ABG20478 21 ABG20476 14 ABC70167 17 ABB86287 17 | | | | prev p < .02 |
| | ABC72655 22 ABF56648 21 ABI36045 23 ABI36044 23 | | | | |
| | ABI36043 27 ABI36042 27 ABI36041 23 ABI36040 21 | | | | |
| | ABI36012 23 ABD16284 22 | | | | |
| 2006 | ABD66293 21 ABG23657 24 ABI16504 27 ABG20472 21 | 64 | 4.1 | 0.3 | low p < .001, |
| | ABG20468 23 ABJ90343 21 ABG45944 19 ABM54180 21 | | | | prev p > .50 |
| | ABM54179 21 ABL31780 23 ABI49415 25 ABI49396 23 | | | | |
| | ABL31766 23 ABL31755 23 ABL31744 23 ABL07030 23 | | | | |
| | ABL07019 23 ABL07008 23 ABK32782 20 ABK32780 21 | | | | |
| | ABK32781 21 ABK32779 21 ABK32778 21 ABK32777 21 | | | | |
| | ABK32776 21 ABK32775 20 ABI49407 23 ABI36480 23 | | | | |
| | ABI36469 23 ABI36450 22 ABI36439 23 ABI36428 23 | | | | |
| | ABI36423 22 ABI36406 22 ABI36395 23 ABI36384 23 | | | | |
| | ABI36373 23 ABI36362 22 ABI36351 23 ABI36340 23 | | | | |
| | ABI36329 22 ABI36318 22 ABI36307 23 ABI36295 23 | | | | |
| | ABI36286 23 ABI36275 23 ABI36198 23 ABI36187 22 | | | | |
| | ABI36177 22 ABI36166 22 ABI36155 22 ABI36144 22 | | | | |
| | ABI36057 23 ABI36056 23 ABI36055 23 ABI36054 23 | | | | |
| | ABI36053 23 ABI36051 23 ABI36050 23 ABI36049 23 | | | | |
| | ABI36048 23 ABI36047 23 ABI36046 23 ABI23979 21 | | | | |

Human H5N1 NS

| | | | | | |
|---|---|---|---|---|---|
| 2004 | AAV35114 2 AAV35113 2 | 2 | 1.7 | 0.0 | low p < .002 |
| 2005 | ABF56654 27 ABF56653 27 | 2 | 3.8 | 0.0 | low p < .005 |
| 2006 | ABI16510 2 ABI16509 2 | 2 | 1.7 | 0.0 | low p < .002 |

Human H5N1 Pa

| | | | | | |
|---|---|---|---|---|---|
| 2003 | BAE07200 27 | 1 | 3.8 | 0.0 | |
| 2004 | ABL67793 27 ABL67792 27 ABL67791 27 ABL67780 31 | 102 | 4.0 | 0.5 | low p < .001 |
| | ABL67769 27 ABE97897 29 ABE97896 29 ABE97895 29 | | | | |
| | ABE97894 29 ABE97893 29 ABE97892 29 ABE97891 29 | | | | |
| | ABE97890 29 ABE97889 29 ABE97888 29 AAV35115 24 | | | | |
| | AAV32651 27 AAV32643 27 AAW59560 1 AAW59557 10 | | | | |
| | AAW59551 27 AAW59549 27 ABE97887 29 ABE97886 29 | | | | |
| | ABE97885 29 ABE97884 29 ABE97883 29 ABE97882 29 | | | | |
| | ABE97881 29 ABE97880 29 ABE97879 29 ABE97878 29 | | | | |
| | ABE97877 29 ABE97876 29 ABE97875 29 ABE97874 29 | | | | |
| | ABE97873 29 ABE97872 29 ABE97871 29 ABE97870 29 | | | | |
| | ABE97869 29 ABE97868 29 ABE97867 29 ABE97866 29 | | | | |
| | ABE97865 29 ABE97864 29 ABE97863 29 ABE97862 29 | | | | |
| | ABE97861 29 ABE97860 29 ABE97859 29 ABE97858 29 | | | | |
| | ABE97857 29 ABE97856 29 ABE97855 29 ABE97854 29 | | | | |
| | ABE97853 29 ABE97852 29 ABE97851 29 ABE97850 29 | | | | |
| | ABE97849 29 ABE97848 29 ABE97847 29 ABE97846 29 | | | | |
| | ABE97845 29 ABE97844 29 ABE97843 29 ABE97842 29 | | | | |
| | ABE97841 29 ABE97840 29 ABE97839 29 ABE97838 29 | | | | |
| | ABE97837 29 ABE97836 29 ABE97835 29 ABE97834 29 | | | | |
| | ABE97833 29 ABE97832 29 ABE97831 29 ABE97830 29 | | | | |
| | ABE97829 29 ABE97828 29 ABE97827 29 ABE97826 29 | | | | |
| | ABE97825 29 ABE97824 29 ABE97823 29 ABE97822 29 | | | | |
| | ABE97821 29 ABE97820 29 ABE97819 29 ABE97818 29 | | | | |
| | ABE97817 29 ABE97816 29 ABE97815 29 ABE97814 29 | | | | |
| | ABE97813 29 ABE97812 29 ABE97811 29 AAV73982 7 | | | | |
| | AAV73977 4 AAV73974 4 | | | | |
| 2005 | ABL67826 27 ABL67815 28 ABL67804 28 ABI36229 33 | 15 | 3.8 | 0.4 | low p > .50, |
| | ABI36224 33 ABI36222 26 ABI36219 26 ABI36212 27 | | | | prev p < .05 |
| | ABI36011 27 ABI36002 27 ABG78563 22 ABG78562 22 | | | | |
| | ABF56655 27 ABD16289 27 ABC72649 24 | | | | |
| 2006 | ABK34973 28 ABL31779 31 ABL31765 27 ABL31754 27 | 47 | 3.8 | 0.3 | low p < .20, |
| | ABL31743 27 ABI49414 27 ABI49395 27 ABL07029 27 | | | | prev p > .50 |
| | ABL07018 31 ABL07007 31 ABI49406 27 ABI36481 26 | | | | |
| | ABI36470 26 ABI36451 27 ABI36440 27 ABI36429 27 | | | | |
| | ABI36419 27 ABI36407 27 ABI36396 32 ABI36385 32 | | | | |
| | ABI36374 32 ABI36363 27 ABI36352 27 ABI36341 27 | | | | |
| | ABI36330 23 ABI36319 23 ABI36308 27 ABI36302 27 | | | | |
| | ABI36291 27 ABI36282 27 ABI36273 27 ABI36267 27 | | | | |
| | ABI36260 27 ABI36256 27 ABI36255 27 ABI36251 27 | | | | |
| | ABI36247 27 ABI36240 27 ABI36238 27 ABI36235 27 | | | | |
| | ABI36197 27 ABI36186 27 ABI36176 27 ABI36165 27 | | | | |
| | ABI36154 27 ABI36143 27 ABI16503 24 | | | | |

Human H5N1 Neuraminidase

| | | | | | |
|---|---|---|---|---|---|
| 2003 | BAE07203 8 | 1 | 1.7 | 0.0 | |
| 2004 | AAS65617 27 AAS65616 24 ABE97722 23 ABE97721 23 | 100 | 5.0 | 0.3 | low p < .001, |
| | ABE97720 23 ABE97719 23 ABE97718 23 ABE97717 23 | | | | prev p < .001 |
| | ABE97716 23 ABE97715 23 ABE97714 23 ABE97713 23 | | | | |
| | ABE97712 23 AAS89006 18 AAS89005 16 AAV32637 24 | | | | |
| | ABL67796 18 ABL67783 18 ABL67772 18 AAV65827 18 | | | | |
| | ABE97692 23 ABE97711 23 ABE97710 23 ABE97709 23 | | | | |
| | ABE97708 23 ABE97707 23 ABE97706 23 ABE97705 23 | | | | |
| | ABE97704 23 ABE97703 23 ABE97702 23 ABE97701 23 | | | | |
| | ABE97700 23 ABE97699 23 ABE97698 23 ABE97697 23 | | | | |
| | ABE97696 23 ABE97695 23 ABE97694 23 ABE97693 23 | | | | |
| | ABE97691 23 ABE97690 23 ABE97689 23 ABE97688 23 | | | | |
| | ABE97687 23 ABE97686 23 ABE97685 23 ABE97684 23 | | | | |
| | ABE97683 23 ABE97682 23 ABE97681 23 ABE97680 23 | | | | |
| | ABE97679 23 ABE97678 23 ABE97677 23 ABE97676 23 | | | | |
| | ABE97675 23 ABE97674 23 ABE97673 23 ABE97672 23 | | | | |
| | ABE97671 23 ABE97670 23 ABE97669 23 ABE97668 23 | | | | |
| | ABE97667 23 ABE97666 23 ABE97665 23 ABE97664 23 | | | | |
| | ABE97663 23 ABE97662 23 ABE97661 23 ABE97660 23 | | | | |
| | ABE97659 23 ABE97658 23 ABE97657 23 ABE97656 23 | | | | |
| | ABE97655 23 ABE97654 23 ABE97653 23 ABE97652 23 | | | | |
| | ABE97651 23 ABE97650 23 ABE97649 23 ABE97648 23 | | | | |
| | ABE97647 23 ABE97646 23 ABE97645 23 ABE97644 23 | | | | |
| | ABE97643 23 ABE97642 23 ABE97641 23 ABE97640 23 | | | | |
| | ABE97639 23 ABE97638 23 ABE97637 23 ABE97636 23 | | | | |
| | ABE97635 23 AAV73978 8 AAV73976 8 AAV73973 18 | | | | |
| 2005 | ABC59832 6 ABB00581 8 ABL67829 18 ABL67818 18 | 18 | 3.0 | 0.8 | low p < .001, |
| | ABL67807 18 ABG78555 8 ABG78554 6 ABC72646 16 | | | | prev p < .001 |
| | ABC70166 8 ABB86286 8 ABF56651 27 ABI36097 13 | | | | |
| | ABI36095 13 ABI36094 13 ABI36085 13 ABI36084 13 | | | | |
| | ABI36014 13 ABD16286 20 | | | | |
| 2006 | ABG23658 12 ABI16506 13 ABG20474 7 ABG20470 8 | 51 | 2.8 | 0.4 | low p < .001, |
| | ABM68050 8 ABM68049 8 ABM68048 8 ABL31782 15 | | | | prev p < .20 |
| | ABL31768 13 ABL31757 13 ABL31746 13 ABI49417 13 | | | | |
| | ABI49398 13 ABL07032 12 ABL07021 15 ABL07010 15 | | | | |
| | ABI49409 13 ABI36476 13 ABI36465 13 ABI36457 13 | | | | |
| | ABI36446 13 ABI36435 13 ABI36426 13 ABI36413 13 | | | | |
| | ABI36402 13 ABI36391 13 ABI36380 13 ABI36369 13 | | | | |
| | ABI36358 13 ABI36347 13 ABI36336 13 ABI36325 13 | | | | |
| | ABI36314 13 ABI36298 13 ABI36287 13 ABI36278 13 | | | | |
| | ABI36200 13 ABI36189 13 ABI36179 13 ABI36168 13 | | | | |
| | ABI36157 13 ABI36146 13 ABI36096 13 ABI36093 13 | | | | |
| | ABI36092 13 ABI36090 13 ABI36089 11 ABI36088 11 | | | | |
| | ABI36087 13 ABI36086 13 ABI23981 7 | | | | |
| 2007 | ABO38180 8 ABM90546 13 ABM90535 13 ABM90524 13 | 13 | 3.0 | 0.5 | low p < .001, |
| | ABM90513 13 ABM90502 15 ABM90491 15 ABM90480 15 | | | | prev p < .05 |
| | ABM90469 15 ABM90458 15 ABM90447 15 ABM90436 15 | | | | |
| | ABQ43810 8 | | | | |

Human H5N1 Pb2 Area

| | | | | | |
|---|---|---|---|---|---|
| 2003 | BAE07198 18 BAE07197 18 | 2 | 2.4 | 0.0 | |
| 2004 | ABL67788 18 ABL67777 18 ABL67766 18 ABF01751 21 | 73 | 2.8 | 0.3 | low p < .001, |
| | ABF01750 21 ABF01749 21 ABF01748 21 ABF01747 21 | | | | prev p < .001 |
| | ABF01746 21 ABF01745 21 ABF01744 21 ABF01743 21 | | | | |
| | AAV35117 18 AAV32653 17 AAV32645 21 ABF01742 21 | | | | |
| | ABF01741 21 ABF01740 21 ABF01739 21 ABF01738 21 | | | | |
| | ABF01737 21 ABF01736 21 ABF01735 21 ABF01734 21 | | | | |
| | ABF01733 21 ABF01732 21 ABF01731 21 ABF01728 21 | | | | |
| | ABF01726 21 ABF01724 21 ABF01722 21 ABF01709 21 | | | | |
| | ABF01708 21 ABF01706 21 ABF01705 21 ABF01704 21 | | | | |
| | ABF01703 21 ABF01702 21 ABF01701 21 ABF01700 21 | | | | |
| | ABF01699 21 ABF01698 21 ABF01697 21 ABF01696 21 | | | | |
| | ABF01695 21 ABF01694 21 ABF01693 21 ABF01692 21 | | | | |
| | ABF01691 21 ABF01690 21 ABF01689 21 ABF01688 21 | | | | |
| | ABF01687 21 ABF01686 21 ABF01685 21 ABF01684 21 | | | | |
| | ABF01683 21 ABF01682 21 ABF01681 21 ABF01680 21 | | | | |
| | ABF01679 21 ABF01678 21 ABF01677 21 ABF01676 21 | | | | |
| | ABF01675 21 ABF01674 21 ABF01673 21 ABF01672 21 | | | | |
| | ABF01670 21 ABF01669 21 ABF01668 21 ABF01667 21 | | | | |
| | AAV73984 12 | | | | |
| 2005 | ABL67823 18 ABL67812 18 ABL67801 18 ABI36228 18 | 16 | 2.4 | 0.4 | low p > .50, |
| | ABI36227 18 ABI36223 18 ABI36218 18 ABI36213 18 | | | | prev p < .001 |
| | ABI36211 19 ABI36008 19 ABI35999 18 ABG78566 13 | | | | |
| | ABF56657 27 ABG78548 13 ABD16291 18 ABC72647 18 | | | | |
| 2006 | ABL31776 18 ABL31773 18 ABL31762 18 ABL31751 18 | 48 | 2.4 | 0.1 | low p > .50, |
| | ABL31740 18 ABI49392 18 ABL07026 18 ABL07015 18 | | | | prev p > .50 |
| | ABL07004 18 ABI49403 18 ABI36482 18 ABI36471 18 | | | | |
| | ABI36454 18 ABI36443 18 ABI36432 18 ABI36422 18 | | | | |
| | ABI36410 18 ABI36399 18 ABI36388 18 ABI36377 18 | | | | |
| | ABI36366 18 ABI36355 17 ABI36344 17 ABI36333 18 | | | | |
| | ABI36322 18 ABI36311 18 ABI36306 18 ABI36305 18 | | | | |
| | ABI36294 18 ABI36285 18 ABI36274 18 ABI36270 18 | | | | |
| | ABI36264 18 ABI36263 18 ABI36259 18 ABI36254 18 | | | | |
| | ABI36248 18 ABI36246 18 ABI36243 18 ABI36239 18 | | | | |
| | ABI36234 18 ABI36194 18 ABI36173 18 ABI36162 18 | | | | |
| | ABI36151 18 ABI36140 18 ABG23659 21 ABI16501 18 | | | | |

human H5N1 matrix area

| | | | | | |
|---|---|---|---|---|---|
| 2003 | BAE07204 3 | 1 | 1.2 | 0.0 | prev p < .05 |
| 2004 | AAV35110 3 AAV32646 3 AAV32638 3 AAV35111 2 | 7 | 1.6 | 0.4 | low p < .04 |
| | AAV32647 2 AAV32639 2 AAV73981 3 | | | | |
| 2005 | ABF01926 3 ABF01924 3 ABF01922 3 ABF01920 3 | 198 | 1.6 | 0.7 | low p < .001, |
| | ABF01918 3 ABF01916 3 ABF01914 3 ABF01912 3 | | | | prev p > .50 |
| | ABF01910 3 ABF01908 3 ABF01906 3 ABF01927 2 | | | | |
| | ABF01925 2 ABF01923 1 ABF01921 2 ABF01919 2 | | | | |
| | ABF01917 2 ABF01915 2 ABF01913 2 ABF01911 2 | | | | |
| | ABF01909 2 ABF01907 2 ABG78552 3 ABG78551 3 | | | | |
| | ABG20477 3 ABF56649 3 ABI36082 5 ABI36080 3 ABI36060 | | | | |
| | 5 ABI36058 3 ABI36015 3 ABI36004 3 ABF01904 3 | | | | |
| | ABF01902 3 ABF01900 3 ABF01898 3 ABF01896 3 | | | | |
| | ABF01894 3 ABF01892 3 ABF01890 3 ABF01888 3 | | | | |
| | ABF01886 3 ABF01884 3 ABF01882 3 ABF01880 3 | | | | |
| | ABF01878 3 ABF01876 3 ABF01874 3 ABF01872 3 | | | | |
| | ABF01870 3 ABF01868 3 ABF01866 3 ABF01864 3 | | | | |
| | ABF01862 3 ABF01860 3 ABF01858 3 ABF01856 3 | | | | |
| | ABF01854 3 ABF01852 3 ABF01850 3 ABF01848 3 | | | | |
| | ABF01846 3 ABF01844 3 ABF01842 3 ABF01840 3 | | | | |
| | ABF01838 3 ABF01836 3 ABF01834 3 ABF01832 3 | | | | |
| | ABF01830 3 ABF01828 3 ABF01826 3 ABF01824 3 | | | | |
| | ABF01822 3 ABF01820 3 ABF01818 3 ABF01816 3 | | | | |
| | ABF01814 3 ABF01812 3 ABF01810 3 ABF01808 3 | | | | |
| | ABF01806 3 ABF01804 3 ABF01802 3 ABF01800 3 | | | | |
| | ABF01798 3 ABF01796 3 ABF01794 3 ABF01792 3 | | | | |
| | ABF01790 3 ABF01788 3 ABF01786 3 ABF01784 3 | | | | |
| | ABF01782 3 ABF01780 3 ABF01778 3 ABF01776 3 | | | | |
| | ABF01774 3 ABF01772 3 ABF01770 3 ABF01768 3 | | | | |
| | ABF01766 3 ABF01764 3 ABF01762 3 ABF01760 3 | | | | |
| | ABF01758 3 ABF01756 3 ABF01754 3 ABF01752 3 | | | | |
| | ABD16285 3 ABC72651 3 ABF56650 2 ABI36083 2 | | | | |
| | ABI36081 4 ABI36061 2 ABI36059 4 ABI36016 4 ABI36005 4 | | | | |
| | ABF01905 2 ABF01903 2 ABF01901 1 ABF01899 2 | | | | |
| | ABF01897 2 ABF01895 2 ABF01893 2 ABF01891 2 | | | | |
| | ABF01889 2 ABF01887 2 ABF01885 2 ABF01883 1 | | | | |
| | ABF01881 1 ABF01879 2 ABF01877 2 ABF01875 2 | | | | |
| | ABF01873 2 ABF01871 2 ABF01869 2 ABF01867 2 | | | | |
| | ABF01865 2 ABF01863 2 ABF01861 1 ABF01859 2 | | | | |
| | ABF01857 2 ABF01855 2 ABF01853 2 ABF01851 2 | | | | |
| | ABF01849 2 ABF01847 2 ABF01845 2 ABF01843 2 | | | | |
| | ABF01841 2 ABF01839 2 ABF01837 2 ABF01835 2 | | | | |
| | ABF01833 2 ABF01831 1 ABF01829 2 ABF01827 1 | | | | |
| | ABF01825 2 ABF01823 2 ABF01821 2 ABF01819 2 | | | | |
| | ABF01817 2 ABF01815 2 ABF01813 2 ABF01811 2 | | | | |
| | ABF01809 2 ABF01807 2 ABF01805 2 ABF01803 2 | | | | |
| | ABF01801 2 ABF01799 2 ABF01797 2 ABF01795 1 | | | | |
| | ABF01793 1 ABF01791 4 ABF01789 2 ABF01787 2 | | | | |
| | ABF01785 4 ABF01783 2 ABF01781 1 ABF01779 2 | | | | |
| | ABF01777 2 ABF01775 1 ABF01773 2 ABF01771 2 | | | | |
| | ABF01769 4 ABF01767 2 ABF01765 2 ABF01763 1 | | | | |
| | ABF01761 1 ABF01759 1 ABF01757 1 ABF01755 4 | | | | |
| | ABF01753 2 ABC72652 2 ABG78553 1 ABG78550 1 | | | | |
| 2006 | ABG23663 3 ABI16508 3 ABG20473 3 ABG20469 3 | 93 | 2.0 | 0.7 | low p < .001, |
| | ABG23664 2 ABL31783 5 ABL31769 5 ABL31758 5 | | | | prev p < .001 |
| | ABL31747 5 ABI49418 5 ABI49399 5 ABL07033 5 ABL07022 | | | | |
| | 5 ABL07011 5 ABI36062 5 ABI49410 5 ABI36483 5 | | | | |
| | ABI36474 5 ABI36463 5 ABI36455 5 ABI36444 5 ABI36433 5 | | | | |
| | ABI36424 3 ABI36411 3 ABI36400 5 ABI36389 5 ABI36378 5 | | | | |
| | ABI36367 3 ABI36356 3 ABI36345 3 ABI36334 3 ABI36323 3 | | | | |
| | ABI36312 5 ABI36296 5 ABI36276 5 ABI36201 5 ABI36190 3 | | | | |
| | ABI36180 3 ABI36169 3 ABI36158 3 ABI36147 3 ABI36078 3 | | | | |
| | ABI36076 3 ABI36074 3 ABI36070 5 ABI36068 5 ABI36066 5 | | | | |
| | ABI36064 5 ABI23980 3 ABL31784 2 ABL31770 2 ABL31759 | | | | |
| | 2 ABL31748 2 ABI49419 2 ABI49400 2 ABL07034 2 | | | | |
| | ABL07023 2 ABL07012 2 ABI36063 2 ABI49411 2 ABI36484 | | | | |
| | 2 ABI36475 2 ABI36464 2 ABI36456 2 ABI36445 2 ABI36434 | | | | |
| | 2 ABI36425 2 ABI36412 2 ABI36401 2 ABI36390 2 ABI36379 | | | | |
| | 2 ABI36368 2 ABI36357 4 ABI36346 4 ABI36335 4 ABI36324 | | | | |
| | 4 ABI36313 2 ABI36297 2 ABI36277 2 ABI36202 2 ABI36191 | | | | |
| | 2 ABI36181 2 ABI36170 2 ABI36159 2 ABI36148 2 ABI36079 | | | | |
| | 4 ABI36077 4 ABI36075 4 ABI36071 2 ABI36069 2 ABI36067 | | | | |
| | 2 ABI36065 2 ABI16507 2 | | | | |

Human H5N1 Nucleocapsid Area

| | | | | | |
|---|---|---|---|---|---|
| 2004 | ABL67795 2 ABL67782 2 ABL67771 2 AAV35112 2 | 4 | 0.4 | 0.0 | |
| 2005 | ABL67828 2 ABL67817 2 ABL67806 2 ABI36110 2 ABI36109 | 10 | 0.4 | 0.0 | low p < .001, |
| | 2 ABI36108 2 ABI36099 2 ABI36098 2 ABI36013 2 ABI36003 2 | | | | prev p > .50 |
| 2006 | ABL31781 2 ABL31767 2 ABL31756 2 ABL31745 2 | 43 | 0.4 | 0.0 | low p < .001, |
| | ABI49416 2 ABI49397 2 ABL07031 2 ABL07020 2 ABL07009 | | | | prev p > .50 |
| | 2 ABI49408 2 ABI36477 2 ABI36466 2 ABI36458 2 ABI36447 | | | | |
| | 2 ABI36436 2 ABI36427 2 ABI36414 2 ABI36403 2 ABI36392 | | | | |
| | 2 ABI36381 2 ABI36370 2 ABI36359 2 ABI36348 2 ABI36337 | | | | |
| | 2 ABI36326 2 ABI36315 2 ABI36299 2 ABI36288 2 ABI36279 | | | | |
| | 2 ABI36199 2 ABI36188 2 ABI36178 2 ABI36167 2 ABI36156 | | | | |
| | 2 ABI36145 2 ABI36107 2 ABI36106 2 ABI36105 2 ABI36104 | | | | |
| | 2 ABI36103 2 ABI36102 2 ABI36101 2 ABI36100 2 | | | | |

Analysis of the number of Replikin sequences present in the areas in the genome adjacent to the pB1 area revealed no more than a two-fold increase in Replikin Count in the seven other areas of the genome as compared to an eight-fold increase (p<0.001) in the Replikin Count in the pB1 area between years 2003 to 2006. The specificity of the localization of the upregulated RPG in the pB1 area is underlined by the fact that other parts of the polymerase gene area of which pB1 is a part, namely, the pB2 and pA gene areas do not have the same amount of increase in Replikin Count even though the gene areas are immediately adjacent to pB1.

In the illustration, the standard deviation of the means is shown in light gray columns on top of the means, rather than in the usual ‘T’ symbols, to emphasize the diverse expanding virus population with regard to the Replikin Count. As Replikin Count increases in a population, a diversity of Replikin Counts may be observed as the lethality and virulence of the virus increases. An increasing standard deviation within a virus population is, therefore, itself an index of viral outbreaks. Here in FIG. 1 and in the figures that follow, small standard deviations from mean Replikin Count are seen to accompany quiescent inter-outbreak periods of the virus.

Examples 1-3 are provided below as examples of analysis of Replikin Peak Genes in sequences publicly available in accession numbers at PubMed. Examples 2 and 3 illustrate how identification of a Replikin Peak Gene allows for a magnification of the effect of increases in Replikin Count in an isolate where the increase may be correlated with and predict increases in virulence and lethality. For example, Example 2 provides a 2003 isolate of H5N1 from Hong Kong with a whole pB1 gene area (SEQ ID NO: 1683) Replikin Count of 2.0 and an RPG Replikin Count of 14.6. Example 3 provides a 2006 isolate from Indonesia with a whole pB1 gene area Replikin Count of 17.8 and an RPG Replikin Count of 22.5. Indonesia experienced a highly lethal outbreak of H5N1 with evidence of human to human transmission in 2007. The high Replikin Counts in isolates from Indonesia in 2006 allowed the inventors to prospectively predict the lethal Indonesian outbreak.

The Replikin count of the whole genome from the 2006 Indonesian isolate demonstrates a significant increase as compared to the 2003 Hong Kong isolate. The isolation of the Replikin peak gene (RPG) area, that is the area of the genome which shows the highest concentration (count) of continuous Replikins per 100 amino acids, magnifies the effect. For this reason, whole genome counts are used for first approximations of Replikin count increases, and where more detailed specific gene areas or open reading frame data is not available. However, when available, the RPG is used for more definitive “higher power” examinations. This is illustrated in FIGS. 1-4.

B. Increasing Replikin Count in RPG of Influenza A Associated with Pandemics and Lethality

The inventors have now associated an increase in Replikin Count in the RPG of Influenza A virus with pandemics, epidemics and lethal outbreaks of influenza. FIG. 2 illustrates an increase in Replikin Count before and accompanying each Influenza A pandemic and outbreak since 1918 and low Replikin Counts during quiescent periods of Influenza A infection and continually in non-lethal Influenza B. The graph provides annual Replikin Counts from 1917-2007 for all Replikin Peak Genes isolated in silico in the pB1 gene area of influenza strains having amino acid or nucleic acid sequences publicly available at PubMed. The total number of sequences analyzed for the data is 14,227. The Replikin Count of each influenza-in-silico isolate was obtained separately and objectively through time for each species by computer software (FluForecast®, available through Replikins LLC, Boston, Mass.). The software queried publicly available sequences at www.pubmed.com. The software measures solely the number of Replikins per 100 amino acids in the publicly available sequences and provides a mean Replikin Count with standard deviation from the mean for all isolates available in a given strain of influenza in a given year.

Over a ninety year period, the graph in FIG. 2 demonstrates an increase in Replikin Count before and accompanying each Influenza A pandemic and outbreak, namely, the 1918 H1N1 pandemic, the 1930's H1N1 epidemic, the 1957H2N2 pandemic, the 1968H3N1 pandemic, the 1977-78H3N2 outbreaks and the H5N1 outbreaks of 1997, 2001-2004 and 2007. In FIG. 2, p values at <0.001 are supportive of the significance of the differences between the pandemic and epidemic groups on the one hand and clinically quiescent periods on the other hand.

Over the same ninety year period, constant low Replikin Counts of less than four may be observed during quiescent non-lethal periods between epidemic outbreaks in all 3 pandemic strains of Influenza A including H1N1, H2N2, H3N2 and low Replikin Counts of less than four may also be observed in human H5N1 and H5N1 in chicken in relatively quiescent periods. Low Replikin Counts may likewise be observed in non-lethal Influenza B through the entire period of observation. In particular, the absence of increase in Replikin Count above five in Influenza B corresponds to the absence of any observed lethal outbreak. As such, during the observation period, Influenza B is always non-lethal. This absence of Replikin Counts of five or more in non-lethal Influenza B provides an important control for the study of Replikin Count as a correlate of lethality. In Influenza A, an increase in Replikin Count indicates an increase in lethality and a decrease in Replikin Count indicates a decrease in lethality.

Analysis of publicly available sequences for isolates of Influenza B virus between 1940 and 2007 is provided below in Table 2. Years in which not data was available are not included in the table.

TABLE 2
Influenza B

| | | | Mean | | |
|---|---|---|---|---|---|
| | | No. of | Replikin | | |
| | | Isolates | Count | | |
| | | per | per | | |
| Year | PubMed Accession Number-Replikin Count | year | year | S.D. | Significance |
| 1940 | NP_056657 14 ABG85176 14 ABG85165 14 P07832 14 | 10 | 1.8 | 0.3 | low p < .001 |
| | BAA00002 14 AAA43767 14 AAF06886 16 AAF06851 10 | | | | |
| | NP_056659 16 NP_056658 10 | | | | |
| 1966 | AAF89738 25 P13872 14 P13871 14 Q9IMP4 25 | 4 | 2.6 | 0.8 | low p < .10 |
| 1969 | ABQ81851 14 | 1 | 1.9 | 0.0 | prev p < .10 |
| 1972 | ABF21251 18 ABF21252 18 | 2 | 2.4 | 0.0 | |
| 1979 | AAF06873 14 AAF06856 10 | 2 | 1.6 | 0.4 | low p < .05, |
| | | | | | prev p < .10 |
| 1984 | AAF06870 14 AAF06888 16 AAF06853 10 | 3 | 1.8 | 0.5 | low p < .02, |
| | | | | | prev p > .50 |
| 1985 | AAF06868 14 AAF06885 16 AAF06850 10 | 3 | 1.8 | 0.5 | low p < .02, |
| | | | | | prev p > .50 |
| 1987 | ABL77253 14 AAF06874 17 AAF06891 14 AAF06857 10 | 4 | 1.8 | 0.4 | low p < .002, |
| | | | | | prev p > .50 |
| 1988 | ABN50611 14 ABL77264 14 AAF06875 14 AAF06860 14 | 9 | 1.8 | 0.3 | low p < .001, |
| | AAF06892 17 AAF06877 14 AAF06858 10 AAF06842 10 | | | | prev p > .50 |
| | P12236 6 | | | | |
| 1989 | ABL77275 14 P21796 5 | 2 | 1.8 | 0.1 | low p < .01, |
| | | | | | prev p > .50 |
| 1990 | ABN58670 16 ABN50633 14 ABN50622 14 ABL76703 14 | 6 | 1.9 | 0.1 | low p < .001, |
| | AAB72043 14 O36430 14 | | | | prev p < .20 |
| 1991 | ABN50644 27 ABN51204 10 ABN51193 10 ABL77286 14 | 4 | 2.0 | 1.1 | low p < .05, |
| | | | | | prev p > .50 |
| 1992 | ABN50655 14 ABL77308 14 ABL77297 14 | 3 | 1.9 | 0.0 | prev p > .50 |
| 1993 | CAA05486 40 ABN50666 14 ABL77341 14 ABL77330 14 | 15 | 2.1 | 1.0 | low p < .001, |
| | ABL77319 14 AAF06869 14 AAF06865 14 AAF06861 14 | | | | prev < .40 |
| | CAG96502 14 AAF06887 17 AAF06882 14 AAF06878 17 | | | | |
| | AAF06852 10 AAF06846 10 AAF06843 10 | | | | |
| 1994 | AAU94857 14 AAU94856 14 AAU94855 14 AAU94854 14 | 29 | 1.8 | 0.2 | low p < .001, |
| | AAU94853 14 AAU94852 14 AAU94851 14 AAU94850 14 | | | | prev p < .40 |
| | AAU94849 14 AAU94848 14 AAU94847 14 AAU94846 14 | | | | |
| | AAU94845 14 AAF89734 14 ABR16004 14 ABN50721 14 | | | | |
| | ABN50710 14 ABN50699 14 ABN50688 14 ABN50677 14 | | | | |
| | ABL77363 14 ABL77352 14 ABL77000 14 AAF06866 15 | | | | |
| | AAF06864 14 AAF06883 17 AAF06881 14 AAF06848 10 | | | | |
| | AAF06847 10 | | | | |
| 1995 | ABR16015 14 ABN50732 14 ABL77385 14 ABL77374 14 | 5 | 1.9 | 0.0 | prev p > .50 |
| | ABL76945 14 | | | | |
| 1996 | ABL76967 14 ABL76714 14 | 2 | 1.9 | 0.0 | |
| 1997 | ABN59454 14 ABN50413 14 ABN50402 14 ABN50391 14 | 20 | 1.8 | 0.2 | low p < .001, |
| | ABL76978 14 ABL76285 14 ABL76274 14 ABL76263 14 | | | | prev p > .50 |
| | AAK95906 14 AAF06867 14 AAF06862 14 CAG96500 14 | | | | |
| | ABI96727 14 ABI96738 13 AAP22114 14 AAP22106 14 | | | | |
| | AAF06884 17 AAF06879 17 AAF06849 10 AAF06844 10 | | | | |
| 1998 | ABN50743 14 ABN50512 14 ABN50457 14 ABL77022 14 | 23 | 1.8 | 0.3 | low p < .001, |
| | ABL77011 14 ABL76989 14 ABL76956 14 ABL76780 14 | | | | prev p > .50 |
| | ABL76769 14 ABL76296 14 AAF06876 14 AAF06872 14 | | | | |
| | AAF06871 14 AAF06863 14 AAU00993 14 AAF06893 17 | | | | |
| | AAF06890 17 AAF06889 17 AAF06880 17 AAF06859 10 | | | | |
| | AAF06855 10 AAF06854 10 AAF06845 10 | | | | |
| 1999 | ABL77055 14 ABL77044 14 ABL77033 14 ABL76813 14 | 14 | 1.9 | 0.2 | low p < .001, |
| | ABL76802 14 ABL76362 14 ABL76351 14 ABL76340 14 | | | | prev p < .40 |
| | ABL76329 14 ABL76318 14 ABL76307 14 ABQ81840 14 | | | | |
| | CAG96499 14 ABI94772 20 | | | | |
| 2000 | ABL77110 14 ABL77066 14 ABL76901 14 ABL76890 14 | 15 | 1.9 | 0.0 | low p < .001, |
| | ABL76879 14 ABL76868 14 ABL76857 14 ABL76824 14 | | | | prev p < .30 |
| | ABL76791 14 ABL76395 14 ABL76384 14 ABL76373 14 | | | | |
| | ABL84349 14 CAG96513 14 AAT69423 14 | | | | |
| 2001 | ABR15982 14 ABO72385 14 ABN50600 14 ABN50567 14 | 26 | 2.0 | 0.3 | low p < .001, |
| | ABN50534 14 ABN50523 14 ABN50490 14 ABN50479 14 | | | | prev p < .05 |
| | ABN50435 14 ABN50424 14 ABL77187 14 ABL77143 14 | | | | |
| | ABL77099 14 ABL77088 14 ABL77077 14 ABL76417 14 | | | | |
| | ABL76406 14 AAT69445 14 CAG96504 14 AAT69434 14 | | | | |
| | CAG96509 14 ABJ09524 14 ABJ09472 15 ABJ15707 16 | | | | |
| | ABI96775 20 ABI96695 16 | | | | |
| 2002 | ABN50754 14 ABN50578 14 ABN50556 14 ABN50545 14 | 32 | 2.0 | 0.3 | low p < .001, |
| | ABL77396 14 ABL77176 14 ABL77165 14 ABL77154 14 | | | | prev p > .50 |
| | ABL77132 14 ABL76483 14 ABL76472 14 ABL76461 14 | | | | |
| | ABL76450 14 ABL76439 10 ABL76428 14 CAG96515 14 | | | | |
| | CAG96514 14 CAG96511 14 CAG96510 14 CAG96503 14 | | | | |
| | CAG96501 14 ABI97312 16 ABJ09504 17 ABJ09486 15 | | | | |
| | ABI98925 16 ABI96765 15 ABK00110 15 ABI97340 15 | | | | |
| | ABI97331 21 ABI97322 21 ABI94786 14 ABI94737 11 | | | | |
| 2003 | ABR15993 14 ABN50589 14 ABL77209 14 ABL77198 14 | 39 | 1.9 | 0.2 | low p < .001, |
| | ABL76835 14 ABL76626 14 ABL76615 14 ABL76604 14 | | | | prev p < .10 |
| | ABL76593 14 ABL76582 14 ABL76571 14 ABL76560 14 | | | | |
| | ABL76549 14 ABL76538 14 ABL76527 14 ABL76516 14 | | | | |
| | ABL76505 14 ABL76494 14 CAG96520 14 CAG96519 14 | | | | |
| | CAG96517 14 CAG96512 14 CAG96508 14 CAG96507 14 | | | | |
| | CAG96506 14 CAG96505 14 ABJ98940 16 ABJ09534 15 | | | | |
| | ABJ09509 15 ABJ09482 15 ABI98936 14 ABI98912 15 | | | | |
| | ABI98908 15 ABK00142 16 ABK00088 8 ABJ80591 8 | | | | |
| | ABJ52572 8 ABJ52553 16 ABI97342 14 | | | | |
| 2004 | ABN50468 14 ABL77231 14 ABL77220 14 ABL77121 14 | 20 | 1.8 | 0.1 | low p < .001, |
| | ABL76923 14 ABL76912 14 ABL76846 14 ABL76758 14 | | | | prev p < .30 |
| | ABL76637 14 CAG96518 14 CAG96516 10 AAT70178 14 | | | | |
| | ABJ16471 15 ABJ09543 14 AAT78590 14 ABK00130 15 | | | | |
| | ABJ52559 14 ABJ15718 15 ABI97302 13 ABI96707 13 | | | | |
| 2005 | ABN50501 14 ABN50446 14 ABL77242 14 ABL76934 14 | 10 | 1.9 | 0.1 | low p < .001, |
| | ABL76692 14 ABL76681 14 ABL76670 14 ABL76659 14 | | | | prev p < .20 |
| | ABL76648 14 ABI96712 14 | | | | |
| 2006 | ABL76747 14 ABL76736 14 ABL76725 14 ABR16026 14 | 4 | 1.9 | 0.0 | low p < .001, |
| | | | | | prev p < .30 |
| 2007 | ZP_01998985 50 EDN71014 50 | 2 | 3.1 | 0.0 | prev p < .001 |

While the Replikin Count in non-lethal Influenza B remains remarkable constant, the Replikin Count in Influenza A shows significant variation that correlates with outbreaks, epidemics and pandemics. For example, mean Replikin Count of the RPG in FIG. 2 may be observed to be greater in the 1918 (H1N1) pandemic than in the 1957 (H2N2) and 1968 (H3N2) pandemics in approximate scale to the mortality rates observed in those three pandemics. The 1918 pandemic is thought to have resulted in 675,000 deaths in the U.S. and 50 million deaths globally. A Replikin Count of 19 in 1917 may be observed in FIG. 2. The 1957 pandemic is thought to have resulted in 70,000 deaths in the U.S. and 1-2 million deaths globally. A Replikin Count of 4 in 1957 with a standard deviation of 4.9 may be observed in FIG. 2. The 1968 pandemic is thought to have resulted in 34,000 deaths in the U.S. with 700,000 deaths globally. A Replikin Count of 7.2 in 1968 with a standard deviation of 8 may be observed in FIG. 2.

The dominance of H5N1 over other Influenza A strains between 1990 and 2007 is also evident in FIG. 2. This dominance in mean Replikin Count is reflected in high global lethality in birds during that time period and outbreaks resulting in human lethality in 1997, 2001-2004 and continuing lethality and possible human-to-human transmission in 2007. The counts for human H5N1 in 2004 through 2007 are increasing and approaching the mean Replikin Count level of the 1918 H1N1 pandemic. The mean Replikin Count of the RPG in chicken H5N1 is also observed to increase to a lesser degree over this time period and observed to decrease in 2007. The standard deviation of the means (SD) for all strains is shown in light grey columns with caps, on top of the column for the mean Replikin count, and emphasizes the broad distribution of Replikin Counts in the RPG of the expanding virus population. This broad distribution of Replikin Counts illustrates rapid changes in distribution of Replikin Counts during the rapid replication that is associated with virus outbreaks. During quiescent periods, the standard deviation is observed to be approximately 10% or less of the mean. In contrast, when an outbreak develops, the standard deviation is observed to be 50% or greater than the mean (the same phenomenon is observed in FIG. 7 for H3N8 equine encephalitis). The data for mean Replikin Count in human H5N1 for 2005, 2006 and 2007 suggest that the current epidemic is not over. For example, in each of the H1N1, H2N2 and H3N2 pandemics, a decline may be observed in Replikin Count prior to the end of the outbreak. This decline prior to end of an outbreak was also seen in the SARS outbreak of 2003. See FIG. 9. As such, since observed mean Replikin Count has not yet begun to decline in H5N1, the current epidemic is expected to continue.

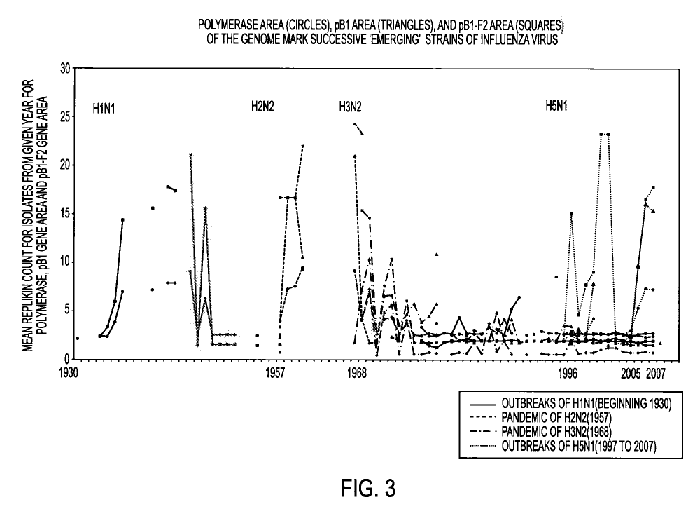

FIG. 3 illustrates successive “emerging” strains of influenza virus between 1930 and 2007. Mean Replikin Counts per year of isolation of various strains of influenza are provided for the polymerase area (marked with circles), the pB1 area (marked with triangles), and the pB1-F2 area (marked with squares). Data for H1N1 and H3N2 continue through 2007. Gaps represent years where no data was available on these genomic areas on PubMed.

Dramatic increases in Replikin Count may be observed just before outbreak in the rebound epidemic of H1N1 beginning in the 1930's, in the pandemics of H2N2 and H2N3, which occurred in 1957 and 1968, respectively, and the outbreaks of H5N1 between 1997 and 2007. The largest increase in Replikin Count may be observed in the pB1-F2 area of the genome, which is contained within the pB1 area of the genome and contains an identified RPG (e.g., SEQ ID NO: 1723). The next largest increase in Replikin Count may be observed in the pB1 area of the genome, which is contained in the polymerase area of the genome. The smallest increase in Replikin Count may be observed in the polymerase area of the genome. It may be observed, therefore, that the Replikin Count becomes magnified as measured within the pB1 area as compared to the polymerase area and within the pB1-F2 area as compared to the pB1 area.

As in FIG. 2, FIG. 3 illustrate the constancy of Replikin Counts during quiescent periods of the strain, and a marked increase in Replikin Peak Gene Replikin Counts one year in advance of, or simultaneous with, outbreaks of specific strains. FIGS. 2 and 3 demonstrate that neither increases in Replikin Count nor outbreaks occur in more than one influenza strain at the same time. The figures further demonstrate a “rise” of H3N2 in 1968 that occurs simultaneous with a “fall” of H2N2.

C. Replikin Count in RPG of H5N1 Directly Correlates with Human Mortality

The inventors have now demonstrated that increased Replikin Counts in the RPG of H5N1 influenza virus (e.g., SEQ ID NO: 1684) may be directly correlated with human mortality. FIG. 4 illustrates the relationship of Replikin Count of the Replikin Peak Gene in human H5N1 to percent human mortality between 2003 and 2007 in human cases of H5N1 infection. An increase in Replikin Count in the Replikin Peak Gene of H5N1 is observed to be quantitatively related to higher mortality in the host. The Replikin Peak Gene in human H5N1 is the pB1 gene area, which has the highest concentration of continuous Replikin sequences in publicly available sequences of the H5N1 genome.

Magnification of Replikin Count may be observed in FIG. 4 when the mean Replikin Count in the whole virus a given year is compared with the mean Replikin Count in the pB1 gene area (identified as the Replikin Peak Gene area of the virus). For example, annual mean Replikin Count in the whole genome increased 33% from 2005 to 2007 while annual mean Replikin Count in the Replikin Peak Gene (pB1 gene area) increased nine-fold from 2003 to 2007 and 222% from 2005 to 2007 with a statistical p value less than 0.001. Annual percent mortality of human H5N1 cases increased approximately 100% from 2005 to 2007. The 2007 data, while unfortunately sparse because of withholding of data by some countries, does not indicate a decrease in whole genome Replikin count or in RPG Replikin Count. A significant decrease in Replikin Count would typically signal the end of an outbreak or epidemic. See, e.g., SARS coronavirus data in FIG. 9. No such decrease has yet to be observed.

FIGS. 16 and 17 likewise demonstrate that increased Replikin Counts in the RPG of H5N1 is more strongly correlated with lethality in a given year than increased Replikin Counts in other portions of the H5N1 genome. The data for FIGS. 16 and 17 are contained in Table 3 below.

TABLE 3
H5N1 Replikin Concentration and Human Mortality

| | 2003 | 2004 | 2005 | 2006 | 2007 |
|---|---|---|---|---|---|
| H5N1 Whole Virus Replikin Count in Humans | 2.2 +/− 1.2 | 2.4 +/− 1.4 | 2.3 +/− 2.6 | 3.8 +/− 4.6 | 3.7 +/− 4.5 |
| H5N1 Polymerase Replikin Count in Humans | 2.6 +/− 0.8 | 2.9 +/− 0.9 | 4.8 +/− 5.0 | 7.4 +/− 7.0 | 7.3 +/− 6.7 |
| H5N1 pB1 gene area (RPG) Replikin Count in Humans | 2.0 +/− 0 | 2.0 +/− 0.1 | 8.0 +/− 7.7 | 16.1 +/− 5.7 | 15.4 +/− 5.9 |
| H5N1 pB2 gene area Replikin Count in Humans | 2.4 +/− 0 | 2.8 +/− 0.3 | 2.4 +/− 0.4 | 2.4 +/− 0.1 | 2.4 +/− 0.3 |
| H5N1 pA gene area Replikin Count in Humans | 3.8 +/− 0 | 4.0 +/− 0.6 | 3.8 +/− 0.4 | 3.8 +/− 0.3 | 4.2 +/− 0.3 |
| H5N1 Human Mortality Percent | | | 45 | 69 | 85 |

In FIG. 16, a correlation was established between human mortality and (1) mean concentration of Replikin sequences in the whole genome, (2) mean concentration of Replikin sequences in the polymerase gene, and (3) mean concentration of Replikin sequences in the Replikin Peak Gene (pB1 gene area) of H5N1 influenza strains. As Replikin concentration increased by these three measures, human mortality was observed to increase. However, while all three measures provided a correlation with human mortality, changes in the Replikin Count in the polymerase gene correlated more significantly with human mortality, and changes in the Replikin Count in the Replikin Peak Gene (pB1 gene area) of the H5N1 genome correlated still more significantly with human mortality. FIG. 16 suggests, therefore, that identification of Replikin Peak Genes within viral genomes improves identification and prediction of virulence and mechanisms of virulence using Replikin concentration data.

FIG. 17 illustrates a significant eight-fold increase in Replikin concentration in the pB1 gene area (Replikin Peak Gene) of isolates of H5N1 while no significant increase is observed in neighboring gene areas of the pB1 gene area, namely, the pA gene area and the pB2 gene area. FIG. 17 illustrates a significant correlation between human mortality and the Replikin Peak Gene (pB1 gene area) of isolates of H5N1 influenza virus. No correlation is observed in neighboring gene areas of the pB1 gene area, namely the pB2 and pA gene areas. In addition to the correlative aspect of the increase in Replikin concentration being related to percent mortality, FIG. 17 provides strong confirmation of the power and validity of the methodology of predicting changes in virulence and outbreaks of virus by monitoring changes in Replikin concentration.

Table 3 provides mortality data for H5N1 infections from 2005 through 2007 and does not include earlier mortality data. Mortality data prior to 2005 has not been included in Table 3 because data prior to 2005 is inconsistent and understood by those of skill in the art to contain errors including errors caused by underreporting. The first generally agreed occasion when there were human deaths caused by proven H5N1 infection was in Hong Kong in 1997-1998. (This is probably incorrect, however, since there probably was mortality between 1959, when H5N1 was first reported, and 1997). The usual figures cited for 1997 are: 30 human cases, 8 deaths with mortality rate of about 27%. The number of cases (morbidity) and the number who died (mortality) that were not reported is unknown, but suspected to be significant. These errors are usually high in geographic areas where the medical care is less structured and scientific and the reporting is incomplete. Press reports between 1998 and 2002 were few, scattered, and not in agreement. Mortality data between 2005 and 2007 appear to be more consistent and have a higher level of reliability. Table 3, therefore, contains data from these years.

D. Replikin Count in RPG Correctly Identifies Host Lethality and Geographic Location of Outbreaks

The inventors have now demonstrated that Replikin Count in a Replikin Peak Gene provides a method for predicting and identifying outbreaks of pathogens such as H5N1 influenza by host and by geographic area. FIG. 5 demonstrates the predictive capacity for identifying outbreaks in particular hosts and FIG. 6 demonstrates the predictive capacity for identifying the lethality of an outbreak in a particular geographic area. The data for FIGS. 5 and 6 are contained in Table 4 below.

TABLE 4
Host and Geographic Predictions
(Replikin Count and Standard Deviation)

| | Goose | Chicken | Duck | Human | Japan | Russia | Egypt | China | Vietnam | Thailand | Indonesia |
|---|---|---|---|---|---|---|---|---|---|---|---|
| 2003 | No | 2 ± 1.1 | 2.1 ± 1.1 | 2.2 ± 1.2 | 2 ± 0 | No | No | 1.9 ± 0.3 | No | 1.9 ± 0.2 | No data |
| | data | | | | | data | data | | data | | |
| 2003 | No | 3 ± 1.5 | 3.8 ± 1.8 | 3.3 ± 1.3 | 2 ± 0 | No | No | 2 ± 0.1 | 2 ± 0.2 | 2 ± 0.2 | 2 ± 0 |
| | data | | | | | data | data | | | | |
| 2005 | 2.6 ± 1.2 | 3.2 ± 2.8 | 4 ± 5 | 3.7 ± 4.1 | 2.1 ± 0.1 | 2 ± 0.1 | No | 2.7 ± 0.5 | 3 ± 0.2 | 4.1 ± 4.7 | 3.9 ± 3.6 |
| | | | | | | | data | | | | |
| 2006 | 2.5 ± 1.2 | 3.2 ± 3.1 | 2.7 ± 1.6 | 5 ± 5.7 | 2 ± 0 | 2 ± 0 | 2 ± 0 | 3 ± 0.2 | No | 6.7 ± 6.7 | 16.7 ± 4.9 |
| | | | | | | | | | data | | |

Increased Replikin Counts in pathogens in particular hosts is predictive of an increase in probability of an outbreak of the pathogen. For example, FIG. 5 illustrates a 2005 through 2007 upregulation of human H5N1 in humans as compared to H5N1 in goose, duck and chicken. Replikin analysis was performed separately for H5N1 Replikin Peak Genes of each host group, namely, goose, duck, chicken and human. Low levels of Replikin Count, below 4, were observed in each host group until 2005-2006, when epidemics increased in Asian countries. While duck H5N1 counts decreased in 2006, Replikin Counts continued to increase in chicken H5N1 in 2006. Human RPG activity was upregulated in 2005-2006 and overtook RPG activity in chickens. This transition of increased Replikin Count from duck to chicken to human is in agreement with epidemiological evidence of the order of transfer of the virus between hosts. Changes in Replikin Count in the Replikin Peak Gene of the H5N1 isolates in FIG. 5 allows for identification of those hosts in which the influenza virus strain is more virulent than other hosts.

Increased Replikin Counts in pathogens in particular geographic areas is predictive of an increase in lethality of the pathogen in the identified geographic area. For example, FIG. 6 illustrates localization of human H5N1 isolates having the highest lethality by measuring mean Replikin Counts in isolates of human H5N1 from different geographic areas isolated in a given year. Replikin analysis was performed separately for human HSN1 RPGs of each country. The results are shown for the Replikin Count for all data available on PubMed each year 2003-2006. Low levels of Replikin count, below 4, were observed in each host group until 2005-2006, when human H5N1 increased in Asian countries. Human RPG activity was upregulated in 2005-2006 most prominently in Indonesia. Using this data, Applicants predicted Indonesia would be the country most likely to first experience increased human mortality. The prediction was proven correct in 2007 when incidence of human morbidity and mortality in the Indonesian outbreak were exceptionally high and evidence of possible human to human transmission was observed. Changes in Replikin Count in the Replikin Peak Gene of the H5N1 isolates such as in FIG. 6 allows for identification of those geographic areas in which the influenza virus strain is more virulent than other geographic areas.

E. Replikin Peak Genes as Predictors of Outbreaks

Identification of the pB1 Replikin Peak Gene as a more significant gene area for changes in Replikin concentration effecting virulence reflects the same phenomenon in equine influenza as demonstrated in the pB1 gene area of H5N1. See FIG. 7 and compare to FIGS. 2 and 3. FIG. 7 additionally demonstrates the cyclical nature of changes in Replikin Count over a period of years. These cycles are like those observed previously for H1N1 since 1918. See FIGS. 2 and 3 and U.S. Pat. No. 7,189,800 (Tables 3-6). It is noteworthy that increases in Replikin Count in virulent influenza isolates have generally ranged between 2 and 5, that is 2- to 3-fold above other influenza isolates. Replikin Counts in the Replikin Peak Gene of virulent isolates, however, have been observed to range between 2 and 20, that is a 10-fold change in concentration. This magnification makes sense in terms of the concentration of the Replikins in the Replikin Peak Gene, rather than an even distribution throughout other parts of the virus genome.

F. Replikin Concentration in Replikin Peak Gene of pB1 Area Correlates with Equine Influenza Epidemics

As with other influenza strains, an increase in Replikin concentration in equine influenza virus (EIV) has likewise been shown to be predictive of an increase in virulence of the virus and allows for prediction of forthcoming outbreaks or increases in morbidity and, in extreme cases, mortality. A review of publicly available amino acid sequences of isolates of EIV that demonstrates an increase in Replikin Count in the genome or a genome segment, or in a protein or protein fragment of the virus over time or between isolates is used as a predictor of an increase in outbreaks and morbidity in horses, donkeys, mules and other effected animals. Publicly available sequences for isolates of EIV from PubMed or other public or private sources may be analyzed by hand or using the FluForecast® search tool. (REPLIKINS LLC, Boston, Mass.).

Applicants have established a correlation between Replikin Count in the pB1 gene area (RPG) in EIV and an increase in virulence of the virus resulting in epidemics. The Applicants have reviewed publicly available amino acid sequences of isolates of EIV having accession numbers at www.pubmed.com and have identified increases in Replikin concentration in the Replikin Peak Gene of the pB1 gene area of the genome of the virus that relate to and predict an increase in outbreaks.

Applicants' initial analysis determined the Replikin Peak Genes within publicly available sequences of the pB1, pB2 and pA proteins of the H3N8 strain of influenza virus by analyzing publicly available sequences for the gene areas of the pB1, pB2 and pA proteins and identifying the protein segment having the highest concentration of continuous Replikin sequences within each gene area.

Applicants then compared the mean Replikin concentration in the identified Replikin Peak Gene for each of the three gene areas for isolates in each year having publicly available sequence information between 1977 and 2007. Applicants further analyzed all publicly available whole genome sequences for H3N8 between 1977 and 2007.

FIG. 7 illustrates a relationship between Replikin Counts of Replikin Peak Genes identified within the pB1, pB2, and pA genomic areas of equine influenza virus 1977-2007 and epidemics of equine encephalitis caused by H3N8 equine influenza. Replikin Count increases in the pB1 gene area are observed to occur one to three years before epidemic outbreaks while no increase in Replikin Count is observed in the pB2 and pA gene areas. Standard deviation of the means is again shown separately (as a clear column) to draw attention to the increase of some individual viruses with higher Replikin counts prior to the maximal Replikin count elevation, followed by viral outbreak.

Replikin Counts of the RPGs of the pA and pB2 genomic areas, which are immediately adjacent to the pB1 area in the H3N8 genome, remain below 5 and do not increase to the extent of the Replikin Count of the RPG of the pB1 area. These observed increases in the pB1 area and absence of increases in the pB2 and pA areas are in direct agreement with the data on H5N1 influenza reflected in FIG. 1.

The range of Replikin Counts in the RPGs of H3N8 may be observed to be similar to the range of Replikin Counts in other Influenza A species. See, e.g., FIG. 2. Further, Replikin Counts in H3N8 during quiescent periods are comparable to Replikin Counts in Influenza B at all observed times and comparable to other influenza species during quiescent periods, that is between lethal outbreaks. Additionally, Replikin Counts in H3N8 during epidemics are comparable to outbreak levels reached prior to epidemics in Influenza A. See, e.g., FIG. 2.

The data for FIG. 7 is provide in Table 4A below, which provides the yearly mean Replikin concentrations (with Standard Deviation) of publicly available peptide sequences of the identified Replikin Peak Gene (RPG) of the pB1 gene area, the yearly mean Replikin concentrations of publicly available peptide sequences of the identified Replikin Peak Gene (RPG) of the pA gene area, and the yearly mean Replikin concentrations of publicly available peptide sequences of the identified Replikin Peak Gene (RPG) of the pB2 gene area.

TABLE 4A
Equine Influenza

| | Replikin | | Replikin | | Replikin | Replikin |
|---|---|---|---|---|---|---|
| | Concentration | | Concentration | | Concentration | Concentration |
| Year | of pB1 Gene | SD | of RPG in pB1 | SD | of RPG in pA | of RPG in pB2 |
| 1972 | 1.8 | 0 | | | | 2.4 |
| 1977 | 16.7 | 0 | 16.7 | 0 | | 2.4 |
| 1978 | 11 | 12.7 | 11 | 12.7 | | 2.4 |
| 1979 | 22.2 | 0 | 22.2 | 0 | | 2.4 |
| 1980 | 6.7 | 8.8 | 6.7 | 8.8 | | 2.3 |
| 1982 | 17.8 | 0 | 17.8 | 0 | | 2.4 |
| 1985 | 20.6 | 2.4 | 20.6 | 2.4 | | 2.4 |
| 1986 | 13.3 | 11.1 | 13.3 | 11.1 | 3.4 | 2.3 |
| 1987 | 19.1 | 2.7 | 19.1 | 2.7 | | 2.4 |
| 1991 | 9.3 | 10.4 | 9.3 | 10.4 | 4.6 | 2.4 |
| 1992 | 15.6 | 0 | 15.6 | 0.2 | | 2.4 |
| 1998 | 2.2 | 0 | 2.2 | 0 | | 3.8 |
| 1999 | 2 | 0 | 2 | 0 | | 0.2 |
| 2000 | | | | | | 0.6 |
| 2001 | 9.9 | 11.2 | 9.9 | 11.2 | 2.2 | 2.4 |
| 2002 | 4.6 | 6.1 | 4.6 | 6.1 | 2.2 | 2.4 |
| 2003 | 2 | 0 | 2 | 0.2 | 3.1 | 2.4 |
| 2004 | 2.2 | 0.2 | 2.2 | 0.2 | 2.2 | 2.4 |
| 2005 | 18.5 | 2.8 | 18.5 | 2.8 | | 2.3 |
| 2006 | | | | | 3.5 | |
| 2007 | | | | | 2.1 | |

In FIG. 7, Series 1 reflects the mean Replikin concentration identified in the Replikin Peak Gene in the pB1 area of the genome. Series 2 reflects the standard deviation from mean Replikin concentration in the pB1 gene area. The large standard deviations in the first column of every pair are noteworthy as the Standard Deviation then drops as the mean Replikin concentration increases. This increase in standard deviation in the Replikin Peak Gene pB1 area probably reflects heterogeneity in the virus population once a more virulent strain of virus having a higher Replikin concentration has become present. The higher standard deviation suggests a more diverse population of the virus in which some members are relatively dormant whereas an increasing number are rapidly replicating. As the “build up” increases prior to the outbreak, more members are increasingly, rapidly replicating, thus raising the mean Replikin concentration. In contrast, as seen in Table 4A above, the stability of the Replikin concentration in neighboring genomic areas such as pA and pB2 demonstrate both the reproducibility of the quantitative measurement of the Replikin concentration, the constancy over many years of the Replikin concentration in dormant areas, and the high degree of specificity of the increases in the pB1 area. The standard deviation then drops as the more virulent strain or strains enters an epidemic stage and less virulent strains (having lower Replikin concentrations) become less competitive and less present as a percentage of isolates in the host population. To the inventors' knowledge no such highly specific changes in virus structure have been observed to correlate with outcomes in the host.

Specifically, Series 3 in FIG. 7 reflects the Replikin concentration identified in the Replikin Peak Gene in the pA gene area of the genome, which neighbors the pB1 gene area. The Replikin concentration of the Replikin Peak Gene in the pA gene area is observed to be remarkably constant over the analyzed years, never going above 5. This constancy stands in marked contrast to the extensive changes in Replikin concentration noted in the pB1 gene area. These control data validate the location of the most significant Replikin Peak Gene for the present isolates of virus in the pB1 gene area. Because the pA gene is right next to the pB1 gene, the differences in magnitude of change in Replikin concentration between these neighboring areas is quite remarkable.

Specifically, Series 4 in FIG. 7 reflects the Replikin concentration identified in the Replikin Peak Gene in the pB2 gene area of the genome, which also neighbors the pB1 gene area. The Replikin concentration of the Replikin Peak Gene in the pB1 gene area is also observed to be remarkably constant over the analyzed years, not going above 4. This constancy again stands in marked contrast to the extensive changes in Replikin concentration noted in the pB1 gene area. Again, the control data validate the location of the most significant Replikin Peak Gene for the present isolates of virus in the pB1 gene area. Because the pB2 gene is right next to the pB1 gene, the differences in change in Replikin concentration between these neighboring areas is also remarkable.

VII. Methods of Predicting and Treating Outbreaks of Foot and Mouth Disease Virus (FMDV) Using RPGs and Related Replikin Sequences

An increase in Replikin concentration in the VP1 protein (containing an RPG of the virus genome) of foot and mouth disease virus (FMDV) is predictive of an increase in virulence and lethality of the virus and allows for prediction of forthcoming outbreaks or increases in virulence or lethality. Applicants have reviewed all publicly available amino acid sequences of isolates of FMDV having accession numbers at www.pubmed.com between 1969 and 2006 and have identified increases in Replikin concentration in the VP1 protein of FMDV (e.g., SEQ ID NO: 157) that relate to and predict certain known outbreaks of Foot and Mouth Disease. FIG. 11 illustrates the correlation of Replikin Count observed in the VP1 protein of isolates of foot and mouth disease virus on a year by year basis and observed outbreaks.

Applicants reviewed Accession No. ABM63320 (SEQ ID NO: 157), which provides the amino acid sequence of the entire serotype-O FMDV VP1 polyprotein, and identified two RPGs. The first RPG begins at amino acid residue 925 and continues through amino acid residue 1018 and was isolated in silico as SEQ ID NO: 124. Five Replikin sequences were isolated (SEQ ID NOS: 125-129) in the first RPG, which gave the first RPG a Replikin Count of 6.3. The first RPG represents the Replikin Peak Gene of a fragment of the VP1 polyprotein.

The second Replikin Peak Gene begins at amino acid residue 1300 and continues through amino acid residue 1481 and was isolated in silico as SEQ ID NO: 130. Twenty-six Replikin were isolated in the second RPG (SEQ ID NOS: 131-156). The second Replikin Peak Gene Area has a Replikin Count of 14.3 and represents the Replikin Peak Gene of the entire reported VP1 polyprotein. Conserved Replikins within the RPG at SEQ ID NO: 130 are also contained, for example, in sequence fragments reported at Accession Nos. ABA46641, AAG43385, AAP81678 and ABG77564. Likewise, parts of the RPG of SEQ ID NO: 124 are contained in these accession numbers.

In the amino-terminal of SEQ ID NO: 157 (Accession No. ABM63320) SEQ ID NOS: 158-160 were isolated as Replikins. In the mid-molecule, SEQ ID NOS: 161-194 were isolated as Replikins. In the carboxy-terminal, SEQ ID NOS: 195-213 were isolated as Replikins. Each of these Replikin sequences is a preferred sequence for immunogenic compositions and vaccines and for other diagnostic, therapeutic and predictive purposes as described herein.

FIG. 11 illustrates the concentration of Replikin sequence observed in the VP1 protein of isolates of the common serotype-O of foot and mouth disease virus having publicly available accession numbers on a year by year basis between 1969 and 2006. Observed European and UK outbreaks of Foot and Mouth Disease are noted and relate to observed increases in Replikin Count prior to disease outbreak.

Prediction of the listed epidemics as well as future outbreaks may be made, for example, by reviewing the Replikin Counts of isolates of FMDV and comparing the Replikin Counts of the VP1 protein or the RPG within the VP1 protein for a particular year with Replikin Counts from other years. A significant increase in Replikin Count from one year to the next and preferably over one, two or three years provides predictive value of an emerging strain of FMDV that may begin an outbreak of Foot and Mouth Disease. A Foot and Mouth Disease outbreak may be predicted within about six months to about one year or more from the observation of a significant increase in Replikin Count.

More preferably, an outbreak of Foot and Mouth Disease may be predicted within about six months to about one year from the observation of a significant increase in Replikin count over two or three years. An outbreak may likewise be predicted within about six months to about one year from the initial observation of a decrease in Replikin Count following a significant increase. Using this method, Applicants predicted the Aug. 3, 2007 outbreak of FMDV in the United Kingdom months prior to the outbreak.

The data for FIG. 11 is provided in Table 5 below. Note that data is available for 1958 and 1962, but was not included in FIG. 11. Note also that no data was available for 1959 through 1961, 1963 through 1968 and 2004.

TABLE 5
FMDV Serotype O Replikin Counts

| | Accession | | | | |
|---|---|---|---|---|---|
| | Records for | | | Significance | Significance |
| | Serotype-O | Replikin | Standard | (compared to lowest | (compared to previous |
| Year | FMDV VP1 | Count | Deviation | value) | year) |
| 1958 | 1 | 3.8 | 0.0 | | |
| 1962 | 172 | 0.8 | 0.2 | low p < 0.001 | |
| 1969 | 1 | 0.4 | 0.0 | | prev p < 0.001 |
| 1970 | 2 | 0.7 | 0.4 | low p < 0.05 | prev p < 0.40 |
| 1971 | 1 | 3.3 | 0.0 | | prev p < 0.05 |
| 1972 | 1 | 0.5 | 0.0 | | |
| 1973 | 1 | 0.5 | 0.0 | | |
| 1974 | 7 | 1.1 | 0.3 | low p < 0.001 | prev p < 0.001 |
| 1975 | 6 | 1.0 | 0.5 | low p < 0.001 | prev p > 0.50 |
| 1976 | 4 | 1.2 | 0.1 | low p < 0.001 | prev p < 0.40 |
| 1977 | 4 | 1.0 | 0.4 | low p < 0.001 | prev p < 0.20 |
| 1978 | 8 | 1.0 | 0.1 | low p < 0.001 | prev p > 0.50 |
| 1979 | 47 | 2.5 | 1.4 | low p < 0.001 | prev p < 0.001 |
| 1980 | 5 | 1.5 | 0.5 | low p < 0.001 | prev p < 0.001 |
| 1981 | 2 | 0.8 | 0.3 | low p < 0.04 | prev p < 0.10 |
| 1982 | 21 | 0.9 | 0.1 | low p < 0.001 | prev p > 0.50 |
| 1983 | 6 | 0.9 | 0.4 | low p < 0.001 | prev p > 0.50 |
| 1984 | 1 | 1.2 | 0.0 | | prev p < 0.10 |
| 1985 | 3 | 1.2 | 0.7 | low p < 0.02 | prev p > 0.50 |
| 1986 | 2 | 0.8 | 0.5 | low p < 0.05 | prev p > 0.50 |
| 1987 | 1 | 3.6 | 0.0 | | prev p < 0.05 |
| 1988 | 3 | 1.2 | 0.0 | | |
| 1989 | 6 | 1.3 | 0.8 | low p < 0.001 | prev p > 0.50 |
| 1990 | 5 | 2.0 | 1.3 | low p < 0.02 | prev p < 0.30 |
| 1991 | 7 | 3.5 | 2.4 | low p > 0.50 | prev p < 0.10 |
| 1992 | 9 | 2.1 | 1.0 | low p < 0.001 | prev p < 0.10 |
| 1993 | 16 | 2.2 | 1.4 | low p < 0.001 | prev p > 0.50 |
| 1994 | 18 | 2.2 | 1.1 | low p < 0.001 | prev p > 0.50 |
| 1995 | 12 | 2.0 | 0.8 | low p < 0.001 | prev p > 0.50 |
| 1996 | 12 | 1.6 | 1.1 | low p < 0.001 | prev p < 0.30 |
| 1997 | 48 | 2.6 | 1.1 | low p < 0.001 | prev p < 0.01 |
| 1998 | 72 | 1.2 | 1.1 | low p < 0.001 | prev p < 0.001 |
| 1999 | 49 | 2.3 | 1.3 | low p < 0.001 | prev p < 0.001 |
| 2000 | 61 | 1.3 | 0.8 | low p < 0.001 | prev p < 0.001 |
| 2001 | 8 | 1.8 | 0.9 | low p < 0.001 | prev p < 0.10 |
| 2002 | 2 | 1.1 | 0.2 | low p < 0.02 | prev p < 0.05 |
| 2003 | 8 | 1.3 | 0.5 | low p < 0.001 | prev p < 0.40 |
| 2005 | 8 | 1.3 | 1.0 | low p < 0.001 | prev p > 0.50 |
| 2006 | 3 | 3.8 | 3.1 | low p > 0.50 | prev p < 0.20 |

A. Prediction using VP1 Protein of All Serotypes

In addition to FMDV VP1 proteins of serotype-O, Applicants also analyzed publicly available sequences for isolates of all reported serotypes of FMDV VP1 protein from PubMed. The data is provided in Table 6 below. Note the increase in Replikin Count correlated with two epidemics in the United Kingdom (and other European countries) in 2001 and in the United Kingdom in 2007. Also note the low Replikin Counts during quiescence. Replikin Count increases from 1.6 in 1998, to 2.5 in 1999, to 2.7 in the year of the epidemic, 2001. Then post-epidemic, three lower Replikin Count years are noted, 1.5 in 2002, 1.5 in 2003, and 1.1 in 2005 (there were no publicly available sequences from 2004). The Replikin Count then rose to 2.8 in 2006 just prior to the outbreak in 2007. Note that the p values are less than 0.001 with respect to previous Replikin Counts.

TABLE 6
FMDV (all isolates)

| | Accession | | | | |
|---|---|---|---|---|---|
| | Records for | | | Significance | Significance |
| | FMDV VP1 | Replikin | Standard | (compared to lowest | (compared to previous |
| Year | protein | Count | Deviation | value) | year) |
| 1998 | 92 | 1.6 | ±1.2 | low p < 0.001 | prev p < 0.001 |
| 1999 | 60 | 2.5 | ±1.3 | low p < 0.001 | prev p < 0.001 |
| 2000 | 115 | 1.7 | ±1.4 | low p < 0.001 | prev p < 0.001 |
| 2001 | 32 | 2.7 | ±1.0 | low p < 0.001 | prev p < 0.001 |
| 2002 | 3 | 1.5 | ±0.8 | low p < 0.05 | prev p < 0.02 |
| 2003 | 10 | 1.5 | ±0.8 | low p < 0.001 | prev p > 0.50 |
| 2005 | 43 | 1.1 | ±0.6 | low p < 0.001 | prev p < 0.10 |
| 2006 | 36 | 2.8 | ±0.9 | low p < 0.002 | prev p < 0.001 |

B. Prediction Using VP1 Protein of Serotype C

Table 7 provides Replikin Count data for isolates of serotype-C FMDV for some years between 1955 and 2006. Note the significant increases over the low value in Replikin Count in 1998 and 1999 (prior to the 2001 epidemic in the UK) and the significant increase over the low value in 2006 (prior to the 2007 outbreak in the UK). Years having no available data are not reflected in the table.

TABLE 7
FMDV Serotype C

| | | | Stan- | | |
|---|---|---|---|---|---|
| | Accession | | dard | Significance | Significance |
| | Records for | Replikin | Devi- | (compared to | (compared to |
| Year | Serotype C | Count | ation | lowest value) | previous year) |
| 1955 | 1 | 2.7 | 0.0 | | |
| 1957 | 1 | 2.8 | 0.0 | | |
| 1979 | 12 | 1.4 | 0.5 | low p < 0.001 | prev p < 0.001 |
| 1982 | 2 | 2.1 | 1.6 | low p > 0.50 | prev p > 0.50 |
| 1988 | 1 | 1.1 | 0.0 | | prev p > 0.50 |
| 1989 | 1 | 1.1 | 0.0 | | |
| 1991 | 3 | 0.5 | 0.0 | low p < 0.001 | prev p < 0.001 |
| 1992 | 2 | 0.5 | 0.0 | low p < 0.001 | prev p > 0.50 |
| 1993 | 5 | 0.5 | 0.0 | low p < 0.001 | prev p < 0.40 |
| 1997 | 2 | 1.4 | 1.3 | low p < 0.30 | prev p < 0.30 |
| 1998 | 2 | 2.9 | 0.0 | | prev p < 0.20 |
| 1999 | 4 | 3.1 | 0.4 | low p < 0.10 | prev p < 0.40 |
| 2006 | 10 | 3.0 | 0.2 | low p < 0.001 | prev p > 0.50 |

The correlation between Replikin concentration and viral outbreaks noted above and illustrated in FIG. 11 provide a method of predicting outbreaks of Foot and Mouth Disease by monitoring increases in Replikin concentration in the VP1 protein of all available FMDV isolates. The method may also employ all available serotype-O isolates or serotype-C isolates of the virus.

The epidemiology and virology FMDV is different from the epidemiology and virology of some other viruses discussed herein such as Influenza virus. Nevertheless, a correlation between increases in Replikin Count in the FMDV VP1 protein and outbreaks of the virus provides compounding data establishing a shared phenomenon of rapid replication and virulence with an overwhelming number of other tested viruses and organisms.

C. Replikins Conserved in Serotype O FMDV RPGs

In serotype-O of FMDV, two conserved Replikin sequences contained within the Replikin Peak Gene are hkqkivapvk (SEQ ID NO: 91) and hpsearhkqkivapvk (SEQ ID NO: 92). A point mutant of the hpsearhkqkivapvk sequence to hptearhkqkivapvk (SEQ ID NO: 93) (mutation underlined) reportedly occurred in isolates from 1967 and 2007. The Replikin sequence hkqkivapvk (SEQ ID NO: 91) has been conserved from 1962 to 2006. The Replikin sequence hpsearhkqkivapvk (SEQ ID NO: 92) has been conserved from 1962 to 2006 except for the point mutation hptearhkqkivapvk (SEQ ID NO: 93), which is present in isolates reportedly having caused the 1967 outbreak (isolate O₁BFS) and now the 2007 outbreak in the United Kingdom. These isolated conserved Replikin sequence are embodiments of the invention of particular preference for predictive, diagnostic and therapeutic capacity.

Table 8 provides the accession numbers of isolates between 1962 and 2006 containing the conserved sequence hkqkivapvk (SEQ ID NO: 91) and the amino acid position within the VP1 protein sequence where the conserved Replikin sequence begins.

TABLE 8
FMDV Conserved SEQ ID NO: 91

| | |
|---|---|
| 1962 | CAC22210 position 202, AAP81678 position 153, AAP81677 position 153, AAP81676 position 153, |
| | AAP81675 position 153, AAP81674 position 153, ABA46701 position 201, ABA46700 position |
| | 201, ABA46699 position 201, ABA46698 position 201, ABA46697 position 201, ABA46696 |
| | position 201, ABA46695 position 201, ABA46693 position 201, ABA46692 position 201, |
| | ABA46691 position 201, ABA46690 position 201, ABA46689 position 201, ABA46688 position |
| | 201, ABA46687 position 201, ABA46686 position 201, ABA46685 position 201, ABA46684 |
| | position 201, ABA46683 position 201, ABA46682 position 201, ABA46681 position 201, |
| | ABA46679 position 201, ABA46678 position 201, ABA46677 position 201, ABA46675 position |
| | 201, ABA46674 position 201, ABA46673 position 201, ABA46672 position 201, ABA46671 |
| | position 201, ABA46670 position 201, ABA46669 position 201, ABA46668 position 201, |
| | ABA46666 position 201, ABA46665 position 201, ABA46664 position 201, ABA46663 position |
| | 201, ABA46662 position 201, ABA46661 position 201, ABA46660 position 201, ABA46659 |
| | position 201, ABA46658 position 201, ABA46657 position 201, ABA46655 position 201, |
| | ABA46654 position 201, ABA46653 position 201, ABA46652 position 201, ABA46651 position |
| | 201, ABA46650 position 201, ABA46649 position 201, ABA46648 position 201, ABA46647 |
| | position 201, ABA46644 position 201, ABA46643 position 201, ABA46642 position 201, |
| | ABA46641 position 201, ABA46640 position 201, ABA46639 position 201, ABA46638 position |
| | 201, ABA46637 position 201, ABA46614 position 201, ABA46613 position 201, ABA46612 |
| | position 201, ABA46611 position 201, ABA46610 position 201, ABA46609 position 201, |
| | ABA46606 position 201, ABA46605 position 201, ABA46604 position 201, ABA46603 position |
| | 201, ABA46602 position 201, ABA46601 position 201, ABA46600 position 201, ABA46597 |
| | position 201, ABA46596 position 201, ABA46594 position 201, ABA46591 position 201, |
| | ABA46590 position 201, ABA46589 position 201, ABA46588 position 201, ABA46586 position |
| | 201, ABA46585 position 201, ABA46583 position 201, ABA46582 position 201, ABA46581 |
| | position 201, ABA46580 position 201, ABA46579 position 201, ABA46578 position 201, |
| | ABA46576 position 201, ABA46574 position 201, ABA46573 position 201, ABA46571 position |
| | 201, ABA46570 position 201, ABA46569 position 201, ABA46568 position 201, ABA46566 |
| | position 201, ABA46565 position 201, ABA46563 position 201, ABA46561 position 201, |
| | ABA46560 position 201, ABA46542 position 201, ABA46541 position 201, ABA46539 position |
| | 201, ABA46538 position 201, ABA46537 position 201, ABA46536 position 201, ABA46535 |
| | position 201, ABA46534 position 201, ABA46533 position 201, ABA46532 position 201, |
| | ABA46531 position 201, ABA46559 position 201, ABA46540 position 201, |
| 1969 | CAB62584 position 724 |
| 1972 | CAC22304 position 202. |
| 1974 | CAC22211 position 202, AAK69575 position 153, AAR85362 position 153, AAR85361 position |
| | 153, AAR22955 position 153, AAR22953 position 153. |
| 1975 | AAK69576 position 153, CAC20174 position 201, AAR85363 position 153, AAG35653 position 724. |
| 1976 | AAR22952 position 153, AAR22933 position 153, AAR22932 position 153. |
| 1977 | AAR22963 position 153, AAR22950 position 153, CAC48179 position 201. |
| 1978 | ABA46745 position 201, ABA46744 position 201, ABA46743 position 201, ABA46742 position |
| | 201, ABA46740 position 201, AAR22930 position 153. |
| 1979 | CAC22173 position 43, AAQ88330 position 153, AAQ88328 position 153, AAQ88327 position 153, |
| | AAQ88325 position 153, AAQ88324 position 153, AAQ88323 position 153, AAQ88322 position |
| | 153, AAQ88321 position 153, AAQ88320 position 153, AAQ88319 position 153, AAQ88318 |
| | position 153, AAQ88317 position 153, AAQ88316 position 153, AAQ88315 position 153, |
| | AAQ88314 position 153, AAQ88313 position 153, AAQ88312 position 153, AAG28368 position |
| | 43, AAG28367 position 43, AAG28366 position 43, AAG28362 position 43, AAG28357 position |
| | 43, AAG28356 position 43, AAG28355 position 43, AAG28354 position 43, AAG28353 position |
| | 43, AAG28352 position 43, AAG28348 position 43. |
| 1980 | AAR22962 position 153, AAR22959 position 153, AAR22941 position 153. |
| 1981 | AAR22951 position 153 |
| 1982 | CAC20178 position 201, AAZ31360 position 201, AAZ31359 position 201, AAZ31358 position |
| | 201, AAZ31357 position 201, AAZ31356 position 201, AAZ31355 position 201, AAZ31354 |
| | position 201, AAZ31353 position 201, AAZ31352 position 201, AAZ31351 position 201, |
| | AAZ31350 position 201, AAZ31349 position 201, AAZ31348 position 201, AAZ31347 position |
| | 201, AAZ31346 position 201, AAZ31345 position 201, AAZ31344 position 201, AAZ31343 |
| | position 201, AAZ31342 position 201. |
| 1983 | AAR22960 position 153, AAR22938 position 153, AAR22937 position 153. |
| 1985 | CAC22326 position 90. |
| 1986 | AAR22954 position 153. |
| 1987 | AAK62003 position 43. |
| 1988 | AAK69568 position 153, AAK69567 position 153. |
| 1989 | CAC22174 position 90, AAR22961 position 153, AAK62024 position 69. |
| 1990 | CAC22178 position 43, CAC22327 position 58. |
| 1991 | CAC22175 position 43, CAC22328 position 62. |
| 1992 | CAC22176 position 43, CAC22240 position 85, CAC48182 position 201. |
| 1993 | CAC22179 position 43, CAC40792 position 201, CAC40789 position 201, CAC40796 position 102. |
| 1994 | CAC22180 position 76, CAC22233 position 62, CAC22227 position 60, CAC22215 position 47, |
| | CAC22208 position 82, CAC22201 position 43, CAC22167 position 43, AAK62012 position 43, |
| | CAC40794 position 102, CAC40790 position 201, CAC40795 position 102, CAC40797 position |
| | 201. |
| 1995 | CAC22231 position 152, CAC22216 position 44, CAC22171 position 103, AAK62022 position 69. |
| 1996 | CAC22194 position 127, CAC51235 position 201, AAR22945 position 153, AAR22942 position |
| | 153, AAK62005 position 69. |
| 1997 | CAC51273 position 201, CAC51268 position 201, CAC51249 position 201, CAC51236 position |
| | 201, AAL05257 position 43, AAL05249 position 43, AAL05248 position 85, AAL05247 position |
| | 62, AAL05246 position 76, AAL05245 position 43, AAL05243 position 56, AAL05242 position 43, |
| | AAL05236 position 43, AAL05235 position 65, AAL05234 position 43, AAL05233 position 43, |
| | AAL05232 position 43, AAL05231 position 43, AAL05230 position 43, AAL05229 position 43, |
| | AAL05228 position 43, AAL05227 position 85, AAL05226 position 43, AAL05225 position 76, |
| | AAL05223 position 43, AAL05222 position 43, AAL05221 position 43, AAL05220 position 122, |
| | AAL05219 position 43, AAL05218 position 52, AAL05217 position 43, AAL05216 position 66, |
| | AAL05214 position 43, AAL05213 position 93, AAL05211 position 58, AAL05207 position 43, |
| | AAL05206 position 62, AAL05205 position 67, AAL05196 position 64. |
| 1998 | CAC22229 position 201, ABI16250 position 201, ABI16249 position 201, ABI16248 position 201, |
| | ABI16247 position 201, ABI16246 position 201, ABI16245 position 201, ABI16244 position 201, |
| | ABI16242 position 201, ABI16241 position 201, ABI16240 position 201, ABI16239 position 201, |
| | ABI16238 position 201, ABI16237 position 201, ABI16236 position 201, ABI16235 position 201, |
| | ABI16234 position 201, ABI16233 position 201, ABI16232 position 201, ABI16231 position 201, |
| | ABI16230 position 201, ABI16229 position 201, ABI16228 position 201, ABI16227 position 201, |
| | CAC51269 position 201, CAC51239 position 201, CAC51238 position 201, AAR85364 position |
| | 153, AAR22957 position 153, AAL05256 position 43, AAL05255 position 43, AAL05254 position |
| | 43, AAL05253 position 43, AAL05250 position 43, AAL05244 position 43, AAL05241 position 43, |
| | AAL05240 position 43, AAL05238 position 43, AAL05237 position 45, AAL05212 position 43. |
| 1999 | CAC22228 position 100, CAC22200 position 100, AAG43385 position 43, CAC51332 position 143, |
| | CAC51270 position 175, CAC51255 position 201, CAC51318 position 201, CAC51247 position |
| | 201, CAC51246 position 201, CAC51245 position 201, CAD62370 position 925, CAD62369 |
| | position 925, CAD62208 position 925, CAC20187 position 201, AAR22956 position 153, |
| | AAR22940 position 153, AAF06146 position 43, AAD41912 position 81, AAD41131 position 81, |
| | AAL05251 position 43, AAL05215 position 43, AAL05210 position 43, AAL05209 position 43, |
| | AAL05208 position 43, AAL05204 position 43, AAL05203 position 45, AAL05202 position 43, |
| | AAL05201 position 43, AAL05200 position 43, AAL05199 position 43, AAL05198 position 43, |
| | AAL05197 position 70, AAL05195 position 59, AAL05194 position 58, AAL05193 position 43, |
| | AAL05192 position 43, AAL05191 position 43. |
| 2000 | CAC22209 position 201, AAL09392 position 153, AAL09391 position 153, AAK69397 position |
| | 153, ABF18551 position 43, ABF18550 position 43, ABF18549 position 43, ABF18548 position 43, |
| | CAC51275 position 201, CAC51271 position 201, CAC51267 position 201, CAC51264 position |
| | 201, CAC51263 position 201, CAC51261 position 201, CAC51258 position 201, CAC51257 |
| | position 201, BAC06475 position 925, CAD62372 position 925, CAD62371 position 925, |
| | AAG27038 position 153, AAG27037 position 153 |
| 2001 | CAD62373 position 925, AAK92375 position 925, CAC35464 position 201, CAC35463 position |
| | 201, CAC35462 position 201, CAC35461 position 201, CAC23917 position 925, CAC86575 |
| | position 925. |
| 2002 | AAR07959 position 153, AAM62134 position 201. |
| 2003 | AAQ93493 position 925, AAR07963 position 153, AAR07962 position 153, AAR07961 position |
| | 153, AAR07960 position 153, AAR07965 position 153, AAR07964 position 153. |
| 2005 | ABD14417 position 201, ABC55721 position 43, CAJ51080 position 201, CAJ51079 position 201, |
| | CAJ51078 position 201, CAJ51077 position 201, CAJ51076 position 201, CAJ51075 position 201. |
| 2006 | ABG77563 position 197, ABG77564 position 30 |

Table 9 provides the accession numbers of FMDV isolates between 1962 and 2006 containing the conserved sequence hpsearhkqkivapvk (SEQ ID NO: 92) or the point mutation hptearhkqkivapvk (SEQ ID NO: 93) and the amino acid position within the VP1 protein sequence where the conserved Replikin sequence begins.

TABLE 9
FMDV SEQ ID NO: 92 OR SEQ ID NO: 93

| | |
|---|---|
| 1962 | AAP81678 position 147, AAP81677 position 147, ABA46700 position 195, ABA46699 position |
| | 195, ABA46698 position 195, ABA46697 Position 195, ABA46696 position 195, ABA46695 |
| | position 195, ABA46693 position 195, ABA46692 position 195, ABA46691 position 195, |
| | ABA46690 position 195, ABA46689 position 195, ABA46688 position 195, ABA46687 position |
| | 195, ABA46686 position 195, ABA46685 position 195, ABA46684 position 195, ABA46683 |
| | position 195, ABA46682 position 195, ABA46681 position 195, ABA46679 position 195, |
| | ABA46678 position 195, ABA46677 position 195, ABA46675 position 195, ABA46673 position |
| | 195, ABA46672 position 195, ABA46671 position 195, ABA46670 position 195, ABA46666 |
| | position 195, ABA46665 position 195, ABA46664 position 195, ABA46663 position 195, |
| | ABA46662 position 195, ABA46661 position 195, ABA46659 position 195, ABA46658 position |
| | 195, ABA46657 position 195, ABA46655 position 195, ABA46654 position 195, ABA46649 |
| | position 195, ABA46648 position 195, ABA46647 position 195, ABA46644 position 195, |
| | ABA46643 position 195, ABA46642 position 195, ABA46640 position 195, ABA46639 position |
| | 195, ABA46638 position 195, ABA46637 position 195, ABA46614 position 195, ABA46613 |
| | position 195, ABA46612 position 195, ABA46611 position 195, ABA46609 position 195, |
| | ABA46606 position 195, ABA46605 position 195, ABA46604 position 195, ABA46603 position |
| | 195, ABA46602 position 195, ABA46601 position 195, ABA46600 position 195, ABA46588 |
| | position 195, ABA46581 position 195, ABA46574 position 195, ABA46573 position 195, |
| | ABA46571 position 195, ABA46570 position 195, ABA46569 position 195, ABA46568 position |
| | 195, ABA46566 position 195, ABA46565 position 195, ABA46563 position 195, ABA46561 |
| | position 195, ABA46539 position 195, ABA46538 position 195, ABA46537 position 195, |
| | ABA46536 position 195, ABA46535 position 195, ABA46531 position 195, ABA46559 position |
| | 195. |
| 1974 | AAR85362 position 147, AAR85361 position 147. |
| 1975 | CAC20174 position 195. |
| 1977 | AAR22963 position 147, AAR22950 position 147. |
| 1978 | ABA46743 position 195, ABA46742 position 195, ABA46740 position 195. |
| 1979 | AAQ88330 position 147, AAQ88328 position 147, AAQ88325 position 147, AAQ88324 position |
| | 147, AAQ88323 position 147, AAQ88321 position 147, AAQ88319 position 147, AAQ88317 |
| | position 147, AAQ88316 position 147, AAQ88315 position 147, AAQ88314 position 147, |
| | AAQ88313 position 147, AAQ88312 position 147, AAG28362 position 37, AAG28355 position 37. |
| 1985 | CAC22326 position 84. |
| 1987 | AAK62003 position 37. |
| 1989 | CAC22174 position 84, AAK62024 position 63. |
| 1990 | CAC22178 position 37. |
| 1991 | CAC22175 position 37, CAC22328 position 56. |
| 1992 | CAC22240 position 79. |
| 1994 | CAC22233 position 56. |
| 1995 | CAC22216 position 38, CAC22171 position 97. |
| 1996 | CAC22194 position 121, CAC51235 position 195, AAR22945 position 147, AAR22942 position 147, |
| | AAK62005 position 63. |
| 1997 | CAC51273 position 195, CAC51268 position 195, CAC51249 position 195, CAC51236 position |
| | 195, AAL05249 position 37, AAL05248 position 79, AAL05247 position 56, AAL05246 position |
| | 70, AAL05245 position 37, AAL05243 position 50, AAL05242 position 37, AAL05236 position 37, |
| | AAL05235 position 59, AAL05234 position 37, AAL05233 position 37, AAL05229 position 37, |
| | AAL05228 position 37, AAL05221 position 37, AAL05207 position 37, AAL05196 position 58. |
| 1998 | ABI16250 position 195, ABI16249 position 195, ABI16248 position 195, ABI16247 position 195, |
| | ABI16246 position 195, ABI16245 position 195, ABI16244 position 195, ABI16242 position 195, |
| | ABI16241 position 195, ABI16240 position 195, ABI16239 position 195, ABI16238 position 195, |
| | ABI16237 position 195, ABI16236 position 195, ABI16235 position 195, ABI16234 position 195, |
| | ABI16232 position 195, ABI16231 position 195, ABI16229 position 195, ABI16227 position 195, |
| | CAC51239 position 195, CAC51238 position 195, AAR22957 position 147, AAL05256 position 37, |
| | AAL05255 position 37, AAL05254 position 37, AAL05253 position 37, AAL05250 position 37, |
| | AAL05244 position 37, AAL05241 position 37, AAL05240 position 37, AAL05238 position 37, |
| | AAL05237 position 39 |
| 1999 | CAC22228 position 94, AAG43385 position 37, CAC51332 position 137, CAC51255 position 195, |
| | CAC51318 position 195, CAC51247 position 195, CAC51246 position 195, CAC51245 position |
| | 195, CAD62370 position 919, CAD62208 position 919, CAC20187 position 195, AAR22956 |
| | position 147, AAF06146 position 37, AAL05251 position 37, AAL05210 position 37, AAL05209 |
| | position 37, AAL05208 position 37, AAL05204 position 37, AAL05203 position 39, AAL05202 |
| | position 37, AAL05201 position 37, AAL05200 position 37, AAL05198 position 37, AAL05195 |
| | position 53, AAL05194 position 52, AAL05193 position 37. |
| 2000 | CAC22209 position 195, AAL09392 position 147, AAL09391 position 147, AAK69397 position |
| | 147, ABF18551 position 37, ABF18550 position 37, ABF18549 position 37, ABF18548 position 37, |
| | CAC51275 position 195, CAC51271 position 195, CAC51267 position 195, CAC51264 position |
| | 195, CAC51263 position 195, CAC51261 position 195, CAC51258 position 195, CAC51257 |
| | position 195, BAC06475 position 919, CAD62372 position 919, CAD62371 position 919, |
| | AAG27038 position 147, AAG27037 position 147, ABA46733 position 195, ABA46732 position |
| | 195, ABA46731 position 195, ABA46730 position 195, ABA46729 position 195, ABA46728 |
| | position 195, ABA46727 position 195, ABA46726 position 195, ABA46725 position 195, |
| | ABA46724 position 195, ABA46722 position 195, ABA46721 position 195, ABA46720 position |
| | 195, ABA46719 position 195, ABA46717 position 194, ABA46716 position 195, ABA46715 |
| | position 195, ABA46714 position 195, ABA46713 position 195, ABA46712 position 195, |
| | ABA46711 position 195, ABA46709 position 195, ABA46708 position 195, ABA46706 position |
| | 195, ABA46705 position 195, ABA46704 position 195, BAB18050 position 195. |
| 2001 | CAD62373 position 919, AAK92375 position 919, CAC35464 position 195, CAC35463 position 195, |
| | CAC35462 position 195, CAC35461 position 195, CAG23917 position 919. |
| 2002 | AAM62134 position 195. |
| 2003 | AAQ93493 position 919. |
| 2005 | ABD14417 position 195, ABC55721 position 37. |
| 2006 | ABG77563 position 191. |

Accession No. AAG43385 (SEQ ID NO: 107) reports an FMDV serotype O isolate from 1999 that partly contains the RPG of SEQ ID NO: 124 and contains the conserved sequence SEQ ID NO: 91. In SEQ ID NO: 107, no Replikin sequences were identified in the amino-terminal. Replikin sequence SEQ ID NO: 108 was identified in the mid-molecule. Replikin sequence SEQ ID NO: 91 was identified in the carboxy-terminus.

Accession No. AAP81678 (SEQ ID NO: 111), reports an FMDV serotype 0 isolate from 1962 that partly contains the RPG of SEQ ID NO: 124 and contains the conserved sequence SEQ ID NO: 91. Accession No. ABA46641 (SEQ ID NO: 114) likewise reports an FMDV serotype O isolate from 1962 that partly contains the RPG of SEQ ID NO: 124 and contains the conserved sequence of SEQ ID NO: 91 and the conserved sequence of SEQ ID NO: 92 but for a single unknown residue at position 199 (SEQ ID NO: 115). In SEQ ID NO: 114, no Replikin sequences were identified in the amino-terminus or mid-molecule portion of the sequence. SEQ ID NOS: 115 and 116 were isolated in the carboxy-terminus.

Accession No. ABG77564 (SEQ ID NO: 118) reports an FMDV serotype O isolated from 2006 that partly contains the RPG of SEQ ID NO: 124 and contains the conserved sequence SEQ ID NO: 91. In SEQ ID NO: 118, no Replikins were identified in the amino terminus of the sequence. SEQ ID NOS: 119-121 and 91 were identified as Replikins in the mid-molecule. And no Replikins were identified in the carboxy terminus.

In addition to the diagnostic power of Replikin technology shown in these examples, it is clear that recognition for the first time of this class of virus peptides, and the discovery that they are related to rapid replication, virus outbreaks and high morbidity and mortality, makes the Replikins, and particularly the Replikin Peak Gene structures illustrated here, new conserved prime targets for treatment and vaccines in FMDV and other viruses. For example, the Replikin sequences (SEQ ID NOS: 91-93) provide invariant targets for such a vaccine. Likewise, the RPGs of SEQ ID NOS: 124 and 130 and the Replikin sequence identified in the accession number sequences (SEQ ID NOS: 108, 115-116 and 119-121) are preferred sequences for immunogenic compositions and vaccines. An embodiment of the invention, therefore, is a vaccine comprising at least one of the sequences SEQ ID NOS: 91-93 or SEQ ID NOS: 108, 115-116 and 119-121 or any combination thereof.

VIII. Methods of Predicting and Treating Outbreaks of West Nile Virus Using RPGs and Related Replikin Sequences

Applicants have now demonstrated a correlation between an increase in Replikin Count in a Replikin Peak Gene of the west nile virus (WNV) (e.g., SEQ ID NO: 245) and outbreaks, morbidity and mortality in the viral disease. See FIG. 12. Applicants have also demonstrated a correlation between Replikin Count in the whole virus genome and morbidity and mortality. See U.S. Prov. Appln. Ser. No. 60/853,744, filed Aug. 16, 2007.

Review of publicly available sequences of isolates of WNV from 1982-2007 revealed a Replikin Peak Gene in the envelope protein of west nile virus that has now been associated with virulence and lethality. In comparison with morbidity and mortality data in the United States between 1999 and 2006, an association between Replikin Count in the envelope protein of west nile virus and morbidity and mortality data is clear. See FIG. 12. Applicants' analysis of a Replikin Peak Gene in an envelope protein sequence of Accession No. ABA54585 (e.g., SEQ ID NO: 245) is provided in Example 7 below.

FIG. 12 illustrates the Replikin Count of Replikins observed in the envelope protein in PubMed accession numbers on a year by year basis between 1982 and 2006. Increases in Replikin Count on a year by year basis are correlatable with both reported morbidity of the virus in the United States and reported mortality from viral infections in the United States.

The data for FIG. 12 is provided in Table 10 below. Years in which no data were available are not included in the table. It may be observed that Replikin Count correlates with changes in both morbidity and mortality in the U.S. population between 1999 and 2006. The data further make clear a relative decrease in Replikin Count in 2004 followed by a time of relative quiescence of the west nile virus in the United States in 2004 and 2005. Additionally, beginning in 2000, morbidity and mortality increases in relation to the increasing Replikin Count.

TABLE 10
WNV Envelope Protein

| | | | Mean | | | Mor- | Mor- |
|---|---|---|---|---|---|---|---|
| Year | PubMed Accession Number-Replikin Count | Isolates | RC | S.D. | Significance | bidity | tality |
| 1982 | 84028435 111 | 1 | 3.2 | 0.0 | | | |
| 1985 | P06935 109 NP_776014 109 NP_041724 109 NP_776013 109 | 6 | 3.2 | 0.0 | low p < | | |
| | NP_776012 109 AAA48498 109 | | | | .001 | | |
| 1988 | P14335 95 BAA00176 95 | 2 | 2.8 | 0.0 | prev | | |
| | | | | | p < .001 | | |
| 1995 | AAW80621 9 | 1 | 4.7 | 0.0 | | | |
| 1996 | P51681 8 | 1 | 2.3 | 0.0 | | | |
| 1998 | AAW81711 107 AAD28624 41 | 2 | 4.2 | 1.5 | low p < | | |
| | | | | | .50, | | |
| | | | | | prev | | |
| | | | | | p < .30 | | |
| 1999 | AAL10755 6 AAL10754 6 AAL10752 6 AAL10751 6 AAL10750 7 | 53 | 3.6 | 0.9 | low p < | 62 | 7 |
| | AAL10749 6 AAG49029 3 AAG49028 3 AAG49027 2 AAD31720 | | | | .005, | | |
| | 32 AAL10753 9 AAF26360 40 AAL10748 6 AAL10747 6 | | | | prev | | |
| | AAL10746 6 AAL10745 6 AAL10744 6 AAL10743 6 AAL10742 6 | | | | p > .50 | | |
| | AAL10741 4 AAL10740 6 AAL10738 6 AAL10737 6 AAL10736 6 | | | | | | |
| | AAL10735 6 AAL10734 6 AAL10733 6 AAL10732 6 AAL10731 6 | | | | | | |
| | AAL10730 6 AAL10729 6 AAL10728 6 AAL10727 6 AAL10725 10 | | | | | | |
| | AAL10724 6 AAL10723 6 id = 15919195 6 id = 12246899 6 | | | | | | |
| | id = 12246897 6 id = 12246895 6 id = 12246893 6 AAG49629 6 | | | | | | |
| | AAG49628 6 AAL10739 6 AAL10726 6 AAD28623 40 AAF20092 | | | | | | |
| | 97 AAG02040 97 AAF18443 97 AAF20205 97 AAF20207 7 | | | | | | |
| | AAF20206 7 AAF20204 7 | | | | | | |
| 2000 | AAK06624 97 AAG02039 98 AAG02038 97 | 3 | 2.8 | 0.0 | low p < | 21 | 2 |
| | | | | | .001, | | |
| | | | | | prev | | |
| | | | | | p < .001 | | |
| 2001 | AAM70028 28 AAL14222 30 AAL14221 30 AAL14220 30 | 129 | 3.6 | 2.0 | low p < | 66 | 9 |
| | AAL14219 30 AAL14218 30 AAL14217 30 AAL14216 30 | | | | .02, | | |
| | AAL14215 30 AAK58104 30 AAK58103 31 AAK58102 30 | | | | prev | | |
| | AAK58101 30 AAK58100 30 AAK58099 31 AAK58098 30 | | | | p < .001 | | |
| | AAK58097 30 AAK58096 30 AAK52303 30 AAK52302 30 | | | | | | |
| | AAK52301 30 AAK52300 30 AAK62766 32 AAK62765 32 | | | | | | |
| | AAK62764 32 AAK62763 32 AAK62762 32 AAK62761 32 | | | | | | |
| | AAK62760 32 AAK62759 32 AAK62758 32 AAK62757 32 | | | | | | |
| | AAK62756 32 AAL07765 6 AAL07764 6 AAL07763 6 AAL07762 6 | | | | | | |
| | AAL07761 6 AAK91592 20 AAM81753 97 AAM81752 97 | | | | | | |
| | AAM81751 97 AAM81750 97 AAM81749 97 AAM21941 32 | | | | | | |
| | AAK67141 7 AAK67140 7 AAK67139 7 AAK67138 7 AAK67137 7 | | | | | | |
| | AAK67136 7 AAK67135 7 id = 14550088 7 id = 14550086 7 | | | | | | |
| | id = 14550084 7 id = 14550082 7 id = 14550080 7 id = 14550078 7 | | | | | | |
| | id = 14550076 7 id = 14550074 7 id = 14550072 7 id = 14550070 7 | | | | | | |
| | AAK67124 3 AAK67123 7 AAK67122 7 AAK67121 7 AAK67120 7 | | | | | | |
| | AAK67119 7 AAK67118 7 AAK67117 7 AAK67116 7 AAK67115 7 | | | | | | |
| | AAK67114 7 AAK67113 7 AAK67112 7 AAK67111 7 AAK67110 7 | | | | | | |
| | AAK67109 7 AAK67108 7 AAK67107 7 AAK67106 7 AAK67105 7 | | | | | | |
| | AAK67104 7 AAK67103 7 AAK67102 7 AAK67101 7 AAK67100 7 | | | | | | |
| | AAK67099 7 AAK67098 7 AAK67097 7 AAK67096 7 AAK67095 7 | | | | | | |
| | AAK67094 7 AAK67093 7 AAK67092 7 AAK67091 7 AAK67090 7 | | | | | | |
| | AAK67089 7 AAK67088 7 AAK67087 7 AAK67086 7 AAK67085 7 | | | | | | |
| | AAK67084 7 AAK67083 7 AAK67082 7 AAK67081 7 AAK67080 7 | | | | | | |
| | AAK67079 7 AAK67078 7 AAK67077 7 AAK67076 7 AAK67075 7 | | | | | | |
| | AAK67074 7 AAK67073 7 AAK67072 7 AAK67071 7 AAK67070 5 | | | | | | |
| | AAK67069 7 AAK67068 7 AAK67067 7 AAK67066 7 AAK67065 7 | | | | | | |
| | AAK67064 7 AAL87748 19 AAL87747 18 AAL87746 19 | | | | | | |
| | AAL87745 18 AAL37596 18 AAM21944 24 | | | | | | |
| 2002 | AAO26579 30 AAO26578 30 AAN77484 3 AAM09856 6 | 17 | 4.8 | 1.4 | low p < | 4,156 | 284 |
| | AAM09855 6 AAM09854 6 AAN85090 97 AAO73303 36 | | | | .001, | | |
| | AAO73302 36 AAO73301 36 AAO73300 36 AAO73299 36 | | | | prev | | |
| | AAO73298 36 AAO73297 36 AAO73296 36 AAO73295 36 | | | | p < .002 | | |
| | AAL87234 96 | | | | | | |
| 2003 | AAP20887 96 AAR17575 32 AAR17574 32 AAR17573 32 | 107 | 5.2 | 1.5 | low p < | 9,862 | 264 |
| | AAR17572 32 AAR17571 32 AAR17570 32 AAR17569 32 | | | | .001, | | |
| | AAR17568 32 AAR17567 32 AAR17566 32 AAR17565 32 | | | | prev | | |
| | AAR17564 32 AAR17563 32 AAR17562 32 AAR17561 32 | | | | p < .30 | | |
| | AAR17560 32 AAR17559 32 AAR17558 32 AAR17557 32 | | | | | | |
| | AAR17556 32 AAR17555 32 AAR17554 32 AAR17553 32 | | | | | | |
| | AAR17552 32 AAR17551 32 AAR17550 32 AAR17549 32 | | | | | | |
| | AAR17548 32 AAR17547 32 AAR17546 32 AAR17545 32 | | | | | | |
| | AAR17544 32 AAR17543 32 AAR17542 32 AAQ87608 16 | | | | | | |
| | AAQ87607 16 AAQ87606 14 AAR10804 6 AAR10803 6 AAR10802 | | | | | | |
| | 6 AAR10801 6 AAR10800 6 AAR10799 6 AAR10798 6 AAR10797 | | | | | | |
| | 6 AAR10796 6 AAR10795 6 AAR10794 6 AAR10793 6 AAR10792 | | | | | | |
| | 6 AAR10791 6 AAR10790 6 AAR10789 6 AAR10788 6 AAR10787 | | | | | | |
| | 6 AAR10786 6 AAR10785 6 AAR10784 6 AAR10783 6 AAR10782 | | | | | | |
| | 6 AAR10781 6 AAR10780 6 AAQ88403 10 AAQ88402 10 | | | | | | |
| | id = 40288320 36 AAQ55854 97 AAR14153 36 id = 92919472 97 | | | | | | |
| | AAR84614 95 AAR06948 36 AAR06947 36 id = 37993725 36 | | | | | | |
| | id = 37993723 36 id = 37993721 36 id = 37993719 36 id = 37993717 36 | | | | | | |
| | AAR06941 36 AAR06940 36 AAR06939 36 AAR06938 36 | | | | | | |
| | AAR06937 36 id = 37993705 35 id = 37993703 36 id = 37993701 36 | | | | | | |
| | id = 37993699 36 id = 37993697 36 AAR06931 36 AAQ00999 100 | | | | | | |
| | AAQ00998 97 AAP22087 97 AAP22086 97 AAP22089 97 | | | | | | |
| | AAP22088 96 AAP85247 6 AAP85246 6 AAP85245 6 AAP85244 6 | | | | | | |
| | AAP85243 6 AAP85242 6 AAP85241 6 AAP85240 6 AAP85239 6 | | | | | | |
| | AAP85238 6 AAP85237 6 AAP78942 95 AAP78941 95 | | | | | | |
| 2004 | 1S6NA 4 AAT11553 32 AAT11552 32 AAT11551 32 AAT11550 32 | 52 | 4.3 | 1.8 | low p < | 2,539 | 100 |
| | AAT11549 32 AAT11548 32 AAT11547 32 AAT11546 32 | | | | .001, | | |
| | AAT11545 32 AAT11544 32 AAT11543 32 AAT11542 32 | | | | prev | | |
| | AAT11541 32 AAT11540 32 AAT11539 32 AAT11538 32 | | | | p < .002 | | |
| | AAT11537 32 AAT11536 32 AAT11535 32 AAT11534 32 | | | | | | |
| | AAS75296 6 AAS75295 6 AAS75294 6 AAS75293 6 AAS75292 6 | | | | | | |
| | AAS75291 6 id = 51095222 108 AAU00153 96 id = 55669122 97 | | | | | | |
| | BAD34490 97 BAD34489 97 BAD34488 97 AAV68177 97 | | | | | | |
| | id = 73913544 106 id = 59876233 97 AAT92099 97 AAT92098 97 | | | | | | |
| | AAT02759 111 AAV52690 96 AAV52689 97 AAV52688 97 | | | | | | |
| | AAV52687 97 AAV49728 6 AAV49727 6 AAV49726 6 AAV49725 | | | | | | |
| | 6 AAV49724 6 AAW56064 97 AAW56066 97 AAW56065 97 | | | | | | |
| | AAW28871 97 | | | | | | |
| 2005 | ABC18309 8 ABC18308 9 ABC02196 3 1ZTXE 5 AAY67877 9 | 119 | 4.4 | 1.9 | low p < | 3,000 | 119 |
| | AAY67876 11 AAY67875 11 AAY67874 8 AAY67873 8 | | | | .001, | | |
| | AAY67872 8 AAY67871 8 AAY67870 8 AAY67869 8 AAY67868 8 | | | | prev | | |
| | AAY67867 8 AAY67866 8 AAY57985 8 ABB01532 97 AAZ32750 | | | | p > .50 | | |
| | 97 AAZ32749 97 AAZ32748 97 AAZ32747 97 AAZ32746 97 | | | | | | |
| | AAZ32745 97 AAZ32744 97 AAZ32743 97 AAZ32742 97 | | | | | | |
| | AAZ32741 97 AAZ32739 97 AAZ32737 97 AAZ32736 97 | | | | | | |
| | AAZ32734 97 AAZ32733 97 id = 71483607 97 id = 71483605 97 | | | | | | |
| | id = 71483603 97 id = 71483601 97 ABC40712 100 ABB01533 101 | | | | | | |
| | AAY55949 97 ABA62343 97 id = 63098704 36 id = 63098702 36 | | | | | | |
| | AAY29684 6 AAY29685 6 AAY29683 6 AAY29682 6 AAY29681 6 | | | | | | |
| | AAY29680 6 AAY29679 6 AAY29678 6 AAY29677 7 AAY29676 7 | | | | | | |
| | id = 84028433 111 id = 76446583 37 ABA43045 37 ABA43044 37 | | | | | | |
| | ABA43043 37 ABA43042 37 ABA43041 37 ABA43040 37 | | | | | | |
| | ABA43039 37 ABA43038 37 ABA43037 37 ABA43036 37 | | | | | | |
| | ABA43035 37 ABA43034 37 ABA43033 37 ABA43032 37 | | | | | | |
| | ABA43031 37 ABA43030 37 ABA43029 37 ABA43028 37 | | | | | | |
| | ABA43027 37 ABA43026 37 ABA43025 37 ABA43024 37 | | | | | | |
| | ABA43023 37 ABA43022 37 ABA43021 37 ABA43020 37 | | | | | | |
| | ABA43019 37 ABA43017 37 ABA43016 37 id = 76446521 37 | | | | | | |
| | id = 76446519 37 id = 76446517 37 id = 76446515 37 id = 76446513 37 | | | | | | |
| | ABA43010 37 ABA43009 37 ABA43008 37 ABA43007 37 | | | | | | |
| | ABA43006 37 id = 76446501 37 id = 76446499 37 id = 76446497 37 | | | | | | |
| | id = 76781572 105 id = 76781570 105 ABA54593 105 ABA54592 105 | | | | | | |
| | ABA54591 105 ABA54590 105 ABA54589 105 ABA54588 105 | | | | | | |
| | ABA54587 105 ABA54586 105 ABA54585 105 ABA54584 105 | | | | | | |
| | ABA54583 105 ABA54582 105 ABA54581 105 ABA54580 105 | | | | | | |
| | ABA54579 105 id = 76781538 105 id = 76781536 105 id = 76781534 | | | | | | |
| | 105 id = 76781532 105 AAY54162 97 | | | | | | |
| 2006 | ABI81406 34 ABI81405 34 ABI81404 34 ABI81403 34 ABI81402 | 279 | 6.4 | 1.4 | low p < | 4269 | 177 |
| | 34 ABI81401 34 ABI81400 34 ABI81399 34 ABI81398 34 | | | | .001, | | |
| | ABI81397 34 ABI81396 34 ABI81395 34 ABI81394 34 ABI81393 | | | | prev | | |
| | 34 ABI81392 34 ABI81391 34 ABI81390 34 ABI81389 34 | | | | p < .001 | | |
| | ABI81388 34 ABI81387 34 ABI81386 34 ABI81385 34 ABI81384 | | | | | | |
| | 34 ABI81383 34 ABI81382 34 ABI81381 34 ABI81380 34 | | | | | | |
| | ABI81379 34 ABI81378 34 ABI81377 34 ABI81376 34 ABI81375 | | | | | | |
| | 34 ABI81374 34 ABI81373 34 ABI81372 34 ABI81371 34 | | | | | | |
| | ABI81370 34 ABI81369 34 ABI81368 34 ABI81367 34 ABI81366 | | | | | | |
| | 34 ABI81365 34 ABI81364 34 ABI81363 34 ABI81362 34 | | | | | | |
| | ABI81361 34 ABI81360 34 ABI81359 34 ABI81358 34 ABI81357 | | | | | | |
| | 34 ABI81356 34 ABI81355 34 ABI81354 34 ABI81353 34 | | | | | | |
| | ABI81352 34 ABI81351 34 ABI81350 34 ABI81349 34 ABI81348 | | | | | | |
| | 34 ABI81347 34 ABI81346 34 ABI81345 34 ABI81344 34 | | | | | | |
| | ABI81343 34 ABI81342 34 ABI81341 34 ABI81340 34 ABI81339 | | | | | | |
| | 34 ABI81338 34 ABI81337 34 ABI81336 34 ABI81335 34 | | | | | | |
| | ABI81334 34 ABI81333 34 ABI81332 34 ABI81331 34 ABI81330 | | | | | | |
| | 34 ABI81329 34 ABI81328 34 ABI81327 34 ABI81326 34 | | | | | | |
| | ABI81325 34 ABI81324 34 ABI81323 34 ABI81322 34 ABI81321 | | | | | | |
| | 34 ABI81320 34 ABI81319 34 ABI81318 34 ABI81317 34 | | | | | | |
| | ABI81316 34 ABI81315 34 ABI81314 34 ABI81313 34 ABI81312 | | | | | | |
| | 34 ABI81310 34 ABI81308 34 ABI81307 34 ABI81306 34 | | | | | | |
| | ABI81305 34 ABI81304 34 ABI81303 34 ABI81302 34 ABI81301 | | | | | | |
| | 34 ABI81300 34 ABI81299 34 ABI81298 34 ABI81297 34 | | | | | | |
| | ABI81296 34 ABI81295 34 ABI81294 34 ABI81293 34 ABI81292 | | | | | | |
| | 34 ABI81291 34 ABI81290 34 ABI81289 34 ABI81288 34 | | | | | | |
| | ABI81287 34 ABI81286 34 ABI81285 34 ABI81284 34 ABI81283 | | | | | | |
| | 34 ABI81282 34 ABI81281 34 ABI81280 34 ABI81279 34 | | | | | | |
| | ABI81278 34 ABI81277 34 ABI81276 34 ABI81275 34 ABI81274 | | | | | | |
| | 34 ABI81273 34 ABI81272 34 ABI81271 34 ABI81270 34 | | | | | | |
| | ABI81269 34 ABI81268 34 ABI81267 34 ABI81266 34 ABI81265 | | | | | | |
| | 34 ABI81264 34 ABI81263 34 ABI81262 34 ABI81260 34 | | | | | | |
| | ABI81259 34 ABI81258 34 ABI81257 34 ABI81256 34 ABI81255 | | | | | | |
| | 34 ABI81254 34 ABI81253 34 ABI81252 34 ABI81251 34 | | | | | | |
| | ABI81250 34 ABI81249 34 ABI81248 34 ABI81247 34 ABI81246 | | | | | | |
| | 34 ABI81245 34 ABI81244 34 ABI81243 34 ABI81242 34 | | | | | | |
| | ABI81241 34 ABI81240 34 ABI81239 34 ABI81238 34 ABI81237 | | | | | | |
| | 34 ABI81236 34 ABI81235 34 ABI81234 34 ABI81233 34 | | | | | | |
| | ABI81232 34 ABI81231 34 ABI81230 34 ABI81229 34 ABI81228 | | | | | | |
| | 34 ABJ90133 32 ABJ90132 32 ABJ90131 32 ABJ90130 32 | | | | | | |
| | ABJ90129 32 ABJ90128 32 ABJ90127 32 ABJ90126 32 ABJ90125 | | | | | | |
| | 32 ABJ90124 32 ABJ90123 32 ABJ90122 32 ABJ90121 32 | | | | | | |
| | ABJ90120 32 ABJ90119 32 ABJ90118 32 ABJ90117 32 ABJ90116 | | | | | | |
| | 32 ABJ90115 32 ABJ90114 32 ABJ90113 32 ABJ90112 32 | | | | | | |
| | ABJ90111 32 ABJ90110 32 ABJ90109 32 ABJ90108 32 ABJ90107 | | | | | | |
| | 32 ABJ90106 32 ABJ90105 32 ABJ90104 32 ABJ90103 32 | | | | | | |
| | ABJ90102 32 ABJ90101 32 ABJ90100 32 ABJ90099 32 ABJ90098 | | | | | | |
| | 32 ABJ90097 32 ABJ90096 32 ABJ90095 32 ABJ90094 32 | | | | | | |
| | ABJ90093 32 ABJ90092 32 ABJ90091 32 ABJ90090 32 ABJ90087 | | | | | | |
| | 32 ABJ90086 32 ABJ90085 32 ABJ90084 32 ABJ90083 32 | | | | | | |
| | ABJ90082 32 ABJ90080 32 ABJ90079 32 ABJ90078 32 ABJ90077 | | | | | | |
| | 32 ABJ90076 32 ABJ90075 32 ABJ90074 32 ABJ90073 32 | | | | | | |
| | ABJ90072 32 ABJ90071 32 ABJ90070 32 ABJ90069 32 ABJ90068 | | | | | | |
| | 32 ABJ90067 32 ABJ90066 32 ABD85083 99 ABD85082 99 | | | | | | |
| | ABD85081 99 ABD85080 99 ABD85079 99 ABD85078 99 | | | | | | |
| | ABD85077 99 ABD85076 99 id = 90025142 99 id = 90025140 99 | | | | | | |
| | id = 90025138 99 id = 90025136 99 id = 90025134 99 ABD85070 99 | | | | | | |
| | ABD85069 99 ABD85068 99 ABD85067 99 ABD85066 99 | | | | | | |
| | ABD85065 99 ABD85064 99 ABG36517 36 ABD19513 97 | | | | | | |
| | ABD19512 96 ABD19511 97 ABD19510 97 ABI26622 40 ABI26621 | | | | | | |
| | 40 id = 89340787 97 id = 89340785 97 id = 89340783 97 ABD67759 97 | | | | | | |
| | ABD67758 97 ABD67757 97 ABD67756 97 ABD19642 97 | | | | | | |
| | id = 87116125 97 id = 87116123 97 2I69A 24 | | | | | | |
| 2007 | ABQ52692 97 ABO69610 36 ABO69609 36 ABO69608 36 | 20 | 5.3 | 0.6 | low p < | | |
| | ABO69607 36 ABO69606 36 ABO69605 36 ABO69604 36 | | | | .001, | | |
| | ABO69603 36 ABO69602 36 ABO69601 36 ABO69600 36 | | | | prev | | |
| | ABO69599 36 ABO69598 36 id = 134285072 36 id = 134285070 36 | | | | p < .001 | | |
| | id = 134285068 36 id = 134285066 36 id = 134285064 36 ABO69592 36 | | | | | | |

Upon analysis of Replikin Counts of publicly available sequences from the entire genome of WNV and comparison with WNV morbidity and mortality data from the United States Center for Disease Control, the applicants observed that the mean Replikin Count of WNV increased significantly between years 2000, 2004, 2005 and 2006, respectively. As seen in Table 11, the mean Replikin Count of 2.8±0 observed in 2000 was found to be significantly different (p<0.001) from the mean Replikin Count of 3.8±1.7 observed in 2004, the mean Replikin Count observed in 3.8±1.7 in 2004 was found to be significantly different (p<0.01) from the mean Replikin Count observed in 4.5±1.8 in 2005, and, finally, the mean Replikin Count observed in 4.5±1.8 in 2005 was found to be significantly different (p<0.001) from the mean Replikin Count observed in 6.0±1.1 in 2006.

TABLE 11
WNV Whole Genome

| | | | | Significance | | |
|---|---|---|---|---|---|---|
| | Accession | | | (compared to | | |
| | Records for | Replikin | Standard | previous listed | | |
| Year | WNV | Count | Deviation | year) | Morbidity | Mortality |
| 2000 | 2 | 2.8 | ±0.0 | | CDC 21 | CDC 2 |
| 2004 | 68 | 3.8 | ±1.7 | prev p < 0.001 | CDC 2,539 | CDC 100 |
| 2005 | 137 | 4.5 | ±1.8 | prev p < 0.01 | CDC 3000 | CDC 119 |
| 2006 | 211 | 6.0 | ±1.1 | prev p < 0.001 | CDC 4269 | CDC 177 |

In the summer of 2007, Applicants reviewed the data for the whole WNV genome in publicly available sequences as provided in Table 11 and expressly predicted that a virulent increase in infection of WNV would likely follow the significant increase observed between each of the analyzed years. Immediately after Applicants' prediction, the California Department of Public Health confirmed Applicants' prediction by reporting that infections of WNV in California through Aug. 2, 2007 had been three times greater than infections seen in the previous year and a health emergency for three California counties was declared.

The epidemiology and virology of WNV is different from the epidemiology and virology of some other viruses discussed herein such as influenza, FMDV, PRRSV and PCV. Nevertheless, a correlation between increases in Replikin Count in the WNV envelope protein and morbidity and mortality provides compounding data establishing a shared phenomenon of rapid replication and virulence with an overwhelming number of other tested viruses and organisms.

In WNV and the other viruses and pathogens described herein, prediction of epidemics and future outbreaks may be made, for example, by (1) reviewing the Replikin Counts of isolates of WNV and identifying a RPG, for example, and RPG in the envelope protein (e.g., SEQ ID NO: 245), (2) comparing the Replikin Counts in the RPG, in the protein or gene area containing the RPG, or in the whole virus genome for a particular year with Replikin Counts from other years. A significant increase in Replikin Count from one year to the next and preferably over one, two or three years provides predictive value of an emerging strain of WNV that may begin an outbreak of more highly virulent WNV. A WNV outbreak may be predicted within about six months to about one year or more from the observation of a significant increase in Replikin Count.

More preferably, an outbreak of WNV may be predicted within about six months to about one year from the observation of a significant increase in Replikin Count over two or three years or, as in inventors prediction in 2007, following the observation of strongly significant increases over several years such as wherein Replikin Counts between 2000, 2004 and 2006 had p values of less than at least 0.001 and frequently less than 0.001. As such, significant increases may be observed over a time period of more than one year, such as three, four, five or more years. An outbreak may likewise be predicted within about six months to about one year from the initial observation of an observable decrease in Replikin Count following a significant increase. Using this method, Applicants prospectively predicted the beginnings of a 2007 outbreak of WNV. The method may also employ isolates of individual strains or isolates of all strains of WNV.

An embodiment of the invention provides a segment of the genome or a protein or segment of a protein of the WNV in which the expressed gene or expressed gene segment has the highest concentration of Replikins, or Replikin Count (number of Replikins per 100 amino acids), when compared to other segments or named genes of the genome, namely the RPG. An RPG (SEQ ID NO: 245) in Accession No. ABA54585 is reported in Example 7 below. Twelve Replikin sequences (SEQ ID NOS: 246-257) are identified in the RPG diagnostic, preventive, therapeutic and predictive applications. These Replikin sequences are preferred embodiments of immunogenic compositions and vaccines. The invention further provides Replikin sequences within the identified RPG that are conserved in the genome over time and, as such, are available as relatively invariant preferred targets for diagnosis and manipulation of rapid replication and virulence in WNV through immunogenic responses and vaccines.

IX. Methods of Predicting and Treating Outbreaks of Porcine Reproductive and Respiratory Syndrome Virus (PRRSV) Using Replikin Sequences

An increase in Replikin concentration in PRRSV is predictive of an increase in virulence of the virus and allows for prediction of forthcoming outbreaks or increases in mortality. A review of publicly available amino acid sequences of isolates of PRRSV that demonstrate an increase in Replikin concentration in the genome or a genome segment, or in a protein or protein fragment of the virus over time or between isolates is used as a predictor of an increase in outbreaks and morbidity and mortality of pigs infected with PRRSV. Publicly available sequences for isolates of PRRSV from PubMed or other public or private sources may be analyzed by hand or using proprietary search tool software (ReplikinForecast™ from REPLIKINS LLC, Boston, Mass.).

The inventors have now identified a Replikin Peak Gene in the nucleocapsid protein of the Porcine Respiratory and Reproductive Syndrome Virus (PRRSV) and have demonstrated a correlation between increased Replikin Count in the nucleocapsid protein of PRRSV between 2004 and 2007 and major outbreaks of PRRSV in China. Example 8.

FIG. 13 illustrates Replikin Counts in the nucleocapsid protein of PRRSV (SEQ ID NO: 353). The Replikin Count is seen to increase between 2004 and 2007. This increase correlates with a major outbreak of Porcine Reproductive and Respiratory Syndrome in China. Further, standard deviation from the mean in 2005 is considerably larger than other years demonstrating a marked increase in Replikin Count was occurring in 2005. The large increase that was occurring in 2005 based on increases in standard deviation is confirmed as an increase in mean Replikin Count in 2006. The large standard deviation observed in 2005 indicates that more members of the class had increasing Replikin Counts. Standard deviation in 2005 was an early warning prior to the increase in the mean count in 2006 and 2007. A similar phenomenon is observable in FIG. 7. These data provide further confirmation of the predictive value of the RPG Replikin Count in viral outbreaks and provide specific support for RPG Replikin Count as a predictive tool in PRRSV and viruses in pigs generally.

The invention provides RPGs and Replikin sequences within the identified RPGs for diagnostic, preventive and therapeutic applications. For example, each Replikin sequences identified within an identified RPG in PRRSV and other viruses, organisms and malignancies is available for diagnostic and therapeutic applications including vaccines, immunogenic compositions and antibody therapies. The entire Replikin Peak Gene sequence or fragments thereof are likewise available for diagnostic, preventive, therapeutic and predictive applications. Further, the presence of the Replikin Peak Gene in an isolate of the virus is indicative of rapid replication.

As discussed herein, applicants have identified RPGs of available PRRSV isolates within the nucleocapsid protein of PRRSV. Identification of these RPGS is different, for example, from the Replikin Peak Gene previously identified by applicants in H5N1 influenza in one polymerase area, namely the RNA-directed RNA polymerase or pB1 protein. Identification of Replikin Peak Genes in different structures of different viruses is made possible through the strict criteria for a Replikin sequence as defined by the applicants. The proprietary software ReplikinForecast™ (licensable from REPLIKINS LLC, Boston, Mass.) provides an efficient survey of publicly available Replikin sequences and identification and isolation in silico of the Replikin Peak Gene.

The size of a Replikin Peak Gene, both in terms of the number of amino acids and the Replikin Count, will depend upon the size of the sequence of the entire genome, protein or fragment thereof that has been isolated and reported. The invention further provides Replikin sequences within the identified Replikin Peak Gene or Area that are conserved in the genome over time and, as such, are available as relatively invariant targets for diagnosis and manipulation of rapid replication and virulence in PRRSV.

Further, the following RPGs have been identified in PRRSV isolates from China reported at Accession Nos. AAM18565, AAP81809 and ABL60920, respectively:

(SEQ ID NO:341)

| | |
|---|---|
| (1) | k⁷ q⁸ q⁹ k¹⁰ k¹¹ k¹² k¹³ g¹⁴ n¹⁵ g¹⁶ q¹⁷ p¹⁸ |
| | v¹⁹ n²⁰ q²¹ l²² c²³ q²⁴ m²⁵ l²⁶ g²⁷ k²⁸ i²⁹ i³⁰ |
| | a³¹ q³² q³³ n³⁴ q³⁵ s³⁶ r³⁷ g³⁸ k³⁹ g⁴⁰ p⁴¹ g⁴² |
| | k⁴³ k⁴⁴ s⁴⁵ k⁴⁶ k⁴⁷ k⁴⁸ n⁴⁹ p⁵⁰ e⁵¹ k⁵² p⁵³ h⁵⁴ |
| | f⁵⁵ p⁵⁶ l⁵⁷ a⁵⁸ t⁵⁹ e⁶⁰ d⁶¹ d⁶² v⁶³ r⁶⁴ h⁶⁵ h⁶⁶ |
| | (China 2000) |

(SEQ ID NO:342)

| | |
|---|---|
| (2) | k⁷ q⁸ q⁹ k¹⁰ r¹¹ k¹² k¹³ g¹⁴ d¹⁵ g¹⁶ q¹⁷ p¹⁸ |
| | v¹⁹ n²⁰ q²¹ l²² c²³ q²⁴ m²⁵ l²⁶ g²⁷ k²⁸ i²⁹ i³⁰ |
| | a³¹ q³² q³³ n³⁴ q³⁵ s³⁶ r³⁷ g³⁸ k³⁹ g⁴⁰ p⁴¹ g⁴² |
| | k⁴³ k⁴⁴ n⁴⁵ k⁴⁶ k⁴⁷ k⁴⁸ n⁴⁹ p⁵⁰ e⁵¹ k⁵² p⁵³ h⁵⁴ |
| | f⁵⁵ p⁵⁶ l⁵⁷ a⁵⁸ t⁵⁹ e⁶⁰ d⁶¹ d⁶² v⁶³ r⁶⁴ h⁶⁵ h⁶⁶ |

(SEQ ID NO:343)

| | |
|---|---|
| (3) | k⁷ q⁸ q⁹ k¹⁰ k¹¹ k¹² k¹³ g¹⁴ n¹⁵ g¹⁶ q¹⁷ p¹⁸ |
| | v¹⁹ n²⁰ q²¹ l²² c²³ q²⁴ m²⁵ l²⁶ g²⁷ k²⁸ i²⁹ i³⁰ |
| | a³¹ q³² q³³ n³⁴ q³⁵ s³⁶ r³⁷ g³⁸ k³⁹ g⁴⁰ p⁴¹ g⁴² |
| | k⁴³ k⁴⁴ n⁴⁵ r⁴⁶ k⁴⁷ k⁴⁸ n⁴⁹ p⁵⁰ e⁵¹ k⁵² p⁵³ h⁵⁴ |
| | f⁵⁵ p⁵⁶ l⁵⁷ a⁵⁸ t⁵⁹ e⁶⁰ d⁶¹ d⁶² v⁶³ r⁶⁴ h⁶⁵ h⁶⁶ |
| (China 2006). | |

The identified RPG sequences are identical across the 2000, 2003 and 2006 isolates except for point mutations at positions 45 and 46 (underlined in bold). These sequences are, therefore, relatively invariant targets for diagnosis and manipulation of rapid replication and virulence in PRRSV and are available as vaccines against the disease.

Point mutations, such as in positions 45 and 46 in the above-listed Chinese isolates, provide excellent predictive capacity. In the highly virulent and fatal Chinese variant disclosed in 2006 at ABL60920 (SEQ ID NO: 343), the asparagine and arginine at positions 45 and 46 are the same residues in the same relative positions as asparagine and arginine at residues 21 and 22 in the RPG of the highly virulent PRRSV 2006 Mexican isolate publicly available at Accession No. ABF 19568 (comparable mutated residues underlined in bold):

(SEQ ID NO:344)

k¹⁴ g¹⁵ p¹⁶ g¹⁷ k¹⁸ k¹⁹ k²⁰ n²¹ r²² k²³ r²⁴ n²⁵
p²⁶ e²⁷ k²⁸ p²⁹ h³⁰ f³¹ p³² l³³ a³⁴ t³⁵ e³⁶ d³⁷
d³⁸ v³⁹ r⁴⁰ h⁴¹ h⁴².

These two RPG sequences are, therefore, especially predictive of virulence and are preferred sequences for immunogenic compositions and vaccines. Identification of these residues in other RPG sequences in PRRSV provides a high likelihood of virulence and an excellent target for attack of the virus through antibody therapies, vaccines and other treatments.

X. Methods of Predicting and Treating Outbreaks of Porcine Circovirus (PCV) Using Replikin Sequences

An increase in Replikin concentration in PCV is predictive of an increase in virulence of the virus and allows for prediction of forthcoming outbreaks or increases in mortality. A review of publicly available amino acid sequences of isolates of PCV that demonstrate an increase in Replikin concentration in the genome or a genome segment, or in a protein or protein fragment of the virus over time or between isolates is used as a predictor of an increase in outbreaks and morbidity and mortality of pigs infected with PCV. Publicly available sequences for isolates of PCV from PubMed or other public or private sources may be analyzed by hand or using software described herein.

Applicants have now established a correlation between Replikin Counts in PCV and an increase in virulence. Applicants reviewed publicly available amino acid sequences of isolates of PCV having accession numbers at www.pubmed.com and identified increases in Replikin Counts in the genome of the virus that predict an increase in outbreaks and mortality of pigs infected with PCV.

The data for FIG. 21 is provided in Table 25 in Example 15 below. A general increase in Replikin Count from 2000 through 2007 is observable and may be correlated with an increase in incidence of and mortality from the disease between 2000 and 2006 as reported in Canada. Further, the very large Replikin Count number in 1997 followed by a marked decrease in 1998 through 2000 may be correlated with the beginning of increased outbreaks in 2000. In other viruses, outbreaks have been observed about 1 to 3 years after a large increase in Replikin Count that is followed by a notable decrease thereafter. See, e.g., FIGS. 2, 3 and 9. The graph in FIG. 21 demonstrates a cyclical pattern of Replikin Counts that is reminiscent of the correlation of Replikin Count with epidemics shown, for example, in influenza and SARS in FIGS. 2, 3 and 9.

In particular, the Replikin Count of PCV is observed at 9.4 (±10.8) in 1997 and decreases rapidly to 2.9 (1.2) in 2000. Replikin Count then rises to 3.5 (±1.4) in 2002 and rises again to 3.9 (±1.2) through 2007. During this time period, the virulence and mortality observed in swine herds in Canada (with additional reported incidence in Central America) were increasing. The large standard deviation seen in 1997-1999 evidences a virus population that is undergoing rapid change in the concentration of Replikin sequences in the genome and points to forthcoming changes in virulence, morbidity and mortality.

Prediction of epidemics and future outbreaks may be made, for example, by reviewing the Replikin Counts of RPGs or other portions of isolates of PCV or PRRSV or other virus or pathogen and comparing the Replikin Counts for a particular year with Replikin Counts from other years. A significant increase in Replikin Count from one year to the next and preferably over one, two or three or more years provides predictive value of an emerging strain of PCV that may begin an outbreak of more highly virulent and/or more highly lethal PCV.

A PCV outbreak may be predicted within about six months to about one year or more from the observation of a significant increase in Replikin Count. More preferably, an outbreak of PCV may be predicted within about six months to about one year from the observation of a significant increase in Replikin Count over two or three years or following the observation of strongly significant increases over several years such as wherein Replikin Counts of PCV between 2000 and 2002 and between 2005 and 2007 increased with p values each year over lowest mean Replikin Count in the series of less than 0.001.

Significant increases may be observed over a time period of more than one year, such as three, four or five years or more. An outbreak may likewise be predicted within about six months to about one year or more from the initial observation of an observable decrease in Replikin Count following a notable increase. For example, the marked decrease from 1997 to 2000 in PCV Replikin Counts predicts the increase of incidence and mortality in viral infections beginning in 2000 and continuing through at least 2006 (morbidity and mortality data for 2007 have not been made available at this time). Using this method, Applicants, for example, prospectively predicted the beginnings of a 2007 outbreak of WNV. See FIG. 12.

The inventors have identified a Replikin Peak Gene in the replicase protein of the Porcine Circovirus (PCV). Examples of the identification of a Replikin Peak Gene (RPG) in an isolate of PCV in Manitoba, Canada in 1997 and an RPG in an isolated of PCV in China in 2007 are provided in Example 9 (SEQ ID NOS: 520 and 525). Example 9 demonstrates comparably high Replikin Counts of the identified RPGs and provides prediction that the isolated strains of the virus have high virulence. Example 9 further provides RPGs and Replikin sequences within the identified RPGs as targets for production of immunogenic compositions and vaccines.

The invention provides Replikin sequences within the identified Replikin Peak Gene gene or gene segment for diagnostic, preventive and therapeutic applications. SEQ ID NOS: 324-328 are Replikin sequences provided in an RPG from Accession No. AAC59472. See Example 9. SEQ ID NOS: 329-340 are provided in an RPG from Accession No. ABP68657. See Example 9. For example, each of the above-listed sequences as Replikin sequences identified within an identified RPG are available for diagnostic and therapeutic applications including vaccines and antibody therapies. The entire Replikin Peak Gene sequence or fragments thereof are likewise available for diagnostic, preventive, therapeutic and predictive applications. Further, the presence of the Replikin Peak Gene in an isolate of the virus is indicative of rapid replication.

Replikin Peak Genes (RPG) have also been identified in PCV isolates in Accession Nos. AAC98885, AAL01075 and ABP68667 (SEQ ID NOS: 481, 438, and 451). See Example 9. For each identified RPG, continuous, non-interrupted and overlapping Replikin sequences have been identified for predictive and therapeutic applications.

Applicants have to date identified RPGs of available PCV isolates both within open reading frame 1 in a putative replicase protein and within open reading frame 11 in a predicted 1.8 kD protein. Identification of Replikin Peak Genes in different structures of different viruses is made possible through the strict criteria for a Replikin sequence as defined by the applicants. The size of a Replikin Peak Gene, both in terms of the number of amino acids and the Replikin Count, will depend upon the size of the sequence of the entire genome, protein or fragment thereof that has been isolated and reported. The invention further provides Replikin sequences within the identified Replikin Peak Gene that are conserved in the genome over time and, as such, are available as relatively invariant targets for diagnosis and manipulation of rapid replication and virulence in PCV.

XI. Conservation of Replikin Structure Relates to Virulence and Lethality

The conservation of any structure is critical to whether that structure provides a stable invariant target to attack and destroy or to stimulate. Replikin sequences have been shown to generally be conserved. When a structure is tied in some way to a basic survival mechanism of the organism, the structures tend to be conserved. A varying structure provides an inconstant target, which is a good strategy for avoiding attackers, such as antibodies that have been generated specifically against the prior structure and thus are ineffective against the modified form. This strategy is used by influenza virus, for example, so that a previous vaccine may be quite ineffective against the current virulent virus.

Certain structures too closely related to survival functions, however, apparently cannot change constantly. An essential component of the Replikin structure is histidine (h), which is known for its frequent binding to metal groups in redox enzymes and is a probable source of energy needed for replication. Since the histidine structure remains constant, Replikin sequence structures remain all the more attractive a target for destruction or stimulation.

A. Replikin Conservation in HIV

Conservation of Replikin sequences has been observed in trans-activator (Tat) proteins in isolates of HIV. Tat (trans-activator) proteins are early RNA binding proteins regulating lentiviral transcription. These proteins are necessary components in the life cycle of all known lentivirases, such as the human immunodeficiency viruses (HIV). Tat is a transcriptional regulator protein that acts by binding to the trans-activating response sequence (TAR) RNA element and activates transcription Initiation and/or elongation from the LTR promoter. HIV cannot replicate without tat, but the chemical basis of this has been unknown. In the HIV tat protein sequence from 89 to 102 residues, we have found a Replikin that is associated with rapid replication in other organisms. The amino acid sequence of this Replikin is hclvckqkkglgisygrkk (SEQ ID NO: 3666) In fact, Applicants found that this Replikin is present in every HIV tat protein. Some tat amino acids are substituted frequently, as shown in Table 12, by alternate amino acids (in small size fonts lined up below the most frequent amino acid, the percentage of conservation for the predominant Replikin (hclvcfqkkglgisygrkk) (SEQ ID NO: 3314). These substitutions have appeared for most of the individual amino acids. However, the key lysine and histidine amino acids within the Replikin sequence, which define the Replikin structure, are conserved 100% in the sequence; while substitutions are common elsewhere in other amino acids, both within and outside the Replikin, none occurs on these key histidine amino acids. The sequences listed in Table 12 are SEQ ID NO: 3314 and the denoted variations of formula peptide SEQ ID NO: 3315.

The substitutions cannot be considered to be at random because amino acids were substituted except for the lysines and histidines which define the Replikin structure. It is not just that lysine per se is “immune” to substitution, because the lysine not 6 to 10 amino acids from another lysine was freely substituted, while those lysines which do define the Replikin structure were not substituted.

TABLE 12
HIV TAT Conservation (SEQ ID NOS: 3314 and 3671)

% Replikin CONSERVATION of each constituent amino acid in the first 117
different isolates of HIY tat protein as reported in PubMed:
38 (100) 57 86 (100) (100) 66 76 (100) 99 5749 (100) 94 (100) 97 98 85 97 99 (100)(100)(100)%
Neighboring
Amino acids tat Replikin
k (c) s y [(h) (c) l v (c) f q k (k) g (l) g i s y g (r) (k) (k)]
below are the amino acid substitutions observed for each amino acid above:

| | | | | | |
|---|---|---|---|---|---|
| h | c f | q i | l h t | a | a l y h q |
| r | w p | l l | i h | q | v |
| y | s | s | l m | r | s |
| i | | s | m s | | |
| s | | r | n | | |
| v | | | | | |
| a | | | | | |
| f | | | | | |
| p | | | | | |
| q | | | | | |

B. Conservation in Replikin Peak Genes in H5N1 in Humans and Chickens

A series of conserved Replikin sequences (SEQ ID NOS: 1-11 and 14) were isolated in silico by Applicants in human and chicken isolates of H5N1 influenza virus. SEQ ID NO: 1 was identified in the following accession numbers in the following years at the following amino acid residue positions: (1997) AAK49342 beginning at position 134, AAK49340, 134, AAF74320, 134, AAF74319, 134, AAF74318, 134, AAF74317, 134, AAK49344, 134, AAK49343, 134, AAK49341, 134, AAK49339, 134, AAK49338, 134; (1998) AAK49345, 134; (2003) BAE07200, 134; (2004) AAW59551, 131, AAW59549, 129, ABE97897, 123, ABE97896, 123, ABE97895, 123, ABE97892, 123, ABE97891, 123, AAV32651, 134, AAV32643, 134; (2005) ABG78563, 109, ABG78562, 109, ABF56657, 127, ABF56656, 127, ABF56655, 127; (2006) ABK34973, 134, ABL31779, 134, ABL31765, 134, ABL31754, 134, ABL07029, 134, ABL07018, 119, ABL07007, 134, ABI49406, 134, ABI36481, 134, ABI36470, 134, ABI36451, 134, and ABI36440, 134.

SEQ ID NO: 11 was identified in the following accession numbers in the following years at the following amino acid residue positions: (2003) BAE07200, beginning at position 19; (2004) AAW59551, 16, AAW59549, 14, ABE97897, 8, ABE97896, 8, ABE97895, 8, ABE97894, 8, ABE97893, 8, ABE97892, 8, ABE97891, 8, ABE97890, 8, ABE97889, 8, ABE97888, 8, AAV35115, 19, AAV32651, 19, AAV32643, 19; (2005) ABC72649, 19, ABF56657, 12, ABF56656, 12, ABF56655, 12; (2006) ABK34973, 19, ABL31779, 19, ABL31765, 19, ABL31754, 19, ABL31743, 19, ABI49414, 19, ABL07029, 19, ABL07018, 4, ABL07007, 19, ABI49406, 19, ABI36481, 19, ABI36470, 19, ABI36451, 19, ABI36440, 19, ABI36429, 19.

SEQ ID NO: 14 was identified in the following accession numbers in 2006 at the following amino acid residue positions: ABL31777, beginning at position 41, ABI49393, 41, ABL07016, 41, ABL07005, 41, ABI49404, 41, ABI36472, 41, ABI36461, 41, ABI36452, 41, ABI36441, 41, and ABI36430, 41.

SEQ ID NO: 14 was isolated in silico from the pB1 gene area sequence disclosed at Accession No. ABI36441 (SEQ ID NO: 15). Replikin sequences (SEQ ID NOS: 16-17) were identified in the amino-terminus. Replikin sequences (SEQ ID NOS: 18-32) were identified in the mid-molecule. No Replikin sequences were identified in the carboxy-terminus. Sixteen Replikin sequences in 90 amino acid residues gave a Replikin Count of 17.8.

SEQ ID NO: 14 was also isolated in silico from Accession No. ABI36430 (SEQ ID NO: 33). Replikin sequences (SEQ ID NOS: 34-35) were identified in the amino-terminus. Replikin sequences (SEQ ID NOS: 36-49) were identified in the mid-molecule. No Replikin sequences were identified in the carboxy-terminus.

SEQ ID NO: 14 was also isolated in silico from Accession No. ABL07027 (SEQ ID NO: 50). Replikin sequences (SEQ ID NOS: 51-52) were identified in the amino-terminus. Replikin sequences (SEQ ID NOS: 53-68) were identified in the mid-molecule. Replikin sequences (SEQ ID NO: 69-71) were identified in the carboxy-terminus.

SEQ ID NO: 2 was identified in the following accession numbers in the following years at the following amino acid residue positions: (1997) Q9WLS3, 184, O89749, 184, AAK49358, 184, AAF74316, 184, AAK49362, 184, AAK49357, 184, AAK49356, 184, CAB95863, 184; (2003) BAE07199, 184; and (2004) ABL97546, 184, ABE97545, 184, ABE97544, 184, ABE97543, 184, ABE97542, 184, ABE97540, 184, ABE97564, 179, ABC72648, 184, ABK34974, 184.

SEQ ID NO: 3 was identified in the following accession numbers in the following years at the following amino acid residue positions: (1997) Q9WLS3, 184, O89749, 184, AAK49358, 184, AAF74316, 184, AAF74315, 184, AAF74314, 184, AAK49362, 184, AAK493761, 184, AAK49359, 184, AAK49357, 184, AAK49356, 184, CAB95863, 184; (1998) AAK49363, 184; (2003) BAE07199, 184; (2004) ABE97546, 184, ABE97545, 184, AGE97544, 184, ABE97543, 184, ABE97542, 184, ABE97541, 184, ABE97540, 184, ABE97539, 184, ABE97538, 184, ABE97537, 184, ABE97536, 184, AAV35116, 184, AAV32644, 184; (2005) ABG78564, 184, ABC72648, 184; and (2006) ABK34974, 184.

SEQ ID NO: 7 was identified in the following accession numbers in the following years at the following amino acid residue positions: (2003) BAE07200, 128; (2004) AAW59551, 125, AAW59549, 123, ABE97897, 117, ABE97896, 117, ABE97895, 117, ABE97894, 117, ABE97893, 117, ABE97892, 117, ABE97891, 117, ABE97890, 117, ABE97889, 117, ABE97888, 117, AAV32651m 128, AAV32643, 128; (2005) ABG78563, 103, ABG78562, 103, ABF56657, 121, ABF56656, 121, ABF56655, 121; and (2006) ABL31779, 128, AB31765, 128, ABL31754, 128, ABL31743, 128, ABI49414, 128, ABI49395, 128, ABL07029, 128, ABI36470, 128, ABI36451, 128, ABI36440, 128, ABI36429, 128.

SEQ ID NO: 8 was identified in the following accession numbers in the following years at the following amino acid residue positions: (1997) Q9WLS3, 184, O89749, 184, AAK49360, 168, AAK49356, 168, AAF74316, 168, AAK49362, 168, AAK49359, 168, AAK49357, 168, AAK49356, 168, CAB5863, 168; (2003) BAE07199, 168; (2004) ABE97546, 168, ABE97545, 168, ABE97544, 168, ABE97543, 168, ABE97542, 168, ABE97541, 168, ABE97539, 168, ABE97538, 168, ABE97537, 168, ABE97536, 168, AAV35116, 168, AAV32644, 168; (2005) ABG78564, 163, ABC72648, 168; and (2006) ABK34974, 168.

The series of conserved Replikin sequences discussed above are preferred embodiments of the invention and are particularly useful as immunogenic compounds and vaccines and the presence of these sequences has particular predictive value for timing, geographic position and lethality of H5N1 outbreaks.

C. Conservation in Replikin Scaffolds in Influenza A strains

Table 13, below, provides support for the role of Replikin Scaffolds as Replikin Peak Genes in lethal outbreaks of influenza in humans and birds. In Table 13, the history of the Goose Replikin and its homologues are tracked from 1917 to the present outbreak of avian H5N1 virus. Table 13 demonstrates conservation of the “scaffold” homology of the Goose Replikin in virulent strains of influenza.

Table 13 illustrates the history, by year or smaller time period, of the existence in the protein structure of the Goose Replikin and its homologues in other influenza Replikins. Table 13 further illustrates the history of amino acid substitutions in those homologues and the conservation of certain amino acids of the Replikin structure which are essential to the definition of a Replikin and the function of rapid replication supplied by Replikins.

Table 13 illustrates a Fixed Replikin Peak Gene Scaffold with ordered non-random substitution in the 90 year conservation of influenza virus Replikin peptides, from a 1917 goose flu and 1918 human pandemic to a 2007H5N1 ‘Bird Flu’ homologue.

The Goose Replikin is a 29 amino acid peptide RPG in the hemagglutinin protein of influenza virus beginning with kk and ending with hh (SEQ ID NO: 3672). Replikins may contain overlapping Replikins. This Replikin Scaffold appears in the virus genome only when the Replikin count rises above 3, and disappears again when the clinical outbreak is over and the Replikin count declines to less than 3.

TABLE 13
Goose Replikin Scaffolds

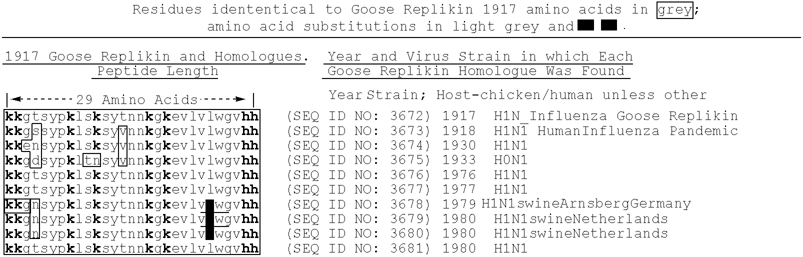

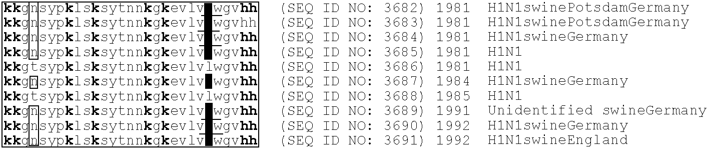

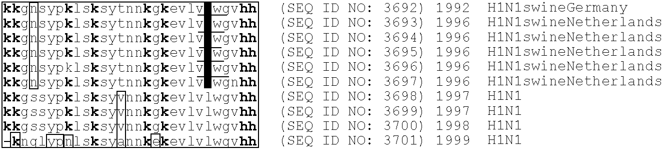

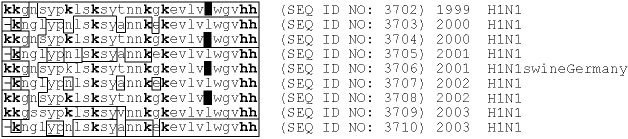

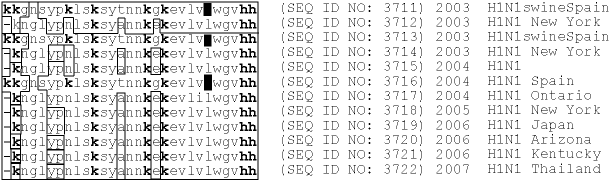

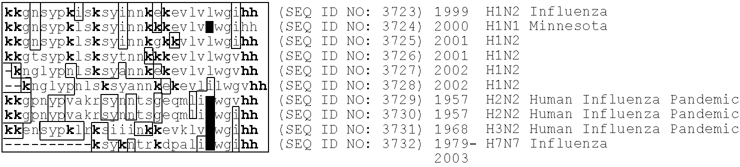

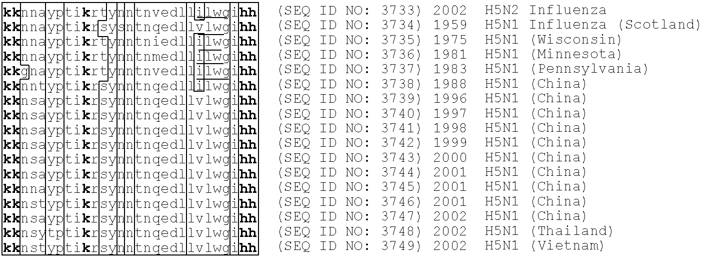

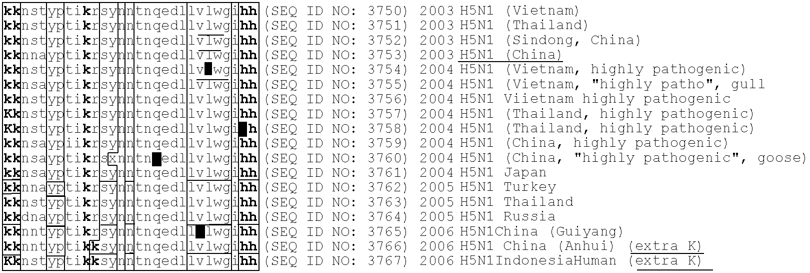

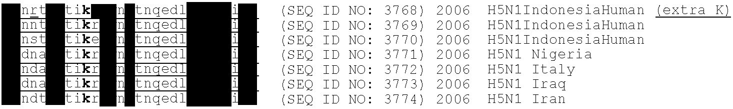

D. Replikin Scaffold in 2007 Isolate of H1N1

A Replikin Scaffold hemagglutinin Replikin Peak Gene has now been identified in one human case of H1N1 isolated in 2007 in Thailand. This evidence suggests H1N1 is making a comeback. The H1N1 Replikin Scaffold that has been identified is knglypnlsksyannkekevlvlwgvhh (SEQ ID NO: 2011), which is associated with a whole hemagglutinin Replikin Count of 8.1, and Replikin Count in the RPG of 28. The Replikin Count in the RPG of the 2007 Thailand isolate is higher than the Replikin Count in the RPG of an H1N1 isolate from the 1918 pandemic, Accession No: IRUZL, which has a Replikin Count in its RPG of 19. Example 5 provides the inventors analysis of the 2007 Thailand isolate.

E. Homologous Replikin Scaffold Sequences in Influenza, WSSV, and TSV

The inventors have further established a relationship between virulent influenza virus and shrimp viruses WSSV and TSV in the Replikin Scaffold portions of the viruses as may be seen in Table 14 below. Although there is extensive substitution, several short Replikins of the Shrimp white spot syndrome virus demonstrates significant homologies to the influenza virus Replikin sequences, especially with regard to length and key lysine (k) and histidine (h) residues. Similar, but less extensive, homologies are seen in taura syndrome virus. These homologies suggest that the sequences are derived from a shared reservoir and/or that similar mechanisms of Replikin production are used in both virus groups.

TABLE 14
Shrimp White Spot and Taura Syndrome Scaffolding

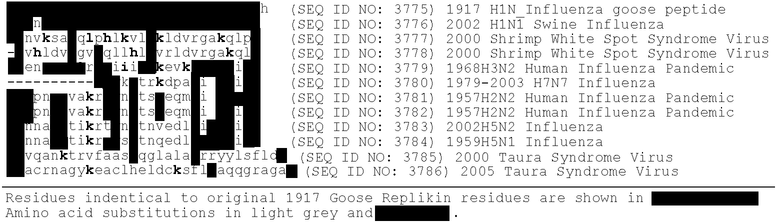

TSV is less virulent than WSSV and the structure of the TSV Replikin Scaffold is less closely related to influenza virus than are the structures of WSSV Replikin Scaffolds. In year 2000, TSV had a Replikin concentration of 2.7. Between 2001 and 2004, TSV had a lower mean Replikin concentration, as low as 0.7, and its Replikin Scaffold disappeared. In 2005 the Replikin Scaffold reappeared, with an increase in lysines and histidines, and a commensurate increase in Replikin concentration to 1.8, followed by an increase in TSV outbreaks in 2006-2007. See Table 19.

F. Replikin Peak Genes Provide Increased Predictive and Therapeutic Capacity

Since the identification of the Replikin structure, correlation between increased concentrations of Replikin sequences and increased replication and virulence has been observed in a range of viruses and organisms. These observations are made more accurate by the present isolation in silico of Replikin Peak Genes. While increased concentration of Replikin sequences in the genome of a virus offers both advance warning and new targets for developing effective methods of predicting and treating viral outbreaks, identification of an increase in concentration of Replikin sequences in a Replikin Peak Gene of a genome or protein heightens the predictive capacity of the change in Replikin concentration and the efficacy of new targets.

For example, more precise predictions of increased virulence are now available through identification of a Replikin Peak Gene in, among other viruses, the H5N1 strain of influenza (FIGS. 1-6), west nile virus (FIG. 12) and foot and mouth disease virus (FIG. 11). In these and other viruses, increased concentration of Replikin sequences in the whole genome, in a protein of the genome, in a Replikin Peak Gene of the genome, or in a protein containing a RPG, offer both advance warning and new targets for developing effective methods of predicting and treating viral outbreaks.

By monitoring changes in concentrations of Replikin sequences in viral genomes generally, emerging viral diseases can be identified in virus reservoirs and vectors in advance of their appearance in animal or human hosts. Identification of the emerging viruses and the Replikin sequences within the virus genome allows for appropriate, advance control efforts, including isolation and quarantine, and provide sufficient time for the synthesis and testing of vaccines specific to the sequences of the emerging virus.

As discussed above, the inventors have identified the pB1 gene area of the H3N8 strains of influenza virus (SEQ ID NO 545) as the region of the genome of the virus having the highest concentration of Replikin sequences. A Replikin Peak Gene has also been identified in H5N1 influenza virus and has been correlated with epidemics, increased virulence, morbidity and human mortality. (FIGS. 1-6) Likewise, a Replikin Peak Gene has been identified in the VP1 protein of foot and mouth disease virus and has been correlated with outbreaks of the virus. FIG. 11. A second Replikin Peak Gene (or Replikin Peak Gene Area) has additionally been identified in a fragment of the VP1 protein of foot and mouth disease virus and two particular Replikin sequences within the Replikin Peak Gene Area of the virus have been correlated with virulence of foot and mouth disease virus (e.g., SEQ ID NOS: 124 and 130). A Replikin Peak Gene has also been identified in west nile virus (e.g., SEQ ID NO: 258). FIG. 12. Replikin Peak Genes have further been identified in the nucleocapsid protein of the porcine reproductive and respiratory syndrome virus and in Porcine Circovirus (e.g., SEQ ID NOS: 341 and 520, respectively). FIGS. 13 and 19.

The invention provides Replikin sequences within the identified Replikin Peak Gene gene or gene segment (gene area) for diagnostic, preventive and therapeutic applications. For example, each Replikin sequence identified within an identified RPG is available for diagnostic and therapeutic applications including vaccines and antibody therapies. The entire Replikin Peak Gene sequence or fragments thereof are likewise available for diagnostic, preventive, therapeutic and predictive applications. Further, the presence of the Replikin Peak Gene in an isolate of the virus is indicative of rapid replication. For each identified RPG, continuous, non-interrupted and overlapping Replikin sequences have been identified for predictive and therapeutic applications. The size of a Replikin Peak Gene or Replikin Peak Gene Area, both in terms of the number of amino acids and the Replikin Count, will depend upon the size of the sequence of the entire genome, protein or fragment thereof that has been isolated and reported.

The invention further provides Replikin sequences within the identified Replikin Peak Gene or Replikin Peak Gene Area that are conserved in the genome over time and, as such, are available as relatively invariant targets for diagnosis and manipulation of rapid replication and virulence in EIV.

Point mutations within an RPG provide excellent predictive capacity when the point mutation is correlated with high virulence and provide an excellent target for attack of the virus through antibody therapies, vaccines and other treatments, as well as excellent predictive capacity when such point mutations are identified in emerging strains of the virus.

A further aspect of the invention provides utilizing software that searches for Replikin Peak Genes and enables the discovery of the point or points in the genome that have the highest concentration of Replikins, the years in which they have occurred, the strain or strains in which they occur, the host or hosts in which they occur, the geographic locations in which they occur, their increase or decrease in the above years, strains, hosts and geographic locations and point or small mutations that are correlatable with virulence.

The in silico detection of the Replikin Peak Gene by software methodology now permits both host and geographic localization of upregulated Replikin gene activity both in viruses and in other organisms. As seen in this study, the annual RPG Replikin analysis, by its correlation with a function such as epidemic outbreak or increase in lethality, can for the first time actually provide evidence for the correlation with the function.

The Replikin count in the whole genome or RPG make possible the prediction in advance of epidemic outbreaks of high mortality infections, such as those caused by influenza viruses, as seen for H5N1 in FIGS. 1-6. Such detection and localization permits advance focused public health preparations for better protection of the host, whether animal or human, and give time for the production and testing of new vaccines. The high Replikin Count of the RPG has now been shown to be associated consistently with a high percent lethality in the host, whether the host is a plant, fish, shrimp, or vertebrate, including human cases of H5N1 bird flu. The increase in count was frequently detected one year or more before the outbreak was clinically apparent (FIGS. 2, 3, 10, 11, 19, etc.). Vaccines may now be produced that directly target rapid replication as represented structurally by the Replikins in the whole genome and concentrated in a Replikin Peak Gene, rather than, as now, being targeted at virus epitopes whose function is unknown.

It may be concluded that Replikins represent a specific class of peptides that are widely distributed, conserved, quantitative markers of lethality. While not wishing to be bound by theory, evidence from the apparent transfer of conserved Replikin structures between strains suggest they may be mobile agents of lethality, transferring horizontally between carrier viruses to reach multicellular hosts, where they may replicate rapidly with lethal consequences. As newly recognized targets for prevention and therapy, Replikins offer a platform from which specifically to control rapid replication and lethality of organisms and cells, without necessarily destroying them.

G. Conserved Replikins in PCV for Diagnostics and Therapies

In review of the publicly available sequences for Porcine Circovirus, the applicants have identified three Replikin sequences from Accession No. ABQ 10608 that are conserved across many isolates from 1997 or 1998 through 2007: kngrsgpqphk (SEQ ID NO: 345); hlqgfanfvkkqtfnk (SEQ ID NO: 346) and kkqtfnkvkwylgarch (SEQ ID NO: 347). Because these sequences are conserved, they have predictive value and provide novel and preferred targets for diagnostic and therapeutic applications such as, for example, vaccines. Furthermore, two of these sequences, hlqgfanfvkkqtfnk (SEQ ID NO: 346) and kkqtfnkvkwylgarch (SEQ ID NO: 347) are contained within the identified RPG of Accession No. ABQ 10608. These sequences, therefore, are of preferred value in predicting virulent strains when such strains contain the sequences. Also, the sequences provide preferred targets for diagnostic and therapeutic applications such as, for example, vaccines. Table 15 provides the accession numbers of isolates of PCV between 1997 and 2007 containing the conserved sequence kngrsgpqphk (SEQ ID NO: 345) and the amino acid position within the PCV protein sequence wherein the conserved Replikin sequence begins.

TABLE 15
Conserved PCV Sequence

| | |
|---|---|
| 1997 | AAC59462 position 5. |
| 1998 | AAC35330 position 5, AAC35320 position 5, AAC35309 position 5, AAC35298 position 5, |
| | CAA11157 position 5, AAC61860 position 5, AAC61741 position 5, AAC61739 position 5, |
| | AAC61737 position 5, AAD03086 position 5, AAD03071 position 5, AAD03061 position 5, |
| | NP_048061 position 5, AAD11928 position 5. |
| 1999 | BAA88133 position 5, AAD50432 position 5, AAD38398 position 5, AAG41226 position 5, |
| | AAD37776 position 5, AAD45580 position 5, AAF35304 position 5, AAF35302 position 5, |
| | AAF35300 position 5, AAF35298 position 5, AAF35296 position 5, AAF35294 position 5, |
| | AAF35292 position 5, AAD12308 position 5. |
| 2000 | CAC41085 position 5, CAC41084 position 5, CAC41083 position 5, AAL09364 position 5, |
| | AAL09363 position 5, AAF87238 position 5, AAF87236 position 5, AAF87234 position 5, |
| | AAF87232 position 5, AAF87230 position 5, AAF87228 position 5. |
| 2001 | AAK60462 position 5, AAL58397 position 5, BAB69441 position 5, BAB69437 position 5, |
| | BAB69432 position 5, AAK56300 position 5, AAK56298 position 5, AAK56296 position 5, |
| | AAL01075 position 5. |
| 2002 | AAM61272 position 5, AAM61262 position 5, AAM61268 position 5, AAM61266 position |
| | 5, AAM61270 position 5, AAM61264 position 5, AAO39760 position 5, AAM21847 |
| | position 5, AAM21846 position 5, AAM21845 position 5, AAM21844 position 5, |
| | AAO24128 position 5, AAO24124 position 5, AAO24122 position 5, AAO23147 position 5, |
| | AAO23145 position 5, AAN81597 position 5, AAN06826 position 5, AAN62769 position 5, |
| | AAN62767 position 5, AAN62765 position 5, AAL69968 position 5, AAM76057 position 5, |
| | Q8BB16 position 5. |
| 2003 | AAP51128 position 5, AAS65993 position 5, AAS65991 position 5, AAS65989 position 5, |
| | AAS65987 position 5, AAS65985 position 5, AAS65983 position 5, AAS65981 position 5, |
| | AAS65979 position 5, AAS65977 position 5, AAS65975 position 5, AAP83635 position 5, |
| | AAP83633 position 5, AAP83631 position 5, AAP83629 position 5, AAP83627 position 5, |
| | AAP83625 position 5, AAP83623 position 5, AAP83621 position 5, AAP83619 position 5, |
| | AAP83617 position 5, AAP83615 position 5, AAP83613 position 5, AAP83611 position 5, |
| | AAP83609 position 5, AAP83607 position 5, AAP83605 position 5, AAP83603 position 5, |
| | AAP83601 position 5, AAP83599 position 5, AAP83597 position 5, AAP83595 position 5, |
| | AAP83593 position 5, AAP83591 position 5, AAR03722 position 5, AAR03720 position 5, |
| | AAR03718 position 5, AAR03716 position 5, AAQ94098 position 5, AAQ94096 position 5, |
| | AAQ94094 position 5, AAQ94092 position 5, AAQ94090 position 5, AAQ94088 position 5, |
| | AAP44188 position 5, AAP44182 position 5, AAR97517 position 5, AAQ96327 position 5, |
| | AAQ23155 position 5, AAP42468 position 5, AAP42466 position 5, AAO61136 position 5, |
| | NP_937956 position 5, AAR03714 position 5. |
| 2004 | AAW78475 position 5, AAW78473 position 5, AAW78471 position 5, AAW78469 position |
| | 5, AAW78467 position 5, AAW78465 position 5, AAW78463 position 5, AAV34139 |
| | position 5, AAU87519 position 5, AAU87515 position 5, AAU87511 position 5, AAU87509 |
| | position 5, AAU34001 position 5, AAT97650 position 5, AAT97648 position 5, AAT97646 |
| | position 5, AAT36358 position 5, AAX49397 position 5, AAU01966 position 5, AAT72901 |
| | position 5, AAT58234 position 5, AAS45844 position 5, AAS45843 position 5, CAJ31064 |
| | position 5, AAU13780 position 5, AAX52911 position 5, AAU87505 position 5, AAT39479 |
| | position 5, AAT39460 position 5, AAT37493 position 5, AAS66198 position 5, AAS66196 |
| | position 5, AAS66194 position 5, AAS66192 position 5, AAS90297 position 5, AAS89260 |
| | position 5, CAF25171 position 5. |
| 2005 | ABJ98317 position 5, AAZ20800 position 5, AAZ20796 position 5, AAZ20794 position 5, |
| | AAW79865 position 5, ABC26025 position 5, ABA40480 position 5, AAZ78351 position 5, |
| | AAY40292 position 5, ABB29423 position 5, ABB29419 position 5, ABB29417 position 5, |
| | ABB29415 position 5, ABB29413 position 5, ABB29411 position 5, ABB29409 position 5, |
| | ABB29407 position 5, ABB29405 position 5, ABB29403 position 5, ABB29401 position 5, |
| | ABB29399 position 5, ABA60807 position 5, ABA60805 position 5, ABA40399 position 5, |
| | ABA40397 position 5, AAX10150 position 5, AAX62053 position 5, AAX62051 position 5, |
| | AAX62049 position 5, AAX62047 position 5, AAX62045 position 5, AAX62043 position 5, |
| | AAX62041 position 5, ABC75103 position 5, ABB20934 position 5, ABA26910 position 5, |
| | ABA26908 position 5, AAY34249 position 5. |
| 2006 | ABI29887 position 5, ABG21279 position 5, ABG21277 position 5, ABG21275 position 5, |
| | ABG21273 position 5, ABG21271 position 5, ABG21269 position 5, ABG21267 position 5, |
| | ABJ98319 position 5, ABI93799 position 5, ABI93797 position 5, ABD59347 position 5, |
| | ABD42928 position 5, ABM88864 position 5, ABM88862 position 5, ABM88860 position 5, |
| | ABI17537 position 5, ABI17535 position 5, ABI17533 position 5, ABI17531 position 5, |
| | ABI17529 position 5, ABI17527 position 5, ABI17525 position 5, ABI17523 position 5, |
| | ABG37023 position 5, ABF71465 position 5. |
| 2007 | ABQ10608 position 5, ABQ10606 position 5, ABQ10604 position 5, ABQ10603 position 5, |
| | ABP68669 position 5, ABP68665 position 5, ABP68661 position 5, ABP68657 position 5, |
| | ABP68655 position 5, ABP68651 position 5, ABP68647 position 5, ABP68645 position 5, |
| | ABP68643 position 5, ABP68641 position 5, ABP68639 position 5, ABP68635 position 5, |
| | ABP68633 position 5, ABP68631 position 5, ABP68629 position 5, ABP68627 position 5, |
| | ABP68625 position 5, ABP68623 position 5, ABP68621 position 5, ABP68619 position 5, |
| | ABP68617 position 5, ABP68615 position 5, ABO38130 position 5, ABM97550 position 5, |
| | ABQ63072 position 5, ABQ63070 position 5, ABQ63068 position 5, ABQ63066 position 5, |
| | ABQ63064 position 5, ABQ63062 position 5, ABQ51920 position 5, ABQ51918 position 5, |
| | ABR14585 position 5, ABP49176 position 5, ABP48091 position 5, ABP48089 position 5, |
| | ABP48087 position 5, ABP48085 position 5, ABP48083 position 5, ABP48081 position 5, |
| | ABO09999 position 5, ABO09997 position 5, ABO09995 position 5, ABO09993 position 5, |
| | ABO09991 position 5, ABO09989 position 5, ABO09987 position 5, ABP23690 position 5. |

Table 16 provides the accession numbers of PCV isolates between 1997 and 2007 containing the conserved sequence hlqgfanfvkkqtfnk (SEQ ID NO: 346) and the amino acid position within the PCV protein sequence wherein the conserved Replikin sequence begins.

TABLE 16
Conserved PCV Sequence

| | |
|---|---|
| 1997 | AAC59462 position 57. |
| 1998 | AAC35330 position 57, AAC35320 position 57, AAC35309 position 57, AAC35298 position |
| | 57, CAA11157 position 57, AAC61860 position 57, AAC61741 position 57, AAC61739 |
| | position 57, AAC61737 position 57, AAD03086 position 57, AAD03071 position 57, |
| | AAD03061 position 57, NP_048061 position 57, AAD11928 position 57. |
| 1999 | BAA88133 position 57, AAD50432 position 57, AAD38398 position 57, AAG41226 position |
| | 57, AAD37776 position 57, AAD45580 position 57, AAF35304 position 57, AAF35302 |
| | position 57, AAF35300 position 57, AAF35298 position 57, AAF35296 position 57, |
| | AAF35294 position 57, AAF35292 position 57, AAD12308 position 57. |
| 2000 | CAC41085 position 57, CAC41084 position 57, AAL09364 position 57, AAL09363 position |
| | 57, AAF87238 position 57, AAF87236 position 57, AAF87234 position 57, AAF87232 |
| | position 57, AAF87230 position 57, AAF87228 position 57. |
| 2001 | AAK60462 position 57, AAL58397 position 57, BAB69441 position 57, BAB69437 position |
| | 57, BAB69432 position 57, AAK56300 position 57, AAK56298 position 57, AAK56296 |
| | position 57, AAL01075 position 57. |
| 2002 | AAM61272 position 57, AAM61262 position 57, AAM61268 position 57, AAM61266 |
| | position 57, AAM61270 position 57, AAM61264 position 57, AAO39760 position 57, |
| | AAM21845 position 57, AAM21844 position 57, AAO24128 position 57, AAO24126 |
| | position 57, AAO24124 position 57, AAO24122 position 57, AAO23147 position 57, |
| | AAO23145 position 57, AAN81597 position 57, AAN06826 position 57, AAN62769 position |
| | 57, AAN62767 position 57, AAN62765 position 57, AAN16398 position 57, AAM83186 |
| | position 57, AAM76057 position 57, Q8BB16 position 57, AAO95302 position 57. |
| 2003 | AAP51128 position 57, AAS65993 position 57, AAS65991 position 57, AAS65989 position |
| | 57, AAS65987 position 57, AAS65985 position 57, AAS65983 position 57, AAS65981 |
| | position 57, AAS65979 position 57, AAS65977 position 57, AAS65975 position 57, |
| | AAP83635 position 57, AAP83633 position 57, AAP83631 position 57, AAP83629 position |
| | 57, AAP83627 position 57, AAP83625 position 57, AAP83623 position 57, AAP83621 |
| | position 57, AAP83619 position 57, AAP83617 position 57, AAP83615 position 57, |
| | AAP83613 position 57, AAP83611 position 57, AAP83609 position 57, AAP83607 position |
| | 57, AAP83605 position 57, AAP83603 position 57, AAP83601 position 57, AAP83599 |
| | position 57, AAP83597 position 57, AAP83595 position 57, AAP83593 position 57, |
| | AAP83591 position 57, AAR03722 position 57, AAR03720 position 57, AAR03718 position |
| | 57, AAR03716 position 57, AAQ94098 position 57, AAQ94096 position 57, AAQ94094 |
| | position 57, AAQ94092 position 57, AAQ94090 position 57, AAQ94088 position 57, |
| | AAP44188 position 57, AAP44185 position 57, AAP44182 position 57, AAR97517 position |
| | 57, AAQ96327 position 57, AAQ23155 position 57, AAP42468 position 57, AAP42466 |
| | position 57, AAP42464 position 57, AAO61136 position 57, NP_937956 position 57, |
| | AAR03714 position 57. |
| 2004 | AAW78475 position 57, AAW78473 position 57, AAW78471 position 57, AAW78469 |
| | position 57, AAW78467 position 57, AAW78465 position 57, AAW78463 position 57, |
| | AAV34139 position 57, AAU87519 position 57, AAU87517 position 57, AAU87515 position |
| | 57, AAU87513 position 57, AAU87511 position 57, AAU87509 position 57, AAU87507 |
| | position 57, AAU34001 position 57, AAU01913 position 57, AAT97650 position 57, |
| | AAT97648 position 57, AAT97646 position 57, AAT97644 position 57, AAT36358 position |
| | 57, AAX49397 position 57, AAU01966 position 57, AAT79579 position 57, AAT72901 |
| | position 57, AAS45844 position 57, AAS45843 position 57, CAJ31064 position 57, |
| | AAU13780 position 57, AAX52911 position 57, AAU87505 position 57, AAT39479 position |
| | 57, AAT39460 position 57, AAT37493 position 57, AAS66198 position 57, AAS66196 |
| | position 57, AAS66194 position 57, AAS66192 position 57, AAS66190 position 57, |
| | AAS90297 position 57, CAF25171 position 57. |
| 2005 | ABJ98317 position 57, ABA29241 position 57, AAZ20802 position 57, AAZ20800 position |
| | 57, AAZ20798 position 57, AAZ20796 position 57, AAZ20794 position 57, AAW79865 |
| | position 57, ABC26025 position 57, ABA40480 position 57, AAZ78351 position 57, |
| | AAX21515 position 57, ABB29423 position 57, ABB29421 position 57, ABB29419 position |
| | 57, ABB29417 position 57, ABB29415 position 57, ABB29413 position 57, ABB29411 |
| | position 57, ABB29409 position 57, ABB29407 position 57, ABB29405 position 57, |
| | ABB29403 position 57, ABB29401 position 57, ABB29399 position 57, ABA60807 position |
| | 57, ABA60805 position 57, ABA60803 position 57, ABA40399 position 57, ABA40397 |
| | position 57, AAZ66792 position 57, AAX10150 position 57, AAX62053 position 57, |
| | AAX62051 position 57, AAX62049 position 57, AAX62047 position 57, AAX62045 position |
| | 57, AAX62043 position 57, AAX62041 position 57, ABC75103 position 57, ABB20934 |
| | position 57, ABA26910 position 57, ABA26908 position 57, AAY34249 position 57. |
| 2006 | ABI29887 position 57, ABG21279 position 57, ABG21277 position 57, ABG21275 position |
| | 57, ABG21273 position 57, ABG21271 position 57, ABG21269 position 57, ABG21267 |
| | position 57, ABJ98319 position 57, ABI93799 position 57, ABI93797 position 57, |
| | ABD59347 position 57, ABD42928 position 57, ABM88864 position 57, ABM88862 position |
| | 57, ABM88860 position 57, ABI17537 position 57, ABI17535 position 57, ABI17533 |
| | position 57, ABI17531 position 57, ABI17529 position 57, ABI17527 position 57, ABI17525 |
| | position 57, ABI17523 position 57, ABG37023 position 57, ABF71465 position 57. |
| 2007 | ABQ10608 position 57, ABQ10606 position 57, ABQ10604 position 57, ABQ10603 position |
| | 57, ABP68669 position 57, ABP68667 position 57, ABP68665 position 57, ABP68663 |
| | position 57, ABP68661 position 57, ABP68659 position 57, ABP68657 position 57, |
| | ABP68655 position 57, ABP68653 position 57, ABP68651 position 57, ABP68649 position |
| | 57, ABP68645 position 57, ABP68643 position 57, ABP68641 position 57, ABP68639 |
| | position 57, ABP68637 position 57, ABP68635 position 57, ABP68633 position 57, |
| | ABP68629 position 57, ABP68619 position 57, ABP68617 position 57, ABP68615 position |
| | 57, ABO38130 position 57, ABM97550 position 57, ABQ63072 position 57, ABQ63070 |
| | position 57, ABQ63068 position 57, ABQ63066 position 57, ABQ63064 position 57, |
| | ABQ63062 position 57, ABQ51920 position 57, ABQ51918 position 57, ABR14585 position |
| | 57, ABP49176 position 57, ABP48091 position 57, ABP48089 position 57, ABP48087 |
| | position 57, ABP48083 position 57, ABP48081 position 57, ABO09997 position 57, |
| | ABO09995 position 57, ABO09993 position 57, ABO09991 position 57, ABO09989 position |
| | 57, ABO09987 position 57, ABP23690 position 57. |

Table 17 provides the accession numbers of PCV isolates between 1998 and 2007 containing the conserved sequence kkqtfnkvkwylgarch (SEQ ID NO: 347) and the amino acid position within the PCV protein sequence wherein the conserved Replikin sequence begins.

TABLE 17
Convserved PCV Sequence

| | |
|---|---|
| 1998 | AAC35330 position 66, AAC35320 position 66, AAC35309 position 66, AAC35298 |
| | position 66, CAA11157 position 66, AAC61860 position 66, AAC61739 position 66, |
| | AAC61737 position 66, AAD03086 position 66, AAD03071 position 66, AAD03061 |
| | position 66, NP_048061 position 66, AAD11928 position 66. |
| 1999 | AAG41226 position 66, AAD37776 position 66, AAD45580 position 66, AAF35304 |
| | position 66, AAF35302 position 66, AAF35300 position 66, AAF35298 position 66, |
| | AAF35296 position 66, AAF35294 position 66, AAF35292 position 66, AAD12308 |
| | position 66. |
| 2000 | CAC41085 position 66, CAC41084 position 66, AAF87238 position 66, AAF87236 |
| | position 66, AAF87234 position 66, AAF87232 position 66, AAF87230 position 66, |
| | AAF87228 position 66. |
| 2001 | AAL58397 position 66, BAB69441 position 66, BAB69437 position 66, BAB69432 |
| | position 66, AAK56300 position 66, AAK56298 position 66, AAK56296 position 66, |
| | AAL01075 position 66. |
| 2002 | AAM61272 position 66, AAM61262 position 66, AAM61268 position 66, AAM61266 |
| | position 66, AAM61270 position 66, AAM61264 position 66, AAO39760 position 66, |
| | AAM21845 position 66, AAM21844 position 66, AAO24128 position 66, AAO24124 |
| | position 66, AAO24122 position 66, AAN81597 position 66, AAN06826 position 66, |
| | AAN16398 position 66, AAM83186 position 66, AAL69968 position 66, AAM76057 |
| | position 66, Q8BB16 position 66. |
| 2003 | AAP51128 position 66, AAS65993 position 66, AAS65991 position 66, AAS65989 |
| | position 66, AAS65987 position 66, AAS65985 position 66, AAS65983 position 66, |
| | AAS65979 position 66, AAS65977 position 66, AAS65975 position 66, AAP83635 |
| | position 66, AAP83633 position 66, AAP83631 position 66, AAP83629 position 66, |
| | AAP83627 position 66, AAP83625 position 66, AAP83623 position 66, AAP83621 |
| | position 66, AAP83619 position 66, AAP83617 position 66, AAP83615 position 66, |
| | AAP83613 position 66, AAP83611 position 66, AAP83609 position 66, AAP83607 |
| | position 66, AAP83605 position 66, AAP83603 position 66, AAP83601 position 66, |
| | AAP83599 position 66, AAP83597 position 66, AAP83595 position 66, AAP83593 |
| | position 66, AAP83591 position 66, AAR03722 position 66, AAR03720 position 66, |
| | AAQ94098 position 66, AAQ94096 position 66, AAQ94094 position 66, AAQ94092 |
| | position 66, AAQ94090 position 66, AAQ94088 position 66, AAP44188 position 66, |
| | AAP44182 position 66, AAQ96327 position 66, AAQ23155 position 66, AAP42466 |
| | position 66, AAP42464 position 66, AAO61136 position 66. |
| 2004 | AAW78479 position 63, AAW78475 position 66, AAW78471 position 66, AAW78469 |
| | position 66, AAW78465 position 66, AAV34139 position 66, AAU87519 position 66, |
| | AAU87511 position 66, AAU87509 position 66, AAU34001 position 66, AAU01913 |
| | position 66, AAT97648 position 66, AAT97644 position 66, AAT36358 position 66, |
| | AAX49397 position 66, AAT72901 position 66, AAS45844 position 66, AAS45843 |
| | position 66, CAJ31064 position 66, AAU87505 position 66, AAT39479 position 66, |
| | AAT39460 position 66, AAT37493 position 66, AAS90297 position 66, AAS89260 |
| | position 66, CAF25171 position 66. |
| 2005 | ABJ98317 position 66, AAZ20802 position 66, AAZ20800 position 66, AAZ20798 |
| | position 66, AAZ20796 position 66, AAZ20794 position 66, AAW79865 position 66, |
| | AAY40292 position 66, ABB29423 position 66, ABB29421 position 66, ABB29419 |
| | position 66, ABB29417 position 66, ABB29415 position 66, ABB29413 position 66, |
| | ABB29411 position 66, ABB29409 position 66, ABB29407 position 66, ABB29405 |
| | position 66, ABB29403 position 66, ABB29401 position 66, ABB29399 position 66, |
| | ABA60807 position 66, ABA60805 position 66, ABA40399 position 66, ABA40397 |
| | position 66, AAZ66792 position 66, AAX10150 position 66, AAX62051 position 66, |
| | AAX62049 position 66, AAX62047 position 66, AAX62045 position 66, AAX62041 |
| | position 66, ABB20934 position 66, ABA26908 position 66, AAY34249 position 66. |
| 2006 | ABI29887 position 66, ABG21279 position 66, ABG21277 position 66, ABG21275 |
| | position 66, ABG21273 position 66, ABG21271 position 66, ABG21269 position 66, |
| | ABG21267 position 66, ABJ98319 position 66, ABI93799 position 66, ABD59347 |
| | position 66, ABD42928 position 66, ABI17537 position 66, ABI17535 position 66, |
| | ABI17533 position 66, ABI17531 position 66, ABI17529 position 66, ABI17527 position |
| | 66, ABI17525 position 66, ABI17523 position 66, ABG37025 position 63, ABG37023 |
| | position 66, ABF71465 position 66. |
| 2007 | ABQ10608 position 66, ABQ10606 position 66, ABQ10604 position 66, ABQ10603 |
| | position 66, ABP68669 position 66, ABP68665 position 66, ABP68661 position 66, |
| | ABP68653 position 66, ABP68651 position 66, ABP68643 position 66, ABP68641 |
| | position 66, ABP68629 position 66, ABP68619 position 66, ABP68617 position 66, |
| | ABP68615 position 66, ABO38130 position 66, ABQ63072 position 66, ABQ63070 |
| | position 66, ABQ63068 position 66, ABQ63066 position 66, ABQ63064 position 66, |
| | ABQ63062 position 66, ABQ51920 position 66, ABQ51918 position 66, ABR14585 |
| | position 66, ABP49176 position 66, ABP48091 position 66, ABP48089 position 66, |
| | ABP48087 position 66, ABP48083 position 66, ABO09999 position 66, ABO09991 |
| | position 66, ABO09989 position 66, ABP23690 position 66. |

XII. Relationship between Replikin Peak Gene and Lethality in Tobacco Mosaic Virus and Lung Malignancy

As established above, the Replikin Peak Gene correlates with activity of viruses such as pandemic influenza, Bird Flu, west nile virus and Bird Flu H5N1, among many others. It has surprisingly now been discovered that the highest activity to date of the Replikin Peak Gene was found in lung cancer (SEQ ID NO: 1741). Although viruses have been amply confirmed to be associated with the causation of several cancers since the work of Rous in sarcoma at the beginning of the last century, and viruses are the basis of current anti-cancer vaccines, how viruses are related to cancer is still not well understood. The antimalignin antibody in serum (AMAS) test is an FDA-permitted Medicare-approved early detection method for cancer that measures production of antibody against peptides containing a key Replikin sequence, namely, the glioma Replikin peptide, kagvaflhkk (SEQ ID NO: 3658), but how AMAS detects cancer regardless of cell type has not been fully understood. Results from separate studies in the areas of viruses and cancer now have converged with the isolation by the inventors of Replikins in both viruses and cancers that are concentrated in proteins where the concentration of Replikins has been related to rapid replication.

Higher Replikin Counts in RPGs have now been associated consistently with a higher percent lethality in the host; whether the host is a plant, fish, shrimp, or vertebrate, including human cases of H5N1 bird flu. The increase in count has frequently been detected one year or more before outbreaks have become clinically apparent. In addition to the correlation of high counts with virulence and lethality, structures specific to Replikins have been found by the inventors. For example, a 29-amino acid Replikin scaffold (beginning with SEQ ID NO: 3672) conserved for 90 years, appeared in the genome of successive influenza virus strains and each of the lethal pandemics and lethal H5N1 outbreaks. Additionally, repeating specific Replikin sequence signatures in RPGs of certain pathogens and malignancies have been identified and correlated with lethality. For example, an identical signature (SEQ ID NO: 1584) was found to repeat eleven times in the RPG of protozoan P. falciparum, 20 times in the RPG of tobacco mosaic virus which included exacerbated cell death in a pepper plant, with exacerbated cell death induced by Tobacco Mosaic virus, and 57 times (by overlapping) within 52 Replikins in the 18 amino acid RPG of non-small cell lung carcinoma.

While the inventors do not wish to be bound by theory, both of the above two studies support the impression that Replikins are mobile agents of lethality. Pathogenic viruses may just be the carrier of the lethal mobile agents. The highest Replikin Count in a Replikin Peak Gene that has to date been observed in highly lethal non-small cell lung cancer. The Replikin Count was observed to be 289 Replikin sequences per 100 amino acids. Other cancers, such as breast and ovarian have likewise been observed to have very high Replikin Counts in their Replikin Peak Genes with counts above 40 Replikin sequences per 100 amino acids. An RPG was identified and a Replikin Count of 129 was observed in Accession No. EAW84344 in lymphoblastic leukemia. An RPG was likewise identified and a Replikin Count of 23 was observed in Accession No. EAX09769 in myeloid leukemia.

Replikins chemically synthesized in the laboratory were found experimentally to be immuno-stimulants, producing strong antibody responses in chickens and rabbits. It appears that the antibodies measured in the AMAS test are against the Replikins' chemistry of rapid replication rather than the histological diagnosis of cancer or the cell type. Thus, for example, histologically proven prostate cancer that is “quiescent” (over 90% of such cancers) has low antibody levels in the AMAS test. But when these cells replicate rapidly, antibody levels measured by the AMAS test increase markedly. AMAS warning frequently precedes detection of the production of Prostate-Specific Antigen (PSA), an antigen that is frequently assayed because of its relationship to prostate cancer. AMAS probably precedes PSA because PSA measures protein fragments, the antigens that must be released by the cancer cells into the blood, while AMAS measures antibody to the peptide changes in the cancer cells, an earlier detectable event.

Since humans are host to and inhabited by thousands of viruses and bacteria that live symbiotically within the body, unless some event like rapid replication creates disease, no pathogenesis exists. Therefore, it may be important to learn how to control symbiosis between host humans and symbiotic viruses and bacteria without necessarily aiming to destroy the organism outright, especially when destruction proves difficult.

Peptides isolated from cancer cells grown in tissue culture have been found to contain Replikin sequences. When stimulated by anoxia, the cell number in these tissue cultures increased five-fold per week. Surprisingly, however, Replikin sequence concentration increased ten-fold per week (twice that of cell number), demonstrating a correlation of Replikin count with rapid replication in cancer tissue culture. When the structure of these Replikin-containing peptides was determined, separately synthesized chemically, and administered to rabbits, the peptides produced specific antimalignin antibodies in abundance. The production of antimalignin antibodies in response to the Replikin-containing peptides provided evidence to close the circle of confirmation that AMAS is measuring Replikins activity in malignancy.

In addition to early detection by the AMAS test of the activity of the group of Replikins that are unique to cancer, Replikins are widely distributed markers of, and probably agents of, lethality. As newly recognized targets for prevention and therapy, Replikins offer a platform from which to control rapid replication and lethality of organisms and cells, without necessarily destroying them.

XIII. Replikin Count Correlates with Virulence and Lethality in Shrimp Taura Syndrome Virus

Applicants have likewise demonstrated in a blind study using an independent laboratory testing taura syndrome virus (TSV) in shrimp that virulence and mortality in shrimp correlates with Replikin Count in TSV. The inventors analyzed the genome of the TSV of four main isolates from Hawaii, Belize, Thailand and Venezuela to provide predictions ranking the virulence and mortality rate of each isolate. An independent laboratory tested each isolate in shrimp and provided blind data on mortality. The data demonstrate a quantitative linear correlation between Replikin concentration and mortality. See Example 18. Despite differences in epidemiology, virology and host, all of these data lend further support for the value of Replikin concentration in predicting outbreaks of pathogens and lethality of pathogens and malignancies.

XIV. Replikin Concentration in Replikin Peak Gene of Ribonucleotide Reductase Gene Area Correlated with a WSSV Epidemic

An increase in Replikin concentration in white spot syndrome virus (WSSV) is predictive of an increase in virulence of the virus and allows for prediction of forthcoming outbreaks or increases in morbidity and, in extreme cases, mortality. A review of publicly available amino acid sequences of isolates of WSSV that demonstrate an increase in Replikin Count in the genome or a genome segment, or in a protein or protein fragment of the virus over time or between isolates is used as a predictor of an increase in outbreaks in shrimp. Publicly available sequences for isolates of WSSV from PubMed or other public or private sources may be analyzed by hand or using proprietary search tool software (ReplikinForecast™ available in the United States from REPLIKINS LLC, Boston, Mass.).

Applicants have established a correlation between Replikin concentrations in WSSV and an increase in virulence of the virus resulting in epidemics. Applicants reviewed publicly available amino acid sequences of isolates of WSSV having accession numbers at www.pubmed.com and have identified a remarkable increase in Replikin concentration in the Replikin Peak Gene of the ribonucleotide reductase gene area of the genome of the virus (e.g., SEQ ID NO: 669). The remarkable increase occurred just prior to a significant outbreak of WSSV in shrimp in 2001. FIG. 18 illustrates a correlation between increases in Replikin Count in WSSV genome in 2000 and a significant outbreak of WSSV in 2001. In 2000, a remarkably high Replikin concentration of 97.6 is observed in WSSV. In the Replikin Peak Gene identified in ribonucleotide reductase in an isolate from 2000, the Replikin concentration spikes as high as 110.7, providing an unmistakable predictive signal for the significant 2001 outbreak of WSSV that followed. Analysis of the ribonucleotide reductase sequence publicly available at Accession No. AAL89390 (SEQ ID NO: 668) is disclosed in Example 10.

A. Analysis of Annual Replikin Count of WSSV

Applicants analyzed publicly available sequences for isolates of WSSV from PubMed. The data is contained in Table 18 and graphically described in FIG. 18. Mean Replikin concentrations were determined for all amino acid sequences for WSSV with accession numbers publicly available at www.pubmed.com. The mean Replikin Count was then determined for all viruses isolated and reported in a particular year. Table WSSV provides the results of the Replikin Count analysis. Years with no data are not included in the table.

TABLE 18
WSSV Replikin Count

| | | | Mean | | |
|---|---|---|---|---|---|
| | PubMed Accession Number-Replikin | No. | Replikin | | |
| Year | Count | Isolates | Count | S.D. | Significance |
| 1995 | CAA88950 18 CAA91970 59 | 2 | 4.4 | 0.6 | low p < .10 |
| 1996 | CAE17687 160 CAB03144 29 | 3 | 6.0 | 2.6 | low p > .50, prev p < .30 |
| | CAB03173 31 | | | | |
| 1998 | ABA54417 48 | 1 | 6.2 | 0.0 | prev p > .50 |
| 2000 | | 529 | 97.6 | 0.0 | low p < .001 |

| | NP_478030 361 NP_478019 361 NP_478001 361 NP_477774 361 NP_477756 361 NP_477753 361 | | | | |
|---|---|---|---|---|---|
| | NP_477809 361 NP_477768 361 NP_477523 361 NP_477959 361 NP_478053 361 NP_478052 361 | | | | |
| | NP_478051 361 NP_478050 361 NP_478049 361 NP_478048 361 NP_478047 361 NP_478046 361 | | | | |
| | NP_478045 361 NP_478044 361 NP_478043 361 NP_478042 361 NP_478041 361 NP_478039 361 | | | | |
| | NP_478038 361 NP_478037 361 NP_478036 361 NP_478035 361 NP_478034 361 NP_478033 361 | | | | |
| | NP_478032 361 NP_478031 361 NP_478029 361 NP_478028 361 NP_478027 361 NP_478026 361 | | | | |
| | NP_478025 361 NP_478024 361 NP_478023 361 NP_478022 361 NP_478021 361 NP_478020 361 | | | | |
| | NP_478018 361 NP_478017 361 NP_478016 361 NP_478015 361 NP_478014 361 NP_478013 361 | | | | |
| | NP_478012 361 NP_478011 361 NP_478010 361 NP_478009 361 NP_478008 361 NP_478007 361 | | | | |
| | NP_478006 361 NP_478005 361 NP_478004 361 NP_478003 361 NP_478002 361 NP_478000 361 | | | | |
| | NP_477999 361 NP_477998 361 NP_477997 361 NP_477996 361 NP_477995 361 NP_477994 361 | | | | |
| | NP_477993 361 NP_477992 361 NP_477991 361 NP_477990 361 NP_477989 361 NP_477988 361 | | | | |
| | NP_477987 361 NP_477986 361 NP_477985 361 NP_477984 361 NP_477983 361 NP_477982 361 | | | | |
| | NP_477981 361 NP_477980 361 NP_477979 361 NP_477978 361 NP_477977 361 NP_477976 361 | | | | |
| | NP_477975 361 NP_477974 361 NP_477973 361 NP_477972 361 NP_477971 361 NP_477970 361 | | | | |
| | NP_477969 361 NP_477968 361 NP_477967 361 NP_477966 361 NP_477965 361 NP_477964 361 | | | | |
| | NP_477963 361 NP_477962 361 NP_477961 361 NP_477960 361 NP_477958 361 NP_477957 361 | | | | |
| | NP_477956 361 NP_477955 361 NP_477954 361 NP_477953 361 NP_477952 361 NP_477951 361 | | | | |
| | NP_477950 361 NP_477949 361 NP_477948 361 NP_477947 361 NP_477946 361 NP_477945 361 | | | | |
| | NP_477944 361 NP_477943 361 NP_477942 361 NP_477941 361 NP_477940 361 NP_477939 361 | | | | |
| | NP_477938 361 NP_477937 361 NP_477936 361 NP_477935 361 NP_477934 361 NP_477933 361 | | | | |
| | NP_477932 361 NP_477931 361 NP_477930 361 NP_477929 361 NP_477928 361 NP_477927 361 | | | | |
| | NP_477926 361 NP_477925 361 NP_477924 361 NP_477923 361 NP_477922 361 NP_477921 361 | | | | |
| | NP_477920 361 NP_477919 361 NP_477918 361 NP_477917 361 NP_477916 361 NP_477915 361 | | | | |
| | NP_477914 361 NP_477913 361 NP_477912 361 NP_477911 361 NP_477910 361 NP_477909 361 | | | | |
| | NP_477908 361 NP_477907 361 NP_477906 361 NP_477905 361 NP_477904 361 NP_477903 361 | | | | |
| | NP_477902 361 NP_477901 361 NP_477900 361 NP_477899 361 NP_477898 361 NP_477897 361 | | | | |
| | NP_477896 361 NP_477895 361 NP_477894 361 NP_477893 361 NP_477892 361 NP_477891 361 | | | | |
| | NP_477890 361 NP_477889 361 NP_477888 361 NP_477887 361 NP_477886 361 NP_477885 361 | | | | |
| | NP_477884 361 NP_477883 361 NP_477882 361 NP_477881 361 NP_477880 361 NP_477879 361 | | | | |
| | NP_477878 361 NP_477877 361 NP_477876 361 NP_477875 361 NP_477874 361 NP_477873 361 | | | | |
| | NP_477872 361 NP_477871 361 NP_477870 361 NP_477869 361 NP_477868 361 NP_477867 361 | | | | |
| | NP_477866 361 NP_477865 361 NP_477864 361 NP_477863 361 NP_477862 361 NP_477861 361 | | | | |
| | NP_477860 361 NP_477859 361 NP_477858 361 NP_477857 361 NP_477856 361 NP_477855 361 | | | | |
| | NP_477854 361 NP_477853 361 NP_477852 361 NP_477851 361 NP_477850 361 NP_477849 361 | | | | |
| | NP_477848 361 NP_477847 361 NP_477846 361 NP_477845 361 NP_477844 361 NP_477843 361 | | | | |
| | NP_477842 361 NP_477841 361 NP_477840 361 NP_477839 361 NP_477838 361 NP_477837 361 | | | | |
| | NP_477836 361 NP_477835 361 NP_477834 361 NP_477833 361 NP_477832 361 NP_477831 361 | | | | |
| | NP_477830 361 NP_477829 361 NP_477828 361 NP_477827 361 NP_477826 361 NP_477825 361 | | | | |
| | NP_477824 361 NP_477823 361 NP_477822 361 NP_477821 361 NP_477820 361 NP_477819 361 | | | | |
| | NP_477818 361 NP_477817 361 NP_477816 361 NP_477815 361 NP_477814 361 NP_477813 361 | | | | |
| | NP_477812 361 NP_477811 361 NP_477810 361 NP_477808 361 NP_477807 361 NP_477806 361 | | | | |
| | NP_477805 361 NP_477804 361 NP_477803 361 NP_477802 361 NP_477801 361 NP_477800 361 | | | | |
| | NP_477799 361 NP_477798 361 NP_477797 361 NP_477796 361 NP_477795 361 NP_477794 361 | | | | |
| | NP_477793 361 NP_477792 361 NP_477791 361 NP_477790 361 NP_477789 361 NP_477788 361 | | | | |
| | NP_477787 361 NP_477786 361 NP_477785 361 NP_477784 361 NP_477783 361 NP_477782 361 | | | | |
| | NP_477781 361 NP_477780 361 NP_477779 361 NP_477778 361 NP_477777 361 NP_477776 361 | | | | |
| | NP_477775 361 NP_477773 361 NP_477772 361 NP_477771 361 NP_477770 361 NP_477769 361 | | | | |
| | NP_477767 361 NP_477766 361 NP_477765 361 NP_477764 361 NP_477763 361 NP_477762 361 | | | | |
| | NP_477761 361 NP_477760 361 NP_477759 361 NP_477758 361 NP_477757 361 NP_477755 361 | | | | |
| | NP_477754 361 NP_477752 361 NP_477751 361 NP_477750 361 NP_477749 361 NP_477748 361 | | | | |
| | NP_477747 361 NP_477746 361 NP_477745 361 NP_477744 361 NP_477743 361 NP_477742 361 | | | | |
| | NP_477741 361 NP_477740 361 NP_477739 361 NP_477738 361 NP_477737 361 NP_477736 361 | | | | |
| | NP_477735 361 NP_477734 361 NP_477733 361 NP_477732 361 NP_477731 361 NP_477730 361 | | | | |
| | NP_477729 361 NP_477728 361 NP_477727 361 NP_477726 361 NP_477725 361 NP_477724 361 | | | | |
| | NP_477723 361 NP_477722 361 NP_477721 361 NP_477720 361 NP_477719 361 NP_477718 361 | | | | |
| | NP_477717 361 NP_477716 361 NP_477715 361 NP_477714 361 NP_477713 361 NP_477712 361 | | | | |
| | NP_477711 361 NP_477710 361 NP_477709 361 NP_477708 361 NP_477707 361 NP_477706 361 | | | | |
| | NP_477705 361 NP_477704 361 NP_477703 361 NP_477702 361 NP_477701 361 NP_477700 361 | | | | |
| | NP_477699 361 NP_477698 361 NP_477697 361 NP_477696 361 NP_477695 361 NP_477694 361 | | | | |
| | NP_477693 361 NP_477692 361 NP_477691 361 NP_477690 361 NP_477689 361 NP_477688 361 | | | | |
| | NP_477687 361 NP_477686 361 NP_477685 361 NP_477684 361 NP_477683 361 NP_477682 361 | | | | |
| | NP_477681 361 NP_477680 361 NP_477679 361 NP_477678 361 NP_477677 361 NP_477676 361 | | | | |
| | NP_477675 361 NP_477674 361 NP_477673 361 NP_477672 361 NP_477671 361 NP_477670 361 | | | | |
| | NP_477669 361 NP_477668 361 NP_477667 361 NP_477666 361 NP_477665 361 NP_477664 361 | | | | |
| | NP_477663 361 NP_477662 361 NP_477661 361 NP_477660 361 NP_477659 361 NP_477658 361 | | | | |
| | NP_477657 361 NP_477656 361 NP_477655 361 NP_477654 361 NP_477653 361 NP_477652 361 | | | | |
| | NP_477651 361 NP_477650 361 NP_477649 361 NP_477648 361 NP_477647 361 NP_477646 361 | | | | |
| | NP_477645 361 NP_477644 361 NP_477643 361 NP_477642 361 NP_477641 361 NP_477640 361 | | | | |
| | NP_477639 361 NP_477638 361 NP_477637 361 NP_477636 361 NP_477635 361 NP_477634 361 | | | | |
| | NP_477633 361 NP_477632 361 NP_477631 361 NP_477630 361 NP_477629 361 NP_477628 361 | | | | |
| | NP_477627 361 NP_477626 361 NP_477625 361 NP_477624 361 NP_477623 361 NP_477622 361 | | | | |
| | NP_477621 361 NP_477620 361 NP_477619 361 NP_477618 361 NP_477617 361 NP_477616 361 | | | | |
| | NP_477615 361 NP_477614 361 NP_477613 361 NP_477612 361 NP_477611 361 NP_477610 361 | | | | |
| | NP_477609 361 NP_477608 361 NP_477607 361 NP_477606 361 NP_477605 361 NP_477604 361 | | | | |
| | NP_477603 361 NP_477602 361 NP_477601 361 NP_477600 361 NP_477599 361 NP_477598 361 | | | | |
| | NP_477597 361 NP_477596 361 NP_477595 361 NP_477594 361 NP_477593 361 NP_477592 361 | | | | |
| | NP_477591 361 NP_477590 361 NP_477589 361 NP_477588 361 NP_477587 361 NP_477586 361 | | | | |
| | NP_477585 361 NP_477584 361 NP_477583 361 NP_477582 361 NP_477581 361 NP_477580 361 | | | | |
| | NP_477579 361 NP_477578 361 NP_477577 361 NP_477576 361 NP_477575 361 NP_477574 361 | | | | |
| | NP_477573 361 NP_477572 361 NP_477571 361 NP_477570 361 NP_477569 361 NP_477568 361 | | | | |
| | NP_477567 361 NP_477566 361 NP_477565 361 NP_477564 361 NP_477563 361 NP_477562 361 | | | | |
| | NP_477561 361 NP_477560 361 NP_477559 361 NP_477558 361 NP_477557 361 NP_477556 361 | | | | |
| | NP_477555 361 NP_477554 361 NP_477553 361 NP_477552 361 NP_477551 361 NP_477550 361 | | | | |
| | NP_477549 361 NP_477548 361 NP_477547 361 NP_477546 361 NP_477545 361 NP_477544 361 | | | | |
| | NP_477543 361 NP_477542 361 NP_477541 361 NP_477540 361 NP_477539 361 NP_477538 361 | | | | |
| | NP_477537 361 NP_477536 361 NP_477535 361 NP_477534 361 NP_477533 361 NP_477532 361 | | | | |
| | NP_477531 361 NP_477530 361 NP_477529 361 NP_477527 361 NP_477526 361 NP_477525 361 | | | | |
| | NP_477524 361 | | | | |
| 2005 | AAZ29239 9 XP_001681561 6 | 2 | 2.6 | 2.4 | low p > 0.20, prev p < 0.001 |
| 2006 | ABM92267 14 ABP01348 1 | 14 | 2.7 | 2.5 | low p < .001, prev p > .50 |
| | ABM64218 6 ABI34434 6 ABI93178 4 | | | | |
| | ABI93177 3 ABI93176 6 ABI93174 12 | | | | |
| | ABQ12866 3 ABD65308 2 ABD65303 | | | | |
| | 1 ABD65302 4 ABD65300 3 | | | | |
| | ABD65298 1 | | | | |
| 2007 | 2ED6_L 1 2ED6_K 1 2ED6_J 1 2ED6_I | 25 | 1.3 | 1.2 | low p < .001, prev p < .05 |
| | 1 2ED6_H 1 2ED6_G 1 2ED6_F 1 | | | | |
| | 2ED6_E 1 2ED6_D 1 2ED6_C 1 | | | | |
| | 2ED6_B 1 2ED6_A 1 ABQ12772 15 | | | | |
| | ABQ12773 3 ABQ12771 6 ABQ12770 | | | | |
| | 9 ABO69369 2 ABO69368 2 ABS00974 | | | | |
| | 5 ABS00973 1 ABQ44211 3 ABQ44210 | | | | |
| | 4 ABP52058 4 ABP52057 1 ABP52054 5 | | | | |

B. Prediction and Treatment of WSSV Outbreaks

Prediction of epidemics and future outbreaks may be made, for example, by reviewing the Replikin Counts of isolates of WSSV and comparing the Replikin Count for a particular year with Replikin Counts from other years. A significant increase in Replikin Count from one year to the next and preferably over one, two, three or five years or more provides predictive value of an emerging strain of WSSV that may begin an outbreak of more highly virulent WSSV. A WSSV outbreak may be predicted within about six months to about one year, to about three, to about five years or more from the observation of a significant increase in Replikin concentration. The outbreak is preferably predicted within about one to about three years and more preferably within about one to about two years. An outbreak of WSSV, therefore, may be predicted within 1 to about 2 years as demonstrated in FIG. 18 wherein an epidemic occurred at about 1 year following a remarkably significant increase in Replikin concentration and in particular in the identified Replikin Peak Gene.

Significant increases may be observed over a time period of more than one year, such as three, four, five or more years. An outbreak may likewise be predicted within about six months to about one year or more from the initial observation of an observable decrease in Replikin concentration following a notable increase.

The correlation between Replikin concentration and viral outbreaks noted above provide a method of predicting outbreaks of WSSV by monitoring increases or decreases in Replikin Count in the RPG of isolates of WSSV. The method may employ isolates of individual strains or isolates of all strains of WSSV.

XV. Replikin Count in TSV Epidemic

An increase in Replikin concentration in taura syndrome virus (TSV) is predictive of an increase in virulence and lethality of the virus and allows for prediction of forthcoming outbreaks or increases in lethality. FIG. 19 illustrates a correlation between increased Replikin Count in the genome of TSV and outbreaks of the virus in 2000 and 2007 in shrimp. The Replikin Count data reflected in the graph is found in Table 19. Significant outbreaks of the disease are noted at years 2000 and 2007. It may be observed from the graph that outbreaks of the virus occur following an increase in Replikin concentration. In year 2000, TSV had a Replikin concentration of 2.7. Between 2001 and 2004, TSV had a lower mean Replikin concentration, as low as 0.7, and an identified Replikin Scaffold disappeared. In 2005 the Replikin Scaffold reappeared, with an increase in lysines and histidines, and a commensurate increase in Replikin concentration to 1.8, followed by an increase in TSV outbreaks in 2006-2007.

TABLE 19
TSV Replikin Count

| | | No. of | Mean Replikin | | |
|---|---|---|---|---|---|
| | | Isolates | Concentration | | |
| Year | PubMed Accession Number-Replikin Count | per year | per year | S.D. | Significance |
| 2000 | NP_149058 70 NP_149057 70 AAK72221 70 | 5 | 2.7 | 1.3 | low p < 0.02 |
| | AAK72220 70 AAG44834 4 | | | | |
| 2001 | AAM73766 7 | 1 | 0.7 | 0.0 | prev p < 0.02 |
| 2002 | AN77089 2 AAN77088 2 AAN77087 2 AAN77086 2 | 8 | 0.7 | 0.4 | low p > 0.50 |
| | AAW32934 2 AAW32932 2 AAW32930 2 AAW32929 1 | | | | |
| 2003 | AAR11292 6 AAR11291 6 AAR11290 6 | 3 | 0.6 | 0.0 | prev p < 0.20 |
| 2004 | AAX07125 2 AAX07117 2 AAT81157 75 AAT81158 75 | 23 | 0.8 | 0.9 | low p < 0.40, |
| | AAX07127 2 AAX07126 2 AAX07124 2 AAX07123 2 | | | | prev p < 0.20 |
| | AAX07122 2 AAX07121 2 AAX07120 2 AAX07119 2 | | | | |
| | AAX07118 2 AAX07116 2 AAX07115 2 AAX07114 2 | | | | |
| | AAX07113 2 AAX07112 2 AAX35819 2 AAX35818 1 | | | | |
| | AAX35817 2 AAX35816 1 AAX35815 2 | | | | |
| 2005 | AAY56364 71 AAY56363 71 AAY44822 1 AAY44821 | 12 | 1.8 | 1.7 | low p < 0.02, |
| | 1 AAY44820 1 AAY44819 1 AAY44818 1 AAY44817 1 | | | | prev p < 0.05 |
| | AAY89097 83 AAY89096 83 ABB17263 63 ABB17264 | | | | |
| | 63 | | | | |

The TSV is less virulent than WSSV and the structure of the TSV Replikin Scaffold is less closely related to influenza virus than are the structures of WSSV Replikin Scaffolds.

XVI. Software

A further aspect of the invention provides utilizing software that searches for Replikin Peak Genes and enables the discovery of the point or points in the genome that have the highest concentration of Replikins, the years in which they have occurred, the strain or strains in which they occur, the host or hosts in which they occur, the geographic locations in which they occur, their increase or decrease in the above years, strains, hosts and geographic location and point or small mutations that are correlatable with virulence.

XVII. SARS Replikin Concentration Correlates with Epidemics

An increase in Replikin concentration in coronaviruses also correlates with the SARS coronavirus epidemic. In particular, as may be seen in FIG. 9, Replikin concentration in Spike and Nucleocapsid Coronavirus Proteins preceded the SARS Coronavirus Epidemic of 2003. In FIG. 9, the x-axis indicates the year and the y-axis indicates the Replikin concentration. The appearance of the SARS outbreak is shown by the shaded area in the graph between 2003 and 2004. The peak of the shaded area represents a total number of eight countries in which the SARS outbreak occurred in 2003. The solid black symbols represent the mean Replikin concentration for spike coronavirus proteins and the vertical black bars represent the standard deviation of the mean.

FIG. 9 shows a remarkable constancy of low coronavirus Replikin concentration between 1995 and 2001 in the spike proteins, followed by a dramatic increase in 2002, one year before the SARS epidemic appeared in 2003. Replikin concentration of the spike proteins in SARS then returned to their normal pre-2003 levels, which correlated with the disappearance of SARS.

XVIII. Passive Immunity

In another aspect of the invention, isolated Replikin peptides may be used to generate antibodies, which may be used, for example to provide passive immunity in an individual. Various procedures known in the art may be used for the production of antibodies to Replikin sequences. Such antibodies include but are not limited to polyclonal, monoclonal, chimeric, humanized, single chain, Fab fragments and fragments produced by a Fab expression library. Antibodies that are linked to a cytotoxic agent may also be generated. Antibodies may also be administered in combination with an antiviral agent. Furthermore, combinations of antibodies to different Replikins may be administered as an antibody cocktail.

Monoclonal antibodies to Replikins may be prepared by using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies.

Antibodies to any peptides observed to be present in an emerging or re-emerging strain of virus and combinations of such antibodies are useful in the treatment and/or prevention of viral infection, especially RPG peptides and Replikin sequences isolated within RPG peptides.

Antibody fragments that contain binding sites for a Replikin may be generated by known techniques. For example, such fragments include but are not limited to F(ab′)2 fragments which can be produced by pepsin digestion of the antibody molecules and the Fab fragments that can be generated by reducing the disulfide bridges of the F(ab′)2 fragments. Alternatively, Fab expression libraries can be generated (Huse et al., 1989, Science, 246:1275-1281) to allow rapid and easy identification of monoclonal Fab fragments with the desired specificity.

In another aspect of the invention, immune serum containing antibodies to one or more Replikins obtained from an individual exposed to one or more Replikins may be used to induce passive immunity in another individual or animal. Immune serum may be administered via i.v. to a subject in need of treatment. Passive immunity also can be achieved by injecting a recipient with preformed antibodies to one or more Replikins. Passive immunization may be used to provide immediate protection to individuals who have been exposed to an infectious organism. Administration of immune serum or preformed antibodies is routine and the skilled practitioner can readily ascertain the amount of serum or antibodies needed to achieve the desired effect. One of the reasons that vaccines directed towards a particular protein antigen of a disease causing agent have not been fully effective in providing protection against the disease (such as foot and mouth vaccine which has been developed against the VP1 protein or large segments of the VP1 protein) is that the best antibodies have not been produced, that is—it is likely that the antibodies to the Replikins have not been produced.

For example, either epitopes other than Replikins present in the larger protein fragments may interfere according to the phenomenon of antigenic primacy and/or because the hydrolysis of larger protein sequences into smaller sequences for processing to produce antibodies results in loss of integrity of any Replikin structure that is present, e.g., the Replikin is cut in two and/or the histidine residue is lost in the hydrolytic processing. The present studies suggest that for a more effective vaccine to be produced, the Replikin sequences, and no other epitope, should be used as the vaccine. For example, a vaccine of the invention can be generated using any one of the Replikin peptides identified by the three-point recognition system. A more preferred vaccine comprises at least one Replikin sequence isolated in an RPG. Another preferred vaccine comprises an RPG peptide. Among the preferred Replikin peptides for use in a virus vaccine are those conserved Replikins observed to “re-emerge” after an absence from the amino acid sequence for one or more years.

The Replikin peptides of the invention, alone or in various combinations are administered to a subject, preferably by i.v. or intramuscular injection, in order to stimulate the immune system of the subject to produce antibodies to the peptide. Generally the dosage of peptides is in the range of from about 0.1 μg to about 10 mg. In another embodiment, the dosage of the peptides is in the range from about 10 μg to about 1 mg. In a preferred embodiment, the dosage of the peptides is in the range from about 50 μg to about 500 μg. The skilled practitioner can readily determine the dosage and number of dosages needed to produce an effective immune response.

XIX. A Control Test of Reliability of Method of Predicting Outbreaks with Replikin Count

Table 3, which contains H5N1 data above, provides Replikin Count data across eight gene areas and an increased correlation is observed between mortality data and the whole virus, the polymerase gene and the pB1 gene area (Replikin Peak Gene). See also, e.g., FIGS. 4, 16 and 17. In addition to the correlative aspect of the increase in Replikin Count and percent mortality, the data in Table 3, and all of the other data contained herein, above provide strong confirmation of the power and validity of the methodology of predicting changes in virulence and outbreaks of virus with changes in Replikin concentration. These data represent an objective test of the method of independently selecting and examining several thousand individual accession numbers within approximately 12 million total accession numbers in PubMed wherein each selection is independently submitted to the PubMed database under a separate request using objective software. If there were not a reliable principle and a reliable method underlying each request, the potential for obtaining random results, or no results, or results which do not track each other at p<0.001 would markedly increase. Table 3 provides results wherein p was less than 0.001 between each group as compared one to another.

In Table 3 the structures that are correlated have, to the knowledge of the Applicants, not been correlated before, that is, the inventors have examined the relationship of one internal virus structure to another internal virus structure or structures (e.g., three-way relationship between whole virus gene area, polymerase and Replikin Peak Gene area) and have examined the external relation of these two or more internal structures to a host result of the virus infection, that is, percent mortality.

Table 3 represents consistent reproducible data, on repeated trials, which is the essence of the reliability of any method. For example, Table 3 provides independent data on (1) whole virus concentration of Replikins, (2) just polymerase concentration of Replikins, and (3) just the Replikin Peak Gene concentration of Replikins. The data is then correlated with H5N1 mortality three times, namely in 2003, 2004 and 2005. The absence of significant changes in the pA and pB2 gene areas provides a control. In each case, the method measures Replikin concentration three ways, each of which correctly predict mortality, independently, thereby confirming the method, and further illustrating in the process, the magnifying function of the Replikin Peak Gene.

### EXAMPLES

### Example 1

### Calculation of Replikin Count of Replikin Peak Gene of an isolate of H3N2 from the pandemic year of 1968

The inventors queried Accession No. ABB54523 at www.pubmed.com. Accession No. ABB54523 discloses the amino acid sequence of SEQ ID NO: 1664, deduced from the genomic information of an H3N2 strain of Influenza A virus isolated in 1968 in Memphis. Upon analysis of SEQ ID NO: 1664, the inventors observed a Replikin Peak Gene having continuous Replikin sequences beginning at residue 15 (histidine) and continuing through residue 85 (lysine) (SEQ ID NO: 1665).

The inventors isolated the RPG (SEQ ID NO: 1665) in silico. SEQ ID NO: 1665 was identified for diagnostic and therapeutic uses in, for example, an immunogenic compound and a therapeutic vaccine compound and as a predictive sequence for lethal outbreaks of influenza. Seventeen Replikin sequences (SEQ ID NOS: 1667-1682) were identified in the RPG of SEQ ID NO: 1665 for diagnostic, therapeutic and predictive uses as described herein. SEQ ID NOS: 1667-1674 were identified in the amino-terminal of the sequence disclosed in Accession No. ABB54523 (SEQ ID NO: 1664), SEQ ID NOS: 1675-1682 were identified in the mid-molecule of the sequence.

The Replikin Count of the amino acid sequence (SEQ ID NO: 1664) disclosed at ABB54523 was seventeen Replikin sequences in 90 total amino acids for a Replikin Count of 18.9. The Replikin Count of the RPG (SEQ ID NO: 1665) was seventeen Replikin sequences in 71 total amino acids for a Replikin Count of 23.9.

### Example 2

### Calculation of Replikin Count of Replikin Peak Gene of an isolate of human H5N1 from 2003

The inventors queried Accession No. BAE07199 at www.pubmed.com. Accession No. BAE07199 discloses an amino acid sequence deduced from the genomic information of the RNA polymerase gene of an H5N1 strain of Influenza A virus isolated in 2003 in Hong Kong. The inventors analyzed the whole pB1 gene area (SEQ ID NO: 1683) of the polymerase sequence. Upon analysis of SEQ ID NO: 1683, the inventors observed a Replikin Peak Gene having continuous Replikin sequences that begin at residue 168 (lysine) and continue through residue 215 (lysine).

The inventors isolated the RPG (SEQ ID NO: 1684) in silico. SEQ ID NO: 1684 was identified for diagnostic and therapeutic uses in, for example, an immunogenic compound and a therapeutic vaccine compound and as a predictive sequence for lethal outbreaks of influenza. Seven Replikin sequences (SEQ ID NOS: 1685-1691) were identified in the RPG of SEQ ID NO: 1684 for diagnostic, therapeutic and predictive uses as described herein. Replikin sequences SEQ ID NOS: 1685-1691 were identified in the amino-terminal of the sequence disclosed in Accession No. BAE07199 (SEQ ID NO: 1683), Replikin sequences SEQ ID NOS: 1692-1694 were identified in the mid-molecule of the sequence, and Replikin sequence SEQ ID NOS: 1695-1699 were identified in the carboxy-terminal of the sequence.

The Replikin Count of the whole pB1 area sequence (SEQ ID NO: 1683) was 15 Replikin sequences in 757 total amino acids for a Replikin Count of 2.0. The Replikin Count of the RPG (SEQ ID NO: 1684) was seven Replikin sequences in 48 total amino acids for a Replikin Count 14.6.

### Example 3

### Calculation of Replikin Count of the pB1 Gene Area and pB1-F2 Sub-Gene Area of an Isolate of Human H5N1 from Indonesia in 2006

The inventors queried Accession No. ABI36257 at www.pubmed.com. Accession No. ABI36257 discloses an amino acid sequence deduced from the genomic information of the pB1 gene area of an H5N1 strain of Influenza A virus isolated in 2006 from Indonesia. The inventors analyzed the pB1-F2 gene area (SEQ ID NO: 1700). Upon analysis of SEQ ID NO: 1700, the inventors observed a Replikin Peak Gene having continuous Replikin sequences that begin at residue 15 (histidine) and continue through residue 85 (lysine) (SEQ ID NO: 1701).

The inventors isolated the RPG (SEQ ID NO: 1701) in silico for diagnostic and therapeutic uses in, for example, an immunogenic compound and a therapeutic vaccine compound and as a predictive sequence for lethal outbreaks of influenza. Sixteen Replikin sequences (SEQ ID NOS: 1702-1717) were identified in the RPG of SEQ ID NO: 1701 for diagnostic, therapeutic and predictive uses as described herein. Replikin sequences SEQ ID NOS: 1702-1703 were identified in the amino-terminal of the sequence of SEQ ID NO: 1701, Replikin sequences SEQ ID NOS: 1704-1717 were identified in the mid-molecule of the sequence, and no Replikin sequences were identified in the carboxy-terminal.

The Replikin Count of the whole pB1-F2 gene area sequence (SEQ ID NO: 1700) was 16 Replikin sequences in 90 total amino acids for a Replikin Count of 17.8. The Replikin Count of the RPG pB1-F2 subgene area (SEQ ID NO: 1701) was 16 Replikin sequences in 71 total amino acids for a Replikin Count 22.57.

### Example 4

### Calculation of Replikin Count of the pB1 gene area and pB1-F2 sub-gene area of an isolate of human H5N1 from Indonesia in 2007

The inventors queried Accession No. ABM90520 at www.pubmed.com. Accession No. ABM90520 discloses an amino acid sequence deduced from the genomic information of the pB1 gene area of an H5N1 strain of Influenza A virus isolated in 2007 in Indonesia. The inventors analyzed the pB1 gene area (SEQ ID NO: 1722). Upon analysis of SEQ ID NO: 1722, the inventors observed a Replikin Peak Gene having continuous Replikin sequences that begin at residue 15 (histidine) and continue through residue 85 (lysine) in the pB1-F2 gene area (SEQ ID NO: 1723)

The inventors isolated the RPG (SEQ ID NO: 1723) in silico. SEQ ID NO: 1723 was identified for diagnostic and therapeutic uses in, for example, an immunogenic compound and a therapeutic vaccine compound and as a predictive sequence for lethal outbreaks of influenza. Sixteen Replikin sequences (SEQ ID NOS: 1724-1739) were identified in the RPG (or pB1-F2 gene subarea) of SEQ ID NO: 1723 for diagnostic, therapeutic and predictive uses as described herein. Replikin sequences SEQ ID NOS: 1724-1725 were identified in the amino-terminal of the sequence of SEQ ID NO: 1723, Replikin sequences SEQ ID NOS: 1726-1739 were identified in the mid-molecule of the sequence, and no Replikin sequences were identified in the carboxy-terminal.

The Replikin Count of the whole pB1-F2 area sequence (SEQ ID NO: 1722) was 16 Replikin sequences in 90 total amino acids for a Replikin Count of 17.8. The Replikin Count of the RPG (SEQ ID NO: 1723) was 16 Replikin sequences in 71 total amino acids for a Replikin Count 22.5.

### Example 5

### Calculation of Replikin Count of the RPG of a 2007H1N1 Isolate from Thailand Having a Replikin Scaffold

The inventors queried Accession No. ABS71678 at www.pubmed.com. Accession No. ABS71678 discloses an amino acid sequence deduced from the genomic information of the hemagglutinin gene area of an H1N1 strain of Influenza A virus isolated in 2007 in Thailand. The inventors analyzed the amino acid sequence provided at ABS71678 (SEQ ID NO: 1995). Upon analysis of SEQ ID NO: 1995, the inventors observed a Replikin Peak Gene having continuous Replikin sequences that begin at residue 143 (histidine) and continue through residue 235 (lysine) (SEQ ID NO: 1996). A Replikin Scaffold, knglypnlsksyannkekevlvlwgvhh (SEQ ID NO: 2011) was observed within the RPG.

The inventors isolated the RPG (SEQ ID NO: 1996) in silico. SEQ ID NO: 1996 was identified for diagnostic and therapeutic uses in, for example, an immunogenic compound and a therapeutic vaccine compound and as a predictive sequence for lethal outbreaks of influenza. Twenty-six Replikin sequences (SEQ ID NOS: 1999-2024) were identified in the RPG of SEQ ID NO: 1996 for diagnostic, therapeutic and predictive uses as described herein. Replikin sequences SEQ ID NOS: 1997-2016 were identified in the amino-terminal of the sequence of SEQ ID NO: 1995, Replikin sequences SEQ ID NOS: 2017-2029 were identified in the mid-molecule of the sequence, and SEQ ID NOS: 2030-2042 were identified in the carboxy-terminal. The Replikin sequences were isolated for diagnostic, therapeutic and predictive uses.

The Replikin Count of the whole hemagglutinin sequence (SEQ ID NO: 1995) was 46 Replikin sequences in 564 total amino acids for a Replikin Count of 8.1. The Replikin Count of the RPG area (SEQ ID NO: 1996) was 26 Replikin sequences in 93 total amino acids for a Replikin Count of 28.

### Example 6

### Replikin Peak Gene Identification in EIV Isolate Reported at Accession No. ABS89395

Applicants reviewed Replikin sequences publicly available at www.pubmed.com to determine the Replikin Peak Gene Area of available isolates. A Replikin Peak Gene was identified in the pB1-F2 gene area of the virus in Accession No. ABS89395 at www.pubmed.com. The following example provides determination of the Replikin Peak Gene in a 2005 isolate of a Maryland strain of H3N8 serotype Influenza A virus.

The inventors queried Accession No. ABS89395 and analyzed the amino acid sequence provided (SEQ ID NO: 545). Upon analysis of the sequence, the inventors observed a Replikin Peak Gene having continuous Replikin sequences that begin at residue 15 (histidine) and continue through residue 85 (lysine) (SEQ ID NO: 546).

The inventors isolated the RPG (SEQ ID NO: 546) in silico and identified the sequence for diagnostic and therapeutic uses in, for example, an immunogenic compound and a therapeutic vaccine compound and as a predictive sequence for lethal outbreaks of tuberculosis. Sixteen Replikin sequences (SEQ ID NOS: 547-562) were identified in the RPG for diagnostic, therapeutic and predictive uses as described herein. Replikin sequences SEQ ID NOS: 547-548 were identified in the amino-terminal of the sequence, Replikin sequences SEQ ID NOS: 549-562 were identified in the mid-molecule of the sequence, and no Replikins were identified in the carboxy-terminal.

The Replikin Count of the whole pB1-F2 sequence (SEQ ID NO: 545) was 16 Replikin sequences in 90 total amino acids for a Replikin Count of 17.8. The Replikin Count of the RPG area (SEQ ID NO: 546) was 16 Replikin sequences in 71 total amino acids for a Replikin Count of 22.5.

### Example 7

### Replikin Peak Gene Identification in West Nile Virus Isolate Reported in Accession No. ABA54585

Applicants reviewed Replikin sequences publicly available at www.pubmed.com to determine the Replikin Peak Gene of an available West Nile Virus (WNV) isolate. The entire envelope protein of WNV was reported at Accession No. ABA54585. A Replikin Peak Gene was identified in the 3,433 amino acid polyprotein sequence of the WNV envelope protein. A Replikin Peak Gene was identified beginning at amino acid residue 2797 extending through amino acid residue 2836 (a total of 40 amino acid residues). The number of Replikin sequences in this section was 12. The Replikin Count (Replikins per 100 amino acids) was 30. The Replikin Peak Gene (RPG) of the envelope protein of WNV is SEQ ID NO: 258 and the RPG contains 12 uninterrupted Replikins (SEQ ID NOS: 246-257).

### Example 8

### Calculation of RPGs in Porcine Respiratory Syndrome Virus

Applicants reviewed Replikin sequences publicly available at www.pubmed.com to determine the Replikin Peak Gene Area of available PRRSV isolates. A Replikin Peak Gene was identified in Accession No. AA043261 from mRNA encoding a reported nucleocapsid protein of a PRRSV isolate from Mexico in 2003. The inventors analyzed the amino acid sequence provided in Accession No. AA043261, which is reported with 123 amino acids within ORF 7 of the virus genome. Upon analysis of the sequence, the inventors observed a Replikin Peak Gene having continuous Replikin sequences that begin at residue 7 (lysine) and continue through residue 66 (histidine).

The inventors isolated the RPG (SEQ ID NO: 394) in silico and identified the sequence for diagnostic and therapeutic uses in, for example, an immunogenic compound and a therapeutic vaccine compound and as a predictive sequence for outbreaks of PRRSV. Seven Replikin sequences (SEQ ID NOS: 395-401) were identified in the RPG for diagnostic, therapeutic and predictive uses as described herein. SEQ ID NO: 395 was identified in the amino-terminal portion of the sequence and SEQ ID NOS: 396-401 were identified in the mid-molecule portion of the sequence.

The Replikin Count of the whole nucleocapsid sequence at Accession No. AA043261 was 7 Replikin sequence in 123 amino acid residues or 5.7. The Replikin Count of the RPG area (SEQ ID NO: 394) was 7 Replikin sequences in 60 total amino acids for a Replikin Count of 11.7.

The asparagine and methionine residues at positions 45 and 46 of the RPG (SEQ ID NO: 394) were identified by the inventors as non-conserved positions within the RPG as compared to other reported nucleocapsid sequences such as Accession No. ABF19568 discussed immediately below. Non-conserved positions within an RPG that are correlated with changes in lethality and/or virulence are particularly useful in methods of the invention to predict outbreaks. The presence of these point mutations in other PRRSV nucleocapsid RPG sequences provides evidence of greater virulence and/or lethality.

A Replikin Peak Gene was also identified in Accession No. ABF19568 from a nucleic acid sequence of a PRRSV 2006 isolate from Mexico. The reported sequence has 99 amino acid residues. The RPG (SEQ ID NO: 402) was isolated in silico and identified for diagnostic and therapeutic uses in, for example, an immunogenic compound and a therapeutic vaccine compound and as a predictive sequence for outbreaks of PRRSV. The total length of the RPG is 29 amino acids. The Replikin Count is 41.4. The Replikin sequences of SEQ ID NOS: 403-414 were identified in the RPG for diagnostic, therapeutic and predictive uses as described herein. SEQ ID NOS: 403-414 were identified in the amino-terminal of the sequence. No Replikin sequences were identified in the mid-molecule or carboxy-terminus.

The glycine, proline and glycine residues at positions 14 through 16 and the asparagine, arginine, lysine, arginine and asparagine residues at positions 21 through 25 were identified within the RPG (SEQ ID NO: 507) as non-conserved positions as compared to other reported nucleocapsid sequences such as Accession No. AA043261 above. Further, as compared to the RPG in Accession No. AA043261 above, the RPG identified in the 2006 Mexico isolate demonstrates a shortening of the RPG and noteworthy condensation of Replikin sequences within the shorter RPG. The result is a remarkable increase in Replikin Count between 2003 and 2006 corresponding to a severe outbreak of PRRSV in Mexico in 2006 with an increase in mortality rate.

Applicants likewise analyzed Accession Nos. AAM18565, AAP81809, ABL60920 having sequences of isolates from China in 2000, 2003, and 2006, respectively, to determine the Replikin Peak Gene of the isolates (SEQ ID NOS: 341, 342, and 343).

A Replikin Peak Gene was identified in Accession No. AAM18565 between residue 7 (lysine) and residue 66 (histidine). The inventors isolated the RPG (SEQ ID NO: 353) in silico and identified the sequence for diagnostic and therapeutic uses in, for example, an immunogenic compound and a therapeutic vaccine compound and as a predictive sequence for outbreaks of PRRSV. Thirteen Replikin sequences (SEQ ID NOS: 354-366) were identified in the RPG for diagnostic, therapeutic and predictive uses as described herein. SEQ ID NOS: 354-357 were identified in the amino-terminal portion of the sequence and SEQ ID NOS: 358-366 were identified in the mid-molecule portion of the sequence.

The Replikin Count of the whole sequence at Accession No. AAM18565 was 13 Replikin sequences within 123 amino acid residues or 10.6. The Replikin Count of the RPG area (SEQ ID NO: 353) was 13 Replikin sequences in 60 total amino acids for a Replikin Count of 21.7.

A Replikin Peak Gene was identified in Accession No. AAP81809 between residue 7 (lysine) and residue 66 (histidine). The inventors isolated the RPG (SEQ ID NO: 367) in silico and identified the sequence for diagnostic and therapeutic uses in, for example, an immunogenic compound and a therapeutic vaccine compound and as a predictive sequence for outbreaks of PRRSV. Thirteen Replikin sequences (SEQ ID NOS: 368-380) were identified in the RPG for diagnostic, therapeutic and predictive uses as described herein. SEQ ID NOS: 368-371 were identified in the amino-terminal portion of the sequence and SEQ ID NOS: 372-380 were identified in the mid-molecule portion of the sequence.

The Replikin Count of the whole sequence at Accession No. AAP81809 was 13 Replikin sequences within 123 amino acid residues or 10.6. The Replikin Count of the RPG area (SEQ ID NO: 367) was 13 Replikin sequences in 60 total amino acids for a Replikin Count of 21.7.

A Replikin Peak Gene was identified in Accession No. ABL60920 between residue 7 (lysine) and residue 66 (histidine). The inventors isolated the RPG (SEQ ID NO: 382) in silico and identified the sequence for diagnostic and therapeutic uses in, for example, an immunogenic compound and a therapeutic vaccine compound and as a predictive sequence for outbreaks of PRRSV. Ten Replikin sequences (SEQ ID NOS: 384-393) were identified in the RPG for diagnostic, therapeutic and predictive uses as described herein. SEQ ID NOS: 384-387 were identified in the amino-terminal portion of the sequence and SEQ ID NOS: 388-393 were identified in the mid-molecule portion of the sequence.

The Replikin Count of the whole sequence at Accession No. ABL60920 was 10 Replikin sequences within 123 amino acid residues or 8.1. The Replikin Count of the RPG area (SEQ ID NO: 367) was 10 Replikin sequences in 60 total amino acids for a Replikin Count of 16.7.

### Example 9

### Calculation of RPG in Porcine Circovirus

Applicants reviewed Replikin sequences publicly available at www.pubmed.com to determine the Replikin Peak Gene of available isolates of PCV. The inventors identified and compared a Replikin Peak Gene (RPG) of a protein fragment at Accession No. AAC59472 of a strain of PCV isolated from infected pigs in Manitoba, Canada in 1997 and a RPG of a putative truncated replicase protein at Accession No. ABP68657 of a strain of PCV isolated from infected pigs in China in 2007. The AAC59472 fragment was identified from nucleic acid encoding a predicted 1.8 kDa protein in open reading frame 11 of the isolate. The ABP68657 putative truncated replicase protein was identified in open reading frame 1 of the isolate.

In Accession No. AAC59472, the inventors identified an RPG (SEQ ID NO: 520) for diagnostic, therapeutic and predictive purposes as described herein. The RPG begins at residue 2 (lysine) and continues through residue 12 (lysine). Four Replikin sequences (SEQ ID NOS: 521-524) were identified for diagnostic, therapeutic and predictive uses as described in herein. The total length of the RPG is 11 amino acids. The Replikin Count is 36.4. The Replikin Count of the entire fragment is four Replikin sequences in fourteen amino acids or 28.6.

In Accession No. ABP68657, the inventors identified an RPG (SEQ ID NO: 525) for diagnostic, therapeutic and predictive purposes as described herein. Thirteen Replikin sequences (SEQ ID NOS: 526-538) were identified for diagnostic, therapeutic and predictive uses as described in herein. The total length of the RPG is 38 amino acids. The Replikin Count is 34.2. The Replikin Count of the entire putative truncated protein is 6.2.

The reported sequence at Accession No. AAC59472 has only 14 amino acid residues. Nevertheless, the high concentration of continuous, non-interrupted and overlapping Replikin sequences within the RPG (Replikin Count 36.4) is a predictor of virulence and provides sequences available as vaccines. In comparison, the RPG of the truncated replicase protein reported at Accession No. ABP68657 has 306 amino acid residues but the identified RPG has 13 Replikin sequences and a comparable Replikin Count of 34.2, which is likewise a predictor of virulence and provides sequences available as vaccines. Likewise, the high Replikin Count RPGs provide a target for production of immunogenic compounds for treatment and prevention of PCV.

A Replikin Peak Gene was identified in an isolate of PCV from 1997 publicly available at Accession No. AAC9885 between residues 4 (lysine) and 99 (histidine). The inventors isolated the RPG (SEQ ID NO: 421) in silico and identified the sequence for diagnostic and therapeutic uses in, for example, an immunogenic compound and a therapeutic vaccine compound and as a predictive sequence for outbreaks of PCV. Fourteen Replikin sequences (SEQ ID NOS: 422-435) were identified in the RPG for diagnostic, therapeutic and predictive uses as described herein. SEQ ID NOS: 422-435 were identified in the amino-terminal portion of the whole sequence disclosed at the accession number and SEQ ID NOS: 436-437 were identified in the mid-molecule portion of the sequence. No Replikin sequences were identified in the carboxy-portion of the sequence.

The Replikin Count of the whole sequence at Accession No. AAC9885 was 16 Replikin sequences within 312 amino acid residues or 5.1. The Replikin Count of the RPG area (SEQ ID NO: 421) was 14 Replikin sequences in 96 total amino acid residues for a Replikin Count of 14.6.

A Replikin Peak Gene was identified in an isolate of PCV from 2001 publicly available at Accession No. AAL01075 between residues 57 (histidine) and 94 (lysine). The inventors isolated the RPG (SEQ ID NO: 438) in silico and identified the sequence for diagnostic and therapeutic uses in, for example, an immunogenic compound and a therapeutic vaccine compound and as a predictive sequence for outbreaks of PCV. Twelve Replikin sequences (SEQ ID NOS: 439-450) were identified in the RPG for diagnostic, therapeutic and predictive uses as described herein. SEQ ID NOS: 439-445 were identified in the amino-terminal portion of the whole sequence disclosed at the accession number and SEQ ID NOS: 446-450 were identified in the mid-molecule portion of the sequence. No Replikin sequences were identified in the carboxy-portion of the sequence.

The Replikin Count of the whole sequence at Accession No. AAC9885 was 12 Replikin sequences within 314 amino acid residues or 3.8. The Replikin Count of the RPG area (SEQ ID NO: 438) was 12 Replikin sequences in 90 total amino acids for a Replikin Count of 13.3.

A Replikin Peak Gene was identified in an isolate of PCV from Canada in 2007 that is publicly available at Accession No. ABP68657. The RPG was identified between residues 57 (histidine) and 94 (lysine). The inventors isolated the RPG (SEQ ID NO: 462) in silico and identified the sequence for diagnostic and therapeutic uses in, for example, an immunogenic compound and a therapeutic vaccine compound and as a predictive sequence for outbreaks of PCV. Fourteen Replikin sequences (SEQ ID NOS: 462-476) were identified in the RPG for diagnostic, therapeutic and predictive uses as described herein. SEQ ID NOS: 462-476 were identified in the amino-terminal portion of the whole sequence disclosed at the accession number and SEQ ID NOS: 477-481 were identified in the mid-molecule portion of the sequence. No Replikin sequences were identified in the carboxy-portion of the sequence.

The Replikin Count of the whole sequence at Accession No. ABP68657 was 19 Replikin sequences within 306 amino acid residues or 6.2. The Replikin Count of the RPG area (SEQ ID NO: 462) was 14 Replikin sequences in 38 total amino acids for a Replikin Count of 36.8.

In Applicants' review of RPGs in publicly available PCV sequences, the inventors identified an RPG from Accession No. ABQ 10608 that contained each of the highly conserved Replikin sequences discussed in Section XI.G. above, namely, kngrsgpqphk (SEQ ID NO: 345); hlqgfanfvkkqtfnk (SEQ ID NO: 346) and kkqtfnkvkwylgarch (SEQ ID NO: 347).

The RPG was identified between residues 57 (histidine) and 94 (lysine). The inventors isolated the RPG (SEQ ID NO: 498) in silico and identified the sequence for diagnostic and therapeutic uses in, for example, an immunogenic compound and a therapeutic vaccine compound and as a predictive sequence for outbreaks of PCV. Six Replikin sequences (SEQ ID NOS: 487-492) were identified in the RPG for diagnostic, therapeutic and predictive uses as described herein. SEQ ID NOS: 486-492 were identified in the amino-terminal portion of the whole sequence disclosed at the accession number and SEQ ID NOS: 493-497 were identified in the mid-molecule portion of the sequence for therapeutic, diagnostic and predictive purposes. No Replikin sequences were identified in the carboxy-portion of the sequence.

The Replikin Count of the whole sequence at Accession No. ABQ10608 was 12 Replikin sequences within 314 amino acid residues or 3.8. The Replikin Count of the RPG area (SEQ ID NO: 498) was six Replikin sequences in 38 total amino acids or 15.8.

### Example 10

### Calculation of RPG in Tuberculosis Pathogen Mycobacterium

The inventors queried Accession No. AAS59518 at www.pubmed.com. Accession No. AAS59518 discloses an amino acid sequence from Mycobacterium mucogenicum strain CIP 105384. The inventors analyzed the amino acid sequence provided at AAS59518 (SEQ ID NO: 2901). Upon analysis of SEQ ID NO: 2901, the inventors observed a Replikin Peak Gene having continuous Replikin sequences that begin at residue 3 (histidine) and continue through residue 88 (histidine) (SEQ ID NO: 3649).

The inventors isolated the RPG (SEQ ID NO: 3659) in silico for diagnostic and therapeutic uses in, for example, an immunogenic compound and a therapeutic vaccine compound and as a predictive sequence for lethal outbreaks of tuberculosis. Twenty-four Replikin sequences (SEQ ID NOS: 2902-2925) were identified in the RPG of SEQ ID NO: 3659 for diagnostic, therapeutic and predictive uses as described herein. Replikin sequences SEQ ID NOS: 2902-2924 were identified in the amino-terminal of the sequence of SEQ ID NO: 2901, Replikin sequences SEQ ID NO: 2925 was identified in the mid-molecule of the sequence, and no Replikins were identified in the carboxy-terminal. All were isolated for diagnostic, therapeutic and predictive purposes.

The Replikin Count of the whole hemagglutinin sequence (SEQ ID NO: 2901) was 24 Replikin sequences in 147 total amino acids for a Replikin Count of 16.3. The Replikin Count of the RPG area (SEQ ID NO: 3659) was 24 Replikin sequences in 87 total amino acids for a Replikin Count of 27.6.

### Example 10

### Determination of Very High Replikin Count in WSSV Ribonucleotide Reductase from Accession No. AAL89390

Replikin concentration was determined for ribonucleotide reductase of a white spot syndrome virus (WSSV) isolate publicly available at Accession No. AAL89390. The amino acid sequence was translated from the total genome of a 2000 isolate of WSSV made publicly available at Accession No. NC 003225.1. The Replikin concentration in the protein was an unusually high 103.8 and the Replikin concentration of the Replikin Peak Gene of the protein was a yet higher 110.7.

The amino acid sequence of the protein publicly available at Accession No. AAL89390 is of particular interest because it demonstrates an overlapping of Replikin sequences that results in very high Replikin concentrations comparable to P. falciparum. The high concentrations of Replikin sequences provide a reservoir for transfer to influenza viruses.

In Accession No. AAL89390, SEQ ID NO: 668 is disclosed as a ribonucleotide reductase protein of white spot syndrome virus. Within SEQ ID NO: 668, the inventors identified a Replikin Peak Gene (SEQ ID NO: 669). The Replikin Peak Gene is observed to occupy most of the disclosed protein of SEQ ID NO: 668. The expansiveness of the Replikin Peak Gene across most of the amino acid sequence of the protein is highly unusual and creates a remarkably high Replikin concentration.

Replikin Count of SEQ ID NO: 668 was determined by dividing the number of Replikin sequences identified in the amino acid sequence of the protein, 497 Replikin sequences, by the total amino acid length of the protein, 479 amino acids, to arrive at 103.8 Replikin sequences per 100 amino acids. The Replikin Count of the RPG of SEQ ID NO: 669 was determined by dividing the number of Replikin sequences identified in the segment of the protein containing the highest concentration of continuous Replikin sequences, 497 Replikin sequences, by the total amino acid length Replikin Peak Gene, 449 amino acids, to arrive at 110.7.

Within the RPG of SEQ ID NO: 669, SEQ ID NOS: 670-1166 were identified as Replikin sequences. SEQ ID NOS: 669-866 were identified in the amino-terminus of the peptide, SEQ ID NOS: 867-1065 were identified in the middle portion, and SEQ ID NOS: 1066-1166 were identified in the carboxy-terminus.

SEQ ID NO: 669 was further observed to contain significant Replikin Scaffold sequences. SEQ ID NOS: 663-667 were identified as Replikin Scaffold repeats and were isolated for diagnostic, therapeutic and predictive uses.

Replikin Count was determined for a functionally undefined protein in the genome of a 2000 isolate WSSV at Accession No. NP 478030 (SEQ ID NO: 1167). The Replikin Count in the protein was again an unusually high 97.6 Replikin sequences per 100 amino acids determined by dividing the number of Replikin sequences identified in the amino acid sequence of the protein, 361 Replikin sequences, by the total amino acid length of the protein, 370 amino acids.

An RPG (SEQ ID NO: 1168) was identified within SEQ ID NO: 1167 between residues 22 (histidine) and 361 (lysine) and is available for diagnostic, therapeutic and predictive uses as described herein. Total Replikin sequences identified in the RPG were 361 with total amino acid residues of 361, for a Replikin Count in the RPG of 100. SEQ ID NOS: 1169-1330 were identified in the amino-terminus of the RPG. SEQ ID NOS: 1331-1465 were identified in the mid-molecule of the RPG and SEQ ID NOS: 1466-1529 were identified in the carboxy-terminus of the RPG. Each Replikin sequence is available for diagnostic, therapeutic and predictive purposes as described herein.

The amino acid sequence of Accession No. NP 478030 is of interest because, like the protein in Accession No. AAL89390, it demonstrates an overlapping of Replikin sequences that results in very high Replikin concentrations comparable to the highly-replicating P. falciparum of malaria. Overlapping Replikin sequences are exceptional targets for therapies such as immunogenic agents and vaccines and have excellent predictive capacities.

In 2006 and 2007 WSSV has been observed to be dormant in shrimp. This continued decline of WSSV into “quiescent” or “dormant” levels in 2006-2007 is demonstrated in mean Replikin Counts for viruses isolated during 2005-2007 that are very low as compared to years wherein the virus demonstrated greater virulence, such as 2001. The continued quiescence in WSSV in 2007 may be contrasted with an observed rising of Replikin concentration in taura syndrome virus Replikin during this period.

As may be seen from the analysis below, Accession Nos. ABS00973 and AAW88445 have low observed Replikin concentrations. ABS00973 contains a single Replikin sequence (SEQ ID NO: 1548) in the entire disclosed amino acid sequence of 240 residues at SEQ ID NO: 1547. The Replikin concentration of Accession No. ABS00973 is an inordinately low 0.5. AAW88445 contains a white spot syndrome virus protein of 261 amino acid residues (SEQ ID NO: 1530). An RPG of 34-105 was identified (SEQ ID NO: 1531). Within the RPG, eleven Replikin sequences were identified (SEQ ID NOS: 1532-1542). SEQ ID NOS: 1532-1542 were identified in the amino-terminus of SEQ ID NO: 1530 and SEQ ID NOS: 1543-1546 were identified in the carboxy-terminus of SEQ ID NO: 1530.

### Example 11

### Calculation of Replikin Count in Accession No. AAM73766 and AAY89096 in Taura Syndrome Virus

The inventors queried Accession No. AAM73766 at www.pubmed.com. Accession No. AAM73766 discloses an amino acid sequence from a 2005 isolate of TSV (SEQ ID NO: 3566). Applicants identified SEQ ID NOS: 3567-3569 as Replikin sequences in the amino-terminus of the sequence and SEQ ID NOS: 3570-3573 as Replikin sequences in the carboxy-terminus of the sequence. Each sequence was isolated in silico for diagnostic, therapeutic and predictive purposes as described herein. No Replikin sequence was identified in the mid-molecule. The Replikin Count of SEQ ID NO: 3566 was seven Replikin sequences in 1011 amino acid residues or 0.7.

The inventors queried Accession No. AAY89096 at www.pubmed.com. Accession No. AAY89096 discloses an amino acid sequence from a 2005 isolate of TSV (SEQ ID NO: 3574). Applicants identified SEQ ID NOS: 3575-3587 in the amino-terminus of the sequence. SEQ ID NOS: 3588-3634 were identified as Replikin sequences in the mid-molecule. And SEQ ID NOS: 3635-3657 were identified as Replikin sequences in the carboxy-terminus of the sequence. Each sequence was isolated in silico for diagnostic, therapeutic and predictive purposes as described herein. Replikin Count of SEQ ID NO: 3574 was 83 Replikin sequences in 2107 amino acid residues or 3.9.

### Example 12

### Analysis of pB1-F2 Gene Area for All Publicly Available Influenza A Strains 2002-2007

The inventors queried www.pubmed.com with the software program FluForecast® available from Replikins LLC of Boston, Mass. to analyze all amino acid sequences from the pB1-F2 gene area of all isolates of Influenza A available between 2002 and 2007. Table 20 provides the results of the query. The data for mean Replikin count for 2005, 2006 and 2007 suggest that the current epidemic is not over. For example, the SARS data in FIG. 9 demonstrates that prior to a decline in epidemic infections, a decrease in Replikin Count is expected. Such a decline is not seen in the data in Table 20.

TABLE 20
PB1-F2 Influenza A 2002-2007

| | | | Mean | | |
|---|---|---|---|---|---|
| | | No. of | Replikin | | |
| | | Isolates | Count | | |
| | | per | per | | |
| Year | PubMed Accession Number-Replikin Count | year | year | S.D. | Significance |
| 2002 | ABD59827 15 ABD59825 15 ABD59823 12 | 3 | 1.8 | 0.2 | low p < .05, prev |
| | | | | | p < .001 |
| 2003 | ABD59830 12 ABK40004 12 AAZ79547 12 AAZ79504 15 | 4 | 1.7 | 0.2 | low p < .005, |
| | | | | | prev p < .30 |
| 2004 | ABD59835 12 ABD59833 12 | 2 | 1.6 | 0.0 | prev p < .30 |
| 2005 | ABI36231 16 ABI36226 16 ABI36221 16 ABI36217 16 | 10 | 9.6 | 8.1 | low p < .02 |
| | ABI36215 15 ABI36210 15 ABI36032 14 ABI36021 14 | | | | |
| | ABI36010 15 ABI36001 14 | | | | |
| 2006 | BAF37385 16 BAE97585 16 ABL31778 16 ABL31775 21 | 48 | 16.6 | 4.7 | low p < .001, |
| | ABL31764 21 ABL31742 16 ABI49394 16 ABL07028 21 | | | | prev p < .01 |
| | ABL07017 16 ABL07006 16 ABI49405 16 ABI36473 16 | | | | |
| | ABI36462 16 ABI36453 16 ABI36442 16 ABI36431 16 | | | | |
| | ABI36421 16 ABI36409 16 ABI36398 16 ABI36387 16 | | | | |
| | ABI36376 16 ABI36365 16 ABI36354 15 ABI36343 15 | | | | |
| | ABI36332 15 ABI36321 15 ABI36310 16 ABI36304 16 | | | | |
| | ABI36293 16 ABI36284 16 ABI36272 16 ABI36269 16 | | | | |
| | ABI36266 16 ABI36262 16 ABI36258 16 ABI36253 16 | | | | |
| | ABI36250 16 ABI36245 14 ABI36242 14 ABI36237 14 | | | | |
| | ABI36233 14 ABI36206 16 ABI36196 16 ABI36185 16 | | | | |
| | ABI36175 16 ABI36164 16 ABI36153 16 ABI36142 16 | | | | |
| 2007 | ABM90520 16 ABM90542 16 ABM90531 16 ABM90509 16 | 11 | 17.8 | 0.0 | low p < .001 |
| | ABM90498 16 ABM90487 16 ABM90476 16 ABM90465 16 | | | | |
| | ABM90454 16 ABM90443 16 ABM90432 16 | | | | |

### Example 13

### H1N1 hemagglutinin (whole hemagglutinin) Replikin Count by Year

The inventors queried www.pubmed.com with the software program FluForecast® available from Replikins LLC of Boston, Mass. to analyze all amino acid sequences of all isolates of H1N1 Influenza A available between 1917 and 2007. Table 21 provides the results of the query.

TABLE 21
H1N1 Influenza A 1918-2007

| | | | Mean | | |
|---|---|---|---|---|---|
| | | No. of | Replikin | | |
| | | Isolates | Count | | |
| | | per | per | | |
| Year | PubMed Accession Number-Replikin Count | year | year | S.D. | Significance |
| 1918 | AAO65768 13 | 1 | 7.0 | 0.0 | |
| 1919 | AAO65769 13 | 1 | 7.0 | 0.0 | |
| 1930 | Q9WCD9 27 AAD25303 27 AAB52905 16 CAA40729 16 | 5 | 4.4 | 0.7 | low p < .001, |
| | P05779 3 | | | | prev p < .001 |
| 1931 | AAA19935 16 ABD79255 27 | 2 | 4.7 | 0.1 | low p < .01, |
| | | | | | prev p < .30 |
| 1933 | ABD77796 31 ABF47955 31 P03454 31 AAA43209 31 P05780 | 6 | 4.4 | 1.7 | low p < .01, |
| | 2 P03470 11 | | | | prev p > .50 |
| 1934 | AAA58799 21 ABP64731 38 ABP64721 35 AAA43661 7 | 14 | 6.1 | 1.3 | low p < .02, |
| | ABO21709 38 ABD77675 38 P03452 38 AAA43194 21 | | | | prev p < .04 |
| | AAM75158 38 YP_163736 38 YP_163735 38 NP_040980 38 | | | | |
| | P03468 11 P06821 6 | | | | |
| 1935 | ABD62781 38 ABW71481 22 ABO38384 41 ABN59412 38 | 4 | 6.1 | 1.5 | low p < .30, |
| | | | | | prev p > .50 |
| 1936 | ABO38351 39 | 1 | 6.9 | 0.0 | prev p < .30 |
| 1937 | AAA67181 14 | 1 | 4.3 | 0.0 | |
| 1938 | AAA67182 18 | 1 | 5.5 | 0.0 | |
| 1939 | AAA67183 4 BAA00718 19 | 2 | 2.3 | 1.5 | low p < .10, |
| | | | | | prev p < .20 |
| 1940 | ABI20826 38 | 1 | 6.7 | 0.0 | prev p < .10 |
| 1941 | | | | | |
| 1942 | ABD62843 28 ABW38010 28 | 2 | 4.9 | 0.0 | |
| 1943 | AAM76687 14 ABD79101 40 AAM76691 9 AAM76688 9 | 7 | 4.1 | 1.7 | low p < .002, |
| | AAM76686 9 ABO38373 28 ABO38054 28 | | | | prev p < .20 |
| 1945 | ABP49327 28 | 1 | 4.9 | 0.0 | prev p < .20 |
| 1946 | ABD79112 38 | 1 | 6.7 | 0.0 | |
| 1947 | AAM76690 10 ABD77807 36 AAM76689 12 AAA67338 36 | 28 | 2.7 | 1.5 | low p < .001, |
| | AAC53844 36 BAA96109 14 AAA67339 8 AAA67340 2 | | | | prev p < .001 |
| | AAA67341 2 AAA67336 3 AAA67337 3 AAB39916 3 | | | | |
| | AAB39915 3 CAA67497 18 CAA67496 18 CAA67499 11 | | | | |
| | CAA67498 11 CAA67500 26 P26070 8 Q8JSD9 10 Q82571 11 | | | | |
| | Q82573 18 P03506 2 Q82570 26 CAB50889 2 CAB50888 2 | | | | |
| | CAB50887 8 CAB50886 26 | | | | |
| 1948 | BAA96110 14 ABN59401 37 | 2 | 5.3 | 1.7 | low p < .40, |
| | | | | | prev p < .05 |
| 1949 | ABN59434 37 | 1 | 6.5 | 0.0 | prev p < .40 |
| 1950 | ABD61735 29 ABP49316 41 P10921 3 | 3 | 5.2 | 2.1 | low p < .20, |
| | | | | | prev p < .30 |
| 1951 | BAA96112 16 BAA96111 10 ABR15808 36 ABQ44471 36 | 6 | 5.6 | 1.5 | low p < .05, |
| | ABQ01311 38 ABP49481 36 | | | | prev p > .50 |
| 1952 | BAA96113 13 | 1 | 3.8 | 0.0 | prev p < .02 |
| 1954 | ABD60966 34 BAA96114 4 ABO52280 34 | 3 | 4.4 | 2.8 | low p < .20, |
| | | | | | prev p > .50 |
| 1955 | BAA96115 6 | 1 | 1.7 | 0.0 | prev p < .20 |
| 1956 | BAA96116 9 AAF99713 18 AAF99712 16 | 3 | 2.9 | 0.3 | low p < .001, |
| | | | | | prev p < .02 |
| 1957 | ABD15258 24 AAG22555 3 BAA96117 8 ABV82573 36 | 4 | 4.3 | 1.6 | low p < .04, |
| | | | | | prev p < .10 |
| 1961 | Q9WCD8 36 AAD25302 36 | 2 | 6.4 | 0.0 | prev p < .05 |
| 1963 | CAA40730 18 | 1 | 5.0 | 0.0 | |
| 1966 | ABV82595 34 | 1 | 6.0 | 0.0 | |
| 1967 | ABV82584 32 | 1 | 5.7 | 0.0 | |
| 1970 | ABR28724 36 | 1 | 6.4 | 0.0 | |
| 1971 | ABR28702 36 | 1 | 6.4 | 0.0 | |
| 1972 | ABF21276 48 ABF21278 48 ABF21277 48 ABF21274 48 | 5 | 8.2 | 0.0 | |
| | ABF21272 48 | | | | |
| 1974 | CAA40728 18 | 1 | 5.0 | 0.0 | |
| 1975 | ABU80188 41 ABR28680 39 ABR28603 39 | 3 | 7.0 | 0.2 | low p > .50, |
| | | | | | prev p < .002 |
| 1976 | ABS18465 32 AAF99717 24 AAF99716 30 AAF99715 27 | 28 | 6.0 | 1.8 | low p < .005, |
| | AAF99714 30 ABQ45533 41 ABW36366 42 ABW36322 41 | | | | prev p < .005 |
| | ABV45838 43 ABR28625 41 ABR28614 43 ABR15819 39 | | | | |
| | ABQ45458 42 ABQ45447 41 ABQ45436 41 ABQ45425 42 | | | | |
| | ABQ45414 42 ABQ44394 40 P26562 31 P03455 40 AAB52910 | | | | |
| | 16 AAD25304 31 AAB50962 18 AAB50961 16 AAB39851 40 | | | | |
| | BAA01280 31 Q76WJ1 6 Q9IGQ0 4 | | | | |
| 1977 | ABD95350 37 ABD60944 37 ABD60933 37 ABW71492 38 | 42 | 6.4 | 1.2 | low p < .005, |
| | ABU80410 41 ABW36410 38 ABW36399 38 ABW36388 38 | | | | prev p < .20 |
| | ABW36377 38 ABV29524 41 ABU80287 38 ABU80254 41 | | | | |
| | ABU80243 41 ABU80232 41 ABU80221 41 ABU80210 38 | | | | |
| | ABU80199 41 ABD95712 37 ABS49921 41 ABR28647 41 | | | | |
| | ABR28581 41 ABR28570 41 ABR28559 41 ABR28548 41 | | | | |
| | ABR28537 43 ABR15874 41 ABR15863 41 ABR15852 41 | | | | |
| | ABR15841 41 ABR15830 41 ABO44134 37 ABB19667 31 | | | | |
| | ABB19529 31 ABB19518 31 P03453 37 AAD25308 30 | | | | |
| | AAA43240 16 AAA43206 27 AAB52908 15 BAF03627 29 | | | | |
| | P35938 3 P03469 8 | | | | |
| 1978 | ABW86585 41 ABW86574 41 ABW71503 41 ABU80265 39 | 21 | 6.4 | 0.8 | low p < .005, |
| | ABR28691 39 ABP49448 37 ABP49338 37 ABO38065 37 | | | | prev p > .50 |
| | ABO32992 37 ABO32981 39 ABN59423 37 ABK79948 37 | | | | |
| | ABG26813 37 ABF47737 37 ABF47726 37 ABF47715 37 | | | | |
| | ABF47704 37 ABF47693 37 AAA74287 16 AAA65552 16 | | | | |
| | AAA65548 16 | | | | |
| 1979 | ABS18464 32 AAA43172 37 ABW36311 37 ABR28636 45 | 11 | 5.8 | 1.6 | low p < .02, |
| | ABQ01322 38 ABN50756 37 ABB19551 30 ABB19540 31 | | | | prev p < .20 |
| | P18875 37 CAA86563 15 P31348 8 | | | | |
| 1980 | AAA16879 31 AAA16880 29 ABS18466 41 AAB50965 16 | 19 | 6.5 | 1.0 | low p < .05, |
| | ABU80276 44 ABS49954 44 ABR28757 44 ABR28746 44 | | | | prev p < .20 |
| | ABR28735 44 ABR28713 39 ABO38362 38 ABO33006 37 | | | | |
| | ABI84478 32 ABF47748 38 Q9WCE3 32 AAB52909 17 | | | | |
| | AAD25309 35 AAD25307 32 CAA40731 20 | | | | |
| 1981 | AAB50964 16 BAA02766 5 AAZ15840 15 AAZ15839 15 | 33 | 5.9 | 1.0 | low p < .001, |
| | ABW36355 41 ABW36344 41 ABW36333 41 ABS49932 41 | | | | prev p < .05 |
| | ABR28669 41 ABR28658 41 ABO52258 38 ABI84617 31 | | | | |
| | ABB21772 31 BAA02767 5 BAA02765 7 Q9WCE1 31 | | | | |
| | AAB52906 22 AAD25301 44 AAD25305 31 CAA86562 14 | | | | |
| | CAA82950 38 AAK51352 20 AAK51351 20 AAK51350 20 | | | | |
| | AAK51349 20 AAK51348 20 AAK51347 20 AAK51346 20 | | | | |
| | AAK51345 20 AAK51344 20 AAK51343 20 AAK51342 20 | | | | |
| | AAK51341 20 | | | | |
| 1982 | P26142 3 ABD95339 38 ABD77818 38 AAA16905 3 | 8 | 5.2 | 2.2 | low p < .05, |
| | ABO52797 42 P10757 44 AAA65553 17 CAC86623 11 | | | | prev p < .30 |
| 1983 | ABG66977 17 ABG66976 17 ABG66975 20 ABG66974 20 | 56 | 6.9 | 0.9 | low p > .50, |
| | ABG66973 20 ABO38340 41 ABO37988 41 ABO33025 42 | | | | prev p < .02 |
| | ABN50917 41 ABN50900 38 ABM66886 41 ABM66908 41 | | | | |
| | ABM66897 41 ABM22235 41 ABM22224 41 ABM22213 41 | | | | |
| | ABM22202 41 ABM22191 41 ABM22180 41 ABM22169 41 | | | | |
| | ABM22158 41 ABL67264 41 ABL67253 41 ABK80047 41 | | | | |
| | ABK80036 41 ABK80025 41 ABK40601 41 ABK40590 41 | | | | |
| | ABK40579 41 ABK40568 41 ABK40557 41 ABK40546 41 | | | | |
| | ABK40534 41 ABK40510 41 ABI92302 38 ABI30378 41 | | | | |
| | ABI20859 41 ABG88344 38 ABG88333 38 ABF47825 41 | | | | |
| | ABF47770 41 ABG79952 41 ABF47847 41 ABF47836 41 | | | | |
| | ABF47759 41 ABF47792 41 ABG26835 41 ABG26824 41 | | | | |
| | ABF47814 41 ABF47803 41 ABF47781 41 BAF63173 20 | | | | |
| | ABW91185 41 AAD25311 34 CAA35094 39 P11485 8 | | | | |
| 1984 | AAA43171 36 AAZ15838 15 ABP49349 39 ABO38406 42 | 9 | 6.0 | 1.0 | low p < .02, |
| | P18876 36 AAB27052 21 AAA65557 21 AAA65556 20 | | | | prev p < .01 |
| | AAA65555 20 | | | | |
| 1985 | CAA91080 27 AAB50966 13 ABW86596 41 ABR29615 41 | 10 | 5.3 | 1.7 | low p < .01, |
| | ABR29605 41 Q9WCE8 32 AAB52907 15 AAD25306 30 | | | | prev p < .30 |
| | AAD25312 32 P31349 8 | | | | |
| 1986 | AAA43236 17 BAA00309 18 BAA00308 17 ABP49360 34 | 13 | 5.5 | 0.7 | low p < .001, |
| | ABO44123 37 ABO38395 37 ABM22246 34 AAC57166 36 | | | | prev p > .50 |
| | BAF63172 16 P12590 17 AAA65547 17 BAA00722 17 | | | | |
| | CAA35097 17 | | | | |
| 1987 | BAA96118 17 AAZ15842 16 ABU80420 41 ABS50111 41 | 17 | 5.9 | 1.0 | low p < .001, |
| | ABR29575 41 ABV29590 33 ABS49943 33 ABQ44416 34 | | | | prev p < .20 |
| | ABG88212 29 ABN50940 34 ABN50928 34 AAD25310 33 | | | | |
| | AAA43680 17 AAA65550 17 AAA65549 17 CAA35095 44 | | | | |
| | P05778 4 | | | | |
| 1988 | CAA91081 38 AAA43238 17 AAA43233 17 AAA43231 17 | 23 | 5.4 | 1.2 | low p < .001, |
| | AAA43170 16 AAA43169 17 AAA43166 17 AAA43161 18 | | | | prev p < .20 |
| | AAA43157 14 ABU80400 40 ABS50121 40 ABR29595 42 | | | | |
| | ABR29585 42 ABB19607 30 P26140 39 AAB52904 18 | | | | |
| | CAA42444 14 ABF71860 39 AAA74300 14 AAA74299 17 | | | | |
| | AAA74298 14 AAA74285 14 AAA65551 17 | | | | |
| 1989 | AAA43168 18 AAA43158 14 AAA58800 21 AAA58801 21 | 15 | 4.0 | 2.3 | low p < .001, |
| | BAA06719 14 BAA96119 14 AAZ15841 20 BAA02768 31 | | | | prev p < .02 |
| | BAA02769 33 BAF63171 13 AAA74286 14 ABG57284 2 | | | | |
| | ABG57283 2 ABG57282 2 ABG57281 2 | | | | |
| 1990 | AAA43235 14 AAA43234 14 AAA43232 14 AAA43190 14 | 23 | 3.9 | 1.2 | low p < .001, |
| | AAA43173 14 AAA43153 14 AAA91616 27 AAZ15844 16 | | | | prev p > .50 |
| | AAZ15843 8 ABG88201 34 AAB57740 16 AAA16778 14 | | | | |
| | AAA16779 14 AAA16815 14 AAA16814 14 AAA16813 14 | | | | |
| | AAA16812 13 AAA16811 14 AAA16810 14 AAA16809 14 | | | | |
| | AAA16808 15 ABG66980 2 ABG66979 2 | | | | |
| 1991 | AAA43225 14 CAA91082 38 AAP34322 36 AAA43167 14 | 36 | 4.8 | 1.8 | low p < .001, |
| | AAA43283 42 BAA96120 14 ABW71521 42 ABR29565 42 | | | | prev p < .02 |
| | ABQ10099 16 ABD60955 33 AAA19934 37 S69887 26 S69888 | | | | |
| | 36 S69889 42 AAB50963 42 CAA91083 34 CAA86560 39 | | | | |
| | CAA86567 17 AAA43142 14 AAA74297 14 AAA74296 14 | | | | |
| | AAA74295 14 AAA74294 14 AAA74293 14 AAA74292 17 | | | | |
| | AAA74291 14 AAA74290 14 AAA74289 14 AAA74288 14 | | | | |
| | AAA65546 14 AAA65545 14 AAA65544 14 CAD29945 19 | | | | |
| | ABG66981 2 ABG66978 2 ABE73717 2 | | | | |
| 1992 | AAB29091 41 BAA05874 14 BAA96121 14 ABB19618 30 | 11 | 5.2 | 2.0 | low p < .01, |
| | AAU09400 12 AAC57167 38 HMIV17 39 CAA86561 39 | | | | prev p > .50 |
| | AAC14275 14 AAA51481 5 AAA72339 41 | | | | |
| 1993 | ABM21960 34 ABI92181 30 AAC57169 35 AAC57168 36 | 8 | 5.9 | 1.0 | low p < .01, |
| | ABO52170 39 AAB50960 31 AAB50958 17 AAB50957 17 | | | | prev p < .30 |
| 1994 | ABS70427 30 AAB03292 42 AAB03291 42 AAB50959 31 | 5 | 6.3 | 1.5 | low p < .30, |
| | CAD29938 14 | | | | prev p > .50 |
| 1995 | AAK70450 37 AAK70449 37 AAP34325 38 AAP34323 20 | 47 | 6.1 | 1.7 | low p < .001, |
| | BAC82887 19 BAC82881 19 BAA96122 19 AAZ17358 16 | | | | prev p > .50 |
| | ABQ10100 19 ABP51995 1 ABG88322 36 ABG26791 37 | | | | |
| | ABF47638 36 ABJ53438 37 ABI92313 40 ABI30367 36 | | | | |
| | ABI20870 31 ABI20837 36 ABG88311 36 ABG88300 36 | | | | |
| | ABF47627 37 ABG47840 36 ABG26780 36 ABF47605 37 | | | | |
| | ABE26991 36 ABE12032 37 ABE11942 36 ABE11922 36 | | | | |
| | ABE11900 36 ABE11889 36 ABE11878 36 ABE11867 37 | | | | |
| | AAL60449 36 AAL60444 36 AAL60443 36 AAK67336 17 | | | | |
| | AAK67335 17 AAK67332 17 AAK67331 17 AAK67330 17 | | | | |
| | ABS70438 30 CAC86625 18 CAC86619 14 CAC86617 18 | | | | |
| | CAD29944 19 CAD29937 1 CAD29936 14 | | | | |
| 1996 | BAC82896 19 BAC82893 19 BAC82884 19 BAA96124 19 | 87 | 7.1 | 1.9 | low p < .40, |
| | BAA96123 19 AAK73345 16 AAK73344 16 AAK73343 16 | | | | prev p < .002 |
| | AAK73342 16 AAK73341 16 AAK73340 16 AAK73339 16 | | | | |
| | AAK73338 16 AAK73337 16 AAK73336 16 AAK73335 16 | | | | |
| | AAK73334 16 AAK73333 16 AAK73332 16 AAK73331 16 | | | | |
| | AAK73330 16 AAK73329 16 AAK73328 16 AAK73327 16 | | | | |
| | AAK73326 12 AAK73325 16 AAK73324 16 AAK73323 16 | | | | |
| | AAK73322 16 AAK73321 16 AAK73320 16 ABO52225 37 | | | | |
| | ABO38010 37 ABN51066 39 ABN50973 37 ABN50962 37 | | | | |
| | ABN50951 36 ABF47649 36 ABM22290 36 ABM22279 37 | | | | |
| | ABM22268 36 ABM22257 36 ABB19571 30 ABJ53504 31 | | | | |
| | ABJ53493 31 ABI95283 31 ABI95272 31 ABI95261 31 | | | | |
| | ABI95250 31 ABI93028 31 ABI21574 31 ABI21563 31 | | | | |
| | ABI21552 31 ABI21541 31 ABI21530 29 ABI21519 31 | | | | |
| | ABI20848 39 ABG47829 36 ABF47660 37 AAK67328 17 | | | | |
| | AAK67327 17 AAK67326 17 AAK67325 17 AAK67324 17 | | | | |
| | AAK67323 22 AAK67322 17 AAP60039 37 AAP60038 39 | | | | |
| | AAP60037 42 AAP60036 37 AAF06947 16 AAF06946 13 | | | | |
| | AAF06945 13 CAC86611 17 AAB81463 23 AAB81462 19 | | | | |
| | AAB81461 19 AAB81460 19 AAB81459 23 AAB81458 19 | | | | |
| | AAB81457 19 AAB81456 19 CAC86616 38 BAF03629 30 | | | | |
| | ABD59847 36 CAD29943 18 CAD29933 14 | | | | |
| 1997 | AAD17229 29 BAA96125 14 ABG26246 19 ABG26245 19 | 28 | 5.7 | 2.0 | low p < .001, |
| | ABG26244 19 ABG26243 19 ABG26242 19 AAQ10369 19 | | | | prev p < .001 |
| | AAQ10368 16 AAQ10367 19 AAK67337 12 AAF87281 48 | | | | |
| | AAF87280 48 AAF87279 42 AAF87278 42 AAF87277 42 | | | | |
| | AAF87276 42 AAF87275 39 AAF87274 40 AAP79975 1 | | | | |
| | AAP79973 1 CAC86608 42 CAC86606 33 ABD59848 34 | | | | |
| | CAD29934 14 CAD29932 14 CAD29928 14 CAC86615 17 | | | | |
| 1998 | AAK70464 22 AAK70459 7 AAK70458 18 AAK70457 31 | 38 | 5.1 | 1.2 | low p < .001, |
| | AAK70456 31 AAK70455 14 AAK70454 29 AAK70453 29 | | | | prev p < .10 |
| | AAK70452 29 AAK70451 31 AAD17218 21 BAC82898 14 | | | | |
| | BAC82877 14 BAC82871 17 BAA96131 14 BAA96126 14 | | | | |
| | AAD17219 21 ABQ10144 13 ABQ10143 18 ABQ10087 16 | | | | |
| | ABB19574 31 AAK67319 12 AAF87284 45 AAF87283 41 | | | | |
| | AAF87282 42 AAT00438 23 CAB42465 30 AAT65329 30 | | | | |
| | AAO88265 20 CAC86609 6 CAC86624 28 CAD29935 17 | | | | |
| | CAD29931 14 CAD29929 13 CAD29927 14 CAD29922 14 | | | | |
| | CAC86335 17 CAC86620 32 | | | | |
| 1999 | AAP34324 31 ABV25643 49 ABV25640 49 ABV25638 49 | 65 | 5.3 | 2.0 | low p < .001, |
| | ABV25637 49 ABV25636 49 ABV25635 49 ABV25634 49 | | | | prev p < .40 |
| | ABQ10137 15 ABL67055 30 ABL67066 30 ABJ53427 30 | | | | |
| | ABG88256 30 BAC82894 17 BAC82892 14 BAC82885 9 | | | | |
| | BAC82883 14 BAC82876 17 BAC82875 14 BAC82873 20 | | | | |
| | BAC82872 14 BAA96128 14 BAA96127 14 ABO21723 19 | | | | |
| | ABK40006 31 ABJ16609 31 AAQ10385 19 AAQ10380 19 | | | | |
| | AAQ10373 19 AAQ10372 19 AAK67343 12 AAK67342 12 | | | | |
| | AAK67341 12 AAK67340 12 AAK67339 12 AAK67334 17 | | | | |
| | AAK67333 12 AAK67329 12 BAF63169 13 BAF63168 13 | | | | |
| | BAF63167 13 BAF63166 13 AAF80098 14 AAF80099 14 | | | | |
| | CAC86337 51 CAC86336 19 CAC86610 51 CAC86622 31 | | | | |
| | CAC86605 19 ABD59849 28 CAD29942 19 CAD29921 14 | | | | |
| | CAD29917 14 CAD29916 14 ABV25653 9 ABV25650 9 | | | | |
| | ABV25648 9 ABV25647 9 ABV25646 9 ABV25645 9 | | | | |
| | ABV25644 9 BAF63165 13 CAC86334 17 CAC86626 14 | | | | |
| | CAC86621 23 | | | | |
| 2000 | ABC66246 1 ABC66232 13 AAY42122 15 AAY42121 15 | 147 | 5.0 | 1.6 | low p < .001, |
| | AAY42120 15 AAY42119 15 AAY42118 15 AAY42117 15 | | | | prev p < .10 |
| | AAY42116 15 AAY42115 15 AAY42114 15 BAC82897 19 | | | | |
| | BAC82895 14 BAC82891 13 BAC82890 14 BAC82889 14 | | | | |
| | BAC82888 13 BAC82886 14 BAC82882 13 BAC82880 14 | | | | |
| | BAC82879 14 BAC82878 14 BAC82874 19 BAC82870 14 | | | | |
| | BAC82865 14 AAN83988 20 BAA96130 19 BAA96129 14 | | | | |
| | AAK40315 14 AAK40318 14 AAK40316 14 AAK40317 14 | | | | |
| | AAK40314 14 AAK40313 14 ABV45849 31 ABU80309 31 | | | | |
| | ABU80298 32 ABS49987 31 ABS49976 31 ABR28801 31 | | | | |
| | ABR28779 31 ABR28768 31 ABR14657 1 ABR14641 1 | | | | |
| | ABR14640 14 ABR14639 14 ABR15918 31 ABR15907 31 | | | | |
| | ABR15896 18 ABR15885 31 ABQ10097 20 ABQ10095 13 | | | | |
| | ABP49382 37 ABP49305 31 ABP49217 31 ABO44046 31 | | | | |
| | ABO21725 19 ABO21724 21 ABO21716 19 ABM22026 39 | | | | |
| | ABL67209 37 ABL67187 37 ABK79970 37 ABK40050 37 | | | | |
| | ABK40039 37 ABK40028 37 AAX56530 41 ABJ53515 37 | | | | |
| | ABJ53449 31 ABJ16730 37 ABJ16719 37 ABJ16642 37 | | | | |
| | ABJ09327 18 ABI95294 18 ABI95217 31 ABG88553 31 | | | | |
| | ABG88542 31 ABG80183 31 ABG80172 31 ABG67477 31 | | | | |
| | ABG48049 31 ABG37362 31 ABF47891 31 ABF47880 31 | | | | |
| | ABF47869 31 ABG72870 1 ABG72869 1 ABG47818 31 | | | | |
| | ABG47807 31 ABE11668 18 ABE11657 31 ABD95031 18 | | | | |
| | ABD95020 31 ABD95009 31 ABD94998 31 ABD94987 31 | | | | |
| | ABD94976 18 ABD94965 31 ABD94756 31 ABD78038 31 | | | | |
| | ABD78027 31 ABD78016 31 ABD78005 31 ABD77994 31 | | | | |
| | ABD77983 31 ABD77972 31 ABD77961 31 ABD77950 31 | | | | |
| | ABD77939 31 ABD77928 33 ABD77917 31 ABD77730 31 | | | | |
| | ABD77719 31 ABD77708 31 ABD63063 31 ABD61540 31 | | | | |
| | ABD61518 31 ABD60900 31 ABD60889 31 ABD60878 31 | | | | |
| | ABD60867 31 ABD60856 31 ABA08497 18 ABA08486 18 | | | | |
| | AAQ10391 2 AAK67344 12 AAK67338 12 AAK67321 12 | | | | |
| | AAK67320 12 AAL15459 27 CAC86333 28 CAC86607 28 | | | | |
| | CAC86612 28 CAD29941 19 CAD29940 19 CAD29939 24 | | | | |
| | CAD29930 1 CAD29926 1 CAD29924 1 CAD29920 16 | | | | |
| | CAD29919 14 CAD29899 14 CAD57622 6 CAC86618 31 | | | | |
| | CAC86614 17 CAC86613 17 CAC18525 12 | | | | |
| 2001 | AAP79964 12 ABC66233 14 BAC82869 1 BAC82868 1 | 193 | 3.2 | 1.6 | low p < .001, |
| | BAC82867 1 BAC82866 1 BAC82864 1 BAC82863 1 | | | | prev p < .001 |
| | BAC82862 14 BAC82861 1 BAC82860 13 BAC82859 14 | | | | |
| | BAC82858 14 BAC82857 14 BAC82846 1 BAC82843 1 | | | | |
| | ABI55088 1 AAZ17359 25 AAY56898 32 ABR28845 18 | | | | |
| | ABR28834 18 ABR14668 14 ABR14667 14 ABR14666 14 | | | | |
| | ABR14665 14 ABR14664 14 ABR14663 13 ABR14662 14 | | | | |
| | ABR14661 14 ABR14660 14 ABR14659 14 ABR14658 14 | | | | |
| | ABR14656 1 ABR14655 1 ABR14654 1 ABR14653 1 | | | | |
| | ABR14652 1 ABR14651 1 ABR14650 1 ABR14649 14 | | | | |
| | ABR14648 14 ABR14647 14 ABR14646 14 ABR14645 14 | | | | |
| | ABR14644 14 ABR14643 14 ABR14642 14 BAF63035 13 | | | | |
| | BAF63032 13 ABQ10092 1 ABQ10091 1 ABQ10090 1 | | | | |
| | ABQ10089 14 ABO38329 18 ABO38318 18 ABO38043 18 | | | | |
| | ABO38032 31 ABO38021 31 ABO32959 18 ABO32948 18 | | | | |
| | ABN51143 18 ABN51077 18 ABM66864 18 ABJ09151 19 | | | | |
| | ABG67491 18 ABG37395 18 ABG37384 18 ABG26945 18 | | | | |
| | ABF82940 18 ABF82929 18 ABF82918 18 ABF82907 18 | | | | |
| | ABF82896 18 ABF82885 18 ABF82874 18 ABF82863 18 | | | | |
| | ABF82852 18 ABF82841 18 ABF82830 18 ABF82819 31 | | | | |
| | ABG72867 1 ABG72866 1 ABF47671 18 ABF47572 18 | | | | |
| | ABF47561 29 ABG37120 18 ABF82684 18 ABF82673 18 | | | | |
| | ABF82662 18 ABF47583 18 ABE12248 18 ABE11856 18 | | | | |
| | ABE11845 18 ABE11834 18 ABE11823 18 ABE11812 18 | | | | |
| | ABE11734 18 ABE11723 18 ABE11712 18 ABE11701 18 | | | | |
| | ABE11690 18 ABE11679 26 ABD95328 18 ABD95317 18 | | | | |
| | ABD95306 18 ABD95295 18 ABD95284 18 ABD95273 18 | | | | |
| | ABD95262 31 ABD95251 18 ABD95240 18 ABD95229 18 | | | | |
| | ABD95218 18 ABD95207 31 ABD95196 18 ABD95185 18 | | | | |
| | ABD95174 18 ABD95163 18 ABD95152 18 ABD95141 18 | | | | |
| | ABD95130 18 ABD95119 18 ABD95108 18 ABD95097 18 | | | | |
| | ABD95086 18 ABD95075 18 ABD95064 18 ABD95053 18 | | | | |
| | ABD95042 18 ABD94811 18 ABD94800 18 ABD94789 18 | | | | |
| | ABD94778 18 ABD78093 18 ABD78082 18 ABD78071 18 | | | | |
| | ABD78060 18 ABD62061 31 ABD60911 31 ABC86237 36 | | | | |
| | ABC40533 18 ABB02814 18 ABA87231 18 ABA87091 18 | | | | |
| | ABC02277 18 ABB82194 18 ABB80045 18 ABB79990 18 | | | | |
| | ABB79979 18 ABB53707 18 ABB02936 18 ABB02924 31 | | | | |
| | ABB02913 18 ABB02825 18 ABA87045 18 ABA43189 18 | | | | |
| | ABA42575 18 ABA42324 31 ABA42258 18 ABA42236 18 | | | | |
| | ABA18037 18 ABA12715 18 ABA08519 31 ABA08464 18 | | | | |
| | AAZ85126 18 AAZ83299 18 AAZ79604 18 AAZ38627 18 | | | | |
| | AAT85679 18 AAU25871 12 AAU25861 12 AAL47667 14 | | | | |
| | AAL47668 19 CAD29925 1 CAD29923 1 CAD29918 14 | | | | |
| | CAD29911 1 CAD29910 1 CAD29909 14 CAD29908 14 | | | | |
| | CAD29907 1 CAD29906 1 CAD29905 19 CAD29904 1 | | | | |
| | CAD29903 14 CAD29902 19 CAD29901 14 CAD29900 1 | | | | |
| | CAD57623 33 CAD57621 6 CAD57620 27 CAD57619 28 | | | | |
| | CAD57617 1 | | | | |
| 2002 | AAU25851 29 BAC82856 1 BAC82855 1 BAC82854 1 | 62 | 2.6 | 2.5 | low p < .001, |
| | BAC82853 1 BAC82852 1 BAC82851 1 BAC82850 1 | | | | prev p < .05 |
| | BAC82849 1 BAC82848 1 BAC82847 1 BAC82845 1 | | | | |
| | BAC82844 1 BAC82842 19 AAP69688 1 AAP69687 1 | | | | |
| | AAP69686 1 AAP69685 1 AAP69684 1 AAP69683 1 | | | | |
| | AAP69682 1 AAP69681 1 AAP69680 12 AAP69679 1 | | | | |
| | AAP69678 3 AAP69677 1 AAP69676 1 AAP69675 1 | | | | |
| | AAP69674 1 AAP69673 1 AAP69692 1 AAP69691 1 | | | | |
| | AAP69690 1 AAP69689 1 ABS76427 18 BAF63057 12 | | | | |
| | BAF63054 12 BAF63050 12 BAF63046 17 BAF63043 13 | | | | |
| | BAF63039 13 ABB19628 31 ABG72868 1 ABA87080 31 | | | | |
| | ABB82216 31 ABB51962 17 AAZ83253 28 AAT12706 29 | | | | |
| | ABS70411 30 ABS70400 30 ABS70389 30 ABS70378 30 | | | | |
| | ABS70367 30 ABS70337 30 ABS70326 30 ABS70316 30 | | | | |
| | ABS70305 30 CAD29958 14 CAD29914 14 CAD57618 28 | | | | |
| | CAD57616 14 CAD35678 19 | | | | |
| 2003 | ABB86907 43 ABB86887 37 ABB86877 37 BAF63102 13 | 94 | 4.9 | 1.1 | low p< .001, |
| | BAF63101 13 BAF63097 13 BAF63096 13 BAF63095 13 | | | | prev p < .001 |
| | BAF63091 13 BAF63090 13 BAF63089 13 BAF63085 13 | | | | |
| | BAF63084 13 BAF63083 13 BAF63082 13 BAF63078 13 | | | | |
| | BAF63075 13 BAF63072 13 BAF63068 13 BAF63064 13 | | | | |
| | BAF63061 13 ABQ10105 16 ABQ10104 14 ABQ10103 14 | | | | |
| | ABQ10102 14 ABO37999 31 ABN51088 31 ABM67051 31 | | | | |
| | ABI96088 14 ABI96127 14 ABI96126 14 ABI96125 14 | | | | |
| | ABI96124 11 ABI96123 14 ABI96122 14 ABI96121 14 | | | | |
| | ABI96120 14 ABI96119 16 ABI96118 17 ABI96117 14 | | | | |
| | ABI96116 14 ABI96115 14 ABI96114 14 ABI96113 14 | | | | |
| | ABI96112 11 ABI96111 14 ABI96110 14 ABI96109 31 | | | | |
| | ABI96108 30 ABI96107 31 ABI96105 14 ABI96103 31 | | | | |
| | ABI96100 14 ABI96098 14 ABI96097 14 ABI96096 14 | | | | |
| | ABI96095 14 ABI96094 14 ABI96093 14 ABI96090 14 | | | | |
| | ABE12634 57 ABD78104 51 ABD60779 28 ABD15515 28 | | | | |
| | ABC41714 31 ABB03123 31 ABA87057 31 AAZ83977 31 | | | | |
| | ABB82205 36 ABB80103 31 ABB53740 31 ABB03145 31 | | | | |
| | ABB02803 31 ABB02792 28 ABA42247 36 ABA18145 31 | | | | |
| | ABA12729 28 ABA12707 31 ABA12696 31 ABA08475 36 | | | | |
| | ABK39995 31 ABB86917 31 1RUZ_M 19 1RUZ_L 19 | | | | |
| | 1RUZ_K 19 1RUZ_J 19 1RUZ_I 19 1RUZ_H 19 1RUY_M 15 | | | | |
| | 1RUY_L 15 1RUY_K 15 1RUY_J 15 IRUY_I 15 1RUY_H 15 | | | | |
| 2004 | ABB86946 40 ABB86937 44 ABS00326 34 ABQ10005 26 | 36 | 5.3 | 1.4 | low p < .001, |
| | ABQ10004 20 ABQ10003 14 ABQ10002 14 ABQ10001 26 | | | | prev p < .10 |
| | ABQ09988 14 ABQ09838 22 ABQ09837 28 ABQ09784 14 | | | | |
| | ABQ09783 29 ABQ09782 29 ABQ09780 27 ABQ09779 29 | | | | |
| | ABI96132 14 ABI96130 14 ABI96129 14 ABI54447 13 | | | | |
| | ABI54446 13 ABI54445 13 ABI54444 13 ABI54443 13 | | | | |
| | ABI54442 13 ABI54441 13 ABI54440 13 ABI54439 13 | | | | |
| | ABI54438 13 ABI54437 13 ABE27153 46 ABC42750 46 | | | | |
| | ABB86929 31 ABB86899 31 AAV68006 15 AAV67984 13 | | | | |
| 2005 | ABJ51893 13 ABI51313 13 ABO52104 30 ABC66245 14 | 82 | 4.9 | 1.5 | low p < .001, |
| | ABC66244 14 ABC66243 14 ABC66242 14 ABC66241 14 | | | | prev p < .10 |
| | ABC66240 14 ABC66239 14 ABC66238 14 ABC66237 14 | | | | |
| | ABC66236 14 ABC66235 14 ABC66234 14 ABW75642 18 | | | | |
| | ABI19015 41 ABK57093 35 ABR28900 31 ABJ51895 13 | | | | |
| | ABJ51894 13 ABJ51892 13 ABJ51891 13 ABJ51890 13 | | | | |
| | BAF63115 13 BAF63111 13 BAF63107 13 BAF63103 13 | | | | |
| | ABQ09953 14 ABQ09950 27 ABQ09949 14 ABQ09948 13 | | | | |
| | ABQ09947 14 ABQ09915 14 ABQ09914 14 ABQ09913 14 | | | | |
| | ABQ09904 14 ABQ09874 14 ABQ09873 14 ABQ09872 14 | | | | |
| | ABQ09871 16 ABP51970 14 ABP49393 31 ABO32970 31 | | | | |
| | ABO32678 37 ABO21731 14 ABO21730 14 ABK40689 31 | | | | |
| | ABJ16686 31 ABJ16675 31 ABJ16664 31 ABJ16653 31 | | | | |
| | ABJ09184 31 ABI96148 14 ABI96147 32 ABI96146 14 | | | | |
| | ABI96145 27 ABI96144 13 ABI96143 29 ABI96142 14 | | | | |
| | ABI96141 14 ABI96140 14 ABI96139 14 ABI96138 14 | | | | |
| | ABI96137 14 ABI96135 14 ABI96134 14 ABI96128 16 | | | | |
| | ABI92379 31 ABI30565 31 ABI22109 31 ABI21233 31 | | | | |
| | ABI21222 31 ABI21211 31 ABI21200 31 ABI21189 31 | | | | |
| | ABG72865 1 ABG72864 1 ABG72863 1 BAE53730 14 | | | | |
| | BAE53729 14 ABB84190 14 | | | | |
| 2006 | BAE96542 25 BAE96541 14 BAE96540 14 BAE96539 25 | 135 | 5.2 | 1.7 | low p < .001, |
| | BAE96538 14 BAE96537 14 BAE96536 25 BAE96535 14 | | | | prev p < .20 |
| | BAE96534 14 BAE96533 14 ABW71294 31 ABW23335 40 | | | | |
| | ABW23328 31 ABW23327 14 ABW23324 27 ABW23321 14 | | | | |
| | ABW23320 31 ABW23318 14 ABW23317 27 ABW23314 30 | | | | |
| | ABW23311 14 ABW23310 14 ABW23308 13 ABW23307 14 | | | | |
| | ABW23306 14 ABW23303 14 ABW23300 14 ABW23296 31 | | | | |
| | ABW23295 31 ABW23294 29 ABW23291 14 ABW23290 31 | | | | |
| | ABW23289 17 ABW23287 14 ABW23286 31 ABW23285 13 | | | | |
| | ABW23284 14 ABW23283 1 ABW23280 14 ABW23279 14 | | | | |
| | ABW23275 14 ABV45654 31 ABV29557 31 ABV29546 31 | | | | |
| | ABV29535 18 ABU99109 44 ABU99069 25 ABU99067 27 | | | | |
| | ABU50589 44 ABU50588 44 ABU50587 44 ABU50586 42 | | | | |
| | ABU50574 18 ABU50573 31 ABU50571 31 ABU50570 31 | | | | |
| | ABU50569 31 ABU50568 31 ABU50567 31 ABU50566 18 | | | | |
| | ABU50565 31 ABU50556 11 ABU50555 26 ABU50554 14 | | | | |
| | ABU50553 14 ABU50552 26 ABU50551 23 ABU50550 23 | | | | |
| | ABU50549 14 ABU50546 17 ABU50545 28 ABU50544 13 | | | | |
| | ABU50543 14 ABU50542 1 ABU50541 13 ABU50540 1 | | | | |
| | ABU50539 14 ABU50538 23 ABU50537 14 ABU50536 1 | | | | |
| | ABU50535 14 ABU50534 31 ABU50518 27 ABU50517 23 | | | | |
| | ABS00315 31 BAF63135 14 BAF63131 14 BAF63127 25 | | | | |
| | BAF63123 18 BAF63119 14 ABQ09984 25 ABQ09981 25 | | | | |
| | ABQ09977 31 ABQ09976 25 ABQ09975 25 ABQ09974 27 | | | | |
| | ABQ09973 25 ABQ09972 25 ABQ09969 27 ABQ09968 25 | | | | |
| | ABQ09961 29 ABQ09960 29 ABQ09958 29 ABQ09957 26 | | | | |
| | ABQ09956 25 ABQ09955 19 ABK79959 31 ABI96174 27 | | | | |
| | ABI96173 14 ABI96172 14 ABI96171 14 ABI96170 14 | | | | |
| | ABI96169 14 ABI96168 14 ABI96167 14 ABI96166 25 | | | | |
| | ABI96165 14 ABI96164 14 ABI96163 14 ABI96162 19 | | | | |
| | ABI96161 14 ABI96160 14 ABI96159 14 ABI96158 14 | | | | |
| | ABI96157 14 ABI96156 14 ABI96155 14 ABI96154 14 | | | | |
| | ABI96153 27 ABI96152 27 ABI96151 14 ABI96150 14 | | | | |
| | ABI96149 27 ABH07371 29 ABH07372 29 | | | | |
| 2007 | ABS71673 46 ABS71672 46 ABS71671 46 ABS71670 46 | 285 | 5.5 | 1.2 | low p < .001, |
| | ABS71669 46 ABS71668 46 ABS71667 46 ABS71666 46 | | | | prev p < .02 |
| | ABS71665 46 ABS71664 46 ABQ52695 35 ABW34451 23 | | | | |
| | ABW86606 31 ABW86552 31 ABW86541 31 ABW86530 31 | | | | |
| | ABW86519 31 ABW86508 31 ABW86497 18 ABW86486 31 | | | | |
| | ABW86475 31 ABW86464 31 ABW86453 31 ABW86442 31 | | | | |
| | ABW86431 31 ABW86420 31 ABW86409 31 ABW86398 18 | | | | |
| | ABW86387 31 ABW86376 31 ABW86365 31 ABW86354 31 | | | | |
| | ABW86343 31 ABW86332 30 ABW86321 18 ABW71470 31 | | | | |
| | ABW71459 31 ABW71448 31 ABW71437 31 ABW71426 31 | | | | |
| | ABW71415 31 ABW71404 31 ABW71393 31 ABW71382 31 | | | | |
| | ABW71371 31 ABW71360 18 ABW71338 31 ABW71327 31 | | | | |
| | ABW71316 31 ABW71305 18 ABW40675 31 ABW40664 18 | | | | |
| | ABW40642 31 ABW40620 31 ABW40609 31 ABW40598 18 | | | | |
| | ABW40576 31 ABW40565 31 ABW40554 30 ABW40543 31 | | | | |
| | ABW40532 31 ABW40521 31 ABW40510 18 ABW40499 31 | | | | |
| | ABW40488 31 ABW40477 31 ABW40466 31 ABW40455 31 | | | | |
| | ABW40444 31 ABW40433 18 ABW40422 31 ABW40411 31 | | | | |
| | ABW40400 31 ABW40389 31 ABW40367 31 ABW40356 31 | | | | |
| | ABW40345 18 ABW40334 18 ABW40312 30 ABW40301 30 | | | | |
| | ABW40290 31 ABW40279 31 ABW40257 31 ABW40235 31 | | | | |
| | ABW40224 31 ABW40213 31 ABW40202 31 ABW40180 31 | | | | |
| | ABW40158 31 ABW40147 31 ABW40125 31 ABW40114 31 | | | | |
| | ABW40103 31 ABW40092 33 ABW40070 43 ABW40059 31 | | | | |
| | ABW40048 33 ABW40037 31 ABW40015 18 ABW40004 31 | | | | |
| | ABW39993 31 ABW39982 31 ABW39971 42 ABW39960 31 | | | | |
| | ABW39949 31 ABW39927 31 ABW39916 40 ABW39905 31 | | | | |
| | ABW39894 31 ABW39883 33 ABW39861 31 ABW39850 40 | | | | |
| | ABW39839 27 ABW39828 46 ABW39817 30 ABW39806 31 | | | | |
| | ABW39777 31 ABW36300 30 ABW36289 31 ABW36278 31 | | | | |
| | ABW36267 31 ABW36256 43 ABW36245 31 ABW36234 31 | | | | |
| | ABW36223 31 ABW36212 31 ABW36201 31 ABW36190 31 | | | | |
| | ABW36179 31 ABW23343 33 ABW23342 44 ABW23341 36 | | | | |
| | ABW23340 31 ABW23339 30 ABW23338 31 ABW23337 31 | | | | |
| | ABW23336 36 ABW23329 14 ABW23326 14 ABW23325 31 | | | | |
| | ABW23323 31 ABW23322 27 ABW23319 31 ABW23316 14 | | | | |
| | ABW23315 40 ABW23313 31 ABW23312 46 ABW23309 14 | | | | |
| | ABW23305 31 ABW23304 31 ABW23302 31 ABW23301 14 | | | | |
| | ABW23299 46 ABW23298 13 ABW23297 1 ABW23293 31 | | | | |
| | ABW23292 18 ABW23288 31 ABW23282 31 ABW23281 31 | | | | |
| | ABW23278 14 ABW23277 31 ABW23276 38 ABW23274 1 | | | | |
| | ABW23273 31 ABV82551 30 ABV45959 31 ABV45948 31 | | | | |
| | ABV45937 31 ABV45926 30 ABV45915 31 ABV45893 30 | | | | |
| | ABV45882 31 ABV45871 31 ABV30624 31 ABV30613 32 | | | | |
| | ABV30602 31 ABV30591 31 ABV30580 31 ABV30569 31 | | | | |
| | ABV30558 31 ABV30547 30 ABV30536 31 ABV30525 31 | | | | |
| | ABV30503 31 ABV30492 31 ABV30459 31 ABV30371 31 | | | | |
| | ABV30360 31 ABV30349 31 ABV30338 31 ABV30327 18 | | | | |
| | ABV30316 31 ABV30305 31 ABV30294 18 ABV30283 31 | | | | |
| | ABV30195 31 ABV30184 31 ABV30173 31 ABV30162 31 | | | | |
| | ABV30151 31 ABV30140 21 ABV30129 31 ABV30107 31 | | | | |
| | ABV30096 31 ABV30085 31 ABV30052 31 ABV30041 31 | | | | |
| | ABV30030 31 ABV30019 31 ABV30008 31 ABV29997 31 | | | | |
| | ABV29986 31 ABV29975 31 ABV29964 31 ABV29953 31 | | | | |
| | ABV29942 30 ABV29920 31 ABV29887 31 ABV29876 31 | | | | |
| | ABV29865 31 ABV29854 31 ABV29843 31 ABV29832 31 | | | | |
| | ABV29799 31 ABV29788 31 ABV29777 30 ABV29766 31 | | | | |
| | ABV29755 31 ABV29744 31 ABV29733 30 ABV29700 31 | | | | |
| | ABV29689 31 ABV29678 31 ABV29667 31 ABV29656 31 | | | | |
| | ABV29645 31 ABV29634 31 ABV29612 31 ABV29601 30 | | | | |
| | ABV29579 35 ABV29568 31 ABU50572 31 ABS71683 46 | | | | |
| | ABS71682 46 ABS71681 46 ABS71680 46 ABS71679 46 | | | | |
| | ABS71678 46 ABS71677 46 ABS71676 46 ABS71675 46 | | | | |
| | ABS71674 46 ABW91636 31 ABW91625 31 ABW91614 31 | | | | |
| | ABW91603 18 ABW91592 31 ABW91581 31 ABW91570 30 | | | | |
| | ABW91559 31 ABW91537 31 ABW91526 30 ABW91515 31 | | | | |
| | ABW91504 31 ABW91493 35 ABW91482 18 ABW91471 31 | | | | |
| | ABW91460 18 ABW91449 18 ABW91427 31 ABW91416 31 | | | | |
| | ABW91405 18 ABW91383 31 ABW91372 31 ABW91361 30 | | | | |
| | ABW91350 31 ABW91339 31 ABW91328 28 ABW91317 31 | | | | |
| | ABW91306 31 ABW91295 31 ABW91284 42 ABW91273 31 | | | | |
| | ABW91218 31 | | | | |

### Example 14

### Replikin Count Analysis in Equine Influenza Virus H3N8

Applicants analyzed publicly available sequences for isolates of EIV from PubMed using proprietary search tool software (ReplikinForecast™ available in the United States from REPLIKINS LLC, Boston, Mass.). The data is contained in Table 22, below, and Table 4, above, and graphically described in FIG. 7.

Table 22 provides the data for Replikin concentration for publicly available sequences of the pB1 gene area of the H3N8 strain of influenza virus from 1963 to 2005. Sequences were publicly available under accession numbers at www.pubmed.com. Standard deviation and significance as compared to the mean Replikin Count of the previous year and of the lowest mean Replikin Count within the data set are also provided along with the mean Replikin Count for each year. Where data was not available in a given year the year is not

TABLE 22
H3N8 pB1

| | | No. of | Mean | | |
|---|---|---|---|---|---|
| | | Isolates | Replikin | | |
| | | per | Count per | | |
| Year | PubMed Accession Number-Replikin Count | year | year | S.D. | Significance |
| 1963 | ABB88376 14 | 1 | 1.8 | 0.0 | |
| 1972 | ABI84585 14 | 1 | 1.8 | 0.0 | |
| 1977 | ABB19675 15 | 1 | 16.7 | 0.0 | |
| 1978 | ABB20350 15 ABB88317 18 | 2 | 11.0 | 12.7 | low p < 0.40, prev p > 0.50 |
| 1979 | ABB87407 20 ABB86793 20 | 2 | 22.2 | 0.0 | prev p < 0.30 |
| 1980 | ABB87797 20 ABB20412 14 BAF32965 14 Q08II5 14 | 5 | 6.7 | 8.8 | low p < 0.20, prev p < 0.01 |
| | Q08II4 5 | | | | |
| 1982 | ABI84945 16 | 1 | 17.8 | 0.0 | prev p < 0.04 |
| 1985 | ABB19731 20 ABB19720 17 | 2 | 20.6 | 2.4 | low p < 0.05, prev p < 0.30 |
| 1986 | ABO52134 17 ABJ09104 25 ABP49539 14 P16505 17 | 5 | 13.3 | 11.1 | low p < 0.05, prev p < 0.20 |
| | AAA43638 17 | | | | |
| 1987 | ABL67228 14 ABL67849 15 ABM66861 20 ABI95482 | 4 | 19.1 | 2.7 | low p < 0.001, |
| | 15 | | | | prev p < 0.30 |
| 1991 | ABM21946 15 ABI84419 15 | 2 | 9.3 | 10.4 | low p < 0.40, prev p < 0.20 |
| 1992 | ABB88191 14 | 1 | 15.6 | 0.0 | prev p > 0.50 |
| 1998 | AAT65279 6 AAT65275 6 AAT65264 6 AAT65263 6 | 6 | 2.2 | 0.0 | low p < 0.001, |
| | AAT65251 6 AAT65249 6 | | | | prev p < 0.001 |
| 1999 | AAZ23576 15 | 1 | 2.0 | 0.0 | prev p < 0.001 |
| 2001 | ABB19766 16 AAN15147 5 | 2 | 9.9 | 11.2 | low p < 0.40, prev p > 0.50 |
| 2002 | AAZ23577 15 ABI47978 14 ABO51892 14 ABO51870 | 5 | 4.6 | 6.1 | low p < 0.30, prev p > 0.50 |
| | 14 AAX23573 15 | | | | |
| 2003 | AAZ23578 15 AAZ23575 15 AAZ23574 15 | 6 | 2.0 | 0.0 | low p < 0.001, |
| | AAZ23573 15 AAZ23572 15 ABB17181 15 | | | | prev p < 0.30 |
| 2004 | AAZ23571 15 ABJ53167 5 ABD27777 6 ABD27776 6 | 5 | 2.2 | 0.2 | low p < 0.01, prev p < 0.02 |
| | ABD27775 6 | | | | |
| 2005 | ABS89395 16 ABR37470 16 ABO52651 16 | 13 | 18.5 | 2.8 | low p < 0.001, |
| | ABO76921 16 ABO52684 16 ABO52640 16 | | | | prev p < 0.001 |
| | ABL67140 16 ABL67118 16 ABK79945 16 ABJ09126 | | | | |
| | 16 ABI92277 16 ABI92266 16 ABO52101 25 | | | | |

As may be seen in Table 22 over the 42 year period for which sequence information is publicly available for H3N8 isolates, the cyclic nature of changes in Replikin concentration becomes evident. Where Replikin concentrations reach a high within the cycle, an epidemic occurs within about 1 to about 2 years. For example, a high of 22.2 Replikin sequences per 100 amino acids in 1979 falls to 2 Replikin sequences per 100 amino acids in 1998 and 1999 with no epidemics reported between 1995 and 2001. The Replikin concentration then appears to be on its way up again in 2001 with epidemics following in the United Kingdom and in Germany in 2002 and 2003, respectively, and then falls back to around 2 in 2003 and 2004 with a marked increase in 2005 to 18.5 that approaches the highs of 1979. Epidemics follow the 2005 increase in Australia, Italy and Japan in 2007.

Table 23 provides the data for Replikin concentration for publicly available sequences of the pB2 gene of the H3N8 strain of influenza virus from 1963 to 2005. Sequences were available under accession numbers at www.pubmed.com. Standard deviation and significance as compared to the mean Replikin Count of the previous year and of the lowest mean Replikin Count within the data set are also provided along with the mean Replikin Count for each year

TABLE 23
H3N8 pB2

| | | | Mean | | |
|---|---|---|---|---|---|
| | | No. of | Replikin | | |
| | | Isolates | Count per | | |
| Year | PubMed Accession Number-Replikin Count | per year | year | S.D. | Significance |
| 1963 | ABB88378 18 | 1 | 2.4 | 0.0 | |
| 1972 | ABI84587 18 | 1 | 2.4 | 0.0 | |
| 1977 | ABB19677 18 | 1 | 2.4 | 0.0 | |
| 1978 | ABB20352 18 ABB88319 18 | 2 | 2.4 | 0.0 | |
| 1979 | ABB87409 18 ABB86795 18 | 2 | 2.4 | 0.0 | |
| 1980 | ABI84488 14 ABB87799 19 BAF32964 18 Q08II6 | 4 | 2.3 | 0.3 | low p > 0.50, prev p > 0.50 |
| | 18 | | | | |
| 1981 | | | | | |
| 1982 | ABI84947 18 | 1 | 2.4 | 0.0 | prev p > 0.50 |
| 1985 | ABB19733 18 ABB19722 18 | 2 | 2.4 | 0.0 | |
| 1986 | ABO52136 18 ABJ09106 18 ABP49541 17 | 5 | 2.3 | 0.1 | low p < 0.30, prev p < 0.30 |
| | AAA43133 18 P26105 18 | | | | |
| 1987 | ABL67230 18 ABL67851 18 ABM66863 18 | 4 | 2.4 | 0.0 | prev p < 0.30 |
| | ABI95484 18 | | | | |
| 1991 | ABM21948 18 ABI84421 18 | 2 | 2.4 | 0.0 | |
| 1992 | ABB88193 18 | 1 | 2.4 | 0.0 | |
| 1998 | AAT65244 18 AAT65240 18 AAT65229 18 | 6 | 3.8 | 0.0 | |
| | AAT65228 18 AAT65216 18 AAT65214 18 | | | | |
| 1999 | AAZ23564 1 | 1 | 0.2 | 0.0 | |
| 2000 | AAG10729 1 | 1 | 0.6 | 0.0 | |
| 2001 | ABB19768 18 | 1 | 2.4 | 0.0 | |
| 2002 | AAZ23563 18 ABI47980 18 ABO51894 18 | 5 | 2.4 | 0.1 | low p < 0.30, prev p < 0.30 |
| | ABO51872 18 AAX23572 18 | | | | |
| 2003 | AAZ23567 18 AAZ23566 18 AAZ23565 18 | 6 | 2.4 | 0.0 | low p < 0.30, prev p > 0.50 |
| | AAZ23562 18 AAZ23561 18 ABB17182 18 | | | | |
| 2004 | AAZ23560 18 | 1 | 2.4 | 0.0 | prev p < 0.30 |
| 2005 | ABS89397 18 ABR37472 18 ABO52653 18 | 13 | 2.3 | 0.1 | low p < 0.10, prev p < 0.10 |
| | ABO76923 16 ABO52686 18 ABO52642 18 | | | | |
| | ABL67142 18 ABL67120 18 ABK79947 18 | | | | |
| | ABJ09128 18 ABI92279 18 ABI92268 16 | | | | |
| | ABO52103 18 | | | | |

A review of the Replikin concentrations of available sequences for the pB2 gene area of the H3N8 strain of influenza virus reveals much less variability in the Replikin concentration through the years. The pB2 Replikin concentration can be considered control data that validate the location of the most significant Replikin Peak Gene for the present isolates of virus in the pB1 gene area. Because the pB2 gene is right next to the pB1 gene, the difference in variability in Replikin Count between these neighboring areas is remarkable.

Data from a review of the Replikin Count of available sequences for the pA gene area of the H3N8 strain of influenza virus may be seen in FIG. 7. The data also reveal much less variability in the Replikin concentration through the years as compared to the pB1 gene area. As with the pB2 Replikin concentration, the pA Replikin concentration can be considered control data that validate the location and uniqueness of the most significant Replikin Peak Gene for the present isolates of virus in the pB1 gene area. The significance of these observations is further increased when it is realized that these quantitative annual measures for each of three areas of the EIV genome are an objective determination by software scanning and counting of the virus proteins of each of the viruses isolated and reported annually at www.pubmed.com.

Applicants analyzed publicly available sequences for isolates of EIV from years 1942 to 2007 and determined the mean whole genome Replikin Count for all isolates having genomic sequences in each year for which they were available.

A list of the accession numbers analyzed by FluForecast® (REPLIKINS LLC, Boston, Mass.) for the presence and concentration of Replikin sequences is provided in Table 24 below. The mean Replikin concentration for each year is provided following the list of accession numbers from isolates in each corresponding year. Standard deviation and significance as compared to the mean Replikin concentration of the previous year and of the lowest mean Replikin concentration within the data set are also provided along with the mean Replikin concentration for each year.

TABLE 24
Equine Influenza Whole Genome

| | | No. of | Mean | | |
|---|---|---|---|---|---|
| | | Isolates | Replikin | | |
| | | per | Count | | |
| Year | PubMed Accession Number-Replikin Count | year | per year | S.D. | Significance |
| 1942 | BAF49412 9 | 1 | 1.9 | 0.0 | |
| 1956 | ABB20499 4 AAA43411 1 ABB20504 2 ABB20503 2 ABB20502 8 | 21 | 2.2 | 1.3 | low p < 0.30 |
| | ABB20500 4 ABB20501 16 AAC57418 16 AAA43290 4 | | | | |
| | AAA43289 4 AAA43140 18 CAA44429 30 AAA43108 3 | | | | |
| | AAC35566 1 AAA52233 8 AAB51006 2 AAB51005 2 P88838 16 | | | | |
| | P26101 30 P26107 18 P16980 8 | | | | |
| 1957 | AAA64363 43 AAA64366 43 AAA64365 43 AAA64364 43 | 5 | 7.4 | 0.6 | low p < 0.001, |
| | AAA64362 36 | | | | prev p < 0.001 |
| 1963 | AAA43114 7 AAA43105 7 CAA44430 30 AAA43164 7 AAA43106 | 13 | 1.6 | 1.7 | low p < 0.40, |
| | 3 AAA43409 1 AAC31272 3 AAC31273 3 Q07579 1 P15658 7 | | | | prev p < 0.001 |
| | P17002 7 P26094 30 P16979 3 | | | | |
| 1964 | CAA43815 32 CAA44432 32 AAC35580 2 P26097 32 P26096 32 | 5 | 4.7 | 2.1 | low p < 0.04, |
| | | | | | prev p < 0.01 |
| 1966 | CAA44433 29 P26098 29 | 2 | 5.1 | 0.0 | prev p > 0.50 |
| 1969 | AAA43429 3 Q07581 3 | 2 | 0.6 | 0.0 | |
| 1971 | AAA43111 12 P17000 12 | 2 | 2.1 | 0.0 | |
| 1972 | AAA43100 12 CAA44434 34 AAA43355 1 Q07576 1 P26103 34 | 6 | 2.8 | 2.6 | low p < 0.40, |
| | P16994 12 | | | | prev p > 0.50 |
| 1973 | AAA43174 32 AAA43141 18 CAA44437 32 CAA44435 32 | 15 | 3.4 | 1.7 | low p < 0.005, |
| | AAA43104 27 AAA43637 17 AAA43457 7 AAB51000 2 | | | | prev p > 0.50 |
| | AAB50999 2 P26099 32 P26095 32 P26106 18 P16504 17 P13168 | | | | |
| | 27 P15673 7 | | | | |
| 1974 | AAA43093 16 P08327 16 | 2 | 3.4 | 0.0 | prev p > 0.50 |
| 1976 | AAA43107 6 AAA43101 6 CAA44436 32 ABF60576 6 P16995 6 | 7 | 2.4 | 2.2 | low p > 0.50, |
| | P26102 32 P16997 6 | | | | prev p < 0.20 |
| 1977 | AAC31296 3 CAA44431 32 AAQ90292 32 AAQ90288 7 | 9 | 3.0 | 2.0 | low p < 0.10, |
| | AAC31297 3 AAC31251 2 AAC31250 2 AAQ90293 16 P26100 32 | | | | prev p > 0.50 |
| 1978 | | | | | |
| 1979 | AAA43427 3 AAC31274 3 AAC31275 3 AAC31249 2 AAC31248 2 | 5 | 1.3 | 0.4 | low p < 0.02, |
| | | | | | prev p < 0.02 |
| 1980 | AAA43109 6 P16998 6 | 2 | 1.1 | 0.0 | prev p < 0.30 |
| 1981 | AAB02560 6 AAC31276 3 AAQ55062 7 AAC31277 3 AAA43245 3 | 7 | 1.0 | 0.3 | low p < 0.001, |
| | P08326 3 Q82559 6 | | | | prev p > 0.50 |
| 1985 | AAQ90291 6 AAQ90289 15 AAQ90290 3 AAA43112 6 AAA43110 | 8 | 1.4 | 1.0 | low p < 0.10, |
| | 4 Q6TXB9 3 P16999 4 Q6TXC0 15 | | | | prev p < .40 |
| 1986 | AAA43292 3 AAA43288 3 AAA43291 3 AAA43287 3 AAA43133 | 23 | 2.0 | 1.2 | low p > 0.50, |
| | 18 AAA43102 6 AAA43113 32 AAA43638 17 AAA43479 15 | | | | prev p < 0.10 |
| | AAA43458 15 AAB51002 2 AAB51001 2 AAA43430 3 NMIVEA | | | | |
| | 16 NMIVEK 3 Q07582 3 P17001 6 P19699 6 P26105 18 P16505 17 | | | | |
| | P13169 32 P67915 15 P67914 15 | | | | |
| 1987 | CAC84083 3 AAA43103 6 CAD23745 2 P16996 6 | 4 | 1.3 | 0.7 | low p < 0.10, |
| | | | | | prev p < 0.05 |
| 1988 | AAC31278 3 CAA74382 3 AAC31279 3 AAC31255 2 AAC31254 2 | 7 | 1.4 | 0.3 | low p < 0.005, |
| | AAC31253 2 AAC31252 2 | | | | prev p > 0.50 |
| 1989 | AAC31280 3 AAA43254 4 AAC31281 3 AAC31257 2 AAC31256 2 | 25 | 1.3 | 0.5 | low p < 0.001, |
| | AAA52247 3 AAA43253 4 AAA43151 12 AAA43374 3 CAA48482 | | | | prev p < 0.30 |
| | 6 HMIVEE 6 HMIVET 6 HMIVE9 6 HMIVE8 4 HMIVE7 6 | | | | |
| | HMIVE6 6 HMIVE5 6 HMIVE4 12 HMIVE3 12 HMIVE2 7 | | | | |
| | HMIVE1 7 Q07578 3 Q08011 6 Q03909 12 P26068 3 | | | | |
| 1990 | AAB36977 9 | 1 | 1.6 | 0.0 | prev p < 0.002 |
| 1991 | ABM21939 3 AAB36980 6 ABM21948 18 ABM21946 15 | 28 | 1.5 | 0.8 | low p < 0.01, |
| | ABM21945 33 ABM21944 2 ABM21943 2 ABM21942 15 | | | | prev p > 0.50 |
| | ABM21940 3 ABM21938 6 AAC31284 3 AAC31282 3 ABM21947 | | | | |
| | 15 ABM21941 3 CAA64893 6 CAA64894 6 AAC31285 3 | | | | |
| | AAC31283 3 AAC31261 2 AAC31260 2 AAC31259 2 AAC31258 2 | | | | |
| | AAA43354 3 AAC31286 3 AAC31287 3 AAC31263 2 AAC31262 2 | | | | |
| | Q07575 3 | | | | |
| 1992 | AAB36979 6 AAC31292 3 AAC31293 3 AAC31269 2 AAC31268 2 | 15 | 1.6 | 1.0 | low p < 0.20, |
| | AAA62470 6 AAC31290 3 AAC31288 3 A45591 30 AAC31291 3 | | | | prev p > 0.50 |
| | AAC31289 3 AAC31267 2 AAC31266 2 AAC31265 2 AAC31264 2 | | | | |
| 1993 | AAB27733 6 AAB36978 6 AAB36975 6 AAC31294 4 S33703 6 | 8 | 1.3 | 0.3 | low p < 0.001, |
| | AAC31295 4 AAC31271 2 AAC31270 2 | | | | prev p < 0.30 |
| 1994 | AAB36976 6 | 1 | 1.1 | 0.0 | prev p < 0.02 |
| 1995 | NP_034974 16 | 1 | 0.6 | 0.0 | |
| 1996 | AAC23906 23 | 1 | 3.5 | 0.0 | |
| 1998 | AAF22345 3 | 1 | 0.9 | 0.0 | |
| 1999 | ABA39843 6 AAZ23576 15 AAZ23564 1 ABA39854 3 | 4 | 1.0 | 0.8 | low p < 0.05, |
| | | | | | prev p > 0.50 |
| 2000 | AAQ18435 3 NP_898880 2 NP_057292 2 | 3 | 0.9 | 0.0 | low p < 0.001, |
| | | | | | prev p > 0.50 |
| 2001 | CAC69619 13 CAC69618 14 CAC69617 13 CAC69616 13 | 37 | 3.1 | 2.0 | low p < 0.001, |
| | CAC69615 14 CAC69614 13 CAC69613 14 CAC69612 33 | | | | prev p < 0.001 |
| | CAC69611 33 CAC69610 29 CAC69609 29 CAC69608 3 | | | | |
| | AC69607 4 CAC69606 4 CAC69605 14 CAC69604 15 CAC69603 | | | | |
| | 10 CAC69602 11 CAC69601 11 CAC69600 11 CAC69599 4 | | | | |
| | CAC69598 3 CAC69597 4 CAC69593 20 CAC69592 18 CAC69589 | | | | |
| | 20 CAC69588 18 ABF69262 3 ABF69265 18 ABF69264 11 | | | | |
| | ABF69263 27 ABF69261 3 ABF69260 3 ABF69259 2 ABF69258 2 | | | | |
| | ABF69256 13 ABF69257 6 | | | | |
| 2002 | AAX23578 3 AAX23579 2 AAX23576 15 AAX23575 6 AAX23574 | 36 | 2.0 | 1.1 | low p > 0.50, |
| | 32 AAX23573 15 AAX23572 18 ABA42430 2 ABA42429 2 | | | | prev p < 0.002 |
| | ABA39845 6 AAZ23582 15 AAZ23577 15 AAZ23563 18 | | | | |
| | AAX23577 3 ABA39853 3 AAM88392 4 AAM88391 4 AAN99161 | | | | |
| | 14 AAN99160 15 AAN99159 10 AAN99158 11 AAN99157 11 | | | | |
| | AAN99156 11 AAN99155 4 AAN99154 4 AAN99153 4 AAN99152 | | | | |
| | 3 AAN99151 4 AAN99147 20 AAN99146 18 AAN99143 20 | | | | |
| | AAN99142 18 AAN99141 6 AAN99140 6 AAN99139 3 AAN99138 3 | | | | |
| 2003 | AAZ23570 32 ABA42439 3 ABA42437 3 ABA42440 3 ABA42438 | 53 | 1.8 | 1.1 | low p > 0.50, |
| | 3 ABA42432 2 ABA42431 2 ABA42428 2 ABA42427 2 ABA39849 | | | | prev p > 0.50 |
| | 6 ABA39848 6 ABA39847 6 ABA39846 6 AAZ23583 15 | | | | |
| | AAZ23581 2 AAZ23578 15 AAZ23575 15 AAZ23574 15 | | | | |
| | AAZ23573 15 AAZ23567 18 AAZ23566 18 AAZ23565 18 | | | | |
| | AAZ23562 18 ABB17177 3 ABA39858 3 ABA39857 2 ABA39856 | | | | |
| | 3 ABA39855 3 ABB17182 18 ABB17181 15 ABB17180 33 | | | | |
| | ABB17179 4 ABB17178 4 ABB17176 3 ABB17175 15 ABB17173 | | | | |
| | 6 ABA42435 3 ABB17174 3 ABA42436 3 ABA42426 2 ABA42425 | | | | |
| | 2 ABA39842 6 AAZ23580 15 AAZ23572 15 AAZ23569 32 | | | | |
| | AAZ23561 18 ABA39852 3 AAQ41575 20 AAQ41574 18 | | | | |
| | AAQ41573 6 AAQ41572 6 AAQ41571 3 AAQ41570 3 | | | | |
| 2004 | ABA42441 3 ABA42433 3 ABA42442 3 ABA42434 3 ABA42424 2 | 38 | 2.0 | 1.2 | low p > 0.50, |
| | ABA42423 2 ABA39850 6 ABA39844 6 AAZ23579 15 AAZ23571 | | | | prev p < 0.40 |
| | 15 AAZ23568 32 AAZ23560 18 ABA39851 5 AAW17902 20 | | | | |
| | AAW17901 18 AAW17900 6 AAW17899 6 AAW17898 3 | | | | |
| | AAW17897 3 AAS32565 14 AAS32564 15 AAS32563 10 | | | | |
| | AAS32562 11 AAS32561 11 AAS32560 11 AAS32559 4 AAS32558 | | | | |
| | 4 AAS32557 4 AAS32556 3 AAS32555 4 AAS32551 20 AAS32550 | | | | |
| | 18 AAS32547 20 AAS32546 18 AAS32545 6 AAS32544 6 | | | | |
| | AAS32543 3 AAS32542 3 | | | | |
| 2005 | ABM47075 6 | 1 | 1.1 | 0.0 | prev p < 0.001 |
| 2006 | ABI00612 2 ABI00611 2 ABI00610 13 ABI00609 14 ABI00608 13 | 30 | 3.5 | 2.1 | low p < 0.001, |
| | ABI00607 14 ABI00606 13 ABI00605 14 ABI00604 13 ABI00603 | | | | prev p < 0.001 |
| | 14 ABI00602 33 ABI00601 33 ABI00600 29 ABI00599 29 | | | | |
| | ABI00598 3 ABI00597 4 ABI00596 4 ABI00595 14 ABI00594 15 | | | | |
| | ABI00593 10 ABI00592 11 ABI00591 11 ABI00590 11 ABI00589 4 | | | | |
| | ABI00588 3 ABI00587 4 ABI00583 20 ABI00582 18 ABI00579 20 | | | | |
| | ABI00578 18 | | | | |
| 2007 | BAF49411 30 ABS92756 20 ABS92755 18 ABS92754 6 ABS92753 | 26 | 2.2 | 1.4 | low p < 0.20, |
| | 6 ABS92752 3 ABS92751 3 ABN35533 14 ABN35532 15 | | | | prev p < 0.005 |
| | ABN35531 10 ABN35530 11 ABN35529 11 ABN35528 11 | | | | |
| | ABN35527 4 ABN35526 4 ABN35525 4 ABN35524 3 ABN35523 4 | | | | |
| | ABN35519 20 ABN35518 18 ABN35515 20 ABN35514 18 | | | | |
| | ABN35513 6 ABN35512 6 ABN35511 3 ABN35510 3 | | | | |

### Example 15

### Analysis of Replikin Count in PCV to Predict Increased Morbidity and Mortality in PCV

Applicants analyzed publicly available sequences for isolates of PCV from www.pubmed.com using proprietary search tool software (ReplikinForecast™ available in the United States from REPLIKINS LLC, Boston, Mass.) from years 1997 to 2007 and determined the mean Replikin Count for all isolates in each of years 1997 through 2007. Applicants then compared the mean Replikin Count for each year with qualitative changes in infection rates and mortality in pigs in Canada.

A list of the accession numbers analyzed for the presence and concentration of Replikin sequences is provided in Table 25 below. The mean Replikin Count for each year is provided following the list of accession numbers from isolates in each corresponding year. Standard deviation and significance as compared to the mean Replikin Count of the previous year and of the lowest mean Replikin Count within the data set are also provided along with the mean Replikin Count for each year.

TABLE 25
PCV

| | Analyzed Accession Numbers with total number of identified Replikin sequences; Replikin |
|---|---|
| Year | Count and Statistical Analysis |
| 1997 | AAC98885 16 AAC59472 4 AAC59466 3 AAC59464 5 AAC59462 9 |
| | Total Number of Accession Numbers: 5 |
| | Mean Replikin Count: 9.4 |
| | Standard Deviation: +/−10.8 |
| | Significance as compared to lowest mean Replikin Count: p < .40 |
| 1998 | AAC35336 2 AAC35332 5 AAC35330 12 AAC35326 2 AAC35322 5 AAC35320 12 |
| | AAC35316 2 AAC35313 3 AAC35311 5 AAC35309 12 AAC35305 2 AAC35302 3 AAC35300 |
| | 5 AAC35298 12 NP_065678 16 CAA11157 12 AAC61865 5 AAC61863 3 AAC61861 5 |
| | AAC61860 18 AAC61741 18 AAC61739 18 AAC61737 18 AAC34819 16 AAF97593 14 |
| | AAD03091 3 AAD03090 2 AAD03087 5 AAD03086 12 AAD03075 4 AAD03080 2 |
| | AAD03073 5 AAD03071 12 AAD03069 2 AAD03065 2 AAD03063 5 AAD03061 12 O56124 |
| | 5 AAC69862 4 NP_047277 4 AAC69861 21 AAC34818 4 NP_047275 21 NP_048062 5 |
| | NP_048061 12 AAD11930 5 AAD11928 12 AAC69863 1 AAC34816 21 |
| | Total Number of Accession Numbers: 49 |
| | Mean Replikin Count: 6.1 |
| | Standard Deviation: +/−3.7 |
| | Significance as compared to lowest mean Replikin Count: p < .05 |
| | Significance as compared to previous mean Replikin Count p < .40 |
| 1999 | BAA88133 12 AAD50432 12 AAD38398 12 AAG41230 2 AAG41228 5 AAG41226 12 |
| | AAD37776 12 AAD45580 16 AAF35304 12 AAF35302 12 AAF35300 12 AAF35298 12 |
| | AAF35296 12 AAF35294 12 AAF35292 12 AAD12314 2 AAD12313 2 AAD12309 6 |
| | AAD12308 12 AAE81207 4 |
| | Total Number of Accession Numbers: 20 |
| | Replikin Count: 5.0 |
| | Standard Deviation: +/−3.3 |
| | Significance as compared to lowest mean Replikin Count: p < 0.50 |
| | Significance as compared to previous mean Replikin Count p > 0.20 |
| 2000 | CAC41085 12 CAC41084 12 CAC41083 11 AAL09364 12 AAL09363 12 AAF87238 12 |
| | AAF87236 12 AAF87234 12 AAF87232 12 AAF87230 12 AAF87228 12 NP_059530 2 |
| | NP_573443 5 NP_150370 4 CAD23544 5 CAC50263 4 AAG30569 3 AAG30566 8 AAG30563 |
| | 8 AAG30560 7 AAG30557 4 AAG30554 4 AAG30551 7 AAG30548 7 CAC24649 35 |
| | AAF74197 2 |
| | Total Number of Accession Numbers: 26 |
| | Replikin Count: 2.9 |
| | Standard Deviation: +/−1.2 |
| | Significance as compared to lowest mean Replikin Count: p < 0.001 |
| | Significance as compared to previous mean Replikin Count p > 0.005 |
| 2001 | AAK60464 5 AAK60462 12 AAL58397 12 BAB69442 5 BAB69441 12 BAB69439 2 |
| | BAB69438 5 BAB69437 12 BAB69435 3 BAB69433 5 BAB69432 12 AAK51544 14 |
| | AAK56300 12 AAK56298 12 AAK56296 12 AAL01081 4 AAL01080 1 AAL01077 5 |
| | AAL01075 12 NP_998971 5 NP_613078 4 AAM00235 4 AAN37998 4 AAN37994 3 |
| | AAN37990 3 AAN37986 3 AAN37982 4 AAN37978 3 AAN37974 4 AAN37970 3 AAN37966 |
| | 4 AAN37962 4 AAN37958 3 AAL13485 3 CAC50253 2 CAC50247 2 |
| | Total Number of Accession Numbers: 36 |
| | Replikin Count: 2.8 |
| | Standard Deviation: +/1.7 |
| | Significance as compared to lowest mean Replikin Count: p < 0.001 |
| | Significance as compared to previous mean Replikin Count p > 0.50 |
| 2002 | AAM61272 12 AAM61262 12 AAM61274 16 AAM61268 12 AAM61266 12 AAM61270 12 |
| | AAM61264 12 AAO24127 1 AAO39760 12 AAM21847 1 AAM21846 1 AAM21845 7 |
| | AAM21844 12 AAO24128 12 AAO24126 11 AAO24124 10 AAO24122 12 AAO23147 12 |
| | AAO23145 12 AAN81597 12 AAN06826 12 AAN62769 12 AAN62767 12 AAN62765 12 |
| | AAN16398 14 AAM83186 11 AAL69968 12 AAN77863 4 AAN77862 2 AAN77861 2 |
| | AAN77860 14 AAN77859 16 AAO39666 16 AAM76057 12 Q8BB16 12 YP_164519 4 |
| | ABA54889 4 ABA54887 4 ABA54885 4 AAR28043 4 AAO95302 12 AAO95299 |
| | Total Number of Accession Numbers: 42 |
| | Replikin Count: 3.5 |
| | Standard Deviation: +/1.4 |
| | Significance as compared to lowest mean Replikin Count: p < 0.001 |
| | Significance as compared to previous mean Replikin Count p > 0.05 |
| 2003 | AAP51128 12 AAS65982 1 AAS65993 12 AAS65991 12 AAS65989 12 AAS65987 12 |
| | AAS65985 12 AAS65983 12 AAS65981 9 AAS65979 12 AAS65977 12 AAS65975 12 |
| | AAP83635 12 AAP83633 12 AAP83631 12 AAP83629 12 AAP83627 12 AAP83625 12 |
| | AAP83623 12 AAP83621 12 AAP83619 12 AAP83617 12 AAP83615 12 AAP83613 12 |
| | AAP83611 12 AAP83609 12 AAP83607 12 AAP83605 12 AAP83603 12 AAP83601 12 |
| | AAP83599 12 AAP83597 12 AAP83595 12 AAP83593 12 AAP83591 12 AAR97518 1 |
| | AAR03722 12 AAR03720 12 AAR03718 12 AAR03716 12 AAQ94098 12 AAQ94096 12 |
| | AAQ94094 12 AAQ94092 12 AAQ94090 12 AAQ94088 12 AAP44190 5 AAP44188 12 |
| | AAP44187 5 AAP44185 12 AAP44184 5 AAP44182 12 AAO61773 12 AAR97517 12 |
| | AAQ96327 12 AAQ23156 5 AAQ23155 12 AAP42468 9 AAP42466 10 AAP42464 12 |
| | AAO61136 12 NP_937956 12 AAR03714 12 Q805H4 16 YP_209622 4 AAQ93492 6 |
| | AAP69227 4 AAR27947 1 AAT00481 5 AAT00473 4 AAT00471 4 AAT00469 4 AAS16932 6 |
| | AAS16931 7 AAS16930 6 AAS16929 6 AAS16928 6 AAS16927 6 AAS16926 6 AAS16925 6 |
| | AAS16924 6 |
| | Total Number of Accession Numbers: 81 |
| | Replikin Count: 3.4 |
| | Standard Deviation: +/1.0 |
| | Significance as compared to lowest mean Replikin Count: p < 0.001 |
| | Significance as compared to previous mean Replikin Count p > 0.50 |
| 2004 | AAU87520 1 AAT97651 2 AAT97647 1 YP_077191 16 AAW78483 16 AAW78481 20 |
| | AAW78479 20 AAW78477 20 AAW78475 12 AAW78473 12 AAW78471 12 AAW78469 12 |
| | AAW78467 12 AAW78465 12 AAW78463 12 AAV34141 20 AAV34139 12 AAU87519 12 |
| | AAU87517 12 AAU87515 12 AAU87513 12 AAU87511 12 AAU87509 12 AAU87507 12 |
| | AAU34001 10 AAU01913 14 AAT97650 12 AAT97648 12 AAT97646 12 AAT97644 12 |
| | AAT77546 1 AAT72755 16 AAT36358 12 AAU13781 1 AAX49397 12 AAU01966 12 |
| | AAT79579 12 AAT72901 12 AAT58234 11 AAS66199 1 AAS66197 1 AAS45844 12 |
| | AAS45843 12 CAJ31064 12 AAU13780 12 AAX52911 12 AAU87505 12 AAT39479 12 |
| | AAT39460 9 AAT37493 9 AAS66198 12 AAS66196 12 AAS66194 12 AAS66192 12 |
| | AAS66190 12 AAS90297 12 AAS89260 12 NP_999004 27 YP_271921 7 CAF25171 12 |
| | AAT51967 3 BAD90990 3 BAD90989 4 BAD90988 4 BAD90987 4 BAD90986 3 BAD90985 |
| | 3 AAS86324 4 AAS93283 6 AAS93279 6 AAS93276 5 AAS93272 9 AAS93268 6 AAS89814 |
| | 4 AAS89813 6 AAS89812 8 AAS89811 6 AAS89810 6 AAS89809 6 AAS89808 8 AAS89807 |
| | 9 AAS89806 8 AAS89805 5 AAS89804 4 AAS89803 4 AAS89802 5 AAS89801 7 AAS89800 |
| | 9 AAS89799 6 AAS89798 11 AAS89797 6 AAS89796 6 AAS89795 S AAS89794 5 AAS89793 |
| | 6 AAS89792 11 AAS89791 9 AAS89790 9 AAS89789 9 AAS89788 6 AAS89787 6 AAS89786 |
| | 6 AAS89785 6 AAS89784 6 AAS89783 6 AAS89782 6 AAS89781 6 |
| | Total Number of Accession Numbers: 107 |
| | Replikin Count: 3.3 |
| | Standard Deviation: +/1.3 |
| | Significance as compared to lowest mean Replikin Count: p < 0.001 |
| | Significance as compared to previous mean Replikin Count p > 0.30 |
| 2005 | ABJ98317 12 ABA29241 12 AAZ20802 11 AAZ20800 10 AAZ20798 11 AAZ20796 12 |
| | AAZ20794 12 AAW79865 9 ABC26025 12 ABA40480 12 AAZ78351 12 AAY40292 12 |
| | AAX21515 12 ABB29423 10 ABB29421 12 ABB29419 12 ABB29417 10 ABB29415 10 |
| | ABB29413 10 ABB29411 10 ABB29409 10 ABB29407 10 ABB29405 10 ABB29403 8 |
| | ABB29401 10 ABB29399 10 ABB36791 1 ABA60807 12 ABA60805 12 ABA60803 11 |
| | ABA40399 12 ABA40397 12 AAZ66792 11 AAX10150 12 AAX62053 16 AAX62051 12 |
| | AAX62049 12 AAX62047 12 AAX62045 12 AAX62043 16 AAX62041 12 ABC75103 12 |
| | ABB20934 12 ABA26910 12 ABA26908 10 AAY34249 12 YP_6109628 AAZ07884 7 |
| | ABB59615 8 ABA39170 3 ABA39166 3 AAX35672 1 ABA39162 4 ABA39158 3 ABA39154 |
| | 4 ABA39150 3 ABA39146 3 ABA39142 3 ABA39138 3 AAZ68049 2 AAZ68045 2 |
| | Total Number of Accession Numbers: 61 |
| | Replikin Count: 3.0 |
| | Standard Deviation: +/1.1 |
| | Significance as compared to lowest mean Replikin Count: p < 0.001 |
| | Significance as compared to previous mean Replikin Count p > 0.20 |
| 2006 | ABG21191 3 ABI29887 12 ABG21279 10 ABG21277 10 ABG21275 10 ABG21273 10 |
| | ABG21271 10 ABG21269 12 ABG21267 12 ABJ98319 12 ABI93799 16 ABI93797 12 |
| | ABD59347 12 ABG48510 14 ABD42928 12 ABM67071 16 ABM88864 12 ABM88862 12 |
| | ABM88860 12 ABI17537 12 ABI17535 12 ABI17533 12 ABI17531 12 ABI17529 12 |
| | ABI17527 12 ABI17525 12 ABI17523 12 ABG37025 16 ABG37023 12 ABD52438 16 |
| | ABG24031 16 ABG24029 16 ABF71465 12 ABF19812 16 ABF19810 14 ABE96824 16 |
| | ABE96822 16 ABE96820 16 ABE96818 13 ABE96816 16 YP_803548 1 YP_803551 5 |
| | ABI54258 5 ABI54255 1 ABK79791 2 ABK79788 2 ABK79785 2 ABK79782 3 ABK79779 7 |
| | ABK79776 9 ABK79773 3 ABI97391 3 ABE03771 4 ABE03767 6 |
| | Total Number of Accession Numbers: 54 |
| | Replikin Count: 3.4 |
| | Standard Deviation: +/1.4 |
| | Significance as compared to lowest mean Replikin Count: p < 0.001 |
| | Significance as compared to previous mean Replikin Count p > 0.10 |

### Example 16

### Repeat KHKK (SEQ ID NO: 1584) Signatures in Lung Cancer and Tobacco Mosaic Virus

Publicly available amino acid sequences at Accession Nos: Q9NS56 and 117607067, for non-small lung cancer and tobacco mosaic virus, respectively, were analyzed for Replikin Peak Genes. The inventors queried Accession No. Q9NS56 at www.pubmed.com. Accession No. Q9NS56 discloses the amino acid sequence of E3 ubiquitin-protein ligase Topors from human chromosome 9 of non-small cell lung cancer (SEQ ID NO: 1740). Upon analysis of SEQ ID NO: 1740, the inventors observed a Replikin Peak Gene having continuous Replikin sequences that begin at residue 880 (lysine) and continue through residue 897 (histidine).

The inventors isolated the RPG (SEQ ID NO: 1741) in silico. SEQ ID NO: 1741 was identified for diagnostic and therapeutic uses in, for example, an immunogenic compound and a therapeutic vaccine compound and as a predictive sequence for lethality. Fifty-two Replikin sequences (SEQ ID NOS: 1886-1937) were identified in the RPG of SEQ ID NO: 1741 for diagnostic, therapeutic and predictive uses as described herein. SEQ ID NOS: 1742-1747 were identified in the amino-terminal of the sequence disclosed in Accession No. Q9NS56 (SEQ ID NO: 1741), SEQ ID NOS: 1748-1780 were identified in the mid-molecule of the sequence, and SEQ ID NOS: 1781-1885 were identified in the carboxy-terminal of the sequence.

The Replikin Count of the amino acid sequence (SEQ ID NO: 1740) disclosed at Q9NS56 was 144 Replikin sequences in 1045 total amino acids for a Replikin Count of 13.8. The Replikin Count of the RPG (SEQ ID NO: 1741) was 52 Replikin sequences in 18 total amino acids for a Replikin Count of 289, the highest count yet observed.

Within the Replikin sequences identified in the RPG (SEQ ID NO: 1741), the KHKK signature was observed 57 times within 52 Replikin sequences. This high concentration of lethal signatures corresponds to the high lethality of non-small cell lung malignancies.

The inventors queried Accession No. 117607067 at www.pubmed.com. Accession No. 117607067 discloses the amino acid sequence of a hot pepper 26S proteasome subunit RPN7 induced by tobacco mosaic virus (SEQ ID NO: 1938). Upon analysis of SEQ ID NO: 1938, the inventors observed a Replikin Peak Gene having continuous Replikin sequences that begin at residue 91 (histidine) and continue through residue 175 (lysine).

The inventors isolated the RPG (SEQ ID NO: 1939) in silico. SEQ ID NO: 1939 was identified for diagnostic and therapeutic uses in, for example, an immunogenic compound and a therapeutic vaccine compound and as a predictive sequence for lethal outbreaks of tobacco mosaic virus. Fifty-four Replikin sequences (SEQ ID NOS: 1941-1994) were identified in the RPG of SEQ ID NO: 1939 for diagnostic, therapeutic and predictive uses as described herein.

SEQ ID NO: 1941 was identified in the amino-terminal of the sequence disclosed in Accession No. 117607067 (SEQ ID NO: 1938), SEQ ID NOS: 1942-1986 were identified in the mid-molecule of the sequence, and SEQ ID NOS: 1987-1994 were identified in the carboxy-terminal of the sequence. Each Replikin sequence was isolated in silico for diagnostic, therapeutic and predictive purposes as described herein including for immunogenic compositions and vaccines.

The Replikin Count of the amino acid sequence (SEQ ID NO: 1938) disclosed at Accession No. 117607067 was 55 Replikin sequences in 179 total amino acids for a Replikin Count of 30.7. The Replikin Count of the RPG (SEQ ID NO: 1939) was 54 Replikin sequences in 89 total amino acids for a Replikin Count of 61.

Within the Replikin sequences identified in 117607067 (SEQ ID NO: 1938), the KHKK (SEQ ID NO: 1584) signature was observed twenty times within 61 Replikin sequences. This high concentration of lethal signatures corresponds to the high lethality of tobacco mosaic virus and connects tobacco mosaic virus through KHKK (SEQ ID NO: 1584) signatures to lethal lung cancer.

As discussed above, repeating signatures such as a “KHKK” (SEQ ID NO: 1584) signature have been observed in Replikin sequences within RPGs of lethal malignancies, viruses and organisms. The KHKK (SEQ ID NO: 1584) signature has been observed eleven times within the RPG of the protozoa that causes most malaria, P. falciparum, 20 times within the RPG of tobacco mosaic virus, which caused exacerbated cell death induced by tobacco mosaic virus, and 57 times in non-small cell lung carcinoma within 52 Replikins observed within the 18 amino acid RPG identified in chromosome 9 of a non-small cell lung carcinoma. The presence of such a high number of KHKK (SEQ ID NO: 1584) signatures within the 18 amino acid RPG of the non-small cell lung carcinoma is explained by overlapping of the signatures. Overlapping of Replikin sequences and repeated signatures such as KHKK (SEQ ID NO: 1584) has now been associated with lethality, virulence and rapid replication. Together, these data indicate that a Replikin gene is quantitatively associated with lethal functions, and may be a mobile agent of lethality transferring between strains and species.

### Example 17

### Repeat KHKK (SEQ ID NO: 1584) Signatures in Malaria

The inventors queried Accession No. P13817 at www.pubmed.com. Accession No. P13817 discloses an amino acid sequence from Plasmodium falciparum. The inventors analyzed the amino acid sequence provided at P13817 (SEQ ID NO: 2043). Upon analysis of SEQ ID NO: 2043, the inventors observed a Replikin Peak Gene having continuous Replikin sequences that begin at residue 323 (histidine) and continue through residue 473 (lysine) (SEQ ID NO: 3659).

The inventors isolated the RPG (SEQ ID NO: 3659) in silico. SEQ ID NO: 3659 was identified for diagnostic and therapeutic uses in, for example, an immunogenic compound and a therapeutic vaccine compound and as a predictive sequence for lethal outbreaks of malaria. Two hundred and thirty-one Replikin sequences (SEQ ID NOS: 2312-2315 and 2317-2544) were identified in the RPG of SEQ ID NO: 3659 for diagnostic, therapeutic and predictive uses as described herein. Replikin sequences SEQ ID NOS: 2044-2077 were identified in the amino-terminal of the sequence of SEQ ID NO: 2043, Replikin sequences SEQ ID NOS: 2079-2080 were identified in the mid-molecule of the sequence, and Replikin sequence SEQ ID NOS: 2081-2315 were identified in the carboxy-terminal.

The Replikin Count of the whole sequence (SEQ ID NO: 2043) was 268 Replikin sequences in 473 total amino acids for a Replikin Count of 56.7. The Replikin Count of the RPG area (SEQ ID NO: 3659) was 231 Replikin sequences in 151 total amino acids for a Replikin Count of 153.

The inventors queried Accession No. A44396 at www.pubmed.com. Accession No. A44396 discloses an amino acid sequence from an ATP-ase-like molecule of P. falciparum isolated in 1993. The inventors analyzed the amino acid sequence provided at A44396 (SEQ ID NO: 2926). Upon analysis of SEQ ID NO: 2926, the inventors observed a Replikin Peak Gene having continuous Replikin sequences that begin at residue 1297 (histidine) and continue through residue 1333 (histidine).

The inventors isolated the RPG (SEQ ID NO: 3661) in silico. SEQ ID NO: 3661 was identified for diagnostic and therapeutic uses in, for example, an immunogenic compound and a therapeutic vaccine compound and as a predictive sequence for lethal outbreaks of malaria. Seventeen Replikin sequences (SEQ ID NOS: 3282-3285, 3287-3291, 3293, 3295, 3299-3300, 3302, 3304, 3306, 3308, 3310-3313 and 3663) were identified in the RPG of SEQ ID NO: 3661 for diagnostic, therapeutic and predictive uses as described herein. Replikin sequences SEQ ID NOS: 2546-2632 were identified in the amino-terminal of the sequence of SEQ ID NO: 2926, Replikin sequences SEQ ID NO: 2633-2720 were identified in the mid-molecule of the sequence, and SEQ ID NOS: 2721-2900 were identified in the carboxy-terminus.

The Replikin Count of the whole ATP-ase sequence (SEQ ID NO: 2926) was 355 Replikin sequences in 1984 total amino acids for a Replikin Count of 17.9. The Replikin Count of the RPG area (SEQ ID NO: 3661) was 15 Replikin sequences in 37 total amino acids for a Replikin Count of 41.

Eleven signature repeat KHKK (SEQ ID NO: 1584) sequences were noted in the Replikin sequences of the RPG. The eleven signature repeats, namely, SEQ ID NOS: 3286, 3292, 3294, 3296, 3298, 3662, 3301, 3303, 3305, 3307, and 3309 are respectfully found within noted Replikin sequences SEQ ID NOS: 3286, 3291, 3293, 3295, 3297, 3299, 3300, 3302, 3204, and 3206.

The presence of such a high number of KHKK (SEQ ID NO: 1584) signatures within the fifteen Replikin sequences in the 37 amino acid RPG of the P. falciparum is explained by overlapping of the signatures. Overlapping of Replikin sequences and repeated signatures such as KHKK (SEQ ID NO: 1584) has now been associated with lethality, virulence and rapid replication as in malaria, which has an exceptionally high rate of replication within its lifecycle.

### Example 18

### Laboratory Demonstration of Relationship of Replikin Count to Percent Mortality in Taura Syndrome Virus Infection in Shrimp

To test further the relationship of Replikins to virulence, the relationship of Replikin count of shrimp viruses to mortality in shrimp was examined in a controlled situation. Based on the hypothesis that the Replikin count of a virus is related to virulence of the virus and the percent mortality of the host, as developed from the evidence on H5N1 virus infections in humans, Applicants tested whether it would be possible to predict solely from the Replikin Count of the amino acid sequence of the whole genome what the order of virulence would be of four strains of the virus. Taura syndrome shrimp virus (TSV), which kills most host shrimp within a few days of infection, was chosen to be studied. The amino acid sequences of four strains of taura syndrome virus (Belize, Thailand, Hawaii, and Venezuela) were analyzed with the FluForecast® software of REPLIKINS LLC, Boston, Mass. and the results held in confidence until the laboratory challenge experiments with the virus were completed, then compared with the percent mortality produced by each strain.

In the laboratory, there was a significant linear correlation between the mortality rates of the host shrimp challenged with each of the four virus strains and the mortality rates predicted earlier by only the Replikin counts of each strain. These data support the conclusion that virus Replikin peptide concentration, in addition to predicting virus outbreaks, relates quantitatively to host mortality rate and to the increase in virulence over time observed.

A. Replikin Analysis

Visual Replikin analysis was performed on the sequence information for the taura syndrome virus isolates from Belize, Thailand, Hawaii, and Venezuela by applying the algorithm defining Replikins with computer access to protein and genomic sequences freely available on PubMed or other public databases. The specific defining algorithm follows: a Replikin is a peptide sequence in a protein or genome, 7 to 50 amino acids long having a terminal lysine and a terminal lysine or histidine, containing at least 2 lysine groups 6 to 10 amino acids apart, at least 1 histidine group, and at least 6% lysine. Overlapping Replikins are common and are counted separately. The quantitative correlations with rapid replication and epidemics and lethality require all components of the algorithm to be in place for each Replikin. Thus for example, if the length and lysine requirements are present but there is no histidine present, the peptide is not a Replikin. Automated Replikin analysis was performed with the FluForecast® software service of Replikins Ltd., Boston, Mass.

B. Identification of the Replikin Peak Gene

The Replikin count was used to identify that area of the genome which had the highest concentration of Replikins, and this area called the Replikin Peak Gene (RPG) area. The further two to eight-fold increase in the Replikin count of the RPG which occurred with outbreaks was further used to confirm the identity of this gene. The function of the gene was therefore used to identify it or isolate it “in silico”.

C. Shrimp Virus Laboratory Methods

At the Aquaculture Pathology Laboratory, Department of Veterinary Science and Microbiology, University of Arizona, Tucson Ariz., small juveniles of specific-pathogen-free Litopenaeus vannamei shrimp per tank, mean weight: 1.8 g, were fed minced TSV-infected tissues (infected separately with each of the 4 isolates originating from Belize, Thailand, Venezuela and Hawaii) for 3 days at 5% of their body weight. These shrimp were maintained with pelleted ration (Rangen 35%) for the following 12 days. Each challenge bioassay of a specific isolate was done in triplicate. During the bioassay period, all tanks were checked daily for dead or moribund shrimp. All mortalities were removed from the tank and frozen. One to three moribund shrimp from each isolate were preserved in Davidson's AFA fixative and processed for routine histology to confirm viral infection. For each isolate, six moribund shrimp were collected during the acute phase infection and total RNA was extracted from their gill tissues with a High Pure RNA tissue kit (Roche). The extracted RNA was analyzed for the presence of TSV by real-time RT-PCR. All tanks were outfitted with an acclimated biological filter and aeration, and were covered with plastic to contain aerosols. The average salinity of the water was 23 ppt and the water temperature was 28° C. The challenge study was terminated after 15 days with live animals counted as survivors.

D. Comparison of Virulence

First mortality was seen on day 2 after exposure to TSV in all 4 isolates. For Belize isolate, most (83%) of shrimp died by day 4 and had a 0% survival at day 11 (FIG. 14A, Table 26). For Thailand isolate, 63% mortalities occurred by day 4 and had 20% survivals at the end of 15-day bioassay (FIG. 14B, Table 26). For Hawaii isolate, mortalities increased starting at day 2 and reached to a peak at day 5; the cumulative survival is 22% at the end (FIG. 14C, Table 26). For Venezuela isolate, mortalities occurred slowly at days 2 and 3 with 22% of shrimp showed mortalities on day 4 and then mortalities were slowing down; there were 42% of shrimp survived in the end (FIG. 14D, Table 26). The time period for reaching 50% mortality caused by TSV infection for the isolate of Belize, Thailand, Hawaii and Venezuela were 2.8, 3.5, 4.5 and 7 days, respectively (Table 26).

FIG. 14 provides data for the cumulative survival of Litopenaeus vannamei challenged with TSV isolates per os with taura syndrome virus isolates from a: Belize; b: Thailand; c: Hawaii; d: Venezuela. The data from FIG. 14 is contained in Table 26 below.

TABLE 26
TSV Challenge

| | GenBank no. | Survival (%) | Day of 50% |
|---|---|---|---|
| TSV isolate | (ORF1) | (Mean) | mortality |
| Belize | AAT81157 | 0 | 2.8 |
| Thailand | AAY56363 | 20 | 3.5 |
| US-Hawaii | AAK72220 | 22 | 4.5 |
| Venezuela | ABB17263 | 42 | 7.0* |

*High variation was observed in Venezuela's triplicate tanks, thus the Day of 50% mortality was determined by Kaplan-Meier survival analysis with the Statistix 8 program.

The correlation of the virulence observed for each of the TSV isolates with the predicted virulence by Replikin Count alone are shown in FIG. 15. FIG. 15A provides data comparing Replikin Counts of the four isolates with the mean day of 50% mortality as gathered in blind studies. FIG. 15B provides data comparing Replikin Counts of the four isolates with mean cumulative mortality as gathered in blind studies. The linear quantitative relationship between the predicted and experimental values is evident. Table 27 below provides the histological data that was gathered for the moribund shrimp to demonstrate TSV infection.

TABLE 27
Histology

| UAZ ID# | TSV Isolate | Days after exposure | TSV lesions¹ | LOS² |
|---|---|---|---|---|
| O6-407J/1 | Belize | 3 | G4 | G4 |
| 06-407F/1 | Thailand | 3 | G4 | G2 |
| 06-407D/1 | Thailand | 4 | G4 | G3 |
| 06-407E/1 | Thailand | 4 | G3 | G2 |
| 06-407A/1 | Hawaii | 4 | G2 | G3 |
| 06-407C/1 | Hawaii | 4 | G2 | G4 |
| 06-407H/1 | Venezuela | 4 | G4 | G2 |

Severity grade:
G1: sign of infection;
G2: moderate signs of infection;
G3: moderate to high signs of infection;
G4: severe infection.
¹TSV lesions = Presence of TSV pathognomonic lesions in the gills, mouth, stomach, intecumental cuticular epithelium, and appendages.
²LOS = presence of lymphoid organ spheroids within the lymphoid organ.

Belize TSV: Acute lesions of diagnostic TSV infection were found in one representative shrimp sample (06-407J/1) at a severity grade of G4. Nuclear pyknosis and karyorrhexis were observed in the cuticular epithelium of the general body surface, appendages, gills, stomach and esophagus. Lymphoid organ spheroids were also found at severity grade G4. Thailand TSV: Severe (G4) TSV infection was detected in 2 out 3 shrimp (06-407D/1, F/1), another shrimp (06-407E/1) showed a moderate to high grade (G3) of infection. Lymphoid organ spheroids were found at severities of G2 and G3. Hawaii TSV: Moderate level (G2) of TSV infection was detected in 2 shrimp (06-407A/1, C/1) collected at day 4. Lymphoid organ spheroids were found at severities of G3 and G4. Venezuela TSV: Severe (G4) TSV infection was detected in one representative shrimp (06-407H/1) sampled at day 4. Lymphoid organ spheroids were found at severity of G2.

The real-time TSV RT-PCR assay was designed specifically for Hawaii TSV and thus a high level (10⁷ copies/μl RNA) of TSV was detected in the Hawaii-TSV challenged shrimp Table 28). The target sequence in 3 other isolates has 2 mis-matched nucleotides with the primers/TaqMan probe. Thus, there is 10 times less quantity of TSV (10⁶ copies/μl RNA) detected in Belize and Thailand samples. The Venezuela samples were detected with 100-100,000 times less: 10²-10⁵ copies/μl RNA; this may be due to both the effect of mismatches and a lower level of infection in the samples analyzed. Nevertheless, all 24 samples (6 from each isolates) were all positive for TSV infection. This confirms that the mortalities observed from bioassays are from TSV infection. The real-time TSV RT-PCR assay data is found below in the Table 28.

TABLE 28
PCR

| TSV isolate | Mean (Range) TSV copies/:1 RNA |
|---|---|
| Belize | 2.7 × 10⁶ (4.8 × 10⁵ − 4.4 × 10⁶) |
| Thailand | 2.7 × 10⁶ (4.3 × 10⁵ − 7.5 × 10⁶) |
| Hawaii | 5.2 × 10⁷ (2.3 × 10⁷ − 7.5 × 10⁷) |
| Venezuela | 6.5 × 10⁵ (6.5 × 10² − 2.0 × 10⁵) |

E. Laboratory Mortality Results Correlated With Replikin Counts

Virulence of 4 TSV isolates (Hawaii, Belize, Thailand and Venezuela) was compared through a per os laboratory infection in juvenile Litopenaeus vannamei (Kona stock, Oceanic Institute, Hawaii). The results showed that the Belize isolate is the most virulent, Thailand is the second, followed by the Hawaii isolate, and the Venezuela isolate is the least virulent. This is based on the analyses of cumulative survivals at the end of the bioassay (p<0.047) and the time when 50% mortality was occurred (p<0.001). That the mortality of the shrimp was caused by TSV infection was confirmed by positive reactions in RT-PCR detection and by the appearance of characteristic lesions observed in histological analysis

F. Laboratory Mortality Results Correlated With Replikin Counts

Experimentally, Replikin Counts alone prospectively correctly predicted: (1) blind in controlled experiments in the laboratory, the order of lethality in shrimp of four strains of taura syndrome virus (FIGS. 15A and B); (2) an increasing H5N1 percent mortality in humans (FIG. 4); and (3) the host (FIG. 5); and (4) the country in which the latter would occur, Indonesia (FIG. 6). For both H5N1 influenza in human hosts, and taura syndrome virus infection in shrimp hosts, evidence in this study demonstrates the quantitative relationship of the virus Replikin Count to the mortality rate in the host. The ability to predict blind is of course one of the more definitive proofs of a relationship; the demonstration of a quantitative linear relationship is even more definitive. Thus, the concentration of a class of specific virus peptides, Replikins, has here been quantitatively correlated with the percent mortality these viruses produce in their respective hosts, namely invertebrate crustacean (shrimp) and vertebrates (humans). To our knowledge, no quantitative correlation of virus structure and host lethality has been reported previously.

### Example 19

### Increased Host Resistance to Taura Syndrome Virus by Administration of Synthetic Replikins

Shrimp cultured using the Challenge Methods described in Example 18 above were exposed in a first experiment for two weeks to synthetic Replikins per os mixed in their feed. The Replikins were peptides specific to Replikin sequences present in the TSV Hawaii strain isolate with which the shrimp were challenged.

In the experiment, mortality was reduced by 50% compared to a control group. The control group was given feed not containing synthetic Replikin sequences. A second control group was fed Replikin sequences synthesized with the covalent binding of additional amino acids to the same synthetic Replikins fed to the shrimp. The covalently “blocked” Replikins did not increase shrimp resistance to the virus in the same experiment demonstrating that the increase in host resistance was specific to the Replikin peptide structure.

Because little is known about the details of the immune system of the shrimp (shrimp appear not to produce antibodies), the phenomenon of “resistance” to infection appears to be based in a “primitive immune system” perhaps similar to the “toll receptor” and related systems. Thus the term “increased resistance” is used for the observed phenomenon and Replikin feed is used rather than “vaccine” for the administered substance which increases resistance.

The surviving shrimp of the first challenge were then set up in a fresh culture, fed for an additional two weeks with feed containing Replikin sequences, then again challenged with the Hawaii strain of taura syndrome virus. The Replikin sequence supplemented feed was maintained while the survivors were again challenged repeatedly by the same virus, in repeated cycles, until 100% of the shrimp survived the TSV challenge.

### Example 20

### Calculation of RPG in Viral Hemorrhagic Disease in Fish

The inventors queried Accession No. ABQ42711 at www.pubmed.com. Accession No. ABQ42711 discloses an amino acid sequence from a glycoprotein in hemorrhagic septicemia virus. Hemorrhagic septicemia virus is a cause of hemorrhagic disease in fish. The inventors analyzed the amino acid sequence provided at ABQ42711 (SEQ ID NO: 3787). Upon analysis, the inventors observed a Replikin Peak Gene having continuous Replikin sequences that begin at residue 81 (histidine) and continue through residue 204 (histidine).

The inventors isolated the RPG in silico for diagnostic and therapeutic uses in, for example, an immunogenic compound and a therapeutic vaccine compound and as a predictive sequence for lethal outbreaks of hemorrhagic disease in fish. Thirty-six Replikin sequences (SEQ ID NOS: 3788-3823) were identified in SEQ ID NO: 3787 for diagnostic, therapeutic and predictive uses as described herein. Replikin sequences SEQ ID NOS: 3788-3795 were identified in the amino-terminal, Replikin sequences SEQ ID NOS: 3796-3815 were identified in the mid-molecule of the sequence, and Replikin sequences 3816-3823 were identified in the carboxy-terminal. All were isolated for diagnostic, therapeutic and predictive purposes.

The Replikin Count of the whole sequence (SEQ ID NO: 3787) was 36 Replikin sequences in 222 total amino acids for a Replikin Count of 16. The highest Replikin Count of an identified RPG area in hemorrhagic septicemia virus was 73 Replikin sequences in 123 total amino acids for a Replikin Count of 59.

The inventors queried publicly available sequences from isolates of hemorrhagic viral disease syndrome in fish from 1990 through 2007. The following table provides the accession numbers queried. The highest Replikin Count of an identified RPG area in hemorrhagic septicemia virus was 73 Replikin sequences in 123 total amino acids for a Replikin Count of 59.

The inventors queried all sequences for hemorrhagic viral disease in fish publicly available at www.pubmed.com between 1990 and 2007 Using FluForecast® (Replikins LLC, Boston, Mass.), the inventors determined the mean Replikin Count in each year from 1990-2007. The data is provided in Table 29. The table does not included years in which no data was available.

TABLE 29
Hemorrhagis septicemia Mean Replikin Counts

| | | | Mean | | |
|---|---|---|---|---|---|
| | | No. of | Replikin | | |
| | | Isolates per | Count | | |
| Year | PubMed Accession Number-Replikin Count | year | per year | S.D. | Signficance |
| 1990 | BAA00591 3 CAA00881 29 CAA52082 33 | 9 | 5.0 | 2.4 | low p < .001 |
| | CAA52081 33 CAA52080 33 CAA52079 33 | | | | |
| | CAA52078 33 CAA52077 33 P24378 3 | | | | |
| 1991 | CAA41859 29 CAA41858 29 CAA01751 4 | 6 | 3.5 | 2.5 | low p < .001, |
| | CAA41930 4 P27663 2 P27662 29 | | | | prev p < .20 |
| 1992 | CAA46926 33 P27371 4 | 2 | 3.7 | 3.9 | low p < .10, |
| | | | | | prev p > .50 |
| 1993 | AAB26115 33 AAT01207 48 AAT01206 48 | 64 | 9.4 | 0.4 | low p < .001, |
| | AAT01205 48 AAT01204 48 AAT01203 48 | | | | prev p < .04 |
| | AAT01202 48 AAT01201 | | | | |
| | 48 AAT01200 48 AAT01199 48 AAT01198 48 | | | | |
| | AAT01197 48 AAT01196 48 AAT01195 48 | | | | |
| | AAT01194 48 AAT01193 | | | | |
| | 48 AAT01192 48 AAT01191 48 AAT01190 48 | | | | |
| | AAT01189 48 AAT01188 48 AAT01187 48 | | | | |
| | AAT01186 48 AAT01185 | | | | |
| | 48 AAT01184 48 AAT01183 48 AAT01182 48 | | | | |
| | AAT01181 48 AAT01180 48 AAT01179 48 | | | | |
| | AAT01178 48 AAT01177 | | | | |
| | 48 AAT01176 48 AAT01175 48 AAT01174 48 | | | | |
| | AAT01173 48 AAT01172 48 AAT01171 48 | | | | |
| | AAT01170 48 AAT01169 | | | | |
| | 48 AAT01168 48 AAT01167 48 AAT01166 48 | | | | |
| | AAT01165 48 AAT01164 48 AAT01163 48 | | | | |
| | AAT01162 48 AAT01161 | | | | |
| | 48 AAT01160 48 AAT01159 48 AAT01158 48 | | | | |
| | AAT01157 48 AAT01156 48 AAT01155 48 | | | | |
| | AAT01154 48 AAT01153 | | | | |
| | 48 AAT01152 48 AAT01151 48 AAT01150 48 | | | | |
| | AAT01149 48 AAT01148 48 AAT01147 48 | | | | |
| | AAT01146 48 AAT01145 48 | | | | |
| 1994 | | | | | |
| 1995 | AAB88231 33 AAB88230 38 AAB8829 41 | 5 | 5.9 | 2.7 | low p < .001, |
| | AAB88232 30 Q96460 6 | | | | prev p < .002 |
| 1997 | AAC24962 3 CAB59222 53 CAB59221 53 | 46 | 8.3 | 4.4 | low p < .001, |
| | CAB07737 53 CAB59220 53 CAB59219 53 | | | | prev p < .05 |
| | CAB59218 53 CAB59217 | | | | |
| | 52 CAB59216 52 CAB59215 52 CAB59214 52 | | | | |
| | CAB59213 52 CAB59212 52 AAB88228 52 | | | | |
| | AAB88227 | | | | |
| | 30 AAB88226 29 AAB88225 30 AAB88224 30 | | | | |
| | AAB88223 23 AAB88222 30 CAB07754 15 | | | | |
| | CAB07753 | | | | |
| | 17 CAB07752 14 CAB07751 7 CAB07750 14 | | | | |
| | CAB07749 14 CAB07748 14 CAB07747 11 | | | | |
| | CAB07746 | | | | |
| | 14 CAB07745 14 CAB07744 14 CAB07743 15 | | | | |
| | CAB07742 15 CAB07741 7 CAB07740 15 | | | | |
| | CAB07739 | | | | |
| | 14 CAB07738 14 CAB07736 11 CAB07734 20 | | | | |
| | CAB07733 11 CAB07732 14 CAB07731 15 | | | | |
| | CAB07730 | | | | |
| | 3 CAB07729 14 CAB07728 14 CAB07727 15 | | | | |
| 1998 | NP_049550 55 NP_049549 55 NP_049548 55 | 20 | 2.9 | 0.6 | low p < .001, |
| | NP_049547 55 NP_049546 55 NP_049545 55 | | | | prev p < .001 |
| | CAB57984 | | | | |
| | 1 CAB44726 55 CAB44725 55 CAB44724 55 | | | | |
| | CAB44723 55 CAB44722 55 CAB44721 55 | | | | |
| | CAB40833 | | | | |
| | 66 CAB40832 66 CAB40831 66 CAB40830 66 | | | | |
| | CAB40829 66 CAB40828 66 CAA08837 58 | | | | |
| 1999 | AAF04486 57 AAF04485 57 AAF04484 57 | 13 | 2.9 | 0.3 | low p < .001, |
| | AAF04483 57 AAF04482 57 AAF04481 57 | | | | prev p > .50 |
| | AAF04480 53 | | | | |
| | AAF04479 53 AAF04478 53 AAF04477 53 | | | | |
| | AAF04476 53 AAF04475 53 BAC29401 14 | | | | |
| 2000 | | | | | |
| 2001 | BAB70674 15 BAB70673 16 BAB70672 16 | 7 | 7.4 | 0.7 | low p < .001, |
| | BAB70671 16 AAL83805 32 AAL83804 35 | | | | prev p < .001 |
| | AAL83803 40 | | | | |
| 2002 | AAN85721 30 CAD31945 13 CAD31944 7 | 7 | 8.1 | 2.5 | low p < .001, |
| | CAD31943 7 CAD31941 7 CAD31924 7 | | | | prev p > .50 |
| | CAD31923 7 | | | | |
| 2003 | ABF17852 20 ABF17851 20 ABF17850 20 | 13 | 8.1 | 3.6 | low p < .001, |
| | ABF17849 20 ABF17848 20 ABF17847 20 | | | | prev p > .50 |
| | ABF17846 | | | | |
| | 20 ABF17845 20 NP_997523 30 NP_997978 41 | | | | |
| | NP_997977 21 NP_001013287 33 NP_891987 29 | | | | |
| 2004 | AAU12246 26 AAU12245 26 AAU12244 26 | 34 | 10.6 | 3.2 | low p < .001, |
| | AAU12243 26 AAU12242 26 AAU12241 26 | | | | prev p < .02 |
| | AAU12240 | | | | |
| | 26 AAU12239 26 AAU12238 26 AAU12237 26 | | | | |
| | AAU12236 26 AAU12235 26 AAU12234 26 | | | | |
| | AAU12233 | | | | |
| | 26 AAU12232 26 AAU12231 26 AAU12230 26 | | | | |
| | AAU12229 26 AAU12228 26 AAU12227 26 | | | | |
| | AAU12226 | | | | |
| | 26 AAU12225 26 AAU12224 26 AAU12223 26 | | | | |
| | AAU12222 26 AAU12221 26 AAU12220 26 | | | | |
| | AAU12219 | | | | |
| | 26 BAD72126 42 BAD72124 30 BAD72123 8 | | | | |
| | BAD72122 16 BAD72121 4 NP_998029 8 | | | | |
| 2005 | CAJ31050 28 CAJ31049 28 CAJ31048 28 | 62 | 5.6 | 0.7 | low p < .001, |
| | CAJ31047 28 CAJ31046 28 CAJ31045 28 | | | | prev p < .001 |
| | CAJ31044 28 CAJ31043 | | | | |
| | 28 CAJ31042 28 CAJ31041 28 CAJ31040 28 | | | | |
| | CAJ31039 28 CAJ31038 28 CAJ31037 28 | | | | |
| | CAJ31036 28 CAJ31035 | | | | |
| | 28 CAJ31034 28 CAJ31033 28 CAJ31032 28 | | | | |
| | CAJ31031 28 CAJ31030 28 CAJ31029 28 | | | | |
| | CAJ31028 28 CAJ31027 | | | | |
| | 28 CAJ31026 28 CAJ31025 28 CAJ31024 28 | | | | |
| | CAJ31023 28 CAJ31022 28 CAJ31021 28 | | | | |
| | CAJ31020 28 CAJ31019 | | | | |
| | 28 CAJ31018 21 CAJ31017 21 CAJ31016 28 | | | | |
| | CAJ31015 28 CAJ31014 28 CAJ31013 28 | | | | |
| | CAJ31012 28 CAJ31011 | | | | |
| | 28 CAJ31010 28 CAJ31009 28 CAJ31008 28 | | | | |
| | CAJ31007 28 CAJ31006 28 CAJ31005 28 | | | | |
| | CAJ31004 28 CAJ31003 | | | | |
| | 28 CAJ31002 28 CAJ31001 28 CAJ31000 28 | | | | |
| | CAJ30999 28 CAJ30998 28 CAJ30997 28 | | | | |
| | CAJ30996 28 CAJ30995 | | | | |
| | 28 CAJ30994 28 CAJ30993 28 CAJ30992 28 | | | | |
| | CAJ30991 28 BAE78962 17 BAE78961 16 | | | | |
| 2006 | ABN13930 16 ABN13929 16 ABN13928 16 | 16 | 7.8 | 2.8 | low p < .001, |
| | ABN13927 16 ABN13926 2 ABD96102 4 | | | | prev p < .002 |
| | ABD64588 47 ABD64587 | | | | |
| | 47 ABD64586 47 ABD64585 47 ABD64584 47 | | | | |
| | ABD64583 47 ABD64582 47 ABD64581 47 | | | | |
| | ABD64580 | | | | |
| | 47 ABD64579 47 | | | | |
| 2007 | ABQ42711 36 | 1 | 16.2 | 0.0 | prev p < .001 |

What is claimed is:

1. A method of identifying a first virus, first organism or first malignancy with a higher lethality than at least one second virus of the same species as the first virus, second organism of the same species as the first organism, or second malignancy of the same species as the first malignancy, which method comprises comparing the Replikin Count of the Replikin Peak Gene of the first virus, first organism or first malignancy to the Replikin Count of the Replikin Peak Gene of at least one second virus, second organism, or second malignancy to determine that the virus, organism or malignancy with the higher Replikin Count is the more lethal.

2. The method of claim 1 wherein said first malignancy is a lung malignancy, a brain malignancy, a breast malignancy, an ovarian malignancy, or a lymph malignancy.

3. The method of claim 2 wherein said first malignancy is a non-small cell lung carcinoma.

4. The method of claim 1 wherein said first organism is a Mycobacterium tuberculosis, Mycobacterium mucogenicum, Staphylococcus aureus, or Plasmodium falciparum.

5. The method of claim 1 wherein said first virus is influenza virus, foot and mouth disease virus, west nile virus, porcine respiratory and reproductive syndrome virus, porcine circovirus, white spot syndrome virus, taura syndrome virus, coronavirus, ebola virus, gemini leaf curl virus, hemorrhagic septicemia virus, or tobacco mosaic virus.

6. The method of claim 5 wherein said first virus is Influenza A virus strain H1N1, H2N2, H3N2, H5N1, or H3N8.

7. An isolated or synthesized Replikin Peak Gene of a virus, organism or malignancy wherein said Replikin Peak Gene is identified as the portion of the genome, protein or protein fragment of a virion of the virus, a cell of the organism or a malignant cell of the malignancy consisting of the highest number of continuous Replikin sequences per 100 amino acids as compared to other portions of the genome, protein or protein fragment of the virion of the virus, the cell of the organism or the malignant cell of the malignancy.

8. The isolated or synthesized Replikin Peak Gene of claim 7 wherein the Replikin Peak Gene is the portion of a protein or protein fragment consisting of the highest number of continuous Replikin sequences per 100 amino acids as compared to all other proteins or protein fragments in the virion of the virus, in the cell of the organism or in the malignant cell of the malignancy.

9. The isolated or synthesized Replikin Peak Gene of claim 7 wherein said Replikin Peak Gene is isolated from a lung malignancy, a brain malignancy, a breast malignancy, an ovarian malignancy, or a lymph malignancy.

10. The isolated or synthesized Replikin Peak Gene of claim 9 wherein said Replikin Peak Gene is isolated from a non-small cell lung carcinoma or glioblastoma multiforme.

11. The isolated or synthesized Replikin Peak Gene of claim 7 wherein said Replikin Peak Gene is isolated from Mycobacterium tuberculosis, Mycobacterium mucogenicum, Staphylococcus aureus, or Plasmodium falciparum.

12. The isolated or synthesized Replikin Peak Gene of claim 7 wherein said Replikin Peak Gene is isolated from influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

13. The isolated or synthesized Replikin Peak Gene of claim 12 wherein the influenza virus is an Influenza A virus.

14. The isolated or synthesized Replikin Peak Gene of claim 13 wherein said Influenza A virus is H1N1, H2N2, H3N2, H5N1 or H3N8.

15. The isolated or synthesized Replikin Peak Gene of claim 13 wherein said Replikin Peak Gene is isolated from the pB1 gene area of an influenza virus.

16. The isolated or synthesized Replikin Peak Gene of claim 12 wherein said virus is a foot and mouth disease virus.

17. The isolated or synthesized Replikin Peak Gene of claim 16 wherein said Replikin Peak Gene is identified within the VP1 gene of said foot and mouth disease virus.

18. The isolated or synthesized Replikin Peak Gene of claim 12 wherein said virus is west nile virus.

19. The isolated or synthesized Replikin Peak Gene of claim 18 wherein said Replikin Peak Gene is isolated from the envelope protein of said west nile virus.

20. The isolated or synthesized Replikin Peak Gene of claim 12 wherein said virus is porcine respiratory and reproductive syndrome virus.

21. The isolated or synthesized Replikin Peak Gene of claim 20 wherein said Replikin Peak Gene is isolated from a nucleocapsid protein of porcine respiratory and reproductive syndrome virus.

22. The isolated or synthesized Replikin Peak Gene of claim 12 wherein said virus is porcine circovirus.

23. The isolated or synthesized Replikin Peak Gene of claim 22 wherein said Replikin Peak Gene is isolated from a replicase protein of porcine circovirus.

24. The isolated or synthesized Replikin Peak Gene of claim 12 wherein said virus is white spot syndrome virus.

25. The isolated or synthesized Replikin Peak Gene of claim 24 wherein said Replikin Peak Gene is isolated from a ribonucleotide reductase protein of white spot syndrome virus.

26. The isolated or synthesized Replikin Peak Gene of claim 12 wherein said virus is tobacco mosaic virus.

27. The isolated or synthesized Replikin Peak Gene of claim 12 wherein said virus is hemorrhagic septicemia virus.

28. The isolated or synthesized Replikin Peak Gene of claim 27 wherein said Replikin Peak Gene is isolated from a glycoprotein in hemorrhagic septicemia virus.

29. The isolated or synthesized Replikin Peak Gene of claim 7 wherein said Replikin Peak Gene comprises a sequence of SEQ ID NO: 1741, SEQ ID NO: 3664, SEQ ID NO: 3660, SEQ ID NO: 3665, SEQ ID NO: 1996, SEQ ID NO: 1665, SEQ ID NO: 1684, SEQ ID NO: 1701, SEQ ID NO: 546, SEQ ID NO: 124, SEQ ID NO: 130, SEQ ID NO: 311, SEQ ID NOS: 341-344, SEQ ID NO: 286, SEQ ID NO: 287, SEQ ID NO: 288, SEQ ID NO: 289, SEQ ID NO: 290, SEQ ID NOS: 233-238, SEQ ID NO: 415, SEQ ID NO: 421, SEQ ID NO: 438, SEQ ID NO: 451, SEQ ID NO: 462, SEQ ID NO: 498, SEQ ID NO: 669, SEQ ID NO: 1168, SEQ ID NO: 1531, SEQ ID NO: 1548, positions 81-204 of SEQ ID NO: 3787, or SEQ ID NO: 1939.

30. An immunogenic composition comprising the isolated or synthesized Replikin Peak Gene of claim 7.

31. The immunogenic composition comprising a Replikin sequence isolated from a Replikin Peak Gene of claim 7 wherein said Replikin sequence is SEQ ID NOS: 2902-2925, SEQ ID NOS: 2312-2544, SEQ ID NOS: 2701-2711, SEQ ID NOS: 2713-2718, SEQ ID NOS: 3282-3285, 3287-3291, 3293, 3295, 3297, 3299, 3300, 3302, 3304, 3306, and 3308, SEQ ID NOS: 1685-1691, SEQ ID NOS: 1702-1717, SEQ ID NO: 106, SEQ ID NO: 112, SEQ ID NO: 113, SEQ ID NOS: 125-129, SEQ ID NOS: 131-156, SEQ ID NOS: 233-244, SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-414, SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484 and SEQ ID NOS: 487-492, SEQ ID NOS: 547-562. SEQ ID NOS: 663-667, SEQ ID NOS: 670-1166, SEQ ID NOS: 1169-1529, SEQ ID NOS: 1532-1542, SEQ ID NO: 1548, positions 81-204 of SEQ ID NO: 3787, or SEQ ID NOS 1637-1663.

32. A computer readable medium having stored thereon instructions which, when executed, cause the processor to perform a method for identifying a Replikin Peak Gene of a virus, organism or malignancy comprising identifying, within amino acid sequences or nucleic acid sequences that encode amino acid sequences of said virus, organism or malignancy, the portion of the genome, or protein or protein fragment of said virus, said organism or said malignancy consisting of the highest number of continuous Replikin sequences per 100 amino acids as compared to other portions of the genome, or protein or protein fragment of the virus, organism or malignancy.

33. The computer readable medium of claim 32 further comprising instructions which, when executed, cause the processor to perform a method for predicting an increase in lethality or virulence of said virus, organism or malignancy that comprises said identified Replikin Peak Gene or an outbreak of said virus or organism that comprises said identified Replikin Peak Gene comprising:
(1) determining that the Replikin Count of said Replikin Peak Gene of claim 32 or that the Replikin Count of a protein or gene area comprising said Replikin Peak Gene of claim 32 is higher than another Replikin Peak Gene or a protein or gene area comprising said other Replikin Peak Gene identified within the genome or within a protein or protein fragment of at least one other virus of the same species as said virus, at least one other organism of the same species as said organism or at least one other malignancy of the same type as said malignancy wherein said other virus, said other organism or said other malignancy is isolated at an earlier time point than said virus, said organism or said malignancy, and
(2) predicting an increase in lethality or virulence of said virus, organism or malignancy or predicting an outbreak of said virus or organsism.

34. A method of predicting the strain, the host or the geographic region of an outbreak or increase in lethality or virulence of a virus or organism comprising
(1) identifying a Replikin Peak Gene or a protein or gene area comprising a Replikin Peak Gene within the genome of a first virus or organism of a first strain, from a first host, or isolated from a first geographic region or within a protein or protein fragment of the first virus or organism that has a higher Replikin Count than a Replikin Peak Gene or protein or gene area comprising a Replikin Peak Gene identified within the genome or within a protein or protein fragment of at least one second virus of the same species as the first virus or at least one second organism of the same species as the first organism wherein said first virus or said first organism is isolated at a later time point than said first virus or said first organism and is the same strain, from the same or another host or isolated from the same or another geographic region as the first virus or first organism, and
(2) predicting an outbreak or an increase in lethality or virulence of said first strain, in said first host, or within said first geographic region of said first virus or organism.

35. The method of claim 34 wherein said protein or gene area comprising said Replikin Peak Gene within the genome of a first virus or organism is identified as having a higher Replikin Count than said protein or gene area comprising a Replikin Peak Gene identified within the genome or within a protein or protein fragment of said at least one second virus or organism.

36. The method of claim 34 wherein said first virus or said first organism is isolated at least six months to three years later than said second organism or said second virus.

37. The method of claim 34 wherein said first organism or said first virus is Mycobacterium tuberculosis, Mycobacterium mucogenicum, Staphylococcus aureus, and Plasmodium falciparum, influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus in tomato plants, hemorrhagic septicemia virus in fish, or tobacco mosaic virus.

38. The method of claim 37 wherein said Staphylococcus aureus is methicillin-resistant.

39. The method of claim 37 wherein said influenza virus is a strain of Influenza A virus.

40. The method of claim 39 wherein said Influenza A virus is H1N1, H2N2, H3N2, H5N1 or H3N8.

41. The method of claim 39 wherein said protein or gene area comprising said Replikin Peak Gene is the pB1 gene area of the influenza virus.

42. The method of claim 37 wherein said protein or gene area is a nucleocapsid protein of said porcine respiratory and reproductive syndrome virus.

43. The method of claim 37 wherein said protein or gene area is a VP1 protein of said foot and mouth disease virus.

44. The method of claim 37 wherein said protein or gene area is an envelope protein of said west nile virus.

45. The method of claim 37 wherein said protein or gene area is a nucleocapsid protein of said porcine reproductive and respiratory syndrome virus.

46. The method of claim 37 wherein said protein or gene area is an ATP-ase of said Plasmodium falciparum.

47. The method of claim 37 wherein said protein or gene area is a replicase protein of said porcine circovirus.

48. The method of claim 37 wherein said protein or gene area is a ribonucleotidease of said white spot syndrome virus.

49. A method of identifying a first virus, organism or malignancy associated with higher lethality, higher virulence or more rapid replication than a second virus of the same species as the first virus, a second organism of the same species as the first organism or a second malignancy of the same type as the first malignancy comprising identifying a Replikin Peak Gene encoded within the genome of at least one virion of the first virus, or at least one cell of the first organism, or at least one malignant cell of the first malignancy, or within a protein or protein fragment of at least one virion of the first virus, or at least one cell of the first organism, or at least one malignant cell of the first malignancy that has a higher Replikin Count than a Replikin Peak Gene identified encoded within the genome of at least one virion of the second virus, or at least one cell of the second organism, or at least one malignant cell of the second malignancy or within a protein or protein fragment of at least one virion of the second virus, or at least one cell of the second organism, or at least one malignant cell of the second malignancy wherein said first virus, first organism or first malignancy has higher lethality, higher virulence or more rapid replication than said second virus, second organism or second malignancy, and wherein the Replikin Peak Gene is defined as a protein or protein fragment having the highest concentration of continuous Replikin sequences per 100 amino acids as compared to the remaining proteins or protein fragments in the same virion of the virus, the same cell of the organism, or the same malignant cell, or the portion of the genome encoding the protein or protein fragment.

50. A method of identifying a first virus, first organism or first malignancy with a higher lethality than at least one second virus of the same species as the first virus, second organism of the same species as the first organism or second malignancy of the same species as the first malignancy comprising comparing the Replikin Count of the whole genome of said virus, organism or malignancy to the Replikin Count of the whole genome of said at least one second virus, second organism, or second malignancy to determine that the virus, organism or malignancy with the higher Replikin Count is the more lethal.

51. The method of claim 50 wherein said first virus is a coronavirus, a foot and mouth disease virus, a white spot syndrome virus, a taura syndrome virus, a porcine circovirus, or an influenza virus.

52. The method of claim 50 wherein said first virus is an H5N1 strain of influenza virus.

53. A method for obtaining an isolated or synthesized Replikin Peak Gene of a virus, organism or malignancy for diagnosis, prevention or treatment of an infection of said virus or said organism or for diagnosis, prevention or treatment of said malignancy comprising:
(1) obtaining a plurality of isolates of virus of the same species, a plurality of organisms of the same species, or a plurality of malignancies of the same type;
(2) analyzing the protein sequences or protein sequence fragments of each individual isolate of the plurality of isolates of virus, a cell of each individual organism of the plurality of organisms, or a malignant cell of each individual malignancy of the plurality of malignancies for the presence and concentration of Replikin sequences;
(3) identifying the protein sequence or the protein sequence fragment having the highest concentration of continuous Replikin sequences in the malignant cell of each individual malignancy, the cell of each individual organism or each individual virus isolate;
(4) selecting the protein sequence or protein sequence fragment having the highest concentration of continuous Replikin sequences among the plurality of isolates of virus, the plurality of organisms, or the plurality of malignancies;
(5) identifying the amino acid sequence of the selected protein sequence or protein sequence fragment as the Replikin Peak Gene of the plurality of virus isolates, organisms or malignancies; and
(6) isolating or synthesizing the identified Replikin Peak Gene of at least one of the plurality of virus isolates, organisms or malignancies wherein the isolated or synthesized identified Replikin Peak Gene is useful for diagnosis, prevention or treatment of said infection of said virus or said organism or said malignancy.

54. An immunogenic composition comprising at least one isolated or synthesized Replikin Peak Gene of claim 53.

55. The immunogenic composition of claim 54 wherein said isolated or synthesized Replikin Peak Gene is isolated from an emerging strain of a virus or organism.

56. The immunogenic composition of claim 54 further comprising a pharmaceutically acceptable carrier.

57. A vaccine comprising at least one isolated or synthesized Replikin Peak Gene of claim 7.

58. The vaccine of claim 57 wherein said at least one isolated or synthesized Replikin Peak Gene is isolated from an emerging strain of virus or organism.

59. A vaccine of claim 57 comprising SEQ ID NO: 1741, SEQ ID NO: 3664, SEQ ID NO: 3660, SEQ ID NO: 3665, SEQ ID NO: 1996, SEQ ID NO: 1665, SEQ ID NO: 1684, SEQ ID NO: 1701, SEQ ID NO: 546, SEQ ID NO: 124, SEQ ID NO: 130, SEQ ID NO: 311, SEQ ID NOS: 341-344, SEQ ID NO: 286, SEQ ID NO: 287, SEQ ID NO: 288, SEQ ID NO: 289, SEQ ID NO: 290, SEQ ID NOS: 233-238, SEQ ID NO: 415, SEQ ID NO: 421, SEQ ID NO: 438, SEQ ID NO: 451, SEQ ID NO: 462, SEQ ID NO: 498, SEQ ID NO: 669, SEQ ID NO: 1168, SEQ ID NO: 1531, SEQ ID NO: 1548, positions 81-204 of SEQ ID NO: 3787, or SEQ ID NO: 1939.

60. The vaccine of claim 57 wherein said isolated or synthesized Replikin Peak Gene is isolated from a virus.

61. The vaccine of claim 60 wherein said isolated or synthesized-Replikin Peak Gene is isolated from influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

62. The vaccine of claim 61 wherein said isolated or synthesized Replikin Peak Gene is isolated from Influenza A.

63. The vaccine of claim 62 wherein said Influenza A is H1N1, H2N2, H3N2, H5N1 or H3N8.

64. The vaccine of claim 57 wherein said isolated or synthesized Replikin Peak Gene is isolated from an organism.

65. The vaccine of claim 64 wherein said isolated or synthesized Replikin Peak Gene is isolated from Mycobacterium tuberculosis, Mycobacterium mucogenicum, Staphylococcus aureus, or Plasmodium falciparum.

66. The vaccine of claim 65 wherein said Staphylococcus aureus is methicillin-resistant.

67. The vaccine of claim 57 wherein said isolated or synthesized Replikin Peak Gene is isolated from a malignancy.

68. The vaccine of claim 67 wherein said isolated or synthesized Replikin Peak Gene isolated is isolated from a lung malignancy, a brain malignancy, a breast malignancy or a lymph malignancy.

69. The vaccine of claim 68 wherein said isolated or synthesized Replikin Peak Gene is isolated from a non-small cell lung carcinoma.

70. The vaccine of claim 68 wherein said isolated or synthesized Replikin Peak Gene is isolated from glioblastoma multiforme.

71. An immunogenic composition comprising a Replikin Peak Gene of claim 29.

72. The immunogenic composition of claim 71 further comprising a pharmaceutically acceptable carrier.

73. An isolated or synthesized Replikin sequence isolated from a protein or protein fragment of the Replikin Peak Gene of claim 7 or isolated from a protein comprising the Replikin Peak Gene of claim 7.

74. The Replikin sequence of claim 73 wherein said Replikin Peak Gene is isolated from Mycobacterium tuberculosis, Mycobacterium mucogenicum, Staphylococcus aureus, or a Plasmodium falciparum.

75. The Replikin sequence of claim 74 wherein said Replikin Peak Gene is isolated from Mycobacterium mucogenicum.

76. The Replikin sequence of claim 75 wherein said Replikin sequence is SEQ ID NOS: 2902-2925.

77. The Replikin sequence of claim 74 wherein said Replikin Peak Gene is isolated from Plasmodium falciparum.

78. The Replikin sequence of claim 77 wherein said Replikin Peak Gene is one of SEQ ID NOS: 2312-2544, SEQ ID NOS: 2701-2711, SEQ ID NOS: 2713-2718, SEQ ID NOS: 3282-3285, 3287-3291, 3293, 3295, 3297, 3299, 3300, 3302, 3304, 3306, or SEQ ID NO: 3308.

79. The Replikin sequence of claim 73 wherein said Replikin Peak Gene is isolated from influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

80. The Replikin sequence of claim 79 wherein said influenza virus is Influenza A virus.

81. The Replikin sequence of claim 80 wherein said Influenza A virus is HIN1, H2N2, H3N2, H5N1 or H3N8.

82. The Replikin sequence of claim 81 wherein said Influenza A virus is H5N1 and the Replikin sequence is one of SEQ ID NOS: 1685-1691, SEQ ID NOS: 1702-1716 or SEQ ID NO: 1717.

83. The Replikin sequence of claim 81 wherein said Influenza A virus is H3N8 and the Replikin sequence is one of SEQ ID NOS: 547-561 or SEQ ID NO: 562.

84. The Replikin sequence of claim 79 wherein said Replikin Peak Gene is isolated from foot and mouth disease virus.

85. The Replikin sequence of claim 84 wherein said Replikin sequence is one of SEQ ID NO: 106, SEQ ID NO: 112, SEQ ID NO: 113, SEQ ID NOS: 125-129, SEQ ID NOS: 131-155 or SEQ ID NO: 156.

86. The Replikin sequence of claim 79 wherein said Replikin Peak Gene is isolated from west nile virus.

87. The Replikin sequence of claim 86 wherein said Replikin sequence is one of SEQ ID NOS: 233-243 or SEQ ID NO: 244.

88. The Replikin sequence of claim 79 wherein said Replikin Peak Gene is isolated from PRRSV.

89. The Replikin sequence of claim 88 wherein said Replikin sequence is one of SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-413 or SEQ ID NO: 414.

90. The Replikin sequence of claim 79 wherein said Replikin Peak Gene is isolated from PCV.

91. The Replikin sequence of claim 90 wherein said Replikin sequence is one of SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484, SEQ ID NOS: 487-491 or SEQ ID NO: 492.

92. The Replikin sequence of claim 79 wherein said Replikin Peak Gene is isolated from white spot syndrome virus.

93. The Replikin sequence of claim 92 wherein said Replikin sequence is one of SEQ ID NOS: 663-667, SEQ ID NOS: 670-1166, SEQ ID NOS: 1169-1529, SEQ ID NOS: 1532-1542 and SEQ ID NO: 1548.

94. A vaccine for prevention and/or treatment of a viral or organismal infection or a malignancy wherein the vaccine comprises at least one isolated or synthesized Replikin sequence within a protein or protein fragment of a Replikin Peak Gene or a protein comprising a Replikin Peak Gene identified in said virus, organism, or malignancy.

95. The vaccine of claim 94 wherein said at least one isolated or synthesized Replikin sequence is one of SEQ ID NOS: 2902-2925, SEQ ID NOS: 2312-2544, SEQ ID NOS: 2701-2711, 2713-2718, SEQ ID NOS: 3282-3285, 3287-3291, 3293, 3295, 3297, 3299, 3300, 3302, 3304, 3306, 3308, SEQ ID NOS: 1685-1691, SEQ ID NOS: 1702-1717, SEQ ID NOS: 547-562, SEQ ID NO: 106, SEQ ID NO: 112, SEQ ID NO: 113, SEQ ID NOS: 125-129, and SEQ ID NOS: 131-156, SEQ ID NOS: 233-244, SEQ ID NOS: 286-290, SEQ ID NOS: 312-323, SEQ ID NOS: 354-366, SEQ ID NOS: 368-380, SEQ ID NOS: 383-393, SEQ ID NOS: 395-401, SEQ ID NOS: 403-414, SEQ ID NOS: 291-307, SEQ ID NOS: 308-310, SEQ ID NOS: 324-327, SEQ ID NOS: 328-340, SEQ ID NOS: 416-419, SEQ ID NOS: 422-437, SEQ ID NOS: 440-445, SEQ ID NOS: 452-457, SEQ ID NOS: 464-476, SEQ ID NOS: 482-484 SEQ ID NOS: 487-492, SEQ ID NOS: 663-667, SEQ ID NOS: 670-1166, SEQ ID NOS: 1169-1529, SEQ ID NOS: 1532-1542, SEQ ID NO: 1548, SEQ ID NOS: 1637-1662, or SEQ ID NO: 1663.

96. The vaccine of claim 94 for prevention and/or treatment of a viral infection.

97. The vaccine of claim 96 wherein said viral infection is caused by influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus, or tobacco mosaic virus.

98. The vaccine of claim 97 wherein the influenza virus is Influenza A virus.

99. The vaccine of claim 98 wherein the Influenza A virus is a H1N1, H2N2, H3N2, H5N1 or H3N8.

100. The vaccine of claim 97 wherein the virus is hemorrhagic septicemia virus.

101. The vaccine of claim 94 for prevention and/or treatment of an organismal infection.

102. The vaccine of claim 101 wherein the organismal infection is caused by Mycobacterium tuberculosis, Mycobacterium mucogenicum, Staphylococcus aureus, or Plasmodium falciparum.

103. The vaccine of claim 102 wherein said Staphylococcus aureus is methicillin-resistant.

104. The vaccine of claim 94 wherein the malignancy is a lung malignancy, a brain malignancy, a breast malignancy, an ovarian malignancy, or a lymph malignancy.

105. The vaccine of claim 104 wherein the malignancy is non-small cell lung carcinoma or glioblastoma multiforme.

106. An immunogenic compound comprising at least one isolated or synthesized Replikin sequence within the protein or protein fragment of a Replikin Peak Gene or within a protein comprising a Replikin Peak Gene wherein said Replikin Peak Gene is identified in a virus, an organism or a malignancy.

107. The immunogenic compound of claim 106 further comprising a pharmaceutically acceptable carrier.

108. A method of stimulating the immune system, comprising administering in an animal at least one isolated or synthesized Replikin sequence identified within a protein or protein fragment of a Replikin Peak Gene or within a protein or gene area comprising a Replikin Peak Gene identified in a virus, organism, or malignancy.

109. The method of claim 108 wherein said animal is a human.

110. An antibody to at least one isolated or synthesized Replikin sequence within a protein or protein fragment of Replikin Peak Gene or within a protein or gene area comprising a Replikin Peak Gene.

111. A method of identifying a lethal strain of malignancy, organism or virus comprising:
(1) obtaining a plurality of isolates of said malignancy, organism or virus;
(2) identifying the Replikin Peak Gene in each isolate of the plurality of isolates of said malignancy, organism or virus;
(3) analyzing the amino acid sequence of a protein or protein fragment of the Replikin Peak Gene of each isolate of the plurality of isolates for the presence and concentration of Replikin sequences;
(4) comparing the concentrations of Replikin sequences in each of the proteins or protein fragments of the Replikin Peak Gene of each isolate of the plurality of isolates to the concentration of Replikin sequences in each of the proteins or protein fragments of the Replikin Peak Gene of each of the other isolates of the plurality of isolates; and
(5) identifying the isolate having the highest concentration of continuous Replikin sequences in the protein or protein fragment of the Replikin Peak Gene as a virulent or lethal strain of said malignancy, organism or virus.

112. A method of selecting a peptide from a malignancy, organism or virus for inclusion in a preventive or therapeutic vaccine or immunogenic compound for a malignancy, organism or virus comprising identifying at least one difference in the amino acid sequence of an otherwise conserved Replikin sequence or Replikin Peak Gene between at least two isolates of said malignancy, organism or virus and correlating the identified at least one difference in the amino acid sequence with the highest virulence, morbidity or host mortality among the at least two isolates and selecting an otherwise conserved Replikin sequence, Replikin Peak Gene or Replikin sequence within a Replikin Peak Gene having the identified at least one amino acid sequence difference as the peptide for inclusion in a preventive or therapeutic vaccine or immunogenic compound.

113. The method of claim 112 further predicting the isolate comprising the selected conserved Replikin sequence or Replikin Peak Gene having the at least one difference in the amino acid sequence to be a lethal isolate of said malignancy, organism or virus.

114. The method of claim 113 wherein said malignancy, organism or virus is a malignancy.

115. The method of claim 114 wherein said malignancy is a lung malignancy, a brain malignancy, a breast malignancy or a lymph malignancy.

116. The method of claim 115 wherein said malignancy is a non-small cell lung carcinoma or a glioblastoma multiforme.

117. The method of claim 112 wherein said malignancy, organism or virus is an organism.

118. The method of claim 117 wherein said organism is Mycobacterium tuberculosis, Mycobaterium mucogenicum, Staphylococcus aureus, or Plasmodium falciparum.

119. The method of claim 118 wherein said Staphylococcus aureus is methicillin-resistant.

120. The method of claim 112 wherein said malignancy, organism or virus is a virus.

121. The method of claim 120 wherein said virus is influenza virus, foot and mouth disease virus, west nile virus, porcine reproductive and respiratory syndrome virus, porcine circovirus, white spot syndrome virus, taura syndrome virus, coronavirus, ebola virus, gemini leaf curl virus, hemorrhagic septicemia virus or tobacco mosaic virus.

122. A method of determining a source of a case of lung malignancy comprising identifying at least one peptide in a Replikin Peak Gene of a lung cancer cell that is also present in a Replikin Peak Gene of an isolate of tobacco mosaic virus, wherein the peptide is involved with the source of the lung malignancy.

123. The method of claim 122 wherein a plurality of peptides is identified in the Replikin Peak Gene of the lung cancer cell wherein each one of the plurality of peptides is also identified in the Replikin Peak Gene of an isolate of tobacco mosaic virus.

124. The method of claim 122 wherein the at least one peptide in the Replikin Peak Gene of the lung cancer cell and the at least one peptide in Replikin Peak Gene of the isolate of tobacco mosaic virus is a peptide of about 10 amino acids or less comprising at least two lysines and at least one histidine.

125. The method of claim 124 wherein the at least one peptide in the Replikin Peak Gene of the lung cancer cell and the at least one peptide in Replikin Peak Gene of the isolate of tobacco mosaic virus is a peptide of about 10 amino acids or less comprising at least three lysines and at least one histidine.

126. The method of claim 125 wherein the at least one peptide in the Replikin Peak Gene of the lung cancer cell and the at least one peptide in the Replikin Peak Gene of the isolate of tobacco mosaic virus is about 7 amino acids or less comprising at least three lysines and at least one histidine.

127. The method of claim 126 wherein the at least one peptide in the Replikin Peak Gene of the lung cancer cell and the at least one peptide in the Replikin Peak Gene of the isolate of tobacco mosaic virus is about 4 amino acids comprising three lysines and one histidine.

128. The method of claim 127 wherein the at least one peptide in the Replikin Peak Gene of the lung cancer cell and in the Replikin Peak Gene of the isolate of tobacco mosaic virus is KHKK (SEQ ID NO: 1584).

129. The method of claim 128 wherein more than one KHKK (SEQ ID NO: 1584) peptide is identified in the Replikin Peak Gene of the lung cancer cell and in the Replikin Peak Gene of the isolate of tobacco mosaic virus.

130. The method of claim 129 wherein at least 10 KHKK (SEQ ID NO: 1584) peptides are identified in the Replikin Peak Gene of the lung cancer cell and at least 10 KHKK (SEQ ID NO: 1584) peptides are identified in the Replikin Peak Gene of the isolate of tobacco mosaic virus.

131. The method of claim 130 wherein at least 20 KHKK (SEQ ID NO: 1584) peptides are identified in the Replikin Peak Gene of the lung cancer cell and at least 20 K-HKK (SEQ ID NO: 1584) peptides are identified in the Replikin Peak Gene of the isolate of tobacco mosaic virus.

132. The method of claim 131 wherein at least 30 KHKK (SEQ ID NO: 1584) peptides are identified in the Replikin Peak Gene of the lung cancer cell and at least 30 KHKK peptides are identified in the Replikin Peak Gene of the isolate of tobacco mosaic virus.

133. The method of claim 132 wherein at least 50 KHKK (SEQ ID NO: 1584) peptides are identified in the Replikin Peak Gene of the lung cancer cell and at least 50 KHKK (SEQ ID NO: 1584) peptides are identified in the Replikin Peak Gene of the isolate of tobacco mosaic virus.

134. A method of identifying a first case of malignancy of the lung having a higher rate of replication, aggressive growth pattern or lethality as compared with a second case of malignancy of the lung comprising identifying a Replikin Peak Gene in a malignant cell from a first case of malignancy of the lung that has a higher Replikin Count in the Replikin Peak Gene than a Replikin Peak Gene identified in a malignant cell from a second case of malignancy of the lung.

135. The method of claim 134 wherein the first and second cases of malignancy of the lung are non-small cell lung malignancies.

136. An isolated or synthesized Replikin Peak Gene in a lung malignancy for diagnosis, prevention or treatment of lung cancer by the method comprising:
(1) obtaining at least one malignant cell from a lung malignancy;
(2) analyzing the protein sequences or protein sequence fragments of the at least one malignant cell for the presence and concentration of Replikin sequences;
(3) identifying the protein sequence or the protein sequence fragment having the highest concentration of continuous Replikin sequences in the at least one malignant cell;
(4) selecting the protein sequence or protein sequence fragment having the highest concentration of continuous Replikin sequences;
(5) identifying the amino acid sequence of the selected protein sequence or protein sequence fragment as the Replikin Peak Gene; and
(6) isolating or synthesizing the identified Replikin Peak Gene of the at least one malignant cell, wherein the isolated or synthesized identified Replikin Peak Gene is useful for diagnosis, prevention or treatment of lung cancer.

137. The method of claim 136 wherein the lung malignancy is a non-small cell lung malignancy.

138. The method of claim 137 wherein at least one Replikin sequence within the protein or protein fragment of the identified Replikin Peak Gene is isolated or synthesized for diagnosis, prevention or treatment of lung cancer.

139. The method of claim 138 wherein the at least one Replikin sequence within the protein or protein fragment of the identified Replikin Peak Gene is one of SEQ ID NOS: 1585-1635 of SEQ ID NO: 1636.

140. An immunogenic composition for prevention and treatment of lung cancer, wherein the immunogenic composition comprises at least one isolated or synthesized Replikin sequence within the protein or protein fragment of an identified Replikin Peak Gene.

141. A method of stimulating the immune system, comprising administering in an animal the at least one isolated or synthesized Replikin sequence identified within the Replikin Peak Gene of the lung malignancy for prevention, treatment or diagnosis of lung cancer in an animal.

142. The method of claim 141, wherein the animal is a human.

143. An antibody to at least one isolated or synthesized Replikin sequence within the Replikin Peak Gene sequence of a lung malignancy.

144. A method of identification of a lethal form of lung cancer comprising:
(1) obtaining at least one malignant cell from a plurality of lung tumors;
(2) identifying the Replikin Peak Gene in the at least one malignant cell of each of the plurality of lung tumors;
(3) analyzing the amino acid sequence of a protein or protein fragment of the Replikin Peak Gene in the at least one malignant cell of each of the plurality of lung tumors for the presence and concentration of Replikin sequences;
(4) comparing the concentrations of Replikin sequences in each of the proteins or protein fragments of the Replikin Peak Gene in the at least one malignant cell of each of the plurality of lung tumors; and
(5) identifying the lung tumor having the highest concentration of continuous Replikin sequences in the protein or protein fragment of the Replikin Peak Gene as a lethal form of lung cancer.

145. A method of identification of a more lethal form of lung cancer among at least two lung cancers, comprising:
(1) obtaining at least one malignant cell from each of at least two lung cancers;
(2) identifying the Replikin Peak Gene in the at least one malignant cell of each of the at least two lung cancers;
(3) analyzing the amino acid sequence of a protein or protein fragment of the Replikin Peak Gene in the at least one malignant cell of each of the at least two lung cancers for the presence and concentration of Replikin sequences;
(4) comparing the concentrations of Replikin sequences in each of the proteins or protein fragments of the Replikin Peak Gene in the at least one malignant cell of each of the at least two lung cancers; and
(5) identifying the lung cancer having the highest concentration of continuous Replikin sequences in the protein or protein fragment of the Replikin Peak Gene as the more lethal form of lung cancer.

146. A method of determining an expected increase in lethality or virulence of a virus or organism which method comprises:
(1) obtaining a plurality of isolates of said virus or organism wherein each isolate is isolated within a known time period and wherein at least two of said isolates is isolated about six months to about 5 years later than at least two other of said isolates;
(2) identifying a Replikin Peak Gene in each isolate of said plurality of isolates;
(3) analyzing the identified Replikin Peak Gene of each isolate of the plurality of isolates to determine the Replikin Count of each Replikin Peak Gene of each isolate of the plurality of isolates, or analyzing a protein, protein fragment, or gene area comprising the identified Replikin Peak Gene of each isolate of the plurality of isolates to determine the Replikin Count of the protein, protein fragment, or gene area of the plurality of isolates;
(4) determining a mean Replikin Count within the Replikin Peak Gene or within the protein, protein fragment, or gene area comprising said identified Replikin Peak Gene for each known time period;
(5) comparing the mean Replikin Count within the Replikin Peak Gene or within the protein, protein fragment, or gene area for each known time period one to another;
(6) identifying an increase in the mean Replikin Count between at least two known time periods; and
(7) identifying an expected increase in lethality or virulence of said virus, or organism within about six months to about three years following said identified increase in the mean Replikin Count.

147. The method of claim 146, wherein the known time period is 1 year.

148. The method of claim 147, wherein the increase in mean Replikin Count occurs over one year.

149. The method of claim 147, wherein the increase in mean Replikin Count occurs over three years.

150. The method of claim 146, wherein said increase in mean Replikin Count is significant between at least two known time periods.

151. The method of claim 149, wherein said increase in mean Replikin Count has a significance of p=<0.001.
